(19) United States
(12) Patent Application Publication
(10) Pub. No.: US 2024/0407418 A1
(43) Pub. Date: Dec. 12, 2024

(54) COMPOSITIONS AND METHODS FOR PRODUCING AROMAS

(71) Applicants: Nourish Ingredients Pty Ltd, Mitchell (AU); COMMONWEALTH SCIENTIFIC AND INDUSTRIAL RESEARCH ORGANISATION, Territory (AU)

(72) Inventors: James Robertson PETRIE, Goulburn (AU); Surinder Pal SINGH, Downer (AU); Anna EL TAHCHY, Moncrieff (AU); Pushkar SHRESTHA, Lawson (AU); Rosangela Aparecida DEVILLA, Casey (AU); Hanh NGUYEN, Harrison (AU); Heinrich KROUKAMP, Waitara (AU)

(73) Assignees: Nourish Ingredients Pty Ltd, Mitchell, AC (AU); COMMONWEALTH SCIENTIFIC AND INDUSTRIAL RESEARCH ORGANISATION, Territory (AU)

(21) Appl. No.: 18/703,299

(22) PCT Filed: Oct. 20, 2022
(86) PCT No.: PCT/AU2022/051260

§ 371 Date: Apr. 19, 2024

(30) Foreign Application Priority Data
Oct. 20, 2021 (AU) 2021903367
Dec. 22, 2021 (AU) 2021904213
Mar. 3, 2022 (AU) 2022900516
May 13, 2022 (AU) 2022901282
Sep. 7, 2022 (AU) 2022902576

(51) Int. Cl. A23L 31/15; A23L 27/00; A23L 27/22; A23L 27/26; A23L 27/30

(52) U.S. Cl. CPC A23L 31/15; A23L 27/22; A23L 27/26; A23L 27/33; A23L 27/88

(57) ABSTRACT

The present invention provides compositions comprising microbial biomass, and food products, beverage products or feedstuffs comprising microbial biomass. Described herein are the uses of said compositions and food products, beverage products or feedstuffs for producing food-like aromas and/or flavours when heated, in particular for undergoing Maillard reactions. Also provided are methods for producing food-like aromas and/or flavours.

### RELATED APPLICATIONS

This application claims priority to Australian Provisional Application No. 2021903367 filed on 20 Oct. 2021, Australian Provisional Application No. 2021904213 filed on 22 Dec. 2021, Australian Provisional Application No. 2022900516 filed on 3 Mar. 2022, Australian Provisional Application No. 2022901282 filed on 13 May 2022 and Australian Provisional Application No. 2022902576 filed on 7 Sep. 2022, the entire contents of which are hereby incorporated herein by reference in their entirety.

### TECHNICAL FIELD

The present invention broadly relates to use of microbial biomass (e.g. Mortierella spp. biomass) in a food product, beverage product or feedstuff, to compositions comprising biomass, and to food products, beverage products or feedstuffs comprising the biomass. The present invention further relates to said compositions and food products, beverage products or feedstuffs for producing food-like aromas and/or flavours when heated, in particular for undergoing Maillard reactions. The present invention further relates to methods of producing food-like aromas and/or flavours.

### BACKGROUND OF THE INVENTION

As the global population surges towards a predicted 9 billion people by 2050, the demand for meat and dairy products for human nutrition is expected to continue to increase. However, meat and dairy production worldwide account for 70% of freshwater consumption, 38% of the total arable land use and contribute 19% of the world's greenhouse gas emissions. There is growing interest in finding alternative sources of protein and fat which have less of an environmental footprint. There is also a growing market worldwide for non-animal sources of high-quality protein and fat, for example from plant sources, which are seen as being more sustainable and environmentally friendly. Cultural and religious reasons have also contributed to growing markets for non-animal proteins. However, many current plant-based alternatives for meat and dairy products use fats made from blends of plant oils such as coconut, soy and palm oils which may give inadequate flavour and function. Fats and oils add flavour, lubricity and texture to foods and contribute to the feeling of satiety upon consumption, and therefore food and beverage products incorporating lipids from animal sources are often still preferred by consumers.

The aroma and flavour characteristics of cooked meat are important factors for the eating quality of meat, correlating highly with acceptance and preference by consumers. The aroma and flavour characteristics come from a large number of volatile and non-volatile compounds which are produced during heating of the meat such as by cooking or roasting (see, for example, the reviews by Dashdorj et al. (2015) and Mottram (1998)). These compounds result from several types of chemical reactions, namely Maillard reactions of amino acids or peptides with reducing sugars, lipid oxidation, the interaction between the Maillard reaction products with the lipid-oxidation products, and degradation of other compounds such as some sulphur-containing compounds during cooking or roasting. The reaction products, particularly the volatile ones, are organic and of low molecular weight, including aldehydes, ketones, alcohols, esters, aliphatic hydrocarbons, thiazoles, oxazoles and pyrazines as well as oxygenated heterocyclic compounds such as lactones and alkylfurans. Many of these compounds do not arise during the cooking of meat-substitutes made with plant proteins and fats such as coconut, soy and palm oils, leading to less consumer acceptance of these non-animal products.

There remains a need for alternative, non-animal products which provide meat-like flavour and aroma, for human nutrition.

### SUMMARY OF THE INVENTION

The present invention is predicated on, at least in part, the unexpected determination that certain biomasses can impart a strong and pleasant food-like, and in particular meat-like, aroma and/or flavour to a food. This can be achieved using relatively little amounts of biomass, thus provided an efficient and cost-effective way to enhance the aroma and flavour of food, feedstuff and beverages. In particular, the inventors have demonstrated that various fungal isolates, and in particular Mortierella spp., are effective as flavour and aroma enhancers.

Thus, in one aspect, provided is a composition capable of producing a food-like aroma and/or flavour when heated, the composition comprising:
a) Mortierella spp. biomass comprising phospholipids;
b) one or more sugars, sugar alcohols, sugar acids, or sugar derivatives; and
c) one or more amino acids or derivatives or salts thereof, or a compound comprising an amino group (e.g. thiamine).

Optionally, the composition comprises less than 5% by weight protein, other than protein provided by the Mortierella spp. biomass.

In some examples, the composition comprises at least about 0.25 mg/mL or mg/g dry Mortierella spp. biomass, based on the volume or weight of the composition excluding the Mortierella spp. biomass. In one example, the composition comprises at least about 10 mg/mL or mg/g dry Mortierella spp. biomass, based on the volume or weight of the composition excluding the Mortierella spp. biomass. In other examples, the composition comprises from about 10 mg/mL or mg/g to about 50 mg/mL or mg/g dry Mortierella spp. biomass or an equivalent amount of wet biomass, based on the volume or weight of the composition excluding the Mortierella spp. biomass. In particular embodiments, the food-like aroma and/or flavour is a meaty aroma and/or flavour.

In one embodiment, the Mortierella spp. is Mortierella alpina, Mortierella elongata or Mortierella isabellina.

In particular examples, the phospholipids comprise one or more esterified ω6 fatty acids, e.g. arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA).

In one embodiment, the one or more sugars, sugar alcohols, sugar acids, or sugar derivatives and the one or more amino acids or derivatives or salts thereof are present in the composition in amounts sufficient to produce a food-like aroma and/or flavour when the composition is heated. In some examples, the one or more sugars, sugar alcohols, sugar acids, or sugar derivatives and the one or more amino acids or derivatives or salts thereof are present in the composition in amounts sufficient to produce one or more volatile compounds selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane when the composition is heated. In one example, the one or more sugars, sugar alcohols, sugar acids, or sugar derivatives and the one or more amino acids or derivatives or salts thereof are present in the composition in amounts sufficient to produce one or more volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol when the composition is heated.

In particular embodiments, the one or more sugars, sugar alcohols, sugar acids, or sugar derivatives are present in the composition in an amount of from about 5 mmol to about 100 mmol per kg or per L of composition, based on the volume or weight of the composition excluding the Mortierella spp. biomass. In further embodiments, the one or more sugars, sugar alcohols, sugar acids, or sugar derivatives are present in the composition in an amount of at least about 15 mmol per kg or per L of composition, based on the volume or weight of the composition excluding the Mortierella spp. biomass.

In one embodiment, the one or more amino acids or derivatives or salts thereof are present in the composition in an amount of from about 5 mmol to about 100 mmol, based on the volume or weight of the composition excluding the Mortierella spp. biomass. In one example, the one or more amino acids or derivatives or salts thereof are present in the composition in an amount of at least about 15 mmol per kg or per L of composition, based on the volume or weight of the composition excluding the Mortierella spp. biomass.

In some examples, the one or more sugars, sugar alcohols, sugar acids or sugar derivatives comprise glucose and/or ribose. In particular examples, the one or more sugars, sugar alcohols, sugar acids or sugar derivatives comprise ribose and glucose.

In further examples, the one or more amino acids or derivatives or salts thereof comprise cysteine and/or cystine. The one or more amino acids may also, or alternatively, comprise glutamic acid or a salt thereof. In some examples, the composition comprises glutamic acid or a salt thereof and a further amino acid, derivative or salt thereof.

The compositions may also comprise any one or more of, or any combination of, a source of iron, a yeast extract, thiamine, herbs and/or spices and an aqueous component. In particular embodiments, the composition does not comprise a yeast extract.

In one embodiment, the composition comprises:
a) Mortierella spp. biomass comprising phospholipids;
b) glucose and/or ribose;
b) cysteine and/or cystine;
d) yeast extract;
e) glutamic acid or a salt thereof;
f) thiamine; and
g) an aqueous component.

In an embodiment, the composition produces a meaty aroma and/or flavour when heated.

In one embodiment, the composition is in the form of a food product, beverage product or feedstuff. Accordingly, the food product, beverage product or feedstuff produces a meaty aroma and/or flavour when heated.

In another embodiment the composition may be mixed with, or added to, a food product, beverage product or feedstuff, for example wherein the composition is in the form of a powder, particulate or granulated mix. The composition may be mixed with, or be added to, the food product, beverage product or feedstuff prior to heating, after heating the composition, and/or after heating the food product, beverage product or feedstuff. Upon heating the composition, or the admixed composition and food product, beverage product or feedstuff, a meaty aroma and/or flavour may be produced.

In another aspect, provided is a food product, beverage product or feedstuff comprising Mortierella spp. biomass comprising phospholipids, or a composition or the invention, optionally wherein the food product, beverage product or feedstuff comprises less than 5% dry Mortierella spp. biomass by weight, or an equivalent amount of wet biomass. In some examples, the food product, beverage product or feedstuff has a meaty aroma and/or flavour. In particular examples, the food product, beverage product or feedstuff produces a meaty aroma and/or flavour when heated. In some embodiments, the Mortierella spp. is Mortierella alpina, Mortierella elongata or Mortierella exigua.

In some embodiments of the food product, beverage product or feedstuff the phospholipids comprise one or more esterified ω6 fatty acids, e.g. arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA).

In some examples, the food product, beverage product or feedstuff is a meat or meat-like product, e.g. a burger, sausage, hot dog, mince or ground meat, steak, streak, strip, fillet, roast, breast, thigh, wing, meatloaf, finger, nugget, cutlet, cube, bacon, soup, gravy, sliced meat, meatballs, fish, fried fish or seafood or imitation thereof. In one embodiment, the food product, beverage product or feedstuff is free from any animal or animal-derived ingredients. In another embodiment, the food product, beverage product or feedstuff comprises an animal or animal-derived ingredient, optionally wherein the animal or animal-derived ingredient is meat.

In particular examples, the food product, beverage product or feedstuff comprises one or more sugars, sugar alcohols, sugar acids, or sugar derivatives; and one or more amino acids or derivatives or salts thereof, or a compound comprising an amino group (e.g. thiamine). In one example, the one or more sugars, sugar alcohols, sugar acids, or sugar derivatives and the one or more amino acids or derivatives or salts thereof are present in the food product, beverage product or feedstuff in amounts sufficient to produce a food-like aroma and/or flavour when the food product, beverage product or feedstuff is heated. In some embodiments, the one or more sugars, sugar alcohols, sugar acids, or sugar derivatives and the one or more amino acids or derivatives or salts thereof are present in the food product, beverage product or feedstuff in amounts sufficient to produce one or more volatile compounds selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane when the food product, beverage product or feedstuff is heated. In further embodiments, the one or more sugars, sugar alcohols, sugar acids, or sugar derivatives and the one or more amino acids or derivatives or salts thereof are present in the food product, beverage product or feedstuff in amounts sufficient to produce one or more volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol when the food product, beverage product or feedstuff is heated.

In some examples, the food product, beverage product or feedstuff comprises an extracted lipid from Mortierella spp. comprising phospholipids.

In one embodiment, the food product, beverage product or feedstuff may comprise about 2.5% or less dry Mortierella spp. biomass by weight, or an equivalent amount of wet biomass.

Also provided is a method of producing a food product, beverage product or feedstuff comprising combining Mortierella spp. biomass comprising phospholipids, or a composition of the present invention, with one or more additional consumable ingredients.

Also provided is a method for producing a food product, beverage product or feedstuff comprising combining a) Mortierella spp. biomass comprising phospholipids; one or more sugars, sugar alcohols, sugar acids, or sugar derivatives; and one or more amino acids or derivatives or salts thereof, or thiamine; or b) a composition according to the invention; with one or more additional consumable ingredients, wherein the food product, beverage product or feedstuff comprises about 2.5% or less dry Mortierella spp. biomass by weight, or an equivalent amount of wet biomass.

Also provided is a method for producing a composition according to the invention, comprising combining a) the Mortierella spp. biomass comprising phospholipids, b) the one or more sugars, sugar alcohols, sugar acids, or sugar derivatives, and c) the one or more amino acids or derivatives or salts thereof, or thiamine, with one or more additional consumable ingredients, wherein the composition comprises about 2.5% or less dry Mortierella spp. biomass by weight or an equivalent amount of wet biomass.

Also provided is a method for producing food-like aromas and/or flavours, comprising heating a composition, or a food product, beverage product or feedstuff of the present invention. Also provided is a method for producing food-like aromas and/or flavours, comprising mixing or adding a composition according to the present invention with a food product, beverage product or feedstuff, and heating. Also provided is a method for producing food-like aromas and/or flavours, comprising heating a composition according to present invention and mixing or adding the heated composition with a food product, beverage product or feedstuff.

In another aspect, provided is a method of imparting a food-like aroma and/or flavour to a food product, beverage product or feedstuff comprising contacting the food product, beverage product or feedstuff with Mortierella spp. biomass comprising phospholipids, or a composition of the invention, and heating the food product, beverage product or feedstuff and Mortierella spp. biomass or composition.

In a further aspect, provided is a method of increasing food-like aromas and/or flavours associated with a food product, beverage product or feedstuff, comprising contacting the food product, beverage product or feedstuff with Mortierella spp. biomass comprising phospholipids, or a composition of the invention, and heating the food product, beverage product or feedstuff and Mortierella spp. biomass comprising phospholipids or composition.

Also provided is a method of increasing food-like aromas and/or flavours associated with a food product, beverage product or feedstuff, comprising: a) heating a composition of the invention; and b) contacting a food product, beverage product or feedstuff with the composition obtained in step a).

In some examples of the above methods, the food product, beverage product or feedstuff is a meat or meat-like product. In further examples, the Mortierella spp. biomass is present in the food product, beverage product or feedstuff or is contacted with the food product, beverage product or feedstuff in an amount of less than 5% dry Mortierella spp. biomass by weight, or an equivalent amount of wet biomass.

In one embodiment, the food-like aroma and/or flavour is a meaty aroma and/or flavour. In particular embodiments, the composition, food product, beverage product or feedstuff is heated to at least about 130° C. and/or for at least about 1 hour.

In a further aspect, provided is a use of Mortierella spp. biomass comprising phospholipids in a composition, food product, beverage product or feedstuff, wherein the composition, food product, beverage product or feedstuff comprises less than 5% dry Mortierella spp. biomass by weight, or an equivalent amount of wet biomass. In some examples, the use is for imparting a food-like (e.g. a meaty or meat-like) aroma and/or flavour to said composition, food product, beverage product or feedstuff.

In some examples, the Mortierella spp. in the composition, food product, beverage product or feedstuff is Mortierella alpina, Mortierella elongata or Mortierella exigua. In particular embodiments, the phospholipids comprise one or more esterified ω6 fatty acids (e.g. arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA)).

In some examples of the uses of the present invention, the food product, beverage product or feedstuff is a meat or meat-like product, e.g. a burger, sausage, hot dog, mince or ground meat, steak, streak, strip, fillet, roast, breast, thigh, wing, meatloaf, finger, nugget, cutlet, cube, bacon, soup, gravy, sliced meat, meatballs, fish, fried fish or seafood or imitation thereof. In one example, the food product, beverage product or feedstuff is free from any animal or animal-derived ingredients. In another example, the food product, beverage product or feedstuff comprises an animal or animal-derived ingredients, optionally wherein the an animal or animal-derived ingredient is meat.

In particular embodiments of the uses of the invention, the food product, beverage product or feedstuff comprises: one or more sugars, sugar alcohols, sugar acids, or sugar derivatives; and one or more amino acids or derivatives or salts thereof, or a compound comprising an amino group (e.g. thiamine). In some examples, the one or more sugars, sugar alcohols, sugar acids, or sugar derivatives and the one or more amino acids or derivatives or salts thereof (or the compound comprising an amino group) are present in the composition, food product, beverage product or feedstuff in amounts sufficient to produce a food-like aroma and/or flavour when the composition, food product, beverage product or feedstuff is heated. In particular examples, the one or more sugars, sugar alcohols, sugar acids, or sugar derivatives and the one or more amino acids or derivatives or salts thereof are present in the food product, beverage product or feedstuff in amounts sufficient to produce one or more volatile compounds selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane when the composition, food product, beverage product or feedstuff is heated. In one example, the one or more sugars, sugar alcohols, sugar acids, or sugar derivatives and the one or more amino acids or derivatives or salts thereof are present in the composition, food product, beverage product or feedstuff in amounts sufficient to produce one or more volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol when the composition, food product, beverage product or feedstuff is heated.

In some examples of the uses of the invention, the composition, food product, beverage product or feedstuff further comprises an extracted lipid from Mortierella spp. comprising phospholipids.

In a particular embodiment of the uses of the invention, the composition, food product, beverage product or feedstuff comprises about 2.5% or less dry Mortierella spp. biomass by weight, or an equivalent amount of wet biomass.

Also provided is an isolated strain of Mortierella sp. selected from:
i) yNI0125 deposited under V21/019953 on 12 Oct. 2021 at the National Measurement Institute Australia;
ii) yNI0126 deposited under V21/019951 on 12 Oct. 2021 at the National Measurement Institute Australia;
iii) yNI0127 deposited under V21/019952 on 12 Oct. 2021 at the National Measurement Institute Australia; and
iv) yNI0132 deposited under V21/019954 on 12 Oct. 2021 at the National Measurement Institute Australia.

Also provided is an isolated strain of Mucor hiemalis, yNI0121, deposited under Deposit Accession number V22/001757 on 4 Feb. 2021 at the National Measurement Institute Australia.

### BRIEF DESCRIPTION OF THE DRAWINGS

Exemplary embodiments of the present disclosure are described herein, by way of non-limiting example only, with reference to the following drawings.

FIG. 1 shows polyunsaturated fatty acid biosynthesis pathways.

FIG. 2 shows a schematic of the pathways for phospholipid synthesis.

FIG. 3 shows the profile of volatile compounds released by heating extracted lipids with a mixture of ribose and cysteine as in Example 5, Experiment 3, as measured by gas chromatography-mass spectrometry (GC-MS). Levels of each of the identified compounds are shown as the area percentage (%) of total identified compounds (left column, YL ARA PL %; right column, YL PL %).

FIG. 4 shows the profile of volatile compounds released by Maillard reactions of mixtures comprising 2.5 or 5.0 mg of 18:0/18:1-phosphatidylcholine (PC) or ARA-PC as described in Example 5, Experiment 5, as measured by gas chromatography-mass spectrometry (GC-MS). For each compound, from left to right: Con-2.5; Con-5.0; ARA PC 2.5; ARA PC 5.0.

FIG. 5 shows the results of a sensory evaluation of meatiness of food samples comprising textured vegetable protein and varying amounts of Mortierella alpina biomass. For each amount (percentage value), columns from left to right: aroma; taste; total.

FIG. 6 shows the results of a sensory evaluation of pleasantness of food samples comprising textured vegetable protein and varying amounts of Mortierella alpina biomass. For each amount (percentage value), columns from left to right: aroma; taste; total.

FIG. 7 shows the combined meatiness and pleasantness results of a sensory evaluation of food samples comprising textured vegetable protein and varying amounts of Mortierella alpina biomass. Top portion of each column represents meatiness total, and bottom portion of each column represents pleasantness total.

FIG. 8 shows the meatiness results of a sensory evaluation of samples comprising a Maillard reaction matrix at varying concentrations and Mortierella alpina biomass. For each concentration, columns from left to right: sniff; taste; total.

FIG. 9 shows the pleasantness results of a sensory evaluation of samples comprising a Maillard reaction matrix at varying concentrations and Mortierella alpina biomass. For each concentration, columns from left to right: sniff; taste; total.

FIG. 10 shows the combined meatiness and pleasantness results of a sensory evaluation of samples comprising a Maillard reaction matrix at varying concentrations and Mortierella alpina biomass.

FIG. 11 shows the combined meatiness and pleasantness results of a sensory evaluation of samples comprising a Maillard reaction with Mortierella alpina biomass or Mortierella isabellina biomass. For each sample, columns from left to right: pleasantness; meatiness; overall.

FIG. 12 shows the results of a sensory evaluation of samples comprising a Maillard reaction with Mortierella alpina biomass and varying amounts of cystine. In A and B, for each sample, columns from left to right: aroma; taste; total. In C, for each sample, columns from left to right: total pleasantness; total meatiness; total score.

FIG. 13 shows the results of a sensory evaluation of food samples comprising a Maillard reaction with Mortierella alpina biomass and varying amounts of cystine. In A and B, for each sample, columns from left to right: aroma; taste; total. In C, for each sample, columns from left to right: total pleasantness; total meatiness; total score.

FIG. 14 shows the results of a sensory evaluation of samples comprising a Maillard reaction with Mortierella alpina biomass and varying amounts of dextrose. In A and B, for each sample, columns from left to right: aroma; taste; total. In C, for each sample, columns from left to right: total pleasantness; total meatiness; total score.

FIG. 15 shows the results of a sensory evaluation of food samples comprising a Maillard reaction with Mortierella alpina biomass and varying amounts of dextrose. In A and B, for each sample, columns from left to right: aroma; taste; total. In C, for each sample, columns from left to right: total pleasantness; total meatiness; total score.

FIG. 16 shows the results of a sensory evaluation of food samples comprising a Maillard reaction with Mortierella alpina biomass and varying combinations of cysteine, cystine, ribose and dextrose. In A and B, for each sample, columns from left to right: aroma; taste; total. In C, for each sample, columns from left to right: total pleasantness; total meatiness; total score.

FIG. 17 illustrates relative amounts of 57 volatile compounds identified by GC-MS in 8 samples (S1 to S8, as defined in Table 37) as described in Example 17.

FIG. 18 shows the results of a sensory evaluation of compositions comprising a Maillard reaction with Mortierella biomass as described in Example 21.

FIG. 19 shows a phylogenetic analysis of Mortierella species as described in Example 22.

### DETAILED DESCRIPTION OF THE INVENTION

### Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the terms “a” and “an” refer to one or to more than one (i.e. to at least one) of the grammatical object of the article. By way of example, “an element” means one element or more than one element.

In the context of this specification, the term “about” is understood to refer to a range of numbers that a person of skill in the art would consider equivalent to the recited value in the context of achieving the same function or result.

In the context of this specification, reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein, the term “and/or” means “and” or “or” or both.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

As used herein, a “lipid” is any of a class of organic compounds that are or comprise fatty acids, which may be esterified or non-esterified, or their derivatives and are insoluble in water but soluble in organic solvents, for example in chloroform. As used herein, the term “extracted lipid” refers to a lipid composition which has been extracted from a microbial cell. The extracted lipid can be a relatively crude composition obtained by, for example, lysing the cells and separating the lipid, or a more purified composition where most, if not all, of one or more or each of the water, nucleic acids, proteins and carbohydrates derived from the cells have been removed. Examples of purification methods are described below. An extracted lipid may comprise, for example, at least about 10%, at least about 20%, at least about 30%, at least about 40%, at least about 50%, at least about 60%, at least about 70%, at least about 80%, at least about 90%, or at least about 95% (w/w) lipid by weight of the composition. In particular embodiments, an extracted lipid comprises between about 10% and 95% lipid by weight, for example between about 10% and about 50%, or about 50% and 95%, lipid by weight. The lipid may be solid or liquid at room temperature (25° C.), or a mixture of the two; when liquid it is considered to be an oil, when solid it is considered to be a fat. In an embodiment, extracted lipid has not been blended with another lipid produced from another source, for example, animal lipid. Alternatively, the extracted lipid may be blended with a different lipid. An extracted lipid may contain all lipids initially present in a microbial cell, or may contain only a fraction of lipids initially present in a microbial cell; for example, an extracted lipid may have been processed to remove some or all of a particular type of lipid, for example to remove some or all neutral lipid (such as triacylglycerols (triglycerides, ‘TAG’) and to retain polar lipids (such as phospholipids).

As used herein, the term “polar lipid” refers to amphipathic lipid molecules having a hydrophilic head and a hydrophobic tail, including phospholipids (e.g. phosphatidylcholine, phosphatidylethanolamine, phosphatidylinositol, phosphatidylserine, phosphatidylglycerol, diphosphatidylglycerols), cephalins, sphingolipids (sphingomyelins and glycosphingolipids), phosphatidic acid, cardiolipin and glycoglycerolipids. Phospholipids are composed of the following major structural units: fatty acids, glycerol, phosphoric acid, and amino alcohols. They are generally considered to be structural lipids, playing important roles in the structure of the membranes of plants, microorganisms and animals. Because of their chemical structure, polar lipids exhibit a bipolar nature, exhibiting solubility or partial solubility in both polar and non-polar solvents.

The term “phospholipid”, as used herein, refers to an amphipathic molecule, having a hydrophilic head and a hydrophobic tail, that has a glycerol backbone esterified to a phosphate “head” group and two fatty acids which provide the hydrophobic tail. The phosphate group can be modified with simple organic molecules such as choline, ethanolamine or serine. Due to their charged headgroup at neutral pH, phospholipids are polar lipids, having some solubility in solvents such as ethanol in addition to solvents such as chloroform. Phospholipids are a key component of all cell membranes. They can form lipid bilayers because of their amphiphilic characteristic. Well known phospholipids include phosphatidylcholine (PC), phosphatidylethanolamine (PE), phosphatidylinositol (PI), phosphatidylserine (PS), phosphatidic acid (PA), phosphatidylglycerol (PG), diphosphatidylglycerols and cardiolipin.

As used herein, the term “non-polar lipid” refers to fatty acids and derivatives thereof which are soluble in organic solvents but insoluble in water. The fatty acids may be free fatty acids and/or in an esterified form. Examples of esterified forms include, but are not limited to, triacylglycerol (TAG), diacylyglycerol (DAG), monoacylglycerol (MAG). Non-polar lipids also include sterols, sterol esters and wax esters. Non-polar lipids are also known as “neutral lipids” or in some contexts referred to as “oils”. Non-polar lipid may be a liquid at room temperature, or a solid, depending on the degree of unsaturation of the fatty acids in the non-polar lipid. Typically, the more saturated the fatty acid content, the higher the melting temperature of the lipid.

As used herein, the term “fatty acid” refers to a carboxylic acid consisting of an aliphatic hydrocarbon chain and a terminal carboxyl group. The hydrocarbon chain can be either saturated or unsaturated. Unsaturated fatty acids include monounsaturated fatty acids having only one carbon-carbon double bond and polyunsaturated fatty acids (PUFA) having at least two carbon-carbon double bonds, typically between 2 and 6 carbon-carbon double bonds. A fatty acid may be a free fatty acid (FFA) or esterified to a glycerol or glycerol-phosphate molecule (for example as a phospholipid), CoA molecule or other headgroup as known in the art.

As used herein, the term “total fatty acid (TFA) content” or variations thereof refers to the total amount of fatty acids in, for example, an extracted lipid or microorganism cell, on a weight basis. The TFA may be expressed as a percentage of the weight of the cell or other fraction, e.g., as a percentage of the polar lipid. Unless otherwise specified, the weight with regard to the cell weight is the dry cell weight (DCW). In an embodiment, TFA content is measured by conversion of the fatty acids to fatty acid methyl esters (FAME) or fatty acid butyl esters (FABE) and measurement of the amount of FAME or FABE by GC, using addition of a known amount of a distinctive fatty acid standard as a quantitation standard in the GC. Typically, the amount and fatty acid composition of lipids or compositions comprising only fatty acids in the range of C10-C24 are determined by conversion to FAME, whereas lipids or compositions comprising fatty acids in the range of C4-C10 are determined by conversion to FABE. TFA therefore represents the weight of just the fatty acids, not the weight of the fatty acids and their linked moieties in the lipid or composition.

“Saturated fatty acids” do not contain any double bonds or other functional groups along the acyl chain. The term “saturated” refers to hydrogen, in that all carbons (apart from the carboxylic acid [—COOH]group) contain as many hydrogens as possible.

“Unsaturated fatty acids” are of similar form to saturated fatty acids, except that one or more alkene functional groups exist along the chain, with each alkene substituting a singly-bonded “—CH2-CH2-” part of the chain with a doubly-bonded “—CH═CH—” portion (that is, a carbon double bonded to another carbon). The two next carbon atoms in the chain that are bound to either side of the double bond can occur in a cis or trans configuration, preferably in the cis configuration.

As used herein, the term “monounsaturated fatty acid” refers to a fatty acid which comprises at least 12 carbon atoms in its carbon chain and only one alkene group (carbon-carbon double bond) in the chain. Monounsaturated fatty acids include C12:1Δ9, C14:1Δ9, C16:1Δ9 (palmitoleic acid), C18:1Δ9 (oleic acid) and C18:1Δ11 (vaccenic acid).

As used herein, the terms “polyunsaturated fatty acid” or “PUFA” refer to a fatty acid which comprises typically at least 12 carbon atoms in its carbon chain and at least two alkene groups (carbon-carbon double bonds). Ordinarily, the number of carbon atoms in the carbon chain of the fatty acids refers to an unbranched carbon chain. Unless stated otherwise, if the carbon chain is branched, the number of carbon atoms excludes those in side groups. In particular, ‘ω6 fatty acids’, ‘omega 6 fatty acids’ or ‘n-6 fatty acids’ (the three terms being used interchangeably herein) have a final desaturation (carbon-carbon double bond) in the sixth carbon-carbon bond from the methyl end of the fatty acid. Examples of ω6 fatty acid include, but are not limited to, arachidonic acid (ARA, C20:4Δ5,8,11,14; ω6), dihomo-gammalinolenic acid (DGLA, C20:3Δ8,11,14; ω6), eicosadienoic acid (EDA, C20:2Δ11,14; ω6), docosatetraenoic acid (DTA, C22:4Δ7,10,13,16; ω6), docosapentaenoic acid-ω6 (DPA-ω6, C22:5Δ4,7,10,13,16; ω6), γ-linolenic acid (GLA, C18:3Δ6,9,12; ω6) and linoleic acid (LA, C18:2Δ9,12; ω6). ω3/omega 3/n-3 fatty acids have a final desaturation (carbon-carbon double bond) in the third carbon-carbon bond from the methyl end of the fatty acid. ω3 fatty acids include, for example, α-linolenic acid (ALA, C18:3Δ9,12,15; ω3), hexadecatrienoic acid (C16:3ω3), eicosapentaenoic acid (EPA, C20:5Δ5,8,11,14,17; ω3), docosapentaenoic acid (DPA, C22:5Δ7,10,13,16,19, ω3), docosahexaenoic acid (DHA, 22:6Δ4,7,10,13,16,19, ω3), eicosatetraenoic acid (ETA, C20:4Δ8,11,14,17; ω3) and eicosatrienoic acid (ETrA, C20:3Δ11,14,17; ω3).

As used herein, “C12:0” refers to lauric acid. As used herein, “C14:0” refers to myristic acid. As used herein, “C15:0” refers to n-pentadecanoic acid. As used herein, “C16:0” refers to palmitic acid. As used herein, “C17:1” refers to heptadecenoic acid. As used herein, “C16:1Δ9” refers to palmitoleic acid, or -hexadec-9-enoic acid. As used herein, “C18:0” refers to stearic acid. As used herein, “C18:1Δ9”, sometimes referred to in shorthand as “C18:1”, refers to oleic acid. As used herein, “C18:1Δ11” refers to vaccenic acid. As used herein, “C20:0” refers to eicosanoic acid. As used herein, “C20:1” refers to eicosenoic acid. As used herein, “C22:0” refers to docosanoic acid. As used herein, “C22:1” refers to erucic acid. As used herein, “C24:0” refers to tetracosanoic acid.

“Triacylglyceride”, “triglyceride” or “TAG” is a glyceride in which the glycerol is esterified with three fatty acids which may be the same (e.g. as in tri-olein) or, more commonly, different. All three of the fatty acids may be different, or two of the fatty acids may be the same and the third is different. In the Kennedy pathway of TAG synthesis, DAG is formed as described below, and then a third acyl group is esterified to the glycerol backbone by the activity of a diglyceride acyltransferase (DGAT). TAG is a form of non-polar lipid. The three acyl groups esterified in a TAG molecule are referred to as being esterified in the sn-1, sn-2 and sn-3 positions, referring to the positions in the glycerol backbone of the TAG molecule. The sn-1 and sn-3 positions are chemically identical, but biochemically the acyl groups esterified in the sn-1 and sn-3 positions are distinct in that separate and distinct acyltransferase enzymes catalyse the esterifications.

“Diacylglyceride”, “diglyceride” or “DAG” is glyceride in which the glycerol is esterified with two fatty acids which may be the same or, preferably, different. As used herein, DAG comprises a hydroxyl group at a sn-1,3 or sn-2 position, and therefore DAG does not include phosphorylated glycerolipid molecules such as PA or PC. In the Kennedy pathway of DAG synthesis, the precursor sn-glycerol-3-phosphate (G3P) is esterified to two acyl groups, each coming from a fatty acid coenzyme A ester, in a first reaction catalysed by a glycerol-3-phosphate acyltransferase (GPAT) at position sn-1 to form LysoPA, followed by a second acylation at position sn-2 catalysed by a lysophosphatidic acid acyltransferase (LPAAT) to form phosphatidic acid (PA). This intermediate is then de-phosphorylated by PAP to form DAG.

As used herein, an “oil” is a composition comprising predominantly lipid and which is a liquid at room temperature.

As used herein, an “oleaginous” cell or microorganism is one that is capable of storing at least 20% lipid, such as for example 20% to 70%, of its cell mass on a dry weight basis. The lipid content may depend on culture conditions, as is known in the art. It is understood that so long as the microorganism is capable of synthesizing and accumulating at least 20% lipid on a dry cell weight basis under at least one set of culture conditions it is regarded as an oleaginous cell, even if under different conditions it accumulates less than 20% lipid.

As used herein, a “heterotrophic” cell is one that is capable of utilizing organic materials as a carbon source for metabolism and growth. Heterotrophic organisms may also be able to grow autotrophically under suitable conditions.

As used herein, “fermentation” refers to a metabolic process that produces chemical changes in organic substrates through the action of enzymes in the cells, under conditions either lacking oxygen or having reduced levels of oxygen relative to air.

As used herein, a “meat-like flavour and/or aroma”, or a “meat-associated flavour and/or aroma” or a “meaty flavour and/or aroma” refers to flavours and/or aromas that are the same as or are similar to one or more meats, such as beef, steak, chicken, for example roasted chicken or chicken skin, pork, lamb, duck, venison, chicken or other meat soup, meat broth or liver. Such aromas are typically detected by human volunteers, for example by a qualified sensory panel. Meat-like or meat-associated flavours and/or aromas can also be detected by assessing volatile compounds arising after the cooking of the composition or food. Volatile compounds indicative of meat-like or meat-associated aromas and flavours are known in the art and include those exemplified herein, including but not limited to 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane.

### Compositions, Food and Beverage Products and Feedstuffs

The present invention relates to the use of microbial biomass comprising phospholipids, for example Mortierella spp. biomass comprising phospholipids, in a composition, food product, beverage product or feedstuff. The present invention further relates to a composition, food product, beverage product or feedstuff comprising the microbial biomass, such as Mortierella spp. biomass, comprising phospholipids. The present invention also relates to a composition that is capable of producing a food-like aroma when heated, wherein the composition comprises a microbial biomass, one or more sugars, sugar alcohols, sugar acids, or sugar derivatives; and one or more amino acids or derivatives thereof.

As would be appreciated, compositions of the invention may include food products, beverage products or feedstuffs. Thus, the term “composition” encompasses non-food compositions and compositions that are food products, beverage products or feedstuffs. In particular embodiments, the compositions are concentrated liquid or solid “flavouring compositions”, which can be added to other ingredients to produce a food product, beverage product or feedstuff with a desired flavour. In other embodiments, the term composition is used interchangeably with food product, beverage product or feedstuff.

In particular embodiments, the invention relates to a composition that is capable of producing a food-like aroma and/or flavour when heated, the composition comprising:
a) biomass, e.g. Mortierella spp. biomass, comprising phospholipids;
b) one or more sugars, sugar alcohols, sugar acids, or sugar derivatives; and
c) one or more amino acids or derivatives thereof, or a compound comprising an amino group (e.g. thiamine).

The compositions, food products, beverage products or feedstuffs of the present disclosure are suitable for human or animal consumption, typically at least human consumption.

The present invention relates to compositions as well as to food products, beverage products or feedstuffs, including food products, beverage products or feedstuffs comprising compositions of the present invention. The compositions of the present invention may be incorporated into food products, beverage products or feedstuffs to provide a desired food-like aroma. The food products, beverage products or feedstuff are suitable for human or animal consumption, typically at least human consumption. A food product, beverage product or feedstuff is a preparation for human or animal consumption which when taken into the body (a) serves to nourish or build up tissues or supply energy; and/or (b) maintains, restores or supports adequate nutritional status or metabolic function. Whilst a “food product” may be generally considered to include solid, semi-solid, or savoury liquid products, a “beverage product” may be generally considered to include liquid drinkable products, and “feedstuff” may be considered to generally include animal, such as livestock food. It will be appreciated that there is overlap in the meaning of the terms “food product”, “beverage product” and “feedstock” and the terms may, in some circumstances, be used interchangeably.

In particular embodiments, the food or beverage product or feedstuff is a meat or fish substitute product, i.e. a food or beverage product intended to imitate a food or beverage product which typically would contain meat or fish, for example for use in a vegetarian or vegan diet. In some alternative embodiments, the food or beverage product or feedstuff may be a product which includes meat or fish, and a composition of the present invention may be included to provide additional or alternative flavours or aromas to the product. For example, the food or beverage product or feedstuff product may comprise meat obtained from an animal and/or cultivated or cultured meat (i.e. meat that has been produced by cultivating animal cells in vitro). In some examples, the food or beverage product or feedstuff product is a blend of meat (e.g. meat obtained from an animal and/or cultivated or cultured meat) and non-animal protein (e.g. plant or mcrobial protein). Suitable food or beverage products or feedstuffs include but are not limited to meat or fish substitutes or meat or fish-based products, soup bases, stew bases, snack foods, bouillon powders, bouillon cubes, flavour packets, seasoning or frozen food products. For example, in some particular embodiments, the food or beverage product may be, or may be intended to imitate, for example, burgers, sausages, hot dogs, mince or ground meat, steaks, streaks, strips, fillets, roasts, breasts, thighs, wings, meatloaf, fingers, nuggets, cutlets, cubes, bacon, soup, gravy, sliced meat, meatballs, fish, fried fish or seafood.

In particularly preferred embodiments, the food product is a meat or meat-like product. A “meat-like product” is readily understood as referring to a product which resembles a meat product but which may not necessarily contain any meat, for example meat-alternative burgers, sausages, ground mince, meatballs, strips or other products. In some examples, the meat-like product comprises no animal products. In other examples, the meat or meat-like product comprises cultivated meat (i.e. meat produced by cultivating animal cells in vitro).

Ingredients and methods for producing food, feedstuffs and beverages, including meat substitutes, are well known in the art (see e.g. WO2008124370, WO2013010042, WO2015153666 and WO2017070303), the entire contents of which are incorporated by reference in their entirety) and can be employed with the micobial biomass (e.g. Mortierella spp biomass) or compositions of the present invention to produce a food, feedstuff or beverage.

Biomass and/or extracted lipids comprising phospholipids disclosed herein and/or compositions of the present invention may be used to modulate the flavour and/or aroma of a food or beverage product or feedstuff, by enhancing or altering the flavour and/or aroma of the food or beverage product or feedstuff. For example, biomass and any extracted lipids comprising phospholipids disclosed herein and/or compositions of the present disclosure may enhance or alter the flavour and/or aroma of a food or beverage product or feedstuff, such as by enhancing meaty, fishy or vegetable flavour and/or aromas or by introducing such flavour and/or aromas to food or beverage products or feedstuffs. In some embodiments, the biomass and any extracted lipids comprising phospholipids disclosed herein, or the compositions, food or beverage products or feedstuffs of the present disclosure are intended to be added as an ingredient to a separate product to enhance or modulate the taste and/or aroma of the separate product to which it is added, for example by enhancing the meatiness or fishiness of the separate product or by altering the aroma or flavour of a product. Biomass and any extracted lipids comprising phospholipids disclosed herein, or compositions, food or beverage products or feedstuffs of the present disclosure can be used to modulate, by enhancing or altering, the taste and/or aroma profile of, for example, meat replicas, meat substitutes, tofu, seitan, mock duck or a gluten based vegetable product, textured vegetable protein such as textured soy protein, pork, fish, lamb, or poultry products such as chicken or turkey products, and can be applied to the other food product before or during cooking. In some embodiments, using the biomass and any extracted lipids comprising phospholipids disclosed herein, or compositions, food or beverage products or feedstuffs described herein can provide a particular meaty taste and smell, for example, the taste and smell of beef, to a non-meat product or to a poultry product.

In particularly preferred embodiments, compositions of the present disclosure comprise less than 20% protein derived from a source other than the Mortierella spp. (or other microbial) biomass, optionally less than 15%, less than 10%, less than 5% or no protein other than protein provided by the Mortierella spp. (or other microorganism) biomass. In contrast, food products, beverage products and feedstuffs of the present disclosure may optionally comprise added protein in an amount of greater than 5%, 10%, 15%, 20%, 25%, 30%, 35% or 40%.

As exemplified herein, the present inventors have found that certain microbial biomasses, including Mortierella spp. biomass comprising phospholipids, in the context of compositions, food products, beverage products or feedstuffs of the invention, produces food-like aromas when heated, especially meaty aromas, thought to be due to occurrence of a Maillard reaction. The Mortierella spp. biomass comprising phospholipids finds use in imparting aroma and/or flavours to, or enhancing aromas and/or flavours of food and beverage products and feedstuffs, especially meaty and fishy aromas and/or flavours, for example in meat- or fish-substitute food products which may be free of animal-derived meat, fish or other animal products. It has been found that the inclusion of Mortierella spp. biomass comprising phospholipids in such compositions or food products, beverage products or feedstuffs is especially effective in producing the food-like aromas such as meaty aromas. The biomass typically comprises whole cells of the microorganism and may be a crude mixture of cells and cell-derived compounds such as lipids, proteins, carbohydrates such as sugars and glucans, and nucleic acids. The cells may be alive, inactivated or dead, or a mixture thereof.

The precise amount of microorganism (e.g. Mortierella spp.or Yarrowia spp.) and/or extracted lipid, preferably phospholipid, in a composition of the present disclosure may be varied depending on, for example, the identity of the microorganism, the form and moisture content of the biomass of the microorganism, the total lipid or phospholipid content and profile contained in the microorganism, the intensity of the desired flavour and/or aroma and the intended use of the composition. In some examples, the compositions (e.g. concentrated flavouring compositions) comprise at least or about 1%, 2%, 3%, 4%, 5%, 5%, 7%, 8%, 9%, 10%, 11%, 12%, 13%, 14% 15%, 16%, 17%, 18%, 19%, 20% or 25% dry biomass, or an equivalent amount of wet biomass. In some examples, the compositions of the present invention comprise between 1% and 50%, between 1% and 40%, between 1% and 30%, between 1% and 20%, between 5% and 30%, between 5% and 20%, or between 5% and 15% dry biomass by weight, or an equivalent amount of wet biomass. In preferred embodiments, food products, beverage products or feedstuffs of the present invention comprise less than 5% dry biomass (e.g. less than 5% dry Mortierella spp. by weight), or an equivalent amount of wet biomass. As demonstrated herein, unpleasant taste profiles can arise when the amount of biomass in the food products, beverage products or feedstuffs is above a certain level. Thus, in some examples, the food products, beverage products or feedstuffs of the present invention comprise less than or about 4.5%, 4%, 3.5%, 3%, 2.5%, 2%, 1.5%, 1%, 0.9%, 0.8%, 0.7%, 0.6%, 0.5%, 0.4%, 0.3%, 0.2% or 0.1% dry biomass (e.g. dry Mortierella spp. biomass) by weight, or an equivalent amount of wet biomass.

In some embodiments, the compositions (e.g. concentrated flavouring compositions) of the present disclosure comprise per gram of dry compositions or slurries, or per mL in the case of liquid compositions, at least about 1 mg wet microorganism (e.g. Mortierella spp.) biomass, in particular at least about 5 mg, preferably at least about 10 mg, more preferably at least about 15 mg wet biomass, for example at least about 20 mg, at least about 25 mg, at least about 30 mg, or at least about 40 mg wet biomass. In embodiments wherein dry biomass is used, the compositions of the present disclosure comprise per gram of dry compositions or slurries, or per mL in the case of liquid compositions, at least about 0.25 mg, at least about 0.5 mg, at least about 1 mg, at least about 1.25 mg, at least about 1.5 mg, at least about 2 mg, at least about 3 mg, at least about 5 mg, at least about 7 mg or at least about 10 mg dry biomass, the weight or volume being measured based on the weight or volume of the composition excluding/before addition of biomass and any extracted lipid. In particular embodiments, the compositions (e.g. concentrated flavouring compositions) of the present disclosure comprise from about 1 mg to about 200 mg wet biomass, for example from about 5 mg to about 200 mg, from about 7 mg to about 200 mg, from about 10 mg to about 200 mg, from about 20 mg to about 200 mg, from about 25 mg to about 200 mg, from about 30 mg to about 200 mg, from about 40 mg to about 200 mg, from about 30 mg to about 175 mg, or from about 40 mg to about 175 mg wet biomass per gram of dry compositions or slurries, or per mL in the case of liquid compositions. In particular embodiments wherein dry biomass is used, the compositions (e.g. concentrated flavouring compositions) of the present disclosure may comprise per gram of dry compositions or slurries, or per mL in the case of liquid compositions, from about 0.25 mg to about 100 mg, for example from about 0.5 mg to about 100 mg, for example from about 1 mg to about 100 mg, for example from about 5 mg to about 100 mg, for example from about 10 mg to about 100 mg, for example from about 10 mg to about 80 mg, for example from about 10 mg to about 70 mg, for example from about 15 mg to about 60 mg, for example from about 10 mg to about 50 mg dry biomass.

According to some embodiments, the compositions may comprise per gram of dry compositions or slurries, or per mL in the case of liquid compositions, for example, at least about 5 mg of phospholipid, extracted from a microorganism (e.g. Mortierella spp.), for example at least about 10 mg or at least about 15 mg of extracted lipid comprising phospholipid, extracted from a microorganism, the weight or volume being measured based on the weight or volume of the composition excluding/before addition of biomass and extracted lipid. According to some embodiments, the composition comprises from about 10 mg to about 100 mg, for example from about 10 mg to about 80 mg, for example from about 10 to about 70 mg, for example from about 10 to 60 mg, particularly preferably about 10 to about 50 mg extracted lipid comprising phospholipid, extracted from a microorganism. According to some embodiments, the compositions of the present disclosure provide at least about 15 mg, for example at least about 20 mg extracted lipid comprising phospholipid, extracted from a microorganism.

Food products, beverage products and feedstuffs of the present disclosure, especially meat or meat-like food products, may comprise, according to preferred embodiments, less than about 5% dry biomass (e.g. Mortierella spp. biomass) by weight, or less than about 20% wet biomass by weight. In some embodiments, the food product, beverage product or feedstuff of the present disclosure, especially a meat or meat-like food product, comprises about 4.5% or less, about 4.0% or less, about 3.5% or less, about 3% or less, about 2.5% or less, about 2% or less, about 1.5% or less, about 1% or less, or about 0.5% or less dry biomass by weight, or about 18% or less, about 16% or less, about 15% or less, about 14% or less, about 12% or less, about 10% or less, about 8% or less, about 6% or less, about 5% or less, about 4% or less, about 3% or less, about 2% or less, or about 1% or less wet biomass by weight. In some particular embodiments, the food product, beverage product or feedstuff of the present disclosure, especially a meat or meat-like food product, comprises about or less than 2.5% dry biomass by weight, or about or less than 10% or less wet biomass by weight.

Food products, beverage products and feedstuffs of the present disclosure, especially meat or meat-like food products, may comprise, according to some embodiments, at least about 0.005%, at least about 0.01%, at least about 0.025%, at least about 0.05%, at least about 0.1%, at least about 0.5%, at least about 1%, or at least about 1.25% dry biomass (e.g. Mortierella spp.biomass) by weight, or at least about 0.05%, at least about 0.1%, at least about 0.25%, at least about 0.5%, at least about 1%, at least about 2.5% or at least about 5% wet biomass by weight. In particular embodiments, the food product, beverage product or feedstuff of the present disclosure, especially a meat or meat-like food product, comprises about at least about 0.025% dry biomass by weight, or at least about 0.1% wet biomass by weight.

Food products, beverage products and feedstuffs of the present disclosure, especially meat or meat-like food products, may comprise, according to some particular embodiments, from about 0.005% to about 5% (or less than about 5%, such as about 4% or 3% or 2% or 1%), from about 0.001% to about 5% (or less than about 5%, such as about 4% or 3% or 2% or 1%), from about 0.025% to about 5% (or less than about 5%, such as about 4% or 3% or 2% or 1%), for example from about 0.05% to about 5% (or less than about 5%, such as about 4% or 3% or 2% or 1%), for example from about 0.1% to about 5% (or less than about 5%, such as about 4% or 3% or 2% or 1%) dry biomass (e.g. Mortierella spp. biomass) by weight, or from about 0.05% to about 20% (or less than about 20%), 0.1% to about 20% (or less than about 20%), for example from about 0.25% to about 20% (or less than about 20%), for example from about 1% to about 20% (or less than about 20%) wet biomass by weight. In some preferred embodiments, the food products, beverage products and feedstuffs of the present disclosure, especially meat or meat-like food products, comprise from about 0.025% to about 5%, from about 0.025% to about 4%, or from about 0.025% to about 3% dry biomass by weight; or from about 0.1% to about 20%, from about 0.1% to about 15%, or from about 0.1% to about 10% wet biomass by weight.

Compositions of the present invention comprise microbial biomass, and in particular Mortierella spp. biomass, comprising phospholipids; one or more sugars, sugar alcohols, sugar acids, or sugar derivatives; and one or more amino acids or derivatives or salts thereof, or a compound comprising an amino group (e.g. thiamine). The presence of one or more sugars, sugar alcohols, sugar acids, or sugar derivatives; and one or more amino acids or derivatives or salts thereof, or a compound comprising an amino group (e.g. thiamine), are thought to assist in Maillard reactions which occur when the composition (or food product, beverage product or feedstuff in which the composition is present) is heated. In some embodiments, biomass comprising phospholipids and/or an extracted lipid comprising phospholipids is used in a food product, beverage product or feedstuff and one or more sugars, sugar alcohols, sugar acids, or sugar derivatives, and one or more amino acids or derivatives or salts thereof or a compound comprising an amino group (e.g. thiamine) are provided by the other ingredients of the food product, beverage product or feedstuff. Reference to preferred features of one or more sugars, sugar alcohols, sugar acids, or sugar derivatives, and one or more amino acids or derivatives or salts thereof when used in a composition of the invention below may be applied to sugars, sugar alcohols, sugar acids, or sugar derivatives, and amino acids or derivatives or salts thereof when present in a food product, beverage product or feedstuff according to the present invention mutatis mutandis.

Suitable sugars, sugar alcohols, sugar acids, or sugar derivatives will be well known to a person skilled in the art. In this context, the sugars, sugar alcohols, sugar acids, or sugar derivatives are suitable for use in Maillard reactions for food, beverage or feed uses. In this context, the sugars, sugar alcohols, sugar acids, or sugar derivatives are a component other than the microorganism or a component thereof, and the amino acids or derivatives or salts thereof, even if the biomass or component thereof itself comprises sugars, sugar alcohols, sugar acids, or sugar derivatives. Suitable sugars, sugar alcohols, sugar acids, and sugar derivatives include glucose, fructose, ribose, sucrose, arabinose, glucose-6-phosphate, fructose-6-phosphate, fructose 1,6-diphosphate, inositol, maltose, molasses, maltodextrin, glycogen, galactose, lactose, ribitol, gluconic acid and glucuronic acid, amylose, amylopectin, or xylose. In particularly preferred embodiments, the one or more sugars, sugar alcohols, sugar acids, or sugar derivatives comprise one or more of ribose, glucose (dextrose), a combination of glucose and fructose, and xylose. In particular embodiments, the compositions, food products, beverage products or feedstuffs of the present invention comprise ribose. In other embodiments, the compositions, food products, beverage products or feedstuffs of the present invention comprise glucose (i.e. dextrose). In other embodiments, the compositions, food products, beverage products or feedstuffs of the present disclosure comprise both glucose and ribose.

According to some embodiments, the one or more sugars, sugar alcohols, sugar acids, or sugar derivatives are present in the composition at an amount of per kg of dry compositions or slurries, or per L in the case of liquid compositions, from about 1 mmol to about 1000 mmol, for example from about 5 mmol to about 500 mmol, about 5 mmol to about 300 mmol, about 20 mmol to about 500 mmol, about 20 mmol to about 300 mmol, about 5 mmol to about 200 mmol, about 5 mmol to about 100 mmol, about 5 mmol to about 80 mmol, from about 5 mmol to about 70 mmol, about 10 mmol to about 70 mmol, about 15 mmol to about 70 mmol, or about 30 mmol to about 60 mmol, the amount being measured based on the weight or volume of the composition excluding/before addition of biomass and any extracted lipids. In some embodiments, the one or more sugars, sugar alcohols, sugar acids, or sugar derivatives are present in the composition at an amount of per kg of dry compositions or slurries, or per L in the case of liquid compositions, of at least about 5 mmol, at least about 10 mmol, at least about 15 mmol, at least about 20 mmol, or at least about 30 mmol, the amount being measured based on the weight or volume of the composition excluding/before addition of biomass and any extracted lipids. In some such embodiments, the one or more sugars, sugar alcohols, sugar acids, or sugar derivatives comprise ribose and/or glucose.

In some embodiments, the one or more sugars, sugar alcohols, sugar acids or sugar derivatives are present in the food, feedstuff or beverage at a total amount of, per kg of dry food or slurry, or per L in the case of liquid foods (e.g. beverages), from about 0.1 mmol to about 100 mmol, from about 0.5 mmol to about 30 mmol, from about 0.5 mmol to about 50 mmol, from about 1 mmol to about 50 mmol, from about 2 mmol to about 40 mmol, from about 2 mmol to about 30 mmol, from about 1 mmol to about 25 mmol, from about 1 mmol to about 20 mmol, from about 1 mmol to about 10 mmol, from about 7 mmol to about 20 mmol, from about 7 mmol to about 15 mmol, the amount being measured based on the weight or volume of the food, feedstuff or beverage excluding/before addition of the microbial biomass and/or lipids. In some examples, the one or more sugars, sugar alcohols, sugar acids, or sugar derivatives are present in the food, feedstuff or beverage at an amount of per kg of dry food, feedstuff or beverage, or per L in the case of liquid food, feedstuff or beverage, of at least about 0.5 mmol, at least about 1 mmol, at least about 1.5 mmol, at least about 2 mmol, or at least about 3 mmol, the amount being measured based on the weight or volume of the composition excluding/before addition of biomass and any extracted lipids. In some embodiments, the one or more sugars, sugar alcohols, sugar acids, or sugar derivatives comprise ribose and/or glucose.

A sugar “derivative” is intended to encompass sugars which includes a modification from a naturally occurring sugar, for example by modification of substituents, such as hydroxyl groups. For example, sugar derivatives may have been modified to include alternative substituents such as amino groups, acid groups, phosphate groups, acetate groups etc. Sugar derivatives include, but are not limited to, amino sugars, deoxy sugars, glycosylamines, and sugar phosphates.

Amino acids or derivatives or salts thereof used in the present invention are suitable for use in Maillard reactions for food, beverage or feed uses. In this context, the amino acids or derivatives or salts thereof are a component other than the microorganism (e.g. Mortierella spp. biomass) or a component thereof, and the sugar, sugar alcohol, sugar acid, or sugar derivative, even if the biomass or component thereof itself comprises amino acids or derivatives or salts thereof. In particular embodiments, the one or more amino acids or derivatives or salts thereof contain a free amino group. Thus, in some embodiments reference to an amino acid or derivative means a free amino acid that is not present in the context of a peptide or protein. Suitable amino acids and derivatives thereof include cysteine, cystine, homocysteine, selenocysteine, a cysteine sulfoxide, allicin, selenocysteine, methionine, isoleucine, leucine, lysine, phenylalanine, threonine, tryptophan, 5-hydroxytryptophan, valine, arginine, histidine, alanine, asparagine, aspartate, glutamate or glutamic acid, glutamine, monosodium glutamate, glycine, proline, serine, taurine and tyrosine. In particularly preferred embodiments, the amino acid is cysteine and/or cystine. In particularly preferred embodiments, the compositions comprise cysteine. In some preferred embodiments, the composition, food product, beverage product or feedstuff comprises glutamic acid or a salt thereof. In some particularly preferred embodiments, the composition, food product, beverage product or feedstuff comprises glutamic acid or a salt thereof (e.g. monosodium glutamate, or MSG) in addition to the one or more amino acids or derivatives or salts thereof; for example, compositions, food products, beverage products or feedstuffs comprise, according to some embodiments, glutamic acid or a salt thereof and cysteine (or cystine) or a salt thereof. In some embodiments, the one or more amino acids or derivatives or salt thereof comprises a sulfur-containing amino acid (e.g. cysteine, methionine, homocysteine, or taurine) or salt. Salts of amino acids which are suitable for human or animal consumption and therefore for incorporation into compositions, food products, beverage products or feedstuffs of the present disclosure will be familiar to and readily selected by a person skilled in the art.

An amino acid “derivative” is intended to encompass amino acids which include a chemical modification, for example by introducing a group in a side chain of an amino acid, such as a nitro group in tyrosine or iodine in a tyrosine, by conversion of a free carboxylic group to an ester group or to an amide group, by converting an amino group to an amide by acylation, by acylating a hydroxy group rendering an ester, by alkylation of a primary amine rendering a secondary amine, or linkage of a hydrophilic moiety to an amino acid side chain. Other derivatives may be obtained by oxidation or reduction of the side-chains of the amino acid. Modification of an amino acid may also include derivation of an amino acid by the addition and/or removal of chemical groups to/from the amino acid, and may include use of an amino amino acid analog (such as a phosphorylated amino acid) or a non-naturally occurring amino acid such as a N-alkylated amino acid (e.g. N-methyl amino acid), D-amino acid, R-amino acid or γ-amino acid. Exemplary derivatives may include derivatives obtained by attachment of a derivative moiety, i.e. a substituent group, to an amino acid. The term “derivative” in the context of amino acids will be readily understood by a skilled person.

According to some embodiments, each of the one or more amino acids or derivatives or salts thereof are present in the composition at an amount of, per kg of dry compositions or slurries, or per L in the case of liquid compositions, from about 1 mmol to about 500 mmol, from about 1 mmol to about 300 mmol, from about 1 mmol to about 200 mmol, from about 2 mmol to about 200 mmol, from about 2 mmol to about 100 mmol, from about 2 mmol to about 200 mmol, from about 5 mmol to about 100 mmol, from about 5 mmol to about 80 mmol, from about 5 mmol to about 70 mmol, from about 10 mmol to about 70 mmol, from about 15 mmol to about 70 mmol, from about 30 mmol to about 60 mmol, from about 1 mM to about 50 mM, or from about 1 30 mM, the amount being calculated based on the weight or volume of the composition excluding/before addition of biomass and any extracted lipid comprising phospholipids. In some embodiments, the one or more amino acids or derivatives or salts thereof are present in the composition at an amount of per kg of dry compositions or slurries, or per L in the case of liquid compositions, of at least about 1 mmol, for example at least about 5 mmol, for example at least about 10 mmol, for example at least about 15 mmol, for example at least about 20 mmol, the amount being measured based on the weight or volume of the composition excluding/before addition of biomass and any extracted lipids. In some such embodiments, the one or more amino acids comprises cysteine or cystine.

According to some embodiments, each of the one or more amino acids or derivatives or salts thereof are present in the food, feedstuff or beverage at a total amount of, per kg of dry composition or slurry, or per L in the case of liquid foods (e.g. beverages), from about 0.1 mmol to about 50 mmol, about 0.1 mmol to about 40 mmol, about 0.1 mmol to about 30 mmol, about 0.5 mmol to about 40 mmol, about 0.5 mmol to about 30 mmol, about 1 mmol to about 10 mmol, about 1.5 mmol to about 10 mmol, about 0.5 to about 5 mmol, about 1 mmol to about 5 mmol, or about 5 to about 10 mmol the amount being calculated based on the weight or volume of the food, feedstuff or beverage excluding/before addition of microbial biomass and/or lipids. In preferred embodiments, the one or more amino acids comprises cysteine and/or cystine.

The one or more sugars, sugar alcohols, sugar acids, or sugar derivatives and one or more amino acids or derivatives or salts thereof or a compound comprising an amino group (e.g. thiamine) are present in the compositions of the present disclosure or the food products, beverage products or feedstuffs of the present disclosure in amounts sufficient to product food-like aromas, such as meat-like aromas, when heat is applied to the compositions, food products, beverage products or feedstuffs. In particular embodiments, the one or more sugars, sugar alcohols, sugar acids, or sugar derivatives and one or more amino acids or derivatives or salts thereof are present in the compositions of the present disclosure or the food products, beverage products or feedstuffs of the present disclosure in amounts sufficient to produce one or more volatile compounds selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane, for example two or more, three or more, four or more or five or more of the aforesaid compounds when heat is applied to the composition, food product, beverage product or feedstuff. In some particular embodiments, the one or more sugars, sugar alcohols, sugar acids, or sugar derivatives and one or more amino acids or derivatives or salts thereof are present in the compositions of the present disclosure or the food products, beverage products or feedstuffs of the present disclosure in amounts sufficient to produce one or more volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol when heat is applied to the composition, food product, beverage product or feedstuff.

In some embodiments, the composition comprises comprise glutamic acid or a salt or derivative thereof (e.g. MSG) in addition to the one or more amino acids or derivatives or salts thereof. In some embodiments, the glutamic acid or salt thereof is present in an amount of, per kg of dry compositions or slurries, or per L in the case of liquid compositions, from about 1 mmol to about 200 mmol or from about 2 mmol to about 100 mmol, for example 2 mmol to about 50 mmol, for example from about 2 mmol to about 40 mmol, for example from about 2 mmol to about 40 mmol, for example from about 5 mmol to about 40 mmol, for example from about 5 mmol to about 30 mmol, the amount being calculated based on the volume of the composition excluding/before addition of biomass and any extracted lipid comprising phospholipids. In some embodiments, the glutamic acid or salt thereof is present in an amount of, per kg of dry compositions or slurries, or per L in the case of liquid compositions, at least about 1 mmol, for example at least about 2 mmol, for example at least about 3 mmol, for example at least about 4 mmol, for example at least about 5 mmol, for example at least about 7 mmol, for example at least about 10 mmol, the amount being measured based on the weight or volume of the composition excluding/before addition of biomass and any extracted lipids. In some embodiments, the glutamic acid salt is monosodium glutamate.

In some embodiments, the food or bereage product or feedstuff of the invention comprises glutamic acid or a salt or derivative thereof (e.g. MSG) in addition to one or more other amino acids or derivatives or salts thereof, and the glutamic acid is present in an amount of, per kg of dry composition or slurry, or per L in the case of liquid compositions (e.g. beverages), from about 0.1 mmol to about 20 mmol, about 0.1 mmol to about 15 mmol, about 0.3 mmol to about 15 mmol, about 0.5 mmol to about 10 mmol, about 0.5 mmol to about 5 mmol, or about 1 mmol to about 5 mmol, the amount being calculated based on the volume of the food, feedstuff or beverage excluding/before addition of microbial biomass and/or lipids.

In some embodiments, the composition comprises glutamic acid or a salt thereof and a further amino acid or salt or derivative thereof selected from cysteine and cystine (or a salt or derivative thereof), wherein the glutamic acid or salt thereof is present in an amount of, per kg of dry compositions or slurries, or per L in the case of liquid compositions, from about 1 mmol to about 200 mmol or from about 2 mmol to about 100 mmol, for example 2 mmol to about 50 mmol, for example from about 2 mmol to about 40 mmol, for example from about 2 mmol to about 40 mmol, for example from about 5 mmol to about 40 mmol, for example from about 5 mmol to about 30 mmol; and the cysteine or cystine (or a salt or derivative thereof) is present in an amount of from about 5 mmol to about 200 mmol or from about 5 mmol to about 100 mmol, for example from about 5 mmol to about 80 mmol, for example from about 5 mmol to about 70 mmol, for example from about 10 mmol to about 70 mmol, for example from about 15 mmol to about 70 mmol, for example from about 30 mmol to about 60 mmol, the amount being calculated based on the weight or volume of the composition excluding/before addition of biomass and any extracted lipid comprising phospholipids. In some embodiments, the composition comprises glutamic acid or a salt thereof and a further amino acid or salt or derivative thereof selected from cysteine and cystine, wherein the glutamic acid or salt thereof is present in an amount of, per kg of dry compositions or slurries, or per L in the case of liquid compositions, at least about 1 mmol, for example at least about 2 mmol, for example at least about 3 mmol, for example at least about 4 mmol, for example at least about 5 mmol, for example at least about 7 mmol, for example at least about 10 mmol, and the cysteine or cystine (or a salt or derivative thereof) is present in an amount of at least about 5 mmol, for example at least about 10 mmol, for example at least about 15 mmol, for example at least about 20 mmol, the amount being calculated based on the weight or volume of the composition excluding/before addition of biomass and any extracted lipid comprising phospholipids.

In some embodiments, the food or beverage product or feedstuff comprises glutamic acid or a salt thereof and a further amino acid or salt or derivative thereof selected from cysteine and cystine (or a salt or derivative thereof), wherein the glutamic acid or salt thereof is present in an amount of, per kg of dry compositions or slurries, or per L in the case of liquid compositions, from about 0.1 mmol to about 20 mmol or from about 0.2 mmol to about 10 mmol, for example 0.2 mmol to about 5 mmol, for example from about 0.2 mmol to about 4 mmol, for example from about 0.5 mmol to about 4 mmol, for example from about 0.5 mmol to about 3 mmol; and the cysteine or cystine (or a salt or derivative thereof) is present in an amount of from about 0.5 mmol to about 50 mmol or from about 0.5 mmol to about 20 mmol, for example from about 0.5 mmol to about 10 mmol, for example from about 0.5 mmol to about 8 mmol, for example from about 0.5 mmol to about 7 mmol, for example from about 1 mmol to about 7 mmol, for example from about 1.5 mmol to about 7 mmol, for example from about 3 mmol to about 6 mmol, the amount being calculated based on the weight or volume of the composition excluding/before addition of biomass and any extracted lipid comprising phospholipids. In some embodiments, the composition comprises glutamic acid or a salt thereof and a further amino acid or salt or derivative thereof selected from cysteine and cystine (or a salt or derivative thereof), wherein the glutamic acid or salt thereof is present in an amount of, per kg of dry compositions or slurries, or per L in the case of liquid, at least about 0.1 mmol, for example at least about 0.2 mmol, for example at least about 0.3 mmol, for example at least about 0.4 mmol, for example at least about 0.5 mmol, for example at least about 0.7 mmol, for example at least about 1 mmol, and the cysteine or cystine (or a salt or derivative thereof) is present in an amount of at least about 0.5 mmol, for example at least about 1 mmol, for example at least about 1.5 mmol, for example at least about 2 mmol, the amount being calculated based on the weight or volume of the composition excluding/before addition of biomass and any extracted lipid comprising phospholipids.

Compositions, food products, beverage products or feedstuffs of the present invention may, according to some preferred embodiments, comprise a source of iron. Iron may enhance the meaty flavour and/or aromas produced by compositions, food products, beverage products or feedstuffs of the present invention. In some embodiments, the source of iron is an iron salt, preferably a ferrous salt. Any iron salt suitable for consumption may be used, and such salts will be familiar to a person skilled in the art, for example a chelated form of iron. In some embodiments, the source of iron is iron (II) fumarate. Iron (II) fumarate is available, for example, as iron tablets from APOHEALTH Pty Ltd (NSW, Australia). The source of iron is a component other than the biomass or a component thereof, the amino acid or salt or derivative thereof, and the sugar, sugar alcohol, sugar acid, or sugar derivative, even if the biomass or component thereof itself comprises iron.

In particular embodiments, the compositions of the present invention comprise a source of iron in an amount equivalent to, per kg of dry compositions or slurries, or per L in the case of liquid compositions, up to about 100 mg of elemental iron. In some embodiments, the compositions comprise a source of iron in an amount equivalent to up to about 50 mg, for example from about 20 to about 50 mg, for example from about 30 to about 40 mg, the concentration being calculated based on the volume of the composition excluding/before addition of biomass and any extracted lipid comprising phospholipids.

In particularly preferred embodiments, the compositions, food products, beverage products or feedstuffs of the present disclosure comprise an aqueous component. Presence of some moisture in the compositions facilitates production of food-like flavour and/or aromas upon heating. An aqueous component may be water. In some embodiments, the aqueous component may be, for example, an aqueous buffer such as a phosphate buffer. In particular embodiments, the compositions, food products, beverage products or feedstuffs of the present disclosure comprise an aqueous component aside from any water contained incidentally in other components, such as any moisture present in microorganism biomass. Compositions of the present disclosure are, in some preferred embodiments, not dry or substantially dry.

In one embodiment, the composition, food product, beverage product or feedstuff is a dry composition. In another embodiment, the composition, food product, beverage product or feedstuff is a liquid composition. In one embodiment, the composition, food product, beverage product or feedstuff is in the form of a powder, solution, suspension, slurry or emulsion. In some embodiments, the composition, food product, beverage product or feedstuff is provided excluding an aqueous component (i.e. a dry composition), and an aqueous component (such as water) is added to the composition, food product, beverage product or feedstuff prior to or together with heating.

In some embodiments, compositions, food products, beverage products or feedstuffs of the present disclosure may further comprise an aqueous buffer. A buffer maintains the pH of the composition, and provides moisture to the composition, food product, beverage product or feedstuff which, as discussed above, facilitates production of food-like flavour and/or aromas upon heating. In some embodiments, the buffer may be a phosphate buffer. In some embodiments, the buffer may be a buffer at a pH of from about 5.0 to about 7, for example from about 5 to about 6, for example at about 5.3 or about 6.0. In particular embodiments, the buffer is a phosphate buffer at a pH of about 6.0.

The compositions, food products, beverage products or feedstuffs of the present invention may further comprise one or more additional components. Such components may be flavour precursors, for example intended to be involved with Maillard reactions occurring when the composition, food product, beverage product or feedstuff is heated. For example, such additional components may include oils (for example vegetable oils), free fatty acids, alpha-hydroxy acids, dicarboxylic acids, nucleosides, nucleotides, vitamins, peptides, protein hydrolysates, extracts, phospholipids, lecithin, carbohydrates, and organic molecules. In particular examples, the compositions of the present invention, which may be flavouring compositions (e.g. to incorporate into a food product, feedstuff or beverage so as to impart a food-like flavour, such as a meat-like flavour), comprise less than 3%, 4%, 5%, 6%, 7%, 8%, 9%, 10%, 15%, 20%, 25%, or 30% protein by weight, other than protein provided by the biomass (e.g. the Mortierella spp. biomass).

In some embodiments, the compositions, food products, beverage products or feedstuffs comprise thiamine or derivatives thereof. For example, the thiamine can be present as the compound containing an amino group and thus enable the Maillard reaction. Accordingly, the the compositions, food products, beverage products or feedstuffs of the present invention may comprise the a) the biomass, b) sugars, sugar alcohols, sugar acids, or sugar derivatives and c) thiamine. In other example, the compositions, food products, beverage products or feedstuffs of the present invention may comprise a) the biomass, b) sugars, sugar alcohols, sugar acids, or sugar derivatives, c) one or more amino acids or derivatives or salts thereof, and d) thiamine. Thiamine may therefore enhance the meaty aroma and/or flavour produced by compositions, food products, beverage products or feedstuffs of the present invention. In some embodiments, thiamine may be present in the compositions, per kg of dry compositions or slurries, or per L in the case of liquid compositions, in an amount of from about 0.1 to about 20 mmol, for example from about 0.1 to about 10 mmol, for example from about 0.5 to about 5 mmol, for example from about 0.5 to about 3 mmol. In some embodiments, thiamine is present in an amount of at least about 0.1 mmol, for example at least about 0.2 mmol, for example at least about 0.3 mmol, for example at least about 0.4 mmol, for example at least about 0.5 mmol, for example at least about 0.7 mmol, the concentration being calculated based on the weight or volume of the composition excluding/before addition of biomass and any extracted lipid comprising phospholipids. In some embodiments, thiamine may be present in the food, feedstuffs or beverages, per kg of dry composition or slurry, or per L in the case of liquid compositions (e.g. beverages), in an amount of from about 0.01 to about 2 mmol, for example about 0.01 to about 1 mol, for example from about 0.05 to about 0.5 mmol, or about 0.1 to about 0.3 mmol, the amount being calculated based on the weight or volume of the food, feedstuff or beverage excluding/before addition of microbial biomass and/or lipids. In some embodiments, thiamine is present in the food, feedstuff or beverages in an amount of at least about 0.01 mmol, for example at least about 0.02 mmol, for example at least about 0.03 mmol, for example at least about 0.04 mmol, for example at least about 0.05 mmol, for example at least about 0.07 mmol, the concentration being calculated based on the weight or volume of the food, feedstuff or beverage excluding/before addition of biomass and any extracted lipid comprising phospholipids.

In some embodiments, the compositions, food products, beverage products or feedstuffs further comprise a yeast extract. In the art of food science, a “yeast extract” is generally understood to refer to the water-soluble portion of autolyzed yeast and is available commercially from various suppliers; see, for example Sigma Aldrich, Catalog No. Y1625 Yeast Extract. A yeast extract does not contain yeast whole cell biomass. Presence of a yeast extract may enhance meaty aromas and/or flavours produced by the composition, food product, beverage product or feedstuff when heated. The yeast extract may be a general unflavoured yeast extract, or may be, for example, a beef flavoured or roast chicken skin flavoured yeast extract. In some embodiments, the composition, food product, beverage product or feedstuff is suitable for producing food-like aromas and/or flavours which are meat-like aromas and/or flavours, and the composition, food product, beverage product or feedstuff comprises a yeast extract. The presence of a yeast extract may enhance meaty aromas and/or flavours produced by compositions, food products, beverage products or feedstuffs of the present disclosure, as observed in the Examples below.

In some embodiments, the yeast extract is present in the composition in an amount of, per kg of dry compositions or slurries, or per L in the case of liquid compositions, from about 10 g to about 200 g, for example from about 15 g to about 200 g, for example from about 20 g to about 200 g, for example from about 30 g to about 200 g, for example from about 40 g to about 200 g, for example from about 50 g to about 200 g, for example from about 50 g to about 180 g, for example from about 60 g to about 180 g, the amount being calculated based on the volume of the composition excluding/before addition of biomass and any extracted lipid from microorganisms. In some embodiments, the yeast extract is present in the composition in an amount of, per kg of dry compositions or slurries, or per L in the case of liquid compositions, at least about 5 g, for example at least about 7 g, for example at least about 10 g, for example at least about 15 g, for example at least about 20 g, for example at least about 25 g, for example at least about 30 g, for example at least about 40 g, for example at least about 50 g, for example at least about 60 g. In particular embodiments, the yeast extract is present in the composition in an amount of, per kg of dry compositions or slurries, or per L in the case of liquid compositions, at least about 30 g.

In some embodiments, the yeast extract is present in the composition in an amount of, per kg of dry compositions or slurries, or per L in the case of liquid compositions, at least about 5 g, for example at least about 7 g, for example at least about 10 g, for example at least about 15 g, for example at least about 20 g, for example at least about 25 g, for example at least about 30 g, for example at least about 40 g, for example at least about 50 g, for example at least about 60 g. In particular embodiments, the yeast extract is present in the composition in an amount of, per kg of dry compositions or slurries, or per L in the case of liquid compositions, at least about 30 g. In some embodiments, the yeast extract is present in the food, feedstuff or beverage in an amount of, per kg of dry compositions or slurries, or per L in the case of liquid, at least about 0.5 g, for example at least about 0.7 g, for example at least about 1 g, for example at least about 1.5 g, for example at least about 2 g, for example at least about 2.5 g, for example at least about 3 g, for example at least about 4 g, for example at least about 5 g, for example at least about 6 g. In particular embodiments, the yeast extract is present in the food, feedstuff or beverage in an amount of, per kg of dry compositions or slurries, or per L in the case of liquid compositions, at least about 3 g.

In some embodiments, the composition, food product, beverage product or feedstuff does not comprise a yeast extract. Since the presence of a yeast extract may enhance meaty aromas and/or flavours produced by the composition, food product, beverage product or feedstuff, a yeast extract maybe omitted when, for example, an alternative food-like flavour and/or aroma is desired, such as a fishy or a vegetable or herby aroma and/or flavour. The absence of a yeast extract may prevent the potential masking of the desired aroma and/or flavour such as a fish-like aroma and/or flavour by meat-like aromas and/or flavours enhanced by the presence of a yeast extract. Accordingly, in some embodiments, the food-like aroma and/or flavour is a fish-like aroma and/or flavour, a vegetable, and/or a herby aroma and/or flavour, and the composition, food product, beverage product or feedstuff does not comprise a yeast extract.

In some embodiments, the compositions, food products, beverage products or feedstuffs further comprise one or more herbs and/or spices. As demonstrated by the Examples herein, compositions comprising herbs, such as for example Fenugreek (Trigonella foenum-graecum), were found in some instances to enhance vegetable, soupy and/or herby flavour and/or aromas produced by the compositions of the present invention. These herby, vegetable and/or soupy flavour and/or aromas may partially or completely mask meaty/fishy aromas and/or flavours in some embodiments, allowing adjustment of overall aromas and/or flavours produced by compositions, food products, beverage products or feedstuffs of the present disclosure. A herb and/or spice is understood in the art to refer to a plant part or extract possessing aromatic properties. Typically, a herb is understood to refer to leafy, green or flowering parts of a plant, whilst a spice is typically understood to refer to other parts of a plant (usually dried), including seeds, bark, roots and fruit. The herb or spice may be in the form of whole plant parts, or chopped, ground or rolled plant parts, or dried, for example as a powder. In particular embodiments, the one or more herbs and/or spices comprise Fenugreek. Fenugreek has also been claimed to contain several bioactive components and can bring health benefits to consumers. In some embodiments, the one or more herbs and/or spices comprise Fenugreek leaf.

In some embodiments, the compositions, foods, feedstuffs or beverages comprise:
a) Mortierella spp. biomass (or other microbial biomass) comprising phospholipids;
b) glucose and/or ribose;
b) cysteine and/or cystine and/or methionine and/or thiamine; and
e) an aqueous component.

In some embodiments, the compositions, foods, feedstuffs or beverages comprise:
a) Mortierella spp. biomass (or other microbial biomass) comprising phospholipids;
b) glucose and/or ribose;
b) cysteine and/or cystine and/or methionine and/or thiamine;
c) glutamic acid or a salt thereof;
and
d) an aqueous component.

In some embodiments, the compositions comprise:
a) Mortierella spp. biomass (or other microbial biomass) comprising phospholipids;
b) glucose and/or ribose;
b) cysteine and/or cystine;
d) yeast extract;
e) glutamic acid or a salt thereof;
f) thiamine; and
g) an aqueous component.

In some embodiments, the compositions comprise:
a) Mortierella spp. biomass (or other microbial biomass) comprising phospholipids;
b) glucose and/or ribose;
b) cysteine and/or cysteine;
d) a source of iron, for example an iron salt;
e) glutamic acid or a salt thereof;
f) thiamine;
g) an aqueous component, for example as an aqueous buffer, for example a phosphate buffer, for example having a pH of from about 5 to about 6, for example of about 5.3 or about 6.0; and
h) optionally a yeast extract.

In some embodiments, the compositions comprise:
a) Mortierella spp. biomass (or other microbial biomass) comprising phospholipids;
b) ribose;
b) cysteine;
d) a source of iron, for example an iron salt;
e) glutamic acid or a salt thereof;
f) thiamine;
g) an aqueous component, for example as an aqueous buffer for example a phosphate buffer, for example having a pH of from about 5 to about 6, for example of about 5.3 or about 6.0; and
h) optionally a yeast extract.

In some embodiments, the composition comprises (aside from the biomass and anyextracted lipid) the components set out in “matrix A” or “matrix B” in Table 1 below, or “matrix C” in Table 2 below (as prepared from the stock ingredients set out below), or the components of Matrix A, B or CB in equivalent concentrations if otherwise prepared.

TABLE 1

| Volume of stock (μL) | Matrix base A | Matrix base B |
|---|---|---|
| Cysteine | 100 | 100 |
| Ribose | 100 | 100 |
| Thiamine | 45.5 | 45.5 |
| Iron | 53.1 | 53.1 |
| Yeast extract (general) | | 400 |
| Glutamic acid | 50 | 50 |
| Buffer pH 6.0 | 651.4 | 251.4 |

Stock ingredients/reagents/chemical solutions to make up matrix base A and B:
50 mM potassium phosphate buffer pH 6.0
100 mM Cysteine
100 mM Ribose
44 mM Thiamine
100 mM Glutamic acid
Iron (Iron tablet, Apohealth), 65.7 mg Fe²+/100 mL
Yeast extract (general, 75 mg/100 mL)

TABLE 2

| Stock solutions | Volume (μL) |
|---|---|
| Cysteine•HCL (400 mM) | 250 |
| Ribose (400 mM) | 250 |
| Thiamine•HCL (44 mM) | 90.9 |
| Yeast extract (general) (30 g/100 mL) | 1000 |
| Monosodium glutamate (400 mM) | 125 |
| Water | 284.1 |
| Total | 2000 |

The present disclosure further relates to a method of producing a food product, beverage product or feedstuff comprising combining biomass and any optional extracted lipids comprising phospholipids disclosed herein or a composition of the present disclosure with one or more additional consumable ingredients. Suitable additional ingredients which may be included in such food products, beverage products or feedstuffs are discussed below. For example, the biomass and any optional extracted lipids comprising phospholipids disclosed herein or composition can be combined with the other consumable ingredient by mixing, applying it to the surface of the other ingredient, or by soaking/marinating the other ingredient. In an embodiment, the food, feedstuff or beverage product is prepared by (a) heating biomass and any optional extracted lipids comprising phospholipids disclosed herein or a composition of the invention and (b) mixing the products from (a) with other food, feedstuff or beverage consumable ingredients, or by (a) mixing biomass and any optional extracted lipids comprising phospholipids disclosed herein or a composition of the present disclosure with other food, feedstuff or beverage consumable ingredients and (b) heating the mixture resulting from (a).

The food product, beverage product or feedstuff may either be in a solid or liquid form, and may be intended to be kept frozen, refrigerated or at room temperature prior to cooking. In some embodiments, the food product, beverage product, feedstuff or composition is provided as a dry product excluding an aqueous component, and an aqueous component (such as water) is added to the food product, beverage product or feedstuff or composition prior to, during or subsequent to heating, especially prior to heating.

In some embodiments, the composition may be in a solid or liquid form, to be admixed with, or added to a food or beverage product or feedstuff prior to heating, or after heating one or both of the composition and the food or beverage product or feedstuff. The composition may be in solid or liquid form, and may represent, for a example, a concentrated mix, to be mixed with or added to a food or beverage product or feedstuff. The mix may be, for example, in the form of a powder, particulate or granulated mix.

The food or beverage product or feedstuff may include edible macronutrients, protein, carbohydrate, vitamins, and/or minerals in amounts desired for a particular use. The amounts of these ingredients will vary depending on whether the composition is intended for use with normal individuals or for use with individuals having specialized needs, such as individuals suffering from metabolic disorders and the like.

According to some particular embodiments, the food or beverage product or feedstuff of the present invention contains no components derived from an animal. In a preferred embodiment, at least some of the ingredients are plant material or material derived from a plant. Such embodiments are advantageously suitable for a vegan or vegetarian diet. In some embodiments, the food or beverage product or feedstuff can be soy-free, wheat-free, yeast-free, MSG-free, and/or free of protein hydrolysis products. The food or beverage product or feedstuff preferably has a food-like taste or aroma, such as a meaty or fishy aroma, as imparted by the biomass and any extracted lipids comprising phospholipids disclosed herein or composition of the present disclosure.

Examples of suitable additional ingredients with nutritional value include, but are not limited to, macronutrients such as edible fats, carbohydrates and proteins. Examples of such edible fats other than phospholipids contained in compositions of the present disclosure include, but are not limited to, palm oil, canola oil, corn oil, sunflower oil, safflower oil, coconut oil, borage oil, fungal oil, black current oil, soy oil, blends thereof and mono- and diglycerides. Examples of carbohydrates include (but are not limited to): glucose, edible lactose, and hydrolyzed starch. Examples of proteins include (but are not limited to) soy proteins, mycoproteins (e.g Rhiza mycoproteins), seitan, pea protein, potato protein, electrodialysed whey, electrodialysed skim milk, milk whey, or the hydrolysates of these proteins. In some examples, the protein is a textured or structured protein product, which comprises protein fiber networks and/or aligned protein fibers that produce meat-like textures. It can be obtained from a dough after application of mechanical energy (e.g., extrusion, spinning, agitating, shaking, shearing, pressure, turbulence, impingement, confluence, beating, friction, wave), radiation energy (e.g., microwave, electromagnetic), thermal energy (e.g., heating, steam texturizing), enzymatic activity (e.g., transglutaminase activity), chemical reagents (e.g., pH adjusting agents, kosmotropic salts, chaotropic salts, gypsum, surfactants, emulsifiers, fatty acids, amino acids), other methods that lead to protein denaturation and protein fiber alignment, or combinations of these methods, followed by fixation of the fibrous and/or aligned structure (e.g., by rapid temperature and/or pressure change, rapid dehydration, chemical fixation, redox), and optional post-processing after the fibrous and/or aligned structure is generated and fixed (e.g., hydrating, marinating, drying, coloring).

With respect to vitamins and minerals, the following may be added to the food or beverage product or feedstuff of the present invention: calcium, phosphorus, potassium, sodium, chloride, magnesium, manganese, iron, copper, zinc, selenium, iodine, and Vitamins A, E, D, C, and the B complex. Other such vitamins and minerals may also be added.

Additional ingredients which may be included in food or beverage products or feedstuffs include food-grade oils such as canola, corn, sunflower, soybean, olive or coconut oil, seasoning agents such as edible salts (e.g., sodium or potassium chloride) or herbs (e.g., rosemary, thyme, basil, sage, or mint), flavouring agents, proteins (e.g., soy protein isolate, wheat gluten, pea vicilin, and/or pea legumin), protein concentrates (e.g., soy protein concentrate), emulsifiers (e.g., lecithin), gelling agents (e.g., k-carrageenan or gelatin), fibers (e.g., bamboo filer or inulin), or minerals (e.g., iodine, zinc, and/or calcium).

Food and beverage products and feedstuffs described herein also can include a natural coloring agent such as turmeric or beet juice, or an artificial coloring agent such as azo dyes, triphenylmethanes, xanthenes, quinines, indigoids, titanium dioxide, red #3, red #40, blue #1, or yellow #5.

Food and beverage products and feedstuffs described herein also can include meat shelf-life extenders such as carbon monoxide, nitrites, sodium metabisulfite, Bombal, vitamin E, rosemary extract, green tea extract, catechins and other anti-oxidants.

The components utilized in the food or beverage product or feedstuff of the present invention can be of semi-purified or purified origin. By semi-purified or purified is meant a material which has been prepared by purification of a natural material or by de novo synthesis.

Food products, feedstuffs, beverage products and compositions described herein can be packaged in various ways, including being sealed within individual packets or shakers, such that the composition can be sprinkled or spread on top of a food product before or during cooking.

Compositions, food products, beverage products and feedstuffs described herein can be assessed for flavour and aroma using human panelists. It will be appreciated that assessment of aromas by panellists will involve a certain degree of subjectivity, and that precise descriptions of aromas and whether they are appealing/unappealing may differ somewhat between panellists. Nonetheless, trends and the general nature of aromas can be effectively assessed by panellists. The evaluations can involve eyeing, feeling, chewing, smelling and tasting of the product to judge product appearance, color, integrity, texture, flavour, and mouth feel, etc., preferably at least smelling the composition, food or beverage product or feedstuff to assess aroma. Panelists can be served samples under red or under white light. A scale can be used to rate the overall acceptability or quality of the food or specific quality attributes such meatiness, texture, and flavour. The compositions, food products, beverage products and feedstuffs can also be presented to animals such as pet animals to assess their attractiveness to those animals.

In some embodiments, a food product, beverage product or feedstuff or composition described herein can be compared to another product (e.g., meat or meat substitute) based upon olfactometer readings. In various embodiments, the olfactometer can be used to assess odor concentration and odor thresholds, odor suprathresholds with comparison to a reference gas, hedonic scale scores to determine the degree of appreciation, or relative intensity of odors.

In some embodiments, volatile chemicals identified using GCMS can be evaluated. For example, a human can rate the experience of smelling the chemical responsible for a certain peak. This information could be used to further refine the profile of flavour and aroma compounds produced by the food products, beverage products, feedstuffs or compositions of the present invention.

The present invention further relates to methods of producing a composition, food product, beverage product or feedstuff, by combining a biomass with any one or more of the ingredients described above, optionally in the amounts as described above.

Food-Like Aromas and/or Flavours

The compositions, food products, beverage products or feedstuffs of the present disclosure produce a food-like flavour and/or aroma, preferably a meat-like flavour and/or aroma, when heated. Heating refers to increasing the temperature of the composition, food products, beverage products or feedstuffs, for example to above room temperature, to any temperature and for any amount of time sufficient to produce food-like flavour and/or aromas. In this context, the temperature is raised high enough and long enough for Maillard reactions to occur between amino groups and sugars in the composition, with additional reactions occurring with lipids, preferably phospholipids, in the composition, food products, beverage products or feedstuffs to produce the food-like flavour and/or aromas. Selection of a suitable temperature and period of time may be readily carried out by the skilled person. As used herein, “heated” or “heating” or similar is to be understood as meaning heating under conditions sufficient for producing a food-like aroma, unless otherwise specified. In the context of a composition to be added to a food product, beverage product or feedstuff, the heat may be applied to the composition of the invention prior to it being contacted with the food product, beverage product or feedstuff or after the application to the food product, beverage product or feedstuff or both. Such heating of the composition, or the food product, beverage product or feedstuff, may take place for example in an oven, frypan, wok or similar, or in a barbeque.

Whilst the precise temperature to which a composition, food product, beverage product or feedstuff should be heated to produce a food-like flavour and/or aroma, preferably a meat-like flavour and/or aroma, may vary depending on, for example, the precise composition and the time for which the composition is heated and the amount of composition being heated, in some embodiments, the compositions or food products, beverage products or feedstuffs produce a food-like flavour and/or aroma when heated to a temperature of at least about 100° C., for example at least about 110° C., for example at least about 120° C. or at least about 130° C., or at least about 140° C. In particular embodiments, the compositions or food products, beverage products or feedstuffs produce a food-like flavour and/or aroma when heated to about 140° C.

Similarly, the compositions and food products, beverage products or feedstuffs of the present disclosure may produce a food-like flavour and/or aroma, preferably a meat-like flavour and/or aroma when heated for varying amounts of time, depending on, for example, the temperature to which the compositions or food products, beverage products or feedstuffs are heated, the precise nature of the composition, food product, beverage product or feedstuff and the amount of composition, food product, beverage product or feedstuff being heated. Nonetheless, in some embodiments the compositions, food products, beverage products or feedstuffs may produce a food-like flavour and/or aroma when heated for at least 5 or at least 10 minutes, for example at least 15 minutes. In some embodiments, the compositions, food products, beverage products or feedstuffs may produce a food-like flavour and/or aroma when heated for at least about 30 minutes, for example at least about 45 minutes. In some embodiments, the compositions, food products, beverage products or feedstuffs may produce a food like flavour and/or aroma when heated for at least about 1 hour, for example about 1 hour. Preferably, the heat is applied for a length of time whereby a burnt flavour and/or aroma is not produced, as is understood by a person of skill in the art.

In some embodiments, the compositions, food products, beverage products or feedstuffs of the present invention may produce a food-like flavour and/or aroma, preferably a meat-like flavour and/or aroma, when heated for at least 5 or at least 10 minutes at a temperature of at least about 100° C. In some embodiments, the compositions, food products, beverage products or feedstuffs of the present invention may produce a food-like flavour and/or aroma when heated for at least 30 minutes at a temperature of at least about 100° C. In some embodiments, the compositions, food products, beverage products or feedstuffs of the present invention may produce a food-like flavour and/or aroma when heated for at least 30 minutes at a temperature of at least about 120° C. In some embodiments, the compositions, food products, beverage products or feedstuffs of the present invention may produce a food-like flavour and/or aroma when heated for at least 30 minutes at a temperature of at least about 130° C. In some embodiments, the compositions, food products, beverage products or feedstuffs of the present invention may produce a food-like flavour and/or aroma when heated for at least 1 hour at a temperature of at least about 130° C. In some embodiments, the compositions, food products, beverage products or feedstuffs of the present invention may produce a food-like flavour and/or aroma when heated for at least 1 hour at a temperature of at least about 140° C. In some particular embodiments, the compositions, food products, beverage products or feedstuffs may produce a food-like flavour and/or aroma when heated for about 1 hour at about 140° C.

It will be appreciated that compositions, food products, beverage products or feedstuffs of the present invention may, according to some embodiments, produce food-like flavours and/or aromas when heated to temperatures and for time periods different to those outlined above, but that, in some embodiments, stronger and/or more desirable food-like flavours and/or aromas may be produced when the compositions, food products, beverage products or feedstuffs are heated to the temperatures discussed above and/or for the time periods discussed above.

The food-like flavours and/or aromas produced by compositions, food products, beverage products or feedstuffs of the present disclosure may, according to preferred embodiments, include a meat-like flavour and/or aroma. In particular embodiments, the food-like flavour and/or aroma may be an aroma of cooked meat or meat-based foods. For example, the food-like flavour and/or aroma may be of beef, steak, chicken, for example roasted chicken or chicken skin, pork, lamb, duck, venison, chicken or other meat soup, meat broth, liver, or generally “meaty”. In some examples, the meat-like flavour or arma is a chicken (e.g. roast chicken or pan-fried chicken), beef (e.g. roast or pan-fried beef), or pork (e.g. roast or pan-fried pork) flavour or aroma. Such aromas are typically detected by human volunteers, for example by a qualified sensory panel. In this context, a composition, food product, beverage product or feedstuff is said to produce a food-like or meat-like flavour and/or aroma when at least one third, for example at least one half, of the number of volunteers on a tasting/smelling panel detect a food-like or meat-like flavour and/or aroma in a double-blind test of the composition, food product, beverage products or feedstuff. It will be appreciated that, in some instances, there will be a degree of variability in how various flavours and/or aromas are perceived by different subjects experiencing those aromas, and subjects may describe precise flavour and/or aromas slightly differently.

The food-like flavours and/or aromas produced by compositions, food products, beverage products or feedstuffs of the present disclosure may, according to some embodiments, include a fish-like flavour and/or aroma, for example a cooked fish flavour and/or aroma, for example a fried fish flavour and/or aroma.

The food-like flavours and/or aromas produced by compositions, food products, beverage products or feedstuffs of the present disclosure may include a vegetable and/or herbal flavour and/or aroma, for example a cooked vegetable and/or herby flavour and/or aroma, for example a soup, mushroom, onion, vegetable, herbal or roasted vegetable flavour and/or aroma.

In some embodiments, the composition, food product, beverage product or feedstuff includes ribose and the food-like flavour and/or aroma includes a meaty, for example cooked meat-like flavour and/or aroma, and/or a fishy, for example a cooked or fried fish-like flavour and/or aroma.

In some embodiments, volatile compounds indicative of meat-like or meat-associated aromas and flavours, include, for example volatile compounds such as 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane. In some examples, volatile compounds indicative of meat-like or meat-associated aromas and flavours, include 2-heptanone, 3-octanone, 2,3-octanedione, 1-pentanol, 1-hexanol, 2-ethyl-1-hexanol, 1-octanol, trans-2-octen-1-ol and 1-nonanol are produced. In other embodiments, volatile compounds indicative of meat-like or meat-associated aromas and flavours, include 1-pentanal, 3-octanone, 2-octen-1-ol, 1-nonanol and 1-octanol, and optionally 1,3-bis(1,1-dimethylethyl)-benzene are produced.

In some embodiments, the composition, food product, beverage product or feedstuff includes glutamic acid, for example glutamic acid in addition to a further amino acid or salt or derivative thereof such as cysteine, and the food-like flavour and/or aroma includes a meaty, for example cooked meat-like, and/or a fishy, for example a cooked or fried fish-like flavour and/or aroma.

In some embodiments, the composition, food product, beverage product or feedstuff includes a yeast extract and the food-like flavour and/or aroma includes a meaty, for example cooked meat-like flavour and/or aroma. In some embodiments, the composition, food product, beverage product or feedstuff does not include a yeast extract and the food-like flavour and/or aroma includes a fish-like, for example cooked fish or fried fish-like, vegetable and/or herby aroma and/or flavour.

In preferred embodiments, the microorganism is Mortierella spp., for example Mortierella alpina, and the food-like flavour and/or aroma includes a meat-like flavour and/or aroma, for example a chicken-like flavour and/or aroma for example a cooked chicken flavour and/or aroma, for example a roast chicken, chicken skin or chicken broth flavour and/or aroma.

In some embodiments, the microorganism is Mortierella spp., for example Mortierella alpina, Mortierella elongata or Mortierella exigua and the food-like flavour and/or aroma includes a meat-like flavour and/or aroma, such as a beef-like flavour and/or aroma.

In some embodiments, the composition, food product, beverage product or feedstuff includes one or more herbs and/or spices, for example fenugreek, for example fenugreek leaf, and the food-like flavour and/or aroma includes a vegetable, soupy and/or herby flavour and/or aroma.

In particular embodiments, compositions, food products, beverage products or feedstuffs of the present disclosure may produce food-like flavours as well as food-like aromas. Such food-like flavours may be flavours corresponding to the food-like aromas disclosed herein. As such, reference to aromas herein may be understood, according to certain aspects, to also refer to aromas and/or flavours where appropriate.

In some embodiments, the biomass and any extracted lipids comprising phospholipids disclosed herein, or composition of the present invention is incorporated into the food or beverage product or feedstuff prior to or during heating, such that when the food or beverage product is heated (for example during cooking), the biomass and any optional extracted lipids comprising phospholipids disclosed herein or composition produces the associated food-like aromas (by way of Maillard and associated reactions). In some embodiments, the biomass and any optional extracted lipids comprising phospholipids disclosed herein, or composition of the present invention is heated prior to incorporation in or addition to a food or beverage product or feedstuff. In some examples, the biomass and optionally extracted lipid have been heated prior to incorporation into the food, such as in the presence of a sugar and an amino acid or derivative, under conditions suitable to produce one or more (e.g. at least or about 2, 3, 4, 5, 6, 7, 8, 9, 10, 11, 12, 13, 14, 15, 16, 17, 18, 19, 20, 21, 22, 23, 24, 25, 26, 26, 28, 29, 30 or 31) volatile compounds indicative of meat-like or meat-associated aromas and flavours, for example volatile compounds such as 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane. In some examples, one or more (e.g. 2, 3, 4, 5, 6, 7, 8 or 9) volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol are produced. In other embodiments, one or more (e.g. 2, 3, 4 or 5) volatile compound(s) selected from 1-pentanal, 3-octanone, 2-octen-1-ol, 1-nonanol and 1-octanol, and optionally 1,3-bis(1,1-dimethylethyl)-benzene are produced. As would be appreciated, the amounts and ratios of various fatty acids (and in particular the ω6 fatty acids (e.g. ARA, GLA, DGLA, EDA, DTA and/or DPA-ω6) in the biomass and optional extracted microbial lipid will change when one or more of these volatile compounds are produced from the reaction between the fatty acids on the polar lipids, the sugar and the amino acid. Consequently, the lipid in the biomass or the lipid in the optional extracted lipid remaining after the reaction can have a different fatty acid profile compared to the “starting” biomass or extracted microbial lipid. Thus, in some examples, a food, beverage or feedstuff of the invention comprises biomass and optionally lipids wherein the biomass and optionally lipids are a product of a reaction between a microbial biomass (e.g. a Mortierella spp biomass) or extracted microbial lipid, an amino acid or derivative, and a sugar under conditions suitable to produce at least two compounds which have a meat-associated flavour and/or aroma. In particular examples, the conditions include heating, such as at a temperature of at least about 100° C., 110° C., 120° C., 130° C. or 140° C., over a period of time (e.g. as described further below) and with sufficient quantities or concentrations of the sugar and amino acid or derivative to produce the volatile compounds.

In some embodiments, heating the composition, food product, beverage product or feedstuff of the present disclosure results in the production of one or more compound(s) which have a food-like aroma, such as a meat-like aroma, preferably volatile compounds. In some particular embodiments, such heating results in production of a greater amount of said one or more compound(s) than heating a food product, beverage product or feedstuff which does not comprise biomass comprising phospholipids disclosed herein or a composition according to the present disclosure.

In one embodiment, applying heat to the composition, food product, beverage product or feedstuff results in the production of two or more (e.g. at least or about 2, 3, 4, 5, 6, 7, 8, 9, 10, 11, 12, 13, 14, 15, 16, 17, 18, 19, 20, 21, 22, 23, 24, 25, 26, 26, 28, 29, 30 or 31) volatile compound(s) selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane. In embodiments, production of three or more, four or more or five or more of the aforesaid compounds result from the application of heat to the composition, food product, beverage product or feedstuff. In other embodiments, one or more (e.g. 2, 3, 4, 5, 6, 7, 8 or 9) volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol are produced. In other embodiments, one or more (e.g. 2, 3, 4 or 5) volatile compound(s) selected from 1-pentanal, 3-octanone, 2-octen-1-ol, 1-nonanol and 1-octanol, and optionally 1,3-bis(1,1-dimethylethyl)-benzene are produced upon heating the composition, food product, beverage product or feedstuff.

Characteristic flavour and fragrance components are mostly produced during the cooking process by chemical reactions molecules including amino acids, fats and sugars which are found in plants as well as meat. Therefore, in some embodiments, a food product, beverage product or feedstuff is tested for similarity to meat during or after cooking. In some embodiments human ratings, human evaluation, olfactometer readings, or GC-MS measurements, or combinations thereof, are used to create an olfactory map of the food or beverage product or feedstuff, for a meat replica. Similarly, an olfactory map of a comparison product, such as meat, can be created. These maps can be compared to assess how similar the cooked food, beverage or feedstuff is to meat.

The present invention further relates to a method of producing food-like flavour and/or aromas, comprising heating a food or beverage product or feedstuff, comprising biomass and any optional extracted lipids comprising phospholipids as disclosed herein or comprising a composition of the present invention.

The present invention further relates to a method of imparting, or increasing, a food-like flavour and/or aroma to a food product, beverage product or feedstuff comprising contacting the food product, beverage product or feedstuff with biomass and any optional extracted lipids comprising phospholipids as disclosed herein or a composition according to the present invention and heating the food product, beverage product or feedstuff and composition or biomass and any optional extracted lipids comprising phospholipids.

The present invention further relates to a method of preparing a food product, beverage product or feedstuff for consumption, the method comprising heating a food product, beverage product or feedstuff of the invention to produce a food-like flavour and/or aroma, for example meaty or fishy flavour and/or aromas.

The present invention further relates to a method of increasing food-like flavours and/or aromas, especially meaty or fishy flavours and/or aromas, such as meaty flavours and/or aromas associated with a food product, beverage product or feedstuff, comprising heating a food product, beverage product ingredient and a composition according to the present invention or biomass and any optional extracted lipids comprising phospholipids as disclosed herein under conditions sufficient to produce a food-like flavour and/or aroma.

The present invention further relates to a method of increasing food-like flavour and/or aromas, especially meaty or fishy flavour and/or aromas, such as meaty flavour and/or aromas associated with a food product, beverage product or feedstuff, comprising contacting the food product, beverage product or feedstuff with a composition according to the present invention or biomass and any optional extracted lipids comprising phospholipids as disclosed herein and heating under conditions sufficient to produce a food-like flavour and/or aroma. In some embodiments, the composition of the present disclosure or the biomass and any optional extracted lipids comprising phospholipids disclosed herein is added to or incorporated into a food or beverage product or feedstuff before heating, and the food or beverage product or feedstuff including the composition or biomass and any optional extracted lipids comprising phospholipids is subsequently heated to product a food-like flavour and/or aroma.

In some alternative embodiments of the present disclosure, biomass and any optional extracted lipids comprising phospholipids as disclosed herein or the composition of the present disclosure is heated before addition to a food or beverage product or feedstuff. The biomass and any optional extracted lipids comprising phospholipids as disclosed herein or composition of the present disclosure may optionally be allowed to cool after heating and before contacting the food product, beverage product or feedstuff. Accordingly, the present disclosure further provides a method of increasing food-like aromas and/or flavours associated with a food product, beverage product or feedstuff, comprising: a) heating a composition according to any one of claims 6 to 30; and then b) contacting a food product, beverage product or feedstuff with the composition obtained in step a).

It will be appreciated that the precise time period and temperature at which a composition, food or beverage product or feedstuff should be heated to produce a food-like flavour and/or aroma will depend on various factors, including the nature of the composition, the nature of the food or beverage product or feedstuff, and the amount of composition or biomass and any optional extracted lipids comprising phospholipids incorporated in the food or beverage product or feedstuff. Throughout the present specification, “heating” is to be understood as meaning “heating under conditions sufficient to produce a food-like flavour and/or aroma”, Nonetheless, in some embodiments, methods of producing food-like flavour and/or aromas may comprise heating the composition, food or beverage product or feedstuff to a temperature of at least about 100° C., for example at least about 110° C., for example at least about 120° C. or at least about 130° C. In particular embodiments, the methods comprise heating the composition, food or beverage product or feedstuff to a temperature of about 140° C.

According to some embodiments, methods of producing food-like flavour and/or aromas may, according to some embodiments may comprise heating the composition, food or beverage product or feedstuff for at least 10 minutes, for example at least 15 minutes. In some embodiments, methods of producing food-like aromas may comprise heating the composition, food or beverage product or feedstuff for at least about 30 minutes, for example at least about 45 minutes. In some embodiments, methods of producing food-like aromas may comprise heating the composition, food or beverage product or feedstuff for at least about 1 hour, for example about 1 hour.

In some embodiments, methods of producing food-like aromas may comprise heating the composition, food or beverage product or feedstuff for at least 10 minutes at a temperature of at least about 100° C. In some embodiments, methods of producing food-like aromas may comprise heating the composition, food or beverage product or feedstuff for about 1 hour at about 140° C.

### Microorganisms

As used herein, the term “microorganism” refers to an organism that is capable of living and reproducing in a single-celled form. The single cells may clump together or associate with other cells in clusters, or may remain attached to sibling or progeny cells, for example as a hyphal or mycelial form for fungi such as moulds. The terms “microorganism” and “microbial cell” may be used interchangeably herein.

A variety of microorganisms can be used in the present invention, whether as microorganism biomass or as a source of phospholipids. In particular embodiments, the microorganism is suitable for fermentation, although it can also be cultured under ambient oxygen concentrations. In particular embodiments, the microorganism is an oleaginous microorganism, preferably an oleaginous eukaryotic microorganism, or is preferably derived from a progenitor oleaginous microorganism such as a progenitor eukaryotic oleaginous microorganism. In another embodiment, the microorganism is a heterotrophic microorganism, preferably a heterotrophic eukaryotic microorganism. The microorganism may, according to some embodiments, have at least two of these features, or may be characterised by all of these features. The microrganim to be used as a source of biomass in accordance with the present invention may be alive, inactivated or dead, or a combination of live inactivated or dead microbial cells may be used. Microorganisms may be inactivated or killed using any technique well known to those skilled in the art, including, for example, heating, pasteurisation and fermentation.

The microorganism used in accordance with the present invention may be a Mortierella spp. For example, the microorganism may be Mortierella elongata, Mortierella alpina, Mortierella exigua or Mortierella isabellina. Other Mortierella spp. include M. humilis, M. camargensi, M. lignicola, M. zonata, M. sepedonioides, M. stylospora, M. polycephala, M. alliacea, M. claussenii, M. globalpina, M. globulifera, M. pusilla, M. strangulata, M. rostafinskii, M. bainieri, M. beljakovae, M. clonocystis, M. epigama, M. gemmifera, M. hyalina, M. hygrophila, M. kuhlmanii, M. marburgensis, M. minutissima, M. nigrescens, M. sarnyensis, M. sclerotiella, M. selenospora, M. polycephala, M. gamsii, M. nantahalensis, M. oligospora, M. parvispora, M. pulcheria, M. reticulata, M. spinosa, and M. umbellate andM. zychae. In one embodiment, the microorganism is not Mortierella isabellina, which has low or undetectable levels of arachidonic acid. In some preferred embodiments, the microorganism is Mortierella alpina, Mortierella exigua or Mortierella elongata. In particularly preferred embodiments, the microorganism is Mortierella alpina. As demonstrated by the Examples below, M. alpina, M. elongata and M. exigua have been incorporated into a composition which is effective in providing food-like aromas, especially meat-like aromas, for example beefy aromas.

The Mortierella spp. used in the present invention may be a wild-type Mortierella spp., for example wild-type Mortierella alpina. Alternatively, the Mortierella spp. used in the present invention may be a genetically modified Mortierella spp.

The Mortierella spp., or other microorganism used in the present invention as described hereinbelow, includes phospholipids. In some preferred embodiments, the Mortierella spp. biomass (or other microbial biomass) comprises at least about 1%, for example at least about 2% phospholipids by weight (as a percentage of dry cell weight). In some particular embodiments, the biomass comprises at least about 3%, for example at least about 4%, for example about 5% phospholipids or greater. In this context, the total fatty acid content of the phospholipids in the microorganism biomass (e.g. Mortierella spp biomass) and/or extracted lipid comprises at least 10% by weight of ω6 fatty acids excluding linoleic acid (LA), more preferably at least 10% by weight ω6 fatty acids having 20 or 22 carbons in their acyl chains. More preferably, the total fatty acid content of the phospholipids in the microorganism (e.g. Mortierella spp) biomass and/or extracted lipid comprises between 10% and 70%, or between 10% and 60%, or between 20% and 70%, or between 20% and 60%, by weight of ω6 fatty acids excluding linoleic acid (LA), even more preferably between 10% and 70%, or between 10% and 60%, or between 20% and 70%, or between 20% and 60%, by weight of ω6 fatty acids having 20 or 22 carbons in their acyl chains.

The amount of phospholipid contained in a microorganism may be measured by extracting the phospholipids as described hereinbelow, and measuring the amount of phospholipid as a proportion of dry cell weight of the microorganism.

In some alternative aspects, biomass from a microorganism other than Mortierella spp., and/or an extracted lipid from a microorganism other than Mortierella spp., is used instead of Mortierella spp. A variety of microorganisms may be used, whether as microorganism biomass or from which to extract phospholipids. In an embodiment the microorganism is a single-celled organism. Examples of microorganisms which may be used in the present invention include bacterial cells and eukaryotic cells such as fungal cells and algal cells. Eukaryotic microorganisms are preferred over bacterial (prokaryotic) microorganisms. In some particular embodiments, the microorganism may be a yeast, such as, but not limited to, Yarrowia spp. such as Yarrowia lipolytica. In particular examples, the yeast has been genetically engineered to synthesise arachidonic acid or has been cultured in arachidonic acid such that arachidonic acid is present in an amount of at least or about 10%, 20%, 30%, 40% or 50% of the total fatty acid content of the polar lipid of the yeast. Other yeasts that can be engineered or cultured in such a way include, but are not limited to, Pichia spp. such as Pichia pastoris, Candida spp. such as Candida rugosa, Aspergillus spp. such as Aspergillus niger, Cryptococcus spp. such as Cryptococcus curvatus, Lipomyces spp. such as Lipomyces starkeyi, Rhodosporidium spp. such as Rhodosporidium toruloides, Rhodotorula spp. such as Rhodotorula glutinis and Trichosporon spp. such as Trichosporon fermentans.

In other embodiments, the microorganism is a fungus other than a Mortierella spp., and in particular a fungus having arachidonic acid present in an amount of at least or about 10%, 20%, 30%, 40% or 50% of the total fatty acid content of the polar lipid of the yeast. Non-limiting examples of such fungi include Pithium spp., such as Pithium ultimum, Pithium debaryanum, and Pithium insidiosum.

In some embodiments, the microorganism is Yarrowia lipolytica strain W29 or genetically-modified derivatives thereof. As demonstrated by the Examples below, such microorganisms are particularly effective in producing food-like, in particular meaty, aromas.

According to some embodiments, the microorganism is an alga, such as a microalga, or Bacillariophyceae. More particularly, the microorganism is an algae with arachidonic acid esterified in polar lipids, preferably esterified in phospholipids, e.g. where arachidonic acid is present in an amount of at least or about 10%, 20%, 30%, 40% or 50% of the total fatty acid content of the polar lipid. Non-limiting examples of such algae include Porphyridium purpureum, Euglena gracilis, Parietochloris incisa, Pavlova lutheri, Porphyridium cruentum, Ceramium rubrum and Rodomella subfusca.

In particular embodiments, the microrganisms utilised in the present invention, such as Mortierella spp, comprise arachidonic acid. In particular embodiments, the arachidonic acid is esterified in polar lipids, preferably esterified in phospholipids. In some examples, the microorganism, such as the Mortierella spp, comprises arachidonic acid esterified in polar lipids, preferably esterified in phospholipids, where arachidonic acid is present in an amount of at least or about 10%, 15%, 20%, 25%, 30%, 35%, 40%, 45% or 50% of the total fatty acid content of the polar lipid. In some examples, arachidonic acid is present in an amount of about 10% to about 60% (e.g. 20% to 50%), or is present as at least about 10%, at least about 15%, at least about 20%, at least about 25%, at least about 30%, at least about 35%, at least about 40%, at least about 45%, at least about 50% or at least about 55%) of the total fatty acid content of the polar lipid. Optionally, other ω6 fatty acids, such as γ-linolenic acid (GLA), dihomo-γ-linolenic acid (DGLA), eicosadienoic acid (EDA), docosatetraenoic acid (DTA) and/or docosapentaenoic acid-ω6 (DPA-ω6), are also present in the polar lipid. In some examples, DGLA is present in an amount of at least 0.1% (e.g. at least 0.2%, 0.5%, 1%, 1.5%, 2%, or 2.5%), or about 0.1% to about 5%, of the total fatty acid content of the polar lipid and GLA is present in an amount of at least 1% (e.g. at least 2%, 3%, 4%, 5% or 6%), or about 1% to about 10%, of the total fatty acid content of the polar lipid.

The microorganisms used in the present invention, typically Mortierella spp., may be prepared by any suitable culture process and conditions. Effective culture conditions are known to those skilled in the art and include, but are not limited to, suitable media, bioreactor, temperature, pH and oxygen conditions that permit desirable phospholipid production. A suitable medium refers to any medium in which a cell is cultured to produce microorganisms as defined herein. Such medium typically comprises an aqueous medium having assimilable carbon, nitrogen and phosphate sources, and appropriate salts, minerals, metals and other nutrients, such as vitamins. Cells defined herein can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

In some embodiments, the microorganism (e.g. Mortierella spp.) has been cultured under conditions which increase or optimise the amount of phospholipid contained therein and/or increase or optimise the amount of ω6 fatty acids esterified in said phospholipids.

In some embodiments, the microorganism has been cultured by a process comprising feeding with an ω6 fatty acid, to enhance the amount of said ω6 fatty acid incorporated into phospholipids in the microorganism. For example, the microorganism, such as Yarrowia spp (e.g. Yarrowia lipolytica) may be cultured by a culturing process, for example a fermentation process, comprising introducing a feed of arachidonic acid (as demonstrated in the examples). Feeding is typically carried out by culturing cells in a medium comprising the ω6 fatty acid, for example one or more of LA, GLA, DGLA, EDA, ARA, DTA or DPAo6. In some embodiments, the feed ω6 fatty acids are free fatty acids or fatty acid salts.

In some alternative embodiments, the microorganism biomass or microorganism from which the extracted lipid is extracted may be Yarrowia lipolytica, for example strain W29, and may be prepared by a culturing process, in particular a fermentation process comprising feeding with arachidonic acid.

In some embodiments, the compositions, food products, beverage products and feedstuffs of the present disclosure comprise biomass of two or more different microorganisms, for example two Mortierella species, or a Mortierella spp and another microorganism.

The present invention involves the use of microorganism biomass, such that compositions, food products, beverage products and feedstuffs of the present invention comprise microorganism biomass. The microorganisms, typically Mortierella spp., may be present as dry biomass or wet biomass (i.e. biomass that retains some moisture and has not been substantially or completely dried of water; typically, biomass containing less than about 10% water by weight may be considered “dry”, whereas biomass containing more than 10% moisture, for example about 70% or more water by weight may be considered “wet”. In typical embodiments, “dry” biomass may be approximately 25% of the mass of “wet” biomass). In the present context and as will be understood in the art “biomass” refers to matter containing at least some whole cells of the microorganisms, rather than only components which have been separated therefrom, but may contain both whole cells and cell components. Microorganisms/biomass, such as obtained by a fermentation process, may have been processed by, for example, washing, drying, heat inactivation, freezing and/or freeze drying, but will still contain at least some, preferably most, of the whole cell material of the microorganism. Biomass may be referred to as “whole cell biomass”, but it will be appreciated that the microorganism cells contained in compositions of the present invention may be present in a disrupted form, for example having undergone physical or chemical lysis; the biomass/microorganism will still contain substantially all of the cell material. “Biomass” and “microorganism” do not refer to, for example, oils or proteins extracted or isolated from microorganisms and separated from the other components of the cells. As demonstrated by the Examples below, compositions comprising microorganisms comprising phospholipids (i.e. microorganism biomass) have been found to be particularly effective in producing an enhanced food-like aroma such as meaty or fishy aromas when heated.

The microorganism included in compositions of the present disclosure may be in suspension, frozen, dried or any other suitable form. The microorganism cells may be alive or dead, or a mix of living and dead cells, for example at least 99% of the cells being dead. The cells may have been heat-treated in order to render them incapable of replicating.

### Phospholipids

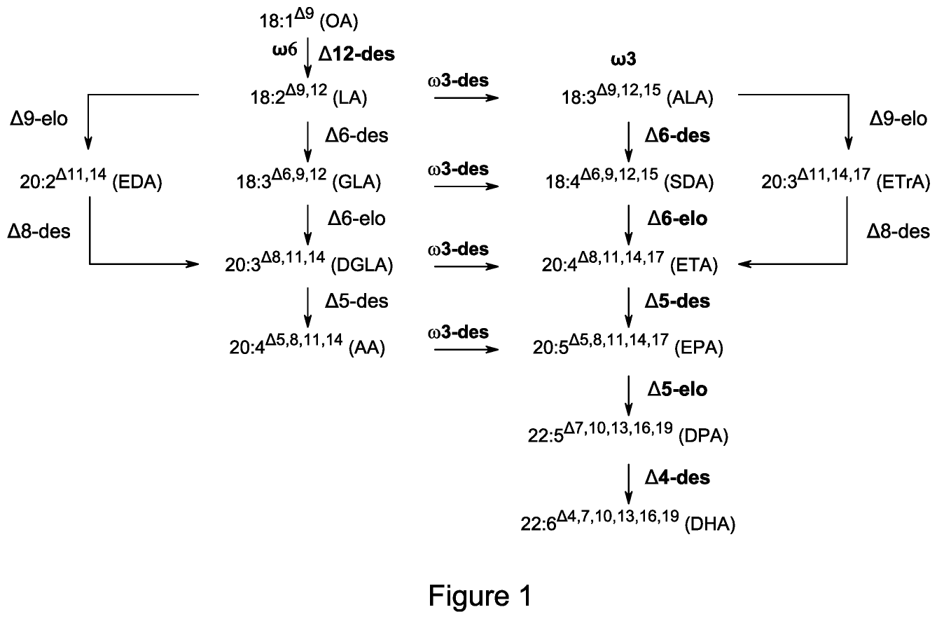

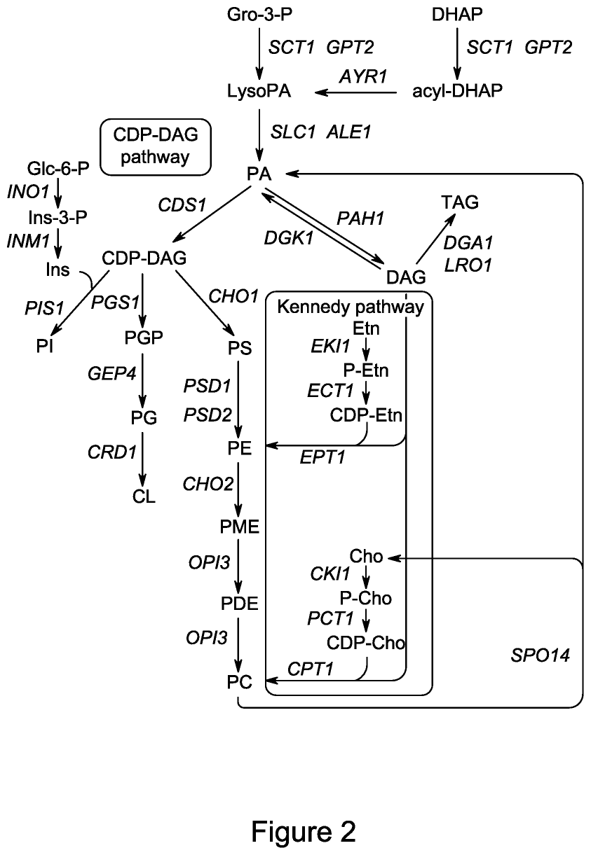

Phospholipids are amphipathic molecules, having a hydrophilic head and a hydrophobic tail, comprising a glycerol backbone esterified to a phosphate “head” group and two fatty acids which provide the hydrophobic tail. According to particularly preferred embodiments, the phospholipids of the present invention (whether as part of a microorganism or extracted from a microorganism) comprise one or more esterified ω6 fatty acids. Biosynthesis of ω6 fatty acids in organisms such as microalgae, mosses and fungi usually occurs as a series of oxygen-dependent desaturation and elongation reactions (FIG. 1).

Examples of ω6 fatty acid include, but are not limited to, arachidonic acid (ARA, C20:4Δ5,8,11,14; ω6), dihomo-gammalinolenic acid (DGLA, C20:3Δ8,11,14; ω6), eicosadienoic acid (EDA, C20:2Δ11,14; ω6), docosatetraenoic acid (DTA, C22:4Δ7,10,13,16; ω6), docosapentaenoic acid-ω6 (DPA-ω6, C22:5Δ4,7,10,13,16; ω6), γ-linolenic acid (GLA, C18:3Δ6,9,12; ω6) and linoleic acid (LA, C18:2Δ9,12; ω6). According to some preferred embodiments, the phospholipids comprise esterified arachidonic acid (ARA, C20:4Δ5,8,11,14; ω6). According to some embodiments, phospholipids comprise esterified docosapentaenoic acid-ω6 (DPA-ω6, C22:5Δ4,7,10,13,16; ω6). According to some embodiments, the phospholipids comprise one or more esterified ω6 fatty acids other than linoleic acid (LA, C18:2Δ9,12; ω6).

According to some embodiments, the ω6 fatty acids comprise arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA). In some embodiments, the ω6 fatty acids comprise two, three or four of arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA).

According to some embodiments, the ω6 fatty acids comprise one or two or all three of eicosadienoic acid (EDA), docosatetraenoic acid (DTA) and docosapentaenoic acid-ω6 (DPA-ω6).

According to particularly preferred embodiments, the ω6 fatty acids comprise arachidonic acid (ARA), or ARA is the predominant ω6 fatty acid in the phospholipid.

In some examples, ARA is present in an amount of about 10% to about 60% of the total fatty acid content of the polar lipid, DGLA is present in an amount of about 0.1% to about 5% of the total fatty acid content of the polar lipid and GLA is present in an amount of about 1% to about 10% of the total fatty acid content of the polar lipid. In other examples, ARA is present in an amount of about 20% to about 50% of the total fatty acid content of the polar lipid, DGLA is present in an amount of about 1% to about 5% of the total fatty acid content of the polar lipid and GLA is present in an amount of about 3% to about 10% of the total fatty acid content of the polar lipid.

According to some preferred embodiments, the phospholipid contains at least about 5% ω6 fatty acids, for example at least about 7%, for example at least about 10%, for example at least about 12%, for example at least about 15%, for example at least about 17%, for example at least about 20% by weight, each as a weight percentage of the total fatty acid content of the phospholipid. In some embodiments, the phospholipid contains at least about 30%, for example at least about 40%, for example at least about 50% ω6 fatty acids.

In some embodiments, amounts of ω6 fatty acids refers to ω6 fatty acids excluding linoleic acid (LA, C18:2Δ9,12; ω6).

According to some embodiments, the sum of the amounts of ARA, DGLA, EDA, DTA, DPA-ω6 and GLA, each as a weight percentage of the total fatty acid content of the phospholipid, in the total fatty acid content of the phospholipids in the microorganism (e.g. Mortierella spp.) biomass and/or the extracted lipid is at least about 5%, for example at least about 10% by weight of the TFA content of the phospholipids. In some embodiments, the sum of the amounts of ARA, DGLA, EDA, DTA, DPA-ω6 and GLA in the phospholipids is between about 10% and about 70%, or between about 10% and about 75% or between about 10% and about 80% by weight of the total fatty acid content of the phospholipid. These amounts of the ω6 fatty acids in the phospholipids of the microorganism or extracted lipid may also apply to the TAG in the microorganism or extracted lipid.

According to some embodiments, the phospholipids comprise at least two, preferably three or all four, of phosphatidylcholine (PC), phosphatidylethanolamine (PE), phosphatidylinositol (PI) and phosphatidylserine (PS), each comprising one or more of ARA, DGLA, EDA, DTA, DPA-ω6 and GLA, and optionally one or more of phosphatidic acid (PA), phosphatidylglycerol (PG) and cardiolipin (Car), each comprising one or more of ARA, DGLA, EDA, DTA, DPA-ω6 and GLA. PC, PE, PI and PS content of phospholipids may be determined by two-dimensional thin layer chromatography (TLC) analysis using two solvent systems as described in Zhou et al (2014), ‘Lipidomic analysis of Arabidopsis seed genetically engineered to contain DHA’, Frontiers in Plant Science, 5, 419 (https://doi.org/10.3389/fpls.2014.00419).

In some embodiments, the content of ω6 fatty acids in the phospholipid which are (i) C20 or C22 fatty acids is about 5% to about 60%, preferably about 10% to about 60% of the total fatty acid content of the phospholipid, and/or (ii) ω6 fatty acids which have 3, 4 or 5 carbon-carbon double bonds, is about 5% to about 70%, preferably about 10% to about 70%, more preferably about 40% to about 70% or about 45% to about 70% or about 50% to about 70% of the total fatty acid content of the phospholipid.

According to some preferred embodiments, the phospholipid contains at least about 10%, for example at least about 15%, for example at least about 20%, for example at least about 25%, for example at least about 30%, for example at least about 35%, for example at least about 40%, for example at least about 45%, for example at least about 50% arachidonic acid (ARA) by weight. In some embodiments, the phospholipid contains at least about 20% of ARA by weight.

For the embodiments referred to herein, the amounts of individual fatty acids in a total fatty acid content in a microorganism sample or a lipid sample is preferably determined by GC analysis of fatty acid methyl esters (FAME) as described in Example 1.

In some embodiments the phospholipids form part of a polar lipid (whether contained within microorganism (e.g. Mortierella spp.) biomass or extracted from a microorganism as an extracted polar lipid or broader lipid), which may comprise, consist essentially of or consist of phospholipids, wherein:
(a) the polar lipid comprises a total fatty acid (TFA) content which comprises ω6 fatty acids, wherein at least some of the ω6 fatty acids are esterified in the form of phospholipids in the polar lipid, and wherein the ω6 fatty acids comprise two, three, four or more fatty acids selected from the group consisting of arachidonic acid (ARA), dihomo-gammalinolenic acid (DGLA), eicosadienoic acid (EDA), docosatetraenoic acid (DTA), docosapentaenoic acid-ω6 (DPA-ω6) and γ-linolenic acid (GLA),
(b) the phospholipids in the polar lipid comprise at least two, preferably three or all four, of phosphatidylcholine (PC), phosphatidylethanolamine (PE), phosphatidylinositol (PI) and phosphatidylserine (PS), each comprising one or more of ARA, DGLA, EDA, DTA, DPA-ω6 and GLA, and optionally one or more of phosphatidic acid (PA), phosphatidylglycerol (PG) and cardiolipin (Car), each comprising one or more of ARA, DGLA, EDA, DTA, DPA-ω6 and GLA,
(c) the polar lipid comprises a total saturated fatty acid content comprising palmitic acid and stearic acid,
(d) the polar lipid comprises a total monounsaturated fatty acid content comprising oleic acid and palmitoleic acid (C16:1Δ9cis), and
(e) ω3 fatty acids are either absent from the polar lipid or are present in a total amount of less than about 3% by weight of the TFA content of the polar lipid, and/or wherein the polar lipid lacks C16:2, C16:3ω3, EPA and DHA.

In some embodiments the phospholipids form part of a polar lipid (whether contained within microorganism biomass or extracted from a microorganism as an extracted polar lipid or broader lipid), which may comprise, consist essentially of or consist of phospholipids, wherein:
(a) the polar lipid comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA), or any combination thereof,
(b) the phospholipids in the polar lipid comprise phosphatidylcholine (PC), phosphatidylethanolamine (PE), phosphatidylinositol (PI) and phosphatidylserine (PS), each comprising one or more of ARA, DGLA, EDA, DTA, DPA-ω6 and GLA, and optionally one or more of phosphatidic acid (PA), phosphatidylglycerol (PG) and cardiolipin (Car), each comprising one or more of ARA, DGLA, EDA, DTA, DPA-ω6 and GLA,
(c) the polar lipid comprises a total saturated fatty acid content comprising palmitic acid and stearic acid, and
(d) the polar lipid comprises a total monounsaturated fatty acid content comprising oleic acid and palmitoleic acid (C16:1Δ9cis).

In some embodiments the phospholipids form part of a polar lipid (whether contained within microorganism biomass or extracted from a microorganism as an extracted polar lipid or broader lipid), which may comprise, consist essentially of or consist of phospholipids, wherein:
(a) the polar lipid comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA), or any combination thereof,
(b) the polar lipid comprises a total saturated fatty acid content comprising palmitic acid and stearic acid,
(c) the polar lipid comprises a total monounsaturated fatty acid content comprising oleic acid and palmitoleic acid (C16:1Δ9cis),
(d) ω3 fatty acids are either absent from the polar lipid or are present in a total amount of less than about 3% by weight of the TFA content of the polar lipid, and/or wherein the polar lipid lacks C16:2, C16:3 ω3, EPA and DHA.

In some embodiments the phospholipids form part of a polar lipid (whether contained within microorganism biomass or extracted from a microorganism as an extracted polar lipid or broader lipid), which may comprise, consist essentially of or consist of phospholipids, wherein:
(a) the polar lipid comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA), or any combination thereof,
(b) the phospholipids in the polar lipid comprise phosphatidylcholine (PC), phosphatidylethanolamine (PE), phosphatidylinositol (PI) and phosphatidylserine (PS), each comprising one or more of ARA, DGLA, EDA, DTA, DPA-ω6 and GLA, and optionally one or more of phosphatidic acid (PA), phosphatidylglycerol (PG) and cardiolipin (Car), each comprising one or more of ARA, DGLA, EDA, DTA, DPA-ω6 and GLA,
(c) the polar lipid comprises a total saturated fatty acid content comprising palmitic acid and stearic acid, and
(d) the polar lipid comprises a total monounsaturated fatty acid content comprising oleic acid and palmitoleic acid (C16:1Δ9cis).

In some embodiments the phospholipids form part of a polar lipid (whether contained within microorganism biomass or extracted from a microorganism as an extracted polar lipid or broader lipid), which may comprise, consist essentially of or consist of phospholipids, wherein:
(a) the polar lipid comprises a total fatty acid (TFA) content which comprises ω6 fatty acids, wherein at least some of the ω6 fatty acids are esterified in the form of phospholipids in the polar lipid, and wherein the ω6 fatty acids comprise one or two or all three of eicosadienoic acid (EDA), docosatetraenoic acid (DTA) and docosapentaenoic acid-ω6 (DPA-ω6),
(b) γ-linolenic acid (GLA) is either absent from the polar lipid or is present in the polar lipid,
(c) the polar lipid comprises a total saturated fatty acid content comprising palmitic acid and stearic acid, and
(d) the polar lipid comprises a total monounsaturated fatty acid content comprising oleic acid and palmitoleic acid (C16:1Δ9cis).

In some embodiments the phospholipids form part of a polar lipid (whether contained within microorganism biomass or extracted from a microorganism as an extracted polar lipid or broader lipid), which may comprise, consist essentially of or consist of phospholipids, wherein:
(a) the polar lipid comprises a total fatty acid (TFA) content which comprises ω6 fatty acids, wherein at least some of the ω6 fatty acids are esterified in the form of phospholipids in the polar lipid, and wherein the ω6 fatty acids comprise two, three, four or more fatty acids selected from the group consisting of arachidonic acid (ARA), dihomo-gammalinolenic acid (DGLA), eicosadienoic acid (EDA), docosatetraenoic acid (DTA), docosapentaenoic acid-ω6 (DPA-ω6) and γ-linolenic acid (GLA),
(b) the polar lipid comprises a total saturated fatty acid content comprising palmitic acid and stearic acid,
(c) the polar lipid comprises a total monounsaturated fatty acid content comprising oleic acid and palmitoleic acid (C16:1Δ9cis), and
(d) the polar lipid lacks C16:2, C16:3ω3, EPA and DHA.

In some embodiments the phospholipids form part of a polar lipid (whether contained within microorganism biomass or extracted from a microorganism as an extracted polar lipid or broader lipid), which may comprise, consist essentially of or consist of phospholipids, wherein:
(a) the polar lipid comprises a total fatty acid (TFA) content which comprises ω6 fatty acids, wherein at least some of the ω6 fatty acids are esterified in the form of phospholipids in the polar lipid, and wherein the ω6 fatty acids of the polar lipid comprise an amount of arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA), or any combination thereof, each amount being expressed as a weight percentage of the total fatty acid content of the polar lipid, whereby the sum of the amounts of ARA, DGLA, EDA, DTA, DPA-ω6 and GLA is at least about 10%,
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis).

In some embodiments the phospholipids form part of a polar lipid (whether contained within microorganism biomass or extracted from a microorganism as an extracted polar lipid or broader lipid), which may comprise, consist essentially of or consist of phospholipids, wherein:
(a) the polar lipid comprises a total fatty acid (TFA) content which comprises the ω6 fatty acids, wherein at least some of the ω6 fatty acids are esterified in the form of phospholipids in the polar lipid, and wherein the ω6 fatty acids of the polar lipid comprise an amount of arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA), or any combination thereof, whereby the sum of the amounts of ARA, DGLA, EDA, DTA, DPA-ω6 and GLA is preferably at least about 5%, more preferably at least about 10%, by weight of the TFA content of the polar lipid,
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis).

In some embodiments the phospholipids form part of a polar lipid (whether contained within microorganism biomass or extracted from a microorganism as an extracted polar lipid or broader lipid), which may comprise, consist essentially of or consist of phospholipids, wherein:
(a) the polar lipid comprises a total fatty acid (TFA) content which comprises the ω6 fatty acids, wherein at least some of the ω6 fatty acids are esterified in the form of phospholipids in the polar lipid, and wherein the ω6 fatty acids comprise one, two, three, four or more fatty acids selected from the group consisting of arachidonic acid (ARA), dihomo-gammalinolenic acid (DGLA), eicosadienoic acid (EDA), docosatetraenoic acid (DTA), docosapentaenoic acid-ω6 (DPA-ω6) and γ-linolenic acid (GLA),
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis).

In one embodiment, if the polar lipid comprises DPA-ω6, one or more or all of GLA, DGLA, EDA, ARA and DTA are also present.

In one embodiment, the polar lipid comprises EDA and one, two or all three of arachidonic acid (ARA), dihomo-gammalinolenic acid (DGLA) and γ-linolenic acid (GLA) esterified in the polar lipid, and wherein the level of EDA in the polar lipid is at least about 1% of the total fatty acid content of the polar lipid.

In one embodiment, the polar lipid lacks one, two, three or all four of C16:2, C16:3ω3, EPA and DHA. In a preferred embodiment, the polar lipid lacks C16:3ω3, EPA and DHA. In a further embodiment, the polar lipid also lacks ALA or has less than 1% ALA.

In one embodiment, the extracted lipid comprises three, four or more fatty acids selected from the group consisting of ARA, DGLA, EDA, DTA, DPA-ω6 and GLA, such as a combination of ARA, DGLA and GLA, or a combination of fatty acids other than ARA, DGLA and GLA, preferably a combination of ARA, DGLA, GLA and at least one of EDA, DTA and DPA-ω6. In an embodiment, the sum total of the amounts of ARA, DGLA, EDA, DTA, DPA-ω6 and GLA is between about 10% and about 70%, or between about 10% and about 75% or between about 10% and about 80%, each amount being expressed as a percentage of the total fatty acid content of the polar lipid. In an embodiment, the ω6 fatty acid that is present in the greatest amount in the total fatty acid content of the polar lipid is not LA, or not ARA. In an embodiment, if the ω6 fatty acid that is present in the greatest amount is GLA or DGLA, the polar lipid comprises one or more of EDA, DTA or DPA-ω6.

In one embodiment, the phospholipids comprise at least two, at least three or all four of phosphatidylcholine (PC), phosphatidylethanolamine (PE), phosphatidylinositol (PI) and phosphatidylserine (PS), each comprising one, two, three or more than three of ARA, DGLA, EDA, DTA, DPA-ω6 and GLA, and optionally one or more or all of phosphatidic acid (PA), phosphatidylglycerol (PG) and cardiolipin (Car), each comprising one, two, three or more than three of ARA, DGLA, EDA, DTA, DPA-ω6 and GLA.

In one embodiment, the polar lipid comprises myristic acid (C14:0) in an amount of less than about 2% by weight of the total fatty acid content of the polar lipid. In a preferred embodiment, the polar lipid comprises myristic acid (C14:0) in an amount of less than about 1% by weight of the total fatty acid content of the polar lipid.

In some embodiments, stearic acid is present at a level of less than about 14% or less than about 12% or less than about 10% of the total fatty acid content of the polar lipid. In preferred embodiments, stearic acid is present at a level of less than about 7% or less than about 6% or less than about 5%, preferably less than 4% or less than 3%, of the total fatty acid content of the polar lipid.

In some embodiments, ARA is present in an amount of about 10% to about 60%, about 10% to about 30%, about 10% to about 25%, about 15% to about 60%, about 20% to about 60%, or about 30% to about 60%, by weight of the TFA content of the polar lipid. In preferred embodiments, ARA is present in an amount of about 20% to about 60%, or about 30% to about 60%, or about 40% to about 60%, or about 50% to about 60%, by weight of the TFA content of the polar lipid. In some embodiments, ARA is present in an amount of at least or about 10%, 15%, 20%, 25% or 30% by weight of the TFA content of the polar lipid.

In one embodiment, the polar lipid comprises one or more or all of EDA, DTA and DPA-ω6.

In one embodiment, if the polar lipid comprises DGLA and ARA, or GLA, DGLA and ARA, then at least one of the following apply:
(a) at least one of EDA, DTA and DPA-ω3 is also present in the polar lipid; and
(b) the ratio of PC to PE or to phospholipids other than PC is less than 3:1, less than 2:1, less than 1.5:1, less than 1.25:1, less than 1:1, between 3:1 and 1:1, between 2:1 and 1:1, or between 3:1 and 0.5:1.

In one embodiment, GLA is present in the polar lipid in an amount which is (i) less than the sum of the amounts of ARA, DGLA, EDA, DTA and DPA-ω6 in the polar lipid, or (ii) one or more of: less than the amount of ARA, less than the amount of DGLA, less than the amount of EDA, less than the amount of DTA and less than the amount of DPA-ω6, or any combination thereof, in the polar lipid.

In some embodiments, the saturated fatty acid content of the polar lipid comprises one or more or all of lauric acid (C12:0), myristic acid (C14:0), a C15:0 fatty acid, C20:0, C22:0 and C24:0, preferably comprising C14:0 and C24:0 or C14:0, C15:0 and C24:0, more preferably comprising C14:0, C15:0 and C24:0 but not C20:0 and C22:0.

In some embodiments, lauric acid and myristic acid are absent from the polar lipid, or lauric acid and/or myristic acid is present in the polar lipid, whereby the sum of the amounts of lauric acid and myristic acid in the polar lipid is less than about 2%, or less than about 1%, preferably less than about 0.5%, more preferably less than about 0.2%, of the total fatty acid content of the polar lipid.

In some embodiments, C15:0 is absent from the polar lipid, or C15:0 is present in the polar lipid in an amount of less than about 3%, preferably less than about 2% or less than about 1%, of the total fatty acid content of the polar lipid.

In some embodiments, palmitic acid is present in the polar lipid in an amount of about 3% to about 45%, or about 10% to about 40%, or about 20% to about 45%, of the total fatty acid content of the polar lipid.

In some embodiments, palmitoleic acid is present in the polar lipid in an amount of about 3% to about 45%, or about 3% to about 25%, or about 3% to about 20%, or about 3% to about 15%, of the total fatty acid content of the polar lipid.

In some embodiments, oleic acid is present in the polar lipid in an amount of about 3% to about 60%, or about 3% to about 40%, or about 3% to about 25%, or about 20% to about 60%, of the total fatty acid content of the polar lipid.

In some embodiments, vaccenic acid is absent from the polar lipid, or vaccenic acid is present in the polar lipid in an amount of less than about 2%, preferably less than about 1% or about 0.5%, of the total fatty acid content of the polar lipid.

In some embodiments, linoleic acid is present in the polar lipid in an amount of about 3% to about 45%, or about 3% to about 30%, or about 3% to about 20%, of the total fatty acid content of the polar lipid.

In some embodiments, γ-linoleic acid is absent from the polar lipid, or γ-linoleic acid is present in the polar lipid in an amount of about 3% to about 12%, or about 3% to about 8%, or about 3% to about 6%, or less than about 3% of the total fatty acid content of the polar lipid.

In some embodiments, eicosadienoic acid is absent from the polar lipid, or eicosadienoic acid is present in the polar lipid in an amount of about 3% to about 12%, or about 3% to about 8%, or about 3% to about 6%, or less than about 3% of the total fatty acid content of the polar lipid.

In some embodiments, dihomo-gammalinolenic acid is absent from the polar lipid, or dihomo-gammalinolenic acid is present in the polar lipid, preferably in an amount of less than about 2%, 0.1% to about 2%, or about 10% to about 60%, of the total fatty acid content of the polar lipid.

In some embodiments, C20:0 and C22:0 are absent from the polar lipid, or C20:0 and/or C22:0 is present in the polar lipid, whereby the sum of the amounts of C20:0 and C22:0 in the polar lipid is less than about 1.0% or less than about 0.5%, preferably less than 0.2%, of the total fatty acid content of the polar lipid.

In some embodiments, C24:0 is absent from the polar lipid, or C24:0 is present in the polar lipid in an amount of less than about 1.0%, less than about 0.5%, preferably less than 0.3% or less than 0.2%, of the total fatty acid content of the polar lipid.

In some embodiments, C17:1 is absent from the polar lipid, or C17:1 is present in the polar lipid in an amount of less than about 5%, preferably less than about 4% or less than about 3%, more preferably less than about 2% of the total fatty acid content of the polar lipid.

In some embodiments, monounsaturated fatty acids which are C20 or C22 fatty acids are absent from the polar lipid, or C20:1 and/or C22:1 is present in the polar lipid, whereby the sum of the amounts of C20:1 and C22:1 in the polar lipid is less than about 1.0%, less than about 0.5%, preferably less than 0.2%, of the total fatty acid content of the polar lipid.

In some embodiments, the content of ω6 fatty acids in the polar lipid which are (i) C20 or C22 fatty acids is about 5% to about 60%, preferably about 10% to about 60% of the total fatty acid content of the polar lipid, and/or (ii) ω6 fatty acids which have 3, 4 or 5 carbon-carbon double bonds, is about 5% to about 70%, preferably about 10% to about 70%, more preferably about 40% to about 70% or about 45% to about 70% or about 50% to about 70% of the total fatty acid content of the polar lipid.

In some embodiments, C16:3ω3 is absent from the polar lipid, or both C16:2 and C16:3 ω3 are absent from the polar lipid.

In some embodiments, the polar lipid or broader extracted microbial lipid comprises PC and/or lacks cyclopropane fatty acids, preferably lacks C15:0c, C17:0c and C19:0c.

The ω6 fatty acid content of phospholipids/polar lipids may be measured, for example, by lipid derivatisation to fatty acid methyl esters (FAME) and subsequent gas chromatography (GC) analysis, as described in Example 1 below.

### Phospholipid Extraction

In addition to microorganism biomass, extracted lipids from such a microorganism comprising phospholipids may be present in compositions, food products, beverage products or feedstuffs in accordance with the present invention. Said extracted lipids are, in accordance with preferred embodiments, typically extracted from Mortierella spp.. The extracted lipid may comprise only polar lipids, for example only phospholipids, or may comprise other lipid fractions. For example the extracted lipid may comprise non-polar lipids in addition to extracted phospholipids, such as TAG, DAG and MAG, or free fatty acids, or any combination thereof. The extracted lipid may comprise phospholipids in isolation, in a vehicle/carrier, and/or as part of a broader extracted lipid, for example a polar lipid fraction, extracted from said microorganism, which may include polar lipids other than phospholipids, for example cephalins, sphingolipids (sphingomyelins and glycosphingolipids), phosphatidic acid, cardiolipin and/or glycoglycerolipids. In embodiments, the extracted lipid in which the phospholipids are present comprises one or more sterols such as, for example from yeast cells, ergosterol and/or ergosterol esters. In some embodiments, the phospholipids are present in a broader extracted lipid comprising polar lipids, and optionally comprising non-polar lipids, wherein, in some embodiments, if present the non-polar lipids are present in the extracted lipid in a lower amount than the polar lipids.

Lipids may be extracted from microorganisms such as Mortierella spp. for use in the present invention according to any suitable process known to a person skilled in the art. Exemplary methods of such extraction are disclosed in Example 1 below. Extraction of the phospholipid from microorganisms disclosed herein, including as a component of a broader lipid fraction, may use analogous methods to those known in the art for lipid extraction from oleaginous microorganisms, such as for example described in Patel et al. (2018) Molecules 23:1562. For example, extraction may be performed by solvent extraction where an organic solvent (e.g., hexane or a mixture of hexane and ethanol, chloroform and/or a mixture of chloroform and methanol) is mixed with at least the biomass of the microorganism, preferably after the biomass is dried and ground, but it can also be performed under wet conditions. The solvent dissolves the lipid in the cells, which solution may then be separated from the biomass by a physical action (e.g., ultrasonication). Ultrasonication is one of the most extensively used pretreatment methods to disrupt the cellular integrity of microbial cells. Other pretreatment methods can include microwave irradiation, high-speed homogenization, high-pressure homogenization, bead beating, autoclaving, and thermolysis. The solvent/lipid solution may be separated from the biomass by, for example, filtration (e.g., with a filter press or similar device) or centrifugation etc. The organic solvent can then be separated from the non-polar lipid (e.g., by distillation). This second separation step yields non-polar lipid from the cells and can yield a re-usable solvent if conventional vapor recovery is employed.

Phospholipids may be separated from a broader lipid fraction extracted from microorganisms by any suitable method, for example by use of solvent extraction as described in Example 2 below. For example, lipids may be extracted from a lipid source by dissolving in ethanol or another alcohol such as isopropanol, evaporating the ethanol or other alcohol, and phospholipids then further separated from neutral lipids by precipitation of phospholipids from cold acetone.

Lipid extracted from the microbial cells may be subjected to normal oil processing procedures. As used herein, the term “purified” when used in connection with lipids disclosed herein means that that the extracted lipid has been subjected to one or more processing steps of increase the purity of the lipid component. For example, a purification step may comprise one or more or all of the group consisting of: degumming, deodorising, decolourising, drying and/or fractionating the extracted oil, as described below. However, as used herein, the term “purified” does not include a transesterification process or other process which alters the fatty acid composition of the lipid or oil of the invention so as to change the fatty acid composition of the total fatty acid content. Expressed in other words, in a preferred embodiment the fatty acid composition of the purified lipid is essentially the same as that of the unpurified lipid.

Degumming is an early step in the refining of lipids in a liquid form (oil) and its primary purpose is the separation of most of the phospholipids from the oil, which may be present as approximately 1-2% of the total extracted lipid. Addition of ˜2% of water, typically containing phosphoric acid, at 70-80° C. to the crude oil results in the separation of most of the phospholipids accompanied by trace metals and pigments. The insoluble material that is removed is mainly a mixture of phospholipids and is also known as lecithin. Degumming can be performed by addition of concentrated phosphoric acid to a crude extracted lipid to convert non-hydratable phosphatides to a hydratable form, and to chelate minor metals that are present. Gum is separated from the oil by centrifugation. If the purified phospholipids are the desired end product, the insoluble material containing the phospholipids may be dried such as, for example, by spray drying.

Alkali refining is one of the refining processes for treating lipid in the form of an oil, sometimes also referred to as neutralization. It usually follows degumming and precedes bleaching. Following degumming, the oil can be treated by the addition of a sufficient amount of an alkali solution to titrate all of the fatty acids and phosphoric acids, and removing the soaps thus formed. Suitable alkaline materials include sodium hydroxide, potassium hydroxide, sodium carbonate, lithium hydroxide, calcium hydroxide, calcium carbonate and ammonium hydroxide. This process is typically carried out at room temperature and removes the free fatty acid fraction. Soap is removed by centrifugation or by extraction into a solvent for the soap, and the neutralised oil is washed with water. If required, any excess alkali in the oil may be neutralized with a suitable acid such as hydrochloric acid or sulphuric acid.

Bleaching is a refining process in which oils are heated at 90-120° C. for 10-30 minutes in the presence of a bleaching earth (0.2-2.0%) and in the absence of oxygen by operating with nitrogen or steam or in a vacuum. This step in oil processing is designed to remove unwanted pigments and the process also removes oxidation products, trace metals, sulphur compounds and traces of soap.

Deodorization is a treatment of oils and fats at a high temperature (200-260° C.) and low pressure (0.1-1 mm Hg). This is typically achieved by introducing steam into the oil at a rate of about 0.1 ml/minute/100 ml of oil. After about 30 minutes of sparging, the oil is allowed to cool under vacuum. The oil is typically transferred to a glass container and flushed with argon before being stored under refrigeration. This treatment improves the colour of the oil and removes a majority of the volatile substances or odorous compounds including any remaining free fatty acids, monoacylglycerols and oxidation products.

As used herein, “transesterification” means a process that exchanges the fatty acids within and between TAGs (interesterification) or phospholipids, or transfers the fatty acids to another alcohol to form an ester. This may initially involve releasing fatty acids from the TAGs or PL as free fatty acids or it may directly produce fatty acid esters, preferably fatty acid methyl esters or ethyl esters. In a transesterification reaction of the TAG or PL with an alcohol such as methanol or ethanol, the alkyl group of the alcohol forms an ester linkage with the acyl groups (including the SCFA) of the TAG.

In some embodiments, both Mortierella spp. biomass (or other microbial biomass) containing phospholipids and an extracted lipid from a microorganism such as Mortierella spp. comprising phospholipids are used in compositions, food products, beverage products and feedstuffs in accordance with the present invention. Such embodiments may provide an enhanced food-like, for example meaty or fishy, aroma. In some such embodiments, the Mortierella spp. biomass present in the composition is the same as the Mortierella spp. from which the phospholipid is extracted. In some alternative embodiments, the Mortierella spp. biomass present in the composition is different from the microorganism, such as the Mortierella spp., from which the extracted lipid comprising phospholipids is extracted.

The microorganism or phospholipid extracted from a microorganism in accordance with the present disclosure is not a ‘yeast extract’ as commonly referred to in the art. The term “yeast extract” is understood in the art to generally refer to the water-soluble portion of autolyzed yeast and typically does not contain phospholipid fractions (see, for example, Sigma Aldrich, Catalog No. Y1625 Yeast Extract). As used herein the term “yeast extract” includes a composition that is sold commercially and labelled as a yeast extract. These are water-soluble fractions of yeast cells comprising amino acids, carbohydrates, vitamins and minerals and are typically sold in a dry powdered form.

### Genetic Modification

In accordance with some embodiments of the present invention, microorganisms may be genetically modified by suitable methods, to contain a desired amount or profile of phospholipids, for example increased amounts of phospholipids/polar lipids and/or increased amounts of ω-6 fatty acid esterified in phospholipids. Thus, the microorganism may comprise one or more genetic modifications providing for: synthesis of, or increased synthesis of, one or more ω6 fatty acids; an increase in total fatty acid synthesis and/or accumulation in the microorganism; an increase in total polar lipid synthesis and/or accumulation in the microorganism; a decrease in TAG synthesis and/or accumulation in the microorganism, or an increase in TAG catabolism, such as an increase in TAG lipase activity; or a reduction in catabolism of total fatty acids.

The genetic modifications may include the introduction of an exogenous polynucleotide, a mutation or a deletion of a gene or regulatory sequence, or any other known genetic modification. Suitable techniques for genetically modifying microorganisms are well known to those in the art. For example, suitable recombinant DNA techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present). Reference is also made to international patent application no. PCT/AU2022/050177, the discosure of which is incorporated herein in tis entirety.

By way of example only, polynucleotides encoding desaturase and elongase enzymes can be used to genetically engineer microorganisms to produce lipids for use in the present invention. The desaturase and elongase proteins, and genes encoding them, that may be used in the invention are any of those known in the art or homologues or derivatives thereof. Reference is also made to international patent application no. PCT/AU2022/050177, the discosure of which is incorporated herein in tis entirety.

As used herein, the term “desaturase” refers to an enzyme which is capable of introducing a carbon-carbon double bond into the acyl group of a fatty acid substrate which is typically in an esterified form such as, for example, acyl-CoA esters. The acyl group may be esterified to a phospholipid such as phosphatidylcholine (PC), or to acyl carrier protein (ACP), or preferably to CoA. The desaturase enzymes that have been shown to participate in ω6 fatty acid biosynthesis belong to the group of so-called “front-end” desaturases.

Fatty acid elongation consists of 4 steps: condensation, reduction, dehydration and a second reduction. In the context of this invention, an “elongase” refers to the polypeptide that catalyses the condensing step in the presence of the other members of the elongation complex, under suitable physiological conditions. It has been shown that heterologous or homologous expression in a cell of only the condensing component (“elongase”) of the elongation protein complex is required for the elongation of the respective acyl chain.

Any embodiment herein shall be taken to apply mutatis mutandis to any other embodiment unless specifically stated otherwise.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The reference in this specification to any prior publication (or information derived from it), or to any matter which is known, is not, and should not be taken as, an acknowledgement or admission or any form of suggestion that prior publication (or information derived from it) or known matter forms part of the common general knowledge in the field of endeavour to which this specification relates.

The present disclosure will now be described with reference to the following specific examples, which should not be construed as in any way limiting the scope of the invention.

### EXAMPLES

### Example 1. Materials and Methods

### Media and Chemicals

YPD medium is a rich medium which contains 10 g/L yeast extract (Sigma Aldrich, Catalog No. Y1625), 20 g/L peptone (Sigma Aldrich, Catalog No. P0556) and 20 g/L glucose (Sigma Aldrich, Catalog No. G7021). YPD plates contain, in addition, 20 g/L agar. SD-Ura medium contained Yeast Synthetic Drop-out Medium (Sigma Catalog No. Y1501) at the recommended amount per litre. This medium was supplemented with uracil as required. SD agar plates contained 6.7 g/L yeast nitrogen base, 20 g/L glucose and 20 g/L agar.

Chemicals were sourced as follows unless stated otherwise: L-cysteine (Sigma, Catalog No. 168149), D-(−) ribose (Sigma, Catalog No. R7500), thiamine hydrochloride (Sigma, Catalog No. 47858), iron fumarate (Fe²⁺, Apohealth, NSW, Australia; Code #MH/Drugs/25-KD/617), L-glutamic acid monosodium salt hydrate (Sigma, Catalog No. G5889), potassium dihydrogen phosphate (Sigma, Catalog No. 1048731000).

### Media for Larger Scale Yeast Cultures

Unless otherwise stated, the medium used for preparing seed cultures for larger scale yeast cultures (2 L or more) was a defined medium (DM-Gluc), having a base medium (BM) containing per litre 10.64 g potassium di-hydrogen orthophosphate (KH₂PO₄), 4.0 g di-ammonium hydrogen orthophosphate ((NH₄)₂HPO₄) and 1.7 g citric acid (monohydrate). These ingredients were dissolved in about 70% of the required volume of water that had been purified by reverse osmosis, adjusted to pH 6.0 with 2 M NaOH, and made up to the required volume using purified water. The BM was sterilised at 121° C. for 20 min and cooled to room temperature. The following ingredients were then added separately (per litre): 30 ml of 660 g/L glucose (autoclaved), to a final concentration of 20 g/L, 10 ml 1 M magnesium sulphate heptahydrate (autoclaved), 10 ml Trace metal solution (see below, filter sterilised), 10 ml of 15 g/L thiamine hydrochloride (filter sterilised) and 3 ml 10% (v/v) Sigma Antifoam 204 (autoclaved).

The fermentation medium (FM) for yeast cultures of 2 L or more in volume also used the BM as base mediumunless otherwise stated. The required volume was added to the bioreactor and sterilised at 121° C. for a 60 min fluid cycle for an autoclavable bioreactor or 30 min for a steam-in-place bioreactor, and cooled to 30° C. The following ingredients were added, per litre of base medium: 121 ml of 660 g/L glucose (autoclaved), giving a final concentration of 80 g/L, 5 ml of 1M magnesium sulphate heptahydrate (autoclaved), 5 ml of Trace metal solution (see below, filter sterilised), 5 ml 15 g/L thiamine hydrochloride (filter sterilised) and 50 ml of 200 g/L ammonium chloride (filter sterilised). The glucose, magnesium, trace metal solution and thiamine solution were mixed and added to the bioreactor together. Once the medium was formulated, the pH was checked, normally slightly less than 6.0. A pH controller was used to add ammonia solution to the medium and bring the pH to 6.0.

Small scale (50 ml) and larger scale yeast cultures of 2 L or more for inducing more TAG synthesis were also grown in a defined medium containing glycerol at 8% (w/v) and having a lower nitrogen content (DM-Glyc-LowN). This medium was the same as DM-Gluc except that the glucose was replaced with 80 g/L glycerol (final concentration) as carbon source and the (NH₄)₂HPO₄ content was reduced to 2.0 g/L or even 0.5 g/L, as stated. For the larger yeast cultures, starter cultures were grown in either YPD medium or SD-Ura medium, with addition of uracil and any amino acids if required, for 24-48 h. A sample of the starter culture was centrifuged and the cells used to inoculate the larger culture. These cultures were incubated for 48-96 h and the pH maintained at 6.0 unless otherwise stated.

The Trace metal stock solution (TMS) used in the media described above contained, per litre: 2.0 g CuSO₄.5H₂, 0.08 g NaI, 3.0 g MnSO₄·H₂O, 0.2 g NaMoO₄.2H₂O, 0.02 g H₃BO₃, 0.5 g CoCl₂.6H₂O, 7.0 g ZnCl₂, 22.0 g FeSO₄·7H₂O, 0.50 g CaSO₄.2H₂O, and 1 ml of sulphuric acid. The reagents were added in the listed order. Addition of the sulphuric acid resulted in dissolution of the calcium sulphate. The trace metal solution was filtered sterilised through a 0.2 μm filter and stored at 2-8° C. in a bottle wrapped in aluminium foil.

One pH control reagent was a phosphoric acid solution (10% w/v), prepared by adding 118 ml of 85% H₃PO₄ to 882 ml of purified water. The solution was sterilised by autoclaving. The other was an ammonia solution (10% v/v), prepared by adding 330 ml of a 30% ammonia solution to 670 ml of purified water. That solution was assumed to be self-sterilising. An antifoam solution was prepared by mixing 100 ml of Sigma antifoam 204 with 900 ml of purified water, providing a concentration of 10%. The mixture was sterilised by autoclaving.

A feed solution was prepared by adding 134 ml of 200 g/L ammonium chloride which had been filter sterilised to 1 L of 660 g/L glucose, and sterilised by autoclaving.

### Microbial Strains

S. cerevisiae strain D5A (ATCC 200062) was used as a yeast for experiments on production of lipids including phospholipids. Several yeast strains of the species Yarrowia lipolytica were also used and were obtained from the American Type Culture Collection (Manassas VA, USA), for example wild-type strain W29 (Casaregola et al., 2000).

The fungal strain described herein as yNI0121 (Mucor hiemalis) has been deposited with National Measurement Institute, Port Melbourne, VIC 3207, Australia on 4 Feb. 2021 under the Budapest Treaty and has been designated the following Deposit Number: yNI0121 Deposit Accession number V22/001757. Fungal strains described herein as yNI0125 (Mortierella elongata), yNI0126 (Mortierella sp.), yNI0127 (Mortierella sp.) and yNI0132 (Mortierella alpina) have been deposited with National Measurement Institute, Port Melbourne, VIC 3207, Australia on 12 Oct. 2021 under the Budapest Treaty and have been designated the following Deposit Numbers: yNI0125 Deposit Accession number V21/019953, yNI0126 Deposit Accession number V21/019951, yNI0127 Deposit Accession number V21/019952, and yNI0132 Deposit Accession number V21/019954.

Growth of S. cerevisiae and Y. lipolytica Cultures for Lipid Analysis

To provide an inoculum for yeast cultures for fatty acid production, extraction and analysis, small-scale cultures of Y. lipolytica or S. cerevisiae were grown in 5 ml or 10 ml of YPD medium at 29° C. for 24 h. For experiments, the inoculum culture was diluted into the growth medium having a volume of, for example, 50-2000 ml to an optical density at 600 nm (OD600) of approximately 0.1. Smaller scale cultures were grown in polypropylene tubes for 10 ml cultures, or glass flasks for larger volumes, the container having a volume at least 5-fold greater than the culture volume. The containers were sealed with 3M micropore surgical tape (Catalog No. 1530-1) tape and incubated in a shaker at a defined temperature of 29° C. unless specified otherwise, at 200 rpm for aeration.

When SD-Ura medium was used, a carbon source such as 2% glycerol or raffinose (w/v) (MP Chemicals, USA, Catalog No. 4010022) was used. Cultures were incubated overnight at 28° C. with shaking for aeration. The inoculum culture was diluted into 10 ml of SD-Ura medium, or other volume as specified, containing 2% (w/v) glycerol or raffinose to provide an initial OD600 of 0.1. The culture in a 50 ml tube or a 250 ml flask was incubated in a shaker at 28° C. at 200 rpm for aeration. The OD600 was checked at time intervals of 15 or 30 min. When the OD600 reached 0.3, exogenous compounds as potential substrates (if any) were added to the medium.

### Feeding Lipid Substrates to the Cells

For substrate feeding experiments, yeast inoculum cultures were diluted into their respective growth media containing 1% tergitol (Sigma Aldrich Catalog No. NP40S) or Tween-100 at an OD600 of 0.1 and incubated with shaking for a period of time, typically 2 h. Lipid substrates such as e.g. fatty acids, oil or oil-hydrolysates were then added to the medium and the cultures further incubated for different time periods. Unless otherwise stated, fatty acid substrates were dissolved in ethanol and provided to the cultures to a final concentration of 0.5 mg/ml, or the sodium salts of the fatty acids were provided in aqueous solution.

### Seed Culture for Larger Scale Cultures

For a primary seed culture, a frozen glycerol stock of the yeast strain was used to inoculate 100 mL of DM in a plastic baffled 1 L Erlenmeyer flask with a vented cap. This was incubated at 28° C. with shaking at 200 rpm for aeration for 24±2 h. The optical density at 600 nm (OD600) was measured at the end of incubation. A secondary seed culture was prepared by using the primary seed culture to inoculate 500 mL of DM in a plastic baffled 2 L Erlenmeyer flask with a vented cap, to a starting OD600 of 0.04. The second seed culture was incubated at 28° C. with shaking at 200 rpm for 16±2 hours. The OD600 was measured at the end of incubation. This culture was used to inoculate the large-scale fermentation.

### Cell Harvesting, Washing and Freeze Drying

Cells from smaller scale cultures were harvested by centrifugation, for example in a 50 ml tube at 4,600 g for 15 min, washed twice with 10 ml and finally washed with 1 ml MilliQ water. For the final wash, where a dry cell weight was to be measured, the cell suspension was transferred to a pre-weighed 2 ml Eppendorf tube, centrifuged, and the cell pellet freeze-dried (VirTis Bench Top freeze dryer, SP Scientific) before weighing and lipid extraction. When lipid substrates such as ARA, DGLA, γ-linolenic acid (GLA) or other fatty acids were added to the growth medium, cell pellets were washed successively with 1 ml of 1% tergitol (v/v), 1 ml of 0.5% tergitol and a final wash with 1 ml water to remove any remaining substrate from the exterior of the cells and freeze-dried as described above. When an oil was added to the growth medium, cells were harvested by centrifugation as above but the cell pellets were washed successively with 5 ml of 10% tergitol (v/v), 5 ml of 5% tergitol, 5 ml of 1% tergitol, 5 ml of 0.5% tergitol and a final wash with 5 ml water to remove any remaining oil from the exterior of the cells. In some cases, microscopic observation after staining with Bodipy confirmed the absence of oil stained at the cell walls. With the final wash, pellets were transferred to pre-weighed 2 ml Eppendorf tubes and freeze-dried before weighing and lipid extraction.

Lipid Extraction from Yeast Cells

In some experiments, total cellular lipid was extracted from yeast cells such as S. cerevisiae or Y. lipolytica by using a method modified from Bligh and Dyer (1959). Approximately 50 mg freeze-dried cells were homogenized with 0.6 ml of a mixture of chloroform/methanol (2/1, v/v) with 0.5 g zirconium oxide beads (Catalog No. ZROB05, Next Advance, Inc., USA) in a 2 ml Eppendorf tube using a Bullet Blender Blue (Next Advance, Inc. USA) at speed 6 for 5 min. The mixture was then sonicated in an ultrasonication water bath for 5 min and 0.3 ml 0.1 M KCl was added. The mixture was shaken for 10 min and centrifuged at 10,000 g for 5 min. The lower, organic phase containing lipid was transferred to a glass vial and remaining lipid was extracted from the upper phase containing the cell debris by mixing it with 0.4 ml chloroform for 20 min and centrifugation. The lower phase was collected and combined with the first extract in the glass vial. The solvent was evaporated from the lipid sample under a flow of nitrogen gas and the extracted lipid resuspended in a measured volume of chloroform. If required, the lipid samples were stored at −20° C. until further analysis.

Lipid Extraction from the Larger Biomass

For the extraction of total lipid from a larger biomass, different methods of cell homogenization were used with larger volumes of the solvents, unless otherwise stated. In one method, cells were homogenized in chloroform/methanol (2/1, v/v) using an Ultra-Turrax T25 homogenizer (IKA Labortechnik Staufen, Germany) for 3 min or times as stated. Further homogenization was carried out for 2 min after adding one volume of 1 M KCl to each mixture. The mixture was centrifuged at 6,000 g for 3 min. The lower phase was transferred to a new tube (Tube B) and the solvent was evaporated under a flow of nitrogen at room temperature. The upper phase was mixed with 1 g of glass beads in a Vibramax mixer for 10 min and with vigorous vortexing for 1 min. One volume of chloroform was added to each tube and mixed again for 3 min. After centrifugation, the lower phase was transferred to Tube B and the solvent was evaporated under a flow of nitrogen gas at room temperature. To extract remaining lipid, the upper phase in Tube A was mixed with another volume of chloroform and mixed for 3 min. After centrifugation, the lower phase was again transferred to Tube B. 0.5 volume each of methanol and 0.1 M KCl were added to Tube B and mixed for 3 min. The lower phase was transferred to a Falcon tube and the solvent was evaporated under a flow nitrogen gas at room temperature. The extracted lipid was dissolved in chloroform/methanol (2/1, v/v) and stored at −20° C.

When hexane was used as the extraction solvent to extract total lipid, a wet biomass of cells was first washed twice with ethanol to remove water. If this was not done, the hexane-water solvent system tended to separate as two phases and could have reduced the extraction efficiency through less mixing. This washing step with ethanol was not required when a hexane/ethanol mixture (60/40 or 40/60 v/v) was used. Similar extraction and disruption methods using solvents were used as described in the Examples.

Lipid Extraction from Fungal Biomass

Unless otherwise stated, the following method was used to extract lipid from biomass of fungi such as Mortierella or Mucor, where the method preferentially extracts the polar lipid including phospholipids (PL) on the basis of differential solubility of PL and neutral lipids, firstly in ethanol as solvent and then in hexane for remaining lipid. Wet fungal biomass of a known weight was washed with ethanol to remove water, then resuspended in ethanol using 2 ml ethanol per g of biomass. The mixture was homogenised using an Ultra-Turrax for 3 min and then sonicated for 5 min. The homogenisation and sonication steps were repeated twice more for a total of three times. A sample was observed by light microscopy to check that mycelial disruption had occurred. The mixture was centrifuged to pellet cellular debris, which was weighed. The ethanol supernatant was collected and the solvent evaporated to recover the polar lipids. The cell debris was mixed with hexane to extract neutral lipids and any remaining polar lipids, using 5 ml of hexane per g of cell debris. The mixture in hexane was homogenised for 3 min using the Ultra-Turrax. The mixture was then shaken for 2 h, centrifuged, and the hexane supernatant collected. The solvent was evaporated to recover the lipid, which containing mostly TAG.

### Lipid Fractionation by Thin Layer Chromatography

To separate different lipid types such as TAG, DAG, free fatty acid (FFA) and polar lipids such as PL at an analytical scale, total lipids were fractionated on thin layer chromatography (TLC) plates (Silica gel 60; Catalog No. 1.05626.0001, MERCK, Darmstadt, Germany) using hexane:diethylether:acetic acid (70/30/1 v/v/v) as the solvent system. A sample of a lipid standard such as 18-6A containing TAG, DAG, FFA and MAG (Nu-Chek Prep Inc, USA) was run in an adjacent lane to identify the different lipid spots. After the chromatography, the plates were sprayed with a primuline (Catalog No. 206865, Sigma, Taufkirchen, Germany) solution prepared at a concentration of 5 mg/100 ml in acetone:water (80/20 v/v) and lipid bands visualised under UV light. The silica with the lipid from each spot was scraped off and transferred to a tube. The lipid fractions were extracted from the silica for derivatisation using methylation and subjected to GC analysis for determining the fatty acid composition and quantitation.

Preparative Scale Fractionation of PL and TAG from Total Lipid by TLC

PL and TAG were fractionated from about 100 mg of total lipid by loading the lipid on 18 cm lines on each of eight TLC plates (Silica gel 60; Catalog No. 1.05626.0001, Merck, Darmstadt, Germany) and chromatographed with a solvent mixture consisting of hexane/diethylether/acetic acid (70:30:1, v:v:v). An aliquot of a lipid standard containing TAG, DAG, FFA and MAG (18-6A; NuChek Inc, USA) was run in parallel to assist with identifying the lipid bands. After staining the plates with primuline and visualisation under UV light, the PL bands located at the origin and the TAG bands having the same mobility as the TAG standard were collected and transferred to Falcon tubes. The lipid/silica samples were extracted with a mixture of 6 ml chloroform and 3 ml methanol, mixing vigorously for 5 min, then adding 3 ml water and further mixing for 5 min. After centrifugation for 5 min at 3,000 g, the lower organic phase was transferred to a new tube. The lower phase was transferred to a Falcon tube after centrifugation at 3,000 rcf for 5 min. The upper phase was mixed with 5 ml chloroform for 5 min to extract any remaining lipid. After centrifugation, the lower phase was combined with the first extract. The solvent was evaporated under a flow of nitrogen gas. The extracted lipid, TAG or PL, was dissolved in a small volume of chloroform and filtered through 0.2 μm micro-spin filter (Chromservis, EU, Catalog No. CINY-02) to remove any particulates. The fatty acid composition and amount of each PL and TAG fraction were determined by preparation of FAME and GC analysis. Such preparations were used, for example, to separate different polar lipid classes such as PC, PE, PI and PS, or in Maillard reactions for aroma tests or for detection of volatile compounds as reaction products.

### Lipid Derivatisation to Fatty Acid Methyl Esters (FAME)

For analysis by GC, fatty acid methyl esters (FAME) were prepared from total extracted lipid or the purified TAG or polar lipid fractions, including PL samples, by treatment with 0.7 ml 1 N methanolic-HCl (Sigma Aldrich, Catalog No. 90964) in a 2 ml glass vial having a PTTE-lined screw cap at 80° C. for 2 h. A known amount of heptadecanoin (Nu-Chek Prep, Inc., Catalog No. N-7-A, Waterville, MN, USA) dissolved in toluene was added to each sample before the treatment as an internal standard for quantification. After the vials were cooled, 0.3 ml of 0.9% NaCl (w/v) and 0.1 ml hexane were added and the mixtures vortexed for 5 min. The mixture was centrifuged at 1,700 g for 5 min and the upper, hexane phase containing the FAME was analysed by GC.

### Analysis and Quantification of FAME by GC

The individual FAMEs were identified and quantified by GC using an Agilent 7890A GC (Palo Alto, California, USA) with a 30 m SGE-BPX70 column (70% cyanopropyl polysilphenylene-siloxane, 0.25 mm inner diameter, 0.25 μm film thickness), a split/splitless injector and an Agilent Technologies 7693 Series auto sampler and injector, and a flame ionisation detector (FID). Samples were injected in split mode (50:1 ratio) at an oven temperature of 150° C. The column temperature was programmed for 150° C. for 1 min, increasing to 210° C. at 3° C./min, holding for 2 min and reaching 240° C. at 50° C./min, then holding at 240° C. for 0.4 min. The injector temperature was set at 240° C. and the detector at 280° C. Helium was used as the carrier gas at a constant flow of 1.0 ml/min. FAME peaks were identified based on retention times of FAME standards (GLC-411, GLC-674; NuChek Inc., USA). Peaks were integrated with Agilent Technologies ChemStation software (Rev B.04.03 (16), Palo Alto, California, USA) based on the response of the known amount of the external standard GLC-411 (NuChek) and C17:0-ME internal standard. The resultant data provide the fatty acid composition on a weight basis, with percentages of each fatty acid (weight %) in a total fatty acid content of 100%. These percentages on a weight basis could readily be converted to percentages on a molar basis (mol %) based on the known molecular weight of each fatty acid.

### Peak Identity by GC-MS

The identities of unknown or uncertain peaks in the GC-FID chromatograms were confirmed by Gas Chromatography Mass Spectrometry (GC-MS) analysis. Samples were run on a GC-MS operating in the Electron Ionization mode at 70 eV to confirm peak identities and to identify possible extra peaks corresponding to possible contamination, degradation products or reagent signals. A Shimadzu GC-MS QP2010 Plus (Shimadzu Corporation, Japan) system coupled to an HTX-Pal liquid auto-sampler was used with the following parameters: 1 or 2 μl injection volume using a split/splitless inlet at a 15:1 split, at a temperature of 250° C. The oven temperature program used was the same as for the GC-FID. MS ion source and interface temperatures were 200° C. and 250° C., respectively. Data were collected at a scan speed of 1000 and scan range from 40 to 500 m/z. Peak separation was provided by a Stabilwax or Stabilwax-DA (Restek/Shimadzu) capillary column (30 m×0.25 mm i.d., 0.25 μm film thickness) using He as a carrier gas at 30 cm/sec. Mass spectra correlations were performed using a NIST library, retention indices and matching retention time of available standards. Identified SCFA was set to be present when S/N ratio were above 10:1. Instrument blanks and procedural blanks were run for quality control purposes.

### Example 2. Lipid Fractionation

Crude lipid preparations may be fractionated with organic solvents to provide purer polar lipids or fractions having mostly neutral (non-polar) lipids including TAG (e.g. U.S. Pat. No. 7,550,616). For example, some reported methods use differential solubility of neutral and polar lipids in organic solvents such as ethanol or acetone. To test some of these methods, fractionation of several lipids having a mixture of substantial neutral and polar lipids was attempted, including egg yolk lipid and krill lipid, as model systems.

The lipids in chicken eggs are present mostly in the yolk fraction which constitutes about 33% lipid by weight. The lipids, which are closely associated with proteins in the yolk, are mostly TAG (66% by weight), with phospholipids (PL, 28%) and cholesterol and its esters (6%) present in lower amounts (Belitz et al., 2009). The PL contains some ω3 and ω6 fatty acids (Gladkowski et al., 2011). Based on the method of Palacios and Wang (2005), Gladkowski et al., (2012) extracted PL from egg yolk with ethanol and then purified the PL by removing neutral lipids by precipitation of the PL with cold acetone.

Fresh egg yolk (17 g), egg lecithin powder (20.4 g; Lesen Bio-Technology Co, Xi'an, China) and krill oil from Euphausia superba (17.7 g) obtained from commercially available krill oil capsules (Bioglan Red Krill Oil; Natural Bio Pty Ltd, Warriewood, NSW, Australia) were each mixed with 60 ml of ethanol and stirred for 30 min. The ethanol supernatant was collected after centrifuging the mixture. The precipitate was extracted twice more, each time with 60 ml ethanol. The extraction mixtures were centrifuged and the ethanol supernatants combined. Each precipitate was retained for extraction of neutral lipids. The ethanol from the combined supernatants was evaporated using a SR-100 rotary evaporator (Buchi, Switzerland) operating at 400 rpm with a vacuum of 15 mbar, with the chiller set at −16° C. and the waterbath at 37° C. This yielded 3.2 g of PL-enriched lipid extract from the 17 g of fresh egg yolk, 5.86 g from the 20.4 g of egg lecithin powder and 17.83 g of enriched PL recovered from the krill oil. The lipid recovered from the krill oil probably still contained a small amount of solvent. Nevertheless, the recovery of essentially 100% indicated that the krill oil from the capsules was highly enriched for PL to begin with.

Aliquots of the recovered lipids were analysed by TLC as described in Example 1 using hexane:diethylether:acetic acid (70:30:1; v/v/v) as solvent. The ethanol extracts from fresh egg yolk and egg yolk lecithin powder were observed to contain substantial amounts of polar lipid as well as a small amount TAG, while the krill oil extract had no detected TAG.

To further purify the polar lipids from the fresh egg yolk, the dried extract was dissolved in 30 ml of hexane and the solution cooled in an ice bath to 0° C. Next, 60 ml of cold acetone (−20° C.) was gradually added to the solution and the mixture kept cold for at least 20 min to precipitate the PL. Other experiments showed that more precipitate formed by keeping the mixtures at 0° C. overnight. The precipitate was collected and dried under vacuum. Samples of the lipid were dissolved in chloroform and analysed by TLC to estimate the polar lipid and TAG contents. The acetone precipitate was shown to have mostly polar lipid with some TAG. To further purify the polar lipid, the precipitate was washed 5 times with 20 ml portions of cold acetone (−20° C.) to remove more of the TAG and other neutral lipids such as cholesterol. The residual solvent was removed from the washed precipitate by rotary evaporation at room temperature for 10 h. The lipid yield was measured gravimetrically and a small aliquot used for analysis of the fatty acid composition by GC quantitation of FAME. From the initial input of 17 g of fresh egg yolk, 1.1 gram of purified polar lipid was recovered. An aliquot of this extracted lipid was analysed by TLC and was observed to be essentially devoid of any neutral lipids, including TAG. These observations were consistent with those reported by Gladkowski et al., (2012) who found their extracts to be 96% pure PL.

Neutral lipid was extracted from the precipitates after the ethanol extraction of the egg yolk and egg yolk powder by extracting the precipitate twice with 50 ml of hexane. The combined hexane solution containing the neutral lipid was washed four times, each time with 50 ml of 90% ethanol. The hexane was then evaporated under reduced pressure to provide the purified neutral lipids from egg yolk.

To determine the fatty acid composition of the extracted lipids, the total fatty acids in aliquots were converted to FAME for GC analysis as described in Example 1. This included the samples (1^{st} ppt) after the ethanol extraction but before the hexane/acetone precipitation, as well as samples (2^{nd} ppt) after the hexane/acetone precipitation. The data are shown in Table 3. The ethanol-soluble lipid isolated from the fresh egg yolk and acetone precipitated lipid purified therefrom contained C16:0 and C18:0 as the main saturated fatty acids. The first lipid precipitate from fresh egg yolk containing 24.7% (C16:0) and 15.6% (C18:0) while the more purified polar lipid contained 27% (C16:0) and 16% (C18:0). The amount of LA in the 2^{nd} precipitate was slightly higher than in the 1^{st} precipitate; LA is present at greater amounts in PL than in TAG. Both fresh egg yolk and the purer polar lipid preparations also contained ω6 and ω3 LC-PUFA. For instance, the fresh egg yolk 1^{st} precipitate contained 5.3% C20:4 (ARA), 2.3% C20:5 (EPA) and 5% C22:6 (DHA) while more purified polar lipid preparation contained 5.3% ARA and 4% DHA. The first precipitate from the krill oil and the more purified polar lipid from the krill oil had C16:0 as their main saturated fatty acid. The krill oil 1^{st} precipitate and the more purified polar lipid also contained substantial amounts of 3 LC-PUFA, namely 1.1% ARA, 34.7% EPA and 19.0% DHA in the 1^{st} precipitate, while the more purified polar lipid contained 1.1% ARA, 48.1% EPA and 25.7% DHA. The precipitated lipid from the egg yolk lecithin powder had 17% C16:0 and 4% C18:0 but was low in the LC-PUFA EPA and DHA. It was considered that the low LC-PUFA content of the lecithin powder was likely due to oxidative breakdown of those polyunsaturated fatty acids during its production or storage.

An alternative method to purify polar lipids by fractionation from a total lipid preparation is to use silica-based column chromatography such as, for example, use of SPE columns (HyperSep aminopropyl, ThermoFisher, UK).

TABLE 3
Fatty acid composition of polar lipids purified from egg yolk and krill oil capsules.
lipid

| | Polar lipid fractions | | | | | | | | | |
|---|---|---|---|---|---|---|---|---|---|---|
| | | | | | | | | | | C18:2 |
| | C12:0 | C14:0 | C15:0 | C15:1 | C16:0 | C16:1 | C18:0 | C18:1 | C18:1Δ11 | (LA) |
| Egg yolk 1^{st} ppt | 0.0 | 0.6 | 0.1 | 0.2 | 24.7 | 2.2 | 15.6 | 26.9 | 0.0 | 14.8 |
| Egg yolk 2^{nd} ppt | 0.0 | 0.2 | 0.1 | 0.2 | 27.1 | 1.0 | 16.5 | 23.6 | 0.0 | 20.2 |
| Krill oil 1^{st} ppt | 0.0 | 3.1 | 0.3 | 0.1 | 25.7 | 1.9 | 1.1 | 6.1 | 0.0 | 2.6 |
| Krill oil 2^{nd} ppt | 0.0 | 4.0 | 0.4 | 0.1 | ? | 0.2 | 1.1 | 8.3 | 0.1 | 3.0 |
| | C18:3 | C19:0 | C20:0 | C20:2 | C20:3 | C20:4ARA | C20:5EPA | C22:2 | C22:6DHA | C24:0 |
| Egg yolk 1^{st} ppt | 0.5 | 0.2 | 0.0 | 0.3 | 0.3 | 5.3 | 2.3 | 0.0 | 5.3 | 0.4 |
| Egg yolk 2^{nd} ppt | 0.4 | 0.0 | 0.0 | 0.4 | 0.3 | 5.3 | ? | 0.0 | 4.2 | 0.1 |
| Krill oil 1^{st} ppt | 1.9 | 0.0 | 0.0 | 0.2 | 0.2 | 0.8 | 34.7 | 1.3 | 19.0 | 0.6 |
| Krill oil 2^{nd} ppt | 2.6 | 0.0 | 0.0 | 0.2 | 0.2 | 1.1 | 48.1 | 1.7 | 25.7 | 0.9 |

### Example 3. Maillard Reactions

The Maillard reaction is a chemical reaction between a reducing sugar and an amino group, for example in a free amino acid, with application of heat. Like caramelisation, it is a form of non-enzymatic browning. In this reaction, the amino group reacts with a carbonyl group of the sugar and produces N-substituted glycosylamine and water. The unstable glycosylamine undergoes an Amadori rearrangement reaction and produces ketosamines. The ketosamines can react further in different ways to produce reductones, diacetyl, aspirin, pyruvaldehyde, and other short-chain hydrolytic fission products. Finally, a furan derivate may be obtained which reacts with other components to polymerize into a dark-coloured insoluble material containing nitrogen.

The outcome of the Maillard reaction depends on temperature, time and pH. For example, the reaction slows at low temperature, low pH and low water activity (Aw) levels. The browning colour occurs more quickly in alkaline conditions because the amino group remains in the basic form. The reaction peaks at intermediate water activities such as Aw of 0.6-0.7. In addition to colour, many volatile aroma compounds are typically formed during the Maillard reaction. Flavour-intensive compounds may be formed in the presence of the sulphur-containing amino acids methionine or cysteine or other sulphur containing compounds such as thiamine. Unsaturated fatty acids and aldehydes formed from fatty acids also contribute to the formation of heterocyclic flavour compounds during the Maillard reaction (Gehard Feiner, 2006). In view of this contribution of unsaturated fatty acids to formation of flavours and aromas, the inventors tested the extracted egg yolk polar lipid preparation from Example 2 as a model system for Maillard reactions.

In an initial experiment, 26 mixtures for Maillard reactions were assembled containing a matrix of components in a base medium and either containing 15 mg of the extracted egg yolk polar lipid (Example 2) or lacking the lipid (controls). The reactions were carried out in 2 ml volumes in 20 ml glass vials with tightly sealing screw top lids. To deposit a precise amount of the extracted lipid into the vials, the lipid was dissolved in hexane at a concentration of 1 mg/μl of the solvent. An aliquot of 50 μL of the lipid solution containing 50 mg enriched polar lipid was pipetted into the vials for reactions having the lipid. The hexane was then evaporated under a nitrogen flow. The other components in each mixture were added to the vials in the following order. Components were added to provide final concentrations of 10 mM xylose as the sugar, 0.1 mM thiamine hydrochloride, and either 5 mM cysteine or 5 mM cystine as a sulphur-containing amino acid. These components were dissolved in a final concentration of 32.6 mM potassium phosphate buffer pH 6.0 or 5.3, prepared from potassium dihydrogen phosphate and dipotassium hydrogen phosphate. Some mixtures also included one or more of 15 mg/mL yeast extract, 3.5 mg/L iron (Fe²⁺) in the form of iron fumarate (Apohealth, NSW, Australia) and 2 mM L-glutamic acid monosodium salt hydrate. The presence or absence of yeast extract was intended to test whether it would either mask, or enhance, the aroma produced from the extracted lipid having PL, or have no effect.

The assembled mixtures were sonicated for 30 min and then heated for 15 min in an oven set at 146° C. During the heat treatment, the vials were tightly sealed. The vials were cooled until warm to the touch about 15 min later, and then opened briefly for sniffing by a panel of 4 volunteers (P1 to P4). These included 2 males and 2 females, ages ranging from 24-65 years. The volunteers did not know the composition of any of the vials prior to sniffing the contents and the vials were sniffed in a random order as selected by the volunteers. The volunteers sniffed coffee beans between sniffing each test sample to reset their olefactory senses. Their descriptions of the aromas were recorded without any comments being shared until the sniffing was completed.

The four participants varied considerably in their descriptions of the detected aromas of the 26 mixtures. Despite these variations, the reaction mixtures containing the added polar lipid preparation were generally recognized as having a more meaty/meat-like aroma compared to the control samples lacking the polar lipid, confirming the role of the lipids in contributing to a meaty aroma following the heating-induced Maillard reactions. Samples containing the yeast extract, the iron fumarate, or both, were identified as having more meat-like or meaty related aromas, described as beef or chicken by 3 of the 4 participants. Therefore, the base composition with those components was selected for further investigation.

Several further experiments were carried out to test variations of the Maillard reaction mixtures in terms of the composition of the base medium. In one experiment, the xylose was substituted with either glucose or ribose as the sugar component. In another experiment, Fenugreek (Trigonella foenum-graecum) leaf power was added to some of the mixtures at 10 mg per 1 ml reaction. Fenugreek leaf powder was tested as this herb has long been used in food cooking to enhance the flavour of dishes such as in curries or in combination with other herbs or spices such as cumin and coriander. Some reaction mixtures contained 30 mg of a yeast extract powder whereas others did not. Control reactions had the same base media compositions but lacked the extracted polar lipid preparation. The reaction mixes were sonicated as a batch by placing the vials in a floating foam and placed in a sonicator (Soniclean, Thermoline) set up at a medium power for 30 min and then heat treated in an oven at 140° C. for about 60 min. The vials containing the reaction mixtures were cooled slowly over about 15 min until warm to the touch. The vials were opened briefly by each of 10 volunteers and the contents sniffed, and their descriptions of the aromas recorded. The volunteers ranging in age from 29 to 65 years and were from a range of ethnic backgrounds. The reactions had been coded with random 3-digit numbers to avoid bias, and the volunteers sniffed coffee beans between vials, as before.

The recorded responses to the sniffing of the reaction mixtures were generally consistent with those of the previous experiment. Most of the mixtures containing the extracted lipid elicited favourable comments, in particular the ones containing ribose rather than glucose for meaty aromas. The use of yeast extract could enhance the meaty aroma but was not considered to generate species-specific aromas e.g. a beef aroma versus a chicken aroma. The addition of the herbal powder, Fenugreek, to the mixtures increased the sensation of a soupy or vegetable aroma with a pleasant vegetable note. It was concluded that a variety of medium compositions and components could be used with the extracted lipid, with ribose preferred over glucose as the sugar component.

### Example 4. Feeding Omega-6 Fatty Acids to Yeasts and Incorporation into Polar Lipids

The inventors produced phospholipids (PL) containing ω6 fatty acids by incorporation into microbial PL, specifically by supplementing Y. lipolytica and S. cerevisiae cultures with the ω6 fatty acids such as ARA, GLA and DGLA. This was initially done by supplying ω6 fatty acids to the microorganisms during growth of the cultures and then extracting lipids from the cells and fractionating them to isolate the polar lipids, including the PL.

Preparation of Fatty Acid Substrate from ARA Oil

As a source of ARA in feeding experiments, an ARA-containing oil was obtained from Jinan Boss Chemical Industry Co., Ltd (China), having 50% ARA in its total fatty acid content (Table 4). The inventors hydrolysed some of the oil to convert its TAG into free fatty acids, as follows. Two similar methods were tested to hydrolyse the TAG in the ARA-rich oil, both using KOH to release the free fatty acids from the glycerol backbone, in a salt form. Method 1 was based on Lipid Analysis book, 2^{nd} edition, Christie. In this method, 0.5 g of the ARA-rich oil was mixed with 1.5 ml 1 M KOH in 95% ethanol for 1 h in a glass tube (A). After cooling the solution, 1 ml water and 1 ml hexane were added to the mixture and vortexed for 5 min. After centrifugation at 1,700 g for 5 min, the upper, hexane phase was transferred to a glass tube (B). To further extract fatty acid, 1 ml hexane was added to the lower phase, vortexed for 5 min, centrifuged for 5 min and the upper phase removed and added to tube B. The solvent from tube B was evaporated under a flow of nitrogen and the dried extract was dissolved in 0.3 ml chloroform. Method 2, based on Salimon 2011, was identical to method 1 except that 0.5 g ARA-rich oil was treated with 1.5 ml 1.75 M KOH in 90% ethanol for 1 h at 65° C. The fatty acids were extracted into hexane as in method 1. Again, the hexane was evaporated under a flow of nitrogen and the dried lipid dissolved in 0.3 ml chloroform. In both methods, the alkali was not neutralised before the hexane extraction, but this was done for later preparations of hydrolysates. However, in this experiment, the hydrolysed fatty acids were isolated by TLC and recovered, so not requiring neutralisation.

To determine the extent of TAG hydrolysis, 10 μl aliquots of the fatty acid preparations were chromatographed on TLC plates (Silica 60, Merck) using hexane/diethylether/acetic acid (70/30/1; v/v/v) as the solvent system as described in Example 1. Both methods provided efficient hydrolysis of the ARA-oil as shown by the presence of bands corresponding to FFA and the absence of bands for TAG on the TLC plate. The fatty acid composition of the hydrolysates and the fatty acids purified by TLC were almost the same as the starting ARA oil, having approximately 51% ARA in the total fatty acid content of the preparations.

For a larger scale hydrolysis of the ARA oil, a third method was tested and proved successful. 50 g of the ARA-rich oil was added to 300 mL of solution containing 1.75 M KOH dissolved in 90% ethanol and mixed well. The solution was heated for 2 h in an oven at 65° C., manually shaking the mixture every 30 min. After cooling the solution to room temperature, the pH was then adjusted to 7 with HCl, to a total volume of 345 mL. A precipitate of 280 mg of KCl with some FFA salt slowly settled when the solution was cooled. To determine the extent of TAG hydrolysis, an aliquot of the supernatant was chromatographed on a TLC plate (Silica 60, Merck) using hexane/diethylether/acetic acid (70/30/1; v/v/v) as the solvent system as before. Efficient hydrolysis of the ARA-oil was established by the presence of bands corresponding to FFA and a much smaller amount of DAG or MAG, and the absence of bands for TAG, on the TLC plate.

The supernatant, having approximately 131 mg/mL of FFA, was used to supplement the yeast cultures. Free fatty acid (FFA) for use in media supplementation experiments were also obtained from NuChek Prep (USA), including γ-linolenic acid (GLA, Catalog No. U-63-A), dihomo-γ-linolenic acid (DGLA, Catalog No. U-69-A), arachidonic acid (ARA, Catalog No. U-71-A), docosatetraenoic acid-N6 (DTA, Catalog No. U-83-A), and docosapentaenoic acid-ω6 (DPAo6, Catalog No. U-102-AX). The free fatty acids were dissolved in ethanol and provided to the cultures to a final concentration of 0.5 mg/ml.

Incorporation of ω6 Fatty Acids into Phospholipids by Supplementation of Culture Medium

To test for incorporation of different ω6 fatty acids into polar lipids, cells of the yeast species Y. lipolytica strain W29 and S. cerevisiae strain INVSc1 were cultured separately in the presence of GLA, DGLA or ARA in the free fatty acid form, or in the absence of added fatty acid. The strains were each inoculated into 20 ml YPD medium in 100 ml bottles. The four media also contained 1% tergitol (NP40) to assist with solubilising the fatty acids. The initial cellular density was at an OD600 of 0.1 and the cultures were incubated at 28° C. with shaking at 200 rpm for aeration. After 2 h of incubation, the fatty acids GLA, DGLA and ARA, each of 99% purity (NuChek Inc, USA) and dissolved in ethanol were added to a final concentration of 0.5 mg/ml and incubation continued. The W29 and INVSc1 cells were harvested after 2 days and 4 days of culturing, respectively, due to their different growth rates. The harvested cells were pelleted by centrifugation at 4,600 g for 15 min. The cell pellets were washed twice to remove any remaining FFA by resuspension in water and centrifugation, and the cell pellets freeze dried. Lipid extraction and analysis of both the content and fatty acid composition of extracted polar lipid and TAG was carried out as described in Example 1.

The data for the fatty acid composition of the polar lipid and TAG fractions from the cells are provided in Table 5 for Y. lipolytica and Table 6 for S. cerevisiae. High levels of incorporation of the different ω6 fatty acids were observed in the polar lipid fraction of Y. lipolytica. The proportion of GLA, DGLA and ARA was 47.1%, 29.4% and 20.5%, respectively, of the total fatty acid content of the polar lipid fraction extracted from those cells. S. cerevisiae exhibited even higher levels of GLA, DGLA or ARA incorporation at 60.7%, 59.6% and 50.8%, respectively, in the polar lipid fraction after 4 days of incubation (Table 6). The TAG fractions from the yeast cells also showed high levels of these ω6 fatty acids. The S. cerevisiae cells exhibited TAG with incorporation of 78.1%, 80.2% and 76.8% of GLA, DGLA and ARA, respectively, indicating high activity of the acyltransferases in S. cerevisiae towards these exogenous ω6 fatty acids and efficient incorporation into TAG. The polar lipid content was higher, at greater than 2.0% of DCW, in Y. lipolytica cells, while S. cerevisiae contained approximately 1% polar lipid by dry weight.

TABLE 4
Fatty acid composition of ARA oil and
hydrolysed preparation from the oil.

| Fatty acid | ARA oil | Hydrolysate preparation |
|---|---|---|
| C12:0 | 0.0 | 0.0 |
| C14:0 | 0.3 | 0.2 |
| C15:0 | 0.1 | 0.1 |
| C16:0 | 9.5 | 8.5 |
| C16:1Δ7 | 0.0 | 0.0 |
| C16:1Δ9 | 0.3 | 0.3 |
| C18:0 | 9.4 | 9.7 |
| C18:1Δ9 | 10.9 | 11.2 |
| C18:1Δ11 | 1.0 | 0.9 |
| C18:2 (LA) | 7.3 | 7.2 |
| C18:3ω6 (GLA) | 2.8 | 2.7 |
| C18:3ω3 (ALA) | 0.1 | 0.1 |
| C20:0 | 0.7 | 0.8 |
| C20:1Δ11 | 0.5 | 0.5 |
| C20:2ω6 | 0.8 | 0.8 |
| C20:3ω6 (DGLA) | 2.6 | 2.5 |
| C20:4ω6 (ARA) | 50.5 | 50.8 |
| C20:3ω3 | 0.0 | 0.0 |
| C22:0 | 1.7 | 2.1 |
| C20:5ω3 (EPA) | 0.1 | 0.1 |
| C22:4ω6 (DTA) | 0.2 | 0.2 |
| C24:0 | 1.2 | 1.4 |

TABLE 5
Fatty acid composition of polar lipids and TAG in Y. lipolytica strain W29 after
culturing with ω6 fatty acids. The percentages are the average of triplicate assays.

| | Polar lipids | | | | Triacylglycerol | | | |
|---|---|---|---|---|---|---|---|---|
| Fatty acid | None | GLA | DGLA | ARA | None | GLA | DGLA | ARA |
| C14:0 | 0.1 | 0.1 | 0.2 | 0.1 | 0.2 | 0.2 | 0.2 | 0.2 |
| C15:0 | 0.5 | 2.1 | 1.3 | 1.0 | 0.5 | 1.2 | 0.8 | 0.8 |
| C16:0 | 8.4 | 19.5 | 12.6 | 13.1 | 15.5 | 14.1 | 15.7 | 19.8 |
| C16:1Δ7 | 1.5 | 0.2 | 0.5 | 0.3 | 0.9 | 0.3 | 0.4 | 0.3 |
| C16:1Δ9 | 15.1 | 5.0 | 11.2 | 11.0 | 9.4 | 3.1 | 5.8 | 6.9 |
| C17:1 | 2.6 | 1.5 | 1.8 | 1. | 1.3 | 0.6 | 0.8 | 0.9 |
| C18:0 | 0.4 | 2.5 | 0.7 | 0.6 | 9.3 | 8.2 | 7.9 | 10.2 |
| C18:1Δ9 | 53.9 | 18.6 | 31.6 | 39.9 | 44.0 | 1 | 22.5 | 28.5 |
| C18:1Δ11 | 1.0 | 0.1 | 0.3 | 0.5 | 0.9 | 0.4 | 0.5 | 0.8 |
| C18:2 (LA) | 16.4 | 2.4 | 8.6 | 9.8 | 10.4 | 2.4 | 5.4 | 5.3 |
| C18:3ω6 (GLA) | 0.0 | 47.1 | 1.6 | 1.2 | 0.0 | 47.7 | 1.4 | 0.9 |
| C20:0 | 0.0 | 0.1 | 0.0 | 0.0 | 0.5 | 0.5 | 0.3 | 0.4 |
| C20:3ω6 (DGLA) | 0.0 | 0.4 | 29.4 | 0.3 | 0.0 | 0.8 | 33.4 | 0.5 |
| C20:4ω6 (ARA) | 0.0 | 0.0 | 0.0 | 20.5 | 0.0 | 0.4 | 0.0 | 16.7 |
| C22:0 | 0.0 | 0.0 | 0.0 | 0.0 | 0.6 | 0.5 | 0.3 | 0.5 |
| C24:0 | 0.2 | 0.4 | 0.2 | 0.2 | 6.4 | 4.0 | 4.6 | 7.1 |
| % of DCW | 1.9 | 2.1 | 2.4 | 2.4 | 0.4 | 0.5 | 0.8 | 0.8 |

TABLE 6
Fatty acid composition of polar lipids and TAG in S. cerevisiae strain INVSc1 after
culturing with ω6 fatty acids. The percentages are the average of triplicate assays.

| | Polar lipids | | | | Triacylglycerol | | | |
|---|---|---|---|---|---|---|---|---|
| Fatty acid | None | GLA | DGLA | ARA | None | GLA | DGLA | ARA |
| C14:0 | 0.5 | 0.3 | 0.8 | 1.3 | 1.0 | 0.5 | 1.0 | 1.3 |
| C15:0 | 0.6 | 1.1 | 1.3 | 2.6 | 0.7 | 0.6 | 0.6 | 1.1 |
| C16:0 | 13.3 | 21.8 | 21.1 | 26.8 | 14.8 | 10.0 | 8.3 | 12.9 |
| C16:1Δ9 | 47.2 | 4.8 | 8.2 | 7.7 | 47.9 | 1.6 | 3.5 | 2.5 |
| C18:0 | 5.5 | 7.8 | 5.6 | 6.4 | 6.2 | 4.6 | 2.6 | 3.6 |
| C18:1Δ9 | 31.6 | 3.2 | 2.8 | 3.3 | 28.2 | 0.8 | 1.4 | 1.0 |
| C18:1Δ11 | 1.3 | 0.1 | 0.1 | 0.1 | 1.2 | 0.0 | 0.1 | 0.0 |
| C18:2 (LA) | 0.0 | 0.2 | 0.1 | 0.2 | 0.0 | 3.5 | 2.3 | 0.1 |
| C18:3ω6 (GLA) | 0.0 | 60.7 | 0.2 | 0.5 | 0.0 | 78.1 | 0.0 | 0.5 |
| C20:3ω6 (DGLA) | 0.0 | 0.1 | 59.6 | 0.3 | 0.0 | 0.3 | 80.2 | 0.2 |
| C20:4ω6 (ARA) | 0.0 | 0.0 | 0.0 | 50.8 | 0.0 | 0.0 | 0.0 | 76.8 |
| % of DCW | 0.8 | 1.0 | 0.9 | 0.9 | 2.4 | 2.8 | 3.0 | 2.7 |

### Larger Scale Production of Phospholipids Having ω6 Fatty Acid (Experiment B005)

In a larger scale experiment with 25 L of culture, wild-type Y. lipolytica strain W29 was grown in a Braun fermenter with the addition of ARA to the medium, seeking to produce more cell biomass, increase the polar lipid:TAG ratio and improve the incorporation of ω6 fatty acid into polar lipids. As the experiment aimed to incorporate ω6 fatty acid into the phospholipids and to provide for a greater ratio of PL:TAG, the fermentation was terminated towards the end of active growth rather than in stationary phase, as follows. The growth medium was based on a rich YPD medium which favoured biomass production rather than TAG production. The base medium contained Yeast Extract at 3 g/L, Malt Extract at 3 g/L, Soy peptone at 5 g/L and dextrose monohydrate as the main carbon source at 10 g/L. The pH was initially adjusted to 6.0. This medium was prepared and sterilised in the fermenter by autoclaving in situ, then cooled by direct cooling to the fermenter jacket. After the medium had cooled to 29° C., ARA was added aseptically by overpressure to the medium in the form of 12.5 g ARA (NuChek) as free fatty acid in 300 ml of 17% Triton-X-100 to give a final concentration in the fermenter of 0.5 g/L ARA and 0.2% Triton-X-100, with further addition of 100 ml of unhydrolyzed ARA oil to provide a concentration of 0.4% (v/v) unhydrolyzed ARA oil in addition to the FFA. A seed culture was prepared in 400 ml YM medium at 29° C. with shaking at 180 rpm overnight, providing an inoculum having an OD600 of 4.23. When the medium temperature was 29° C., 400 mL of the seed culture was transferred to the fermenter by overpressure, providing an initial cell density (OD600) of 0.07 by calculation.

The initial fermentation parameters at inoculation were DO at 7.92, pH 7.01, air introduction at 10 ml/min, agitation at 5% of full speed, and back pressure at 11 psi. The initial OD600 was 3.35, almost entirely from the surfactant/oil emulsion, so DO, citric acid production and pH changes were tracked to follow logarithmic growth. In particular, these parameters were followed after about 15 h post inoculation for signs that log-growth was slowing. Agitation and air flow were low to avoid excessive foaming from the surfactant. The backpressure (11 psi) was applied to ensure good oxygen transfer at the low agitation speed. The pH was not controlled. Almost no antifoam (20% Silfax D3 food grade) was used during this experiment.

According to citric acid production, exponential growth started 6-7 h after inoculation and began to slow 16 h after inoculation when the broth was chilled and the cells harvested by centrifugation. The growth may have slowed due to carbon limitation or because it reached a sub-optimal pH. The start medium contained 10 g/L glucose and 3 g/L maltose and if all was consumed at maximal yield, the yeast cell density was expected to be about 6.5 g/L assuming 50% yield. The DCW at harvest was 4.2 g/L. This demonstrated that carbon limitation had likely not been reached which was consistent with the objective to harvest the cells at late log-phase to avoid carbon limitation and subsequent digestion of ARA-PL. The culture was terminated at late logarithmic growth phase to maximise polar lipid content and ARA incorporation and was not heat treated at the end of the fermentation. At harvest, the cells were budding as observed by light microscopy and there were very few that stained with Methylene Blue, so the oil content and therefore the TAG content was low as intended. A final yield of 294 g of wet paste was obtained from the 21 L of culture, with approximately 72% water content i.e. approximately 28% w/w solids. The cell paste was frozen and then freeze dried in 3 batches to yield 73 g of dry yeast cake. The dry yeast cake was milled to a fine powder and dispensed as 3 portions—a 3 g portion for lipid analysis, a 35 g portion for food application trials and a 35 g portion for further processing to yield a crude lipid fraction.

Lipid was extracted from 35 g of yeast powder by adding 900 mL of 60% hexane/40% dry ethanol in a 1 L bottle. The bottle was shaken in an orbital shaker at 180 rpm for 4 h at 29° C. The yeast powder was well suspended in the solvent using this approach. After 4 h of extraction, the solvent was filtered into a glass flask using a ceramic Buchner funnel and a glass filter (Advantec GA-100, 125 mm diameter). Some yeast debris bypassed the filter so the solution was re-filtered by gravity into a 2 L round bottom flask. The solvent was evaporated under vacuum to a final volume of approximately 20 mL and transferred to a glass culture tube for shipment.

As shown in Table 9, the fatty acid composition of the polar lipid fraction from the extracted lipid included 16.4% ARA, as well as 25% of LA. There were also smaller amounts of the other ω6 fatty acids GLA, EDA and DGLA present in the total fatty acid content, and a trace amount of the ω3 fatty acid ALA. Monounsaturated fatty acids were present included 32.7% oleic acid, the most prevalent fatty acid in the polar lipid fraction, and 7.4% palmitoleic acid. Saturated fatty acids (SFA) were present at lower amounts, predominantly palmitic acid at 12.7% and surprisingly low levels of stearate at 0.5% in the total fatty acid content of the polar lipid fraction. In contrast, the fatty acid composition of the TAG fraction was different, including 22.1% ARA. Other ω6 fatty acids were either absent or lower than in the polar lipids, for example LA at 16.7%. Again, oleic acid was the predominant fatty acid in the TAG fraction. In this experiment, where the inventors intended to not produce much TAG through the culture conditions used, the TAG content was indeed low, with a favourable polar lipid:TAG ratio of about 20 in the total lipid content.

### Further Larger Scale Production in Yarrowia of Phospholipids Having ω6 Fatty Acid (B009)

Several experiments were carried out in a similar manner to B005 at the 25 L scale except with some modifications to the culture medium and conditions in an attempt to increase the biomass yield per litre while maintaining the level of incorporation of ARA into PL after supplementation. In experiment B009, three different fungal lipases (100 mg each) were added to the culture medium with the aim of assisting with hydrolysis of the ARA oil and incorporation of the ARA, even though Y. lipolytica is known to produce and excrete TAG lipases. Additionally, the ARA as FFA and the ARA unhydrolysed oil were first mixed with the 200 mL inoculum and then delivered to the fermenter. The non-ionic surfactant Triton X-100 was therefore added to the YPD broth before sterilisation, at the same final concentration as previously used (0.2% v/v), and autoclaved in situ with the broth.

The dissolved oxygen (DO) probe provided unexpectedly low readings 20 min post inoculation, hence the pH, OD and dry weight were the only parameters used to monitor growth of the culture in this experiment. The pH of the culture medium was not controlled in this experiment, falling from pH 6.7 to 3.3 at 16 h due acid production from cellular metabolism. The cell density (dry weight) was 9.4 g/L at 16 h, while optical density of washed cell samples increased from 0.1 to 29.3 at time 0 and 16 h, respectively. There was no bacterial growth observed during the fermentation process as determined by tests for coliforms and Salmonella, and aerobic plate count. The culture was chilled at 16 h post inoculation, the cells harvested and the cell pellets washed three times with cold deionised water. The cell paste was then heat treated at a temperature above 76° C. and below 82° C. for 3 min, aiming to inactivate the cells, then chilled by immersing the container in a water bath with ice. The fermentation terminated at 16 h produced a wet cell paste of 1390 g having a dry cell weight of 236 g. The cell paste was freeze dried.

Lipid was extracted from biomass samples using 25 mL 60% hexane:40% ethanol as solvent per gram of the freeze-dried cells, for 3.5 h at 30° C. The solvent extracts were evaporated under vacuum at 50° C. and then dried under CO₂ gas at 10 L/min. The total lipid content of the 16 h freeze-dried sample was 4.6% on a dry weight basis. The extracted lipid was resuspended in chloroform at a concentration of 200 mg/mL and chromatographed on a TLC plate as before. The TLC results showed substantial amounts of polar lipid had been extracted from the 16 h cells. The ARA levels in the lipid extracted from the biomass when analysed by GC were 7.7% and 2.6% in TAG and PL, respectively, and 2.4% and 2.5% of the total fatty acid content in the TAG and polar lipid fractions, respectively. In this B009 experiment, the biomass production was much greater, but the ARA incorporation rate was reduced. There therefore appeared to be an inverse relationship between the amount of biomass produced and the level of ARA incorporation.

### Experiments B012 and B013

The previous experiments at 25 L scale with Y. lipolytica strain W29 were all cultured in YPD broth with the addition of 0.2 mL/L Triton X-100 to solubilise 100 mL of ARA oil and 10 g ARA as FFA. All of the ferments were terminated at about 16 h. Those experiments varied in terms of lipase addition, cell density at harvest and ARA levels in the polar lipid of the harvested biomass. In experiment B012, the lipases were omitted from the culture, backpressure was set to 15 psi and airflow at 12, to provide about 10 ppm dissolved oxygen during culturing. The cell density (OD600) of the inoculum was 9.19, so 200 mL was added to the 25 L medium in the fermenter to achieve a starting OD600 calculated at 0.08. The ARA oil and FFA were added as before. The pH dropped from an initial 7.08 at 0 h to 4.63 at 15.68 h but then started to increase in the last 30 min of the culturing. At this point, the culture might have reached stationary phase and glucose was depleted. After the exhaustion of glucose, the cells might have started breaking down phospholipids for maintenance. It was therefore considered important to harvest the culture before it reached stationary phase. The optical density, calculated at TO and corrected by washing the cells with water at 16 h, increased from 0.08 to 27.4 at 16 h, yielding a culture density of 9 g/L on a dry weight basis.

The cell biomass was harvested from the culture and the pellets washed twice with cold deionized water. The washed cells were heat inactivated at a temperature of approximately 95° C. for 3 min, then chilled by immersing the container in a water bath with ice. The heat inactivation of the yeast cells was successful as shown by a lack of viable cells when plated. In this experiment, 225 g of dry cell biomass was generated. Total lipid was extracted from biomass samples and analysed as before. The freeze-dried cells contained about 4.7% crude lipid. The polar lipid fraction from this experiment had 4.1% ARA and the TAG fraction had 4.0% ARA as a percentage of the total fatty acid content of those fractions (Table 8). The total lipid also had less TAG, MAG and FFA than in previous experiments, as shown by TLC. This was taken as an indication that the cells took up the ARA and incorporated it into PL in cell membranes under the prescribed culture conditions, however, the PL might have been broken down to some extent to maintain cellular activities due to glucose depletion in the medium.

Another experiment (B013) was carried out with the following adjustments to the culture conditions: the starter culture OD600 was between 4 and 5, the ARA FFA and the ARA oil were formulated with 5% Triton X-100 as a concentrated pre-mix and then added to the fermenter prior to inoculation, the pH trend was used to estimate the optimal harvest point by monitoring it to be greater than 4.0. The pH trend was closely monitored from 14 h to ensure culture termination and cell harvest before glucose exhaustion occurred and the pH started to rise. To make the culture medium for this experiment, 50 mL Triton X-100 was dissolved in 1 L deionized water and autoclaved. The Triton X-100 separated from the water as the sterilised solution cooled overnight and needed to be warmed to about 50° C. to re-dissolve it, with shaking. Once the Triton X-100 was fully dissolved, it was vigorously mixed with 10.0 g ARA and 100 mL ARA oil to form an emulsion and then pumped into the fermenter. Lastly, 400 mL inoculum culture was transferred to the fermenter by overpressure. The calculated culture density (OD600) at inoculation was 0.07.

During the culturing, the dissolved oxygen level dropped to zero at 6 h post inoculation under the initial set up conditions of airflow at 10 L/m, pressure 10 psi and DO 15.9. The temperature gradually dropped from 28° C. to 23° C. overnight as the culture density was insufficient to generate heat. The reduced temperature was likely beneficial in decreasing the culture growth rate shown by the gradual decrease in pH decline. At 14 h, the airflow, stirring rate and backpressure were changed to increase the DO and the temperature was also increased. The OD600 was 7.4 at 14 h, therefore, the fermentation was extended by 2 hours until the OD600 was above 10 and the pH began to stabilise at pH 5. The culture was run without pH control for 16 hours, the pH naturally falling from pH 6.96 to 5.07 due to acid production from cellular metabolism. The cell density (dry weight) was 5.27 g/L at 16 h, while the OD600 increased from 0.07 to 12.1 at 16 h. The culture assimilated 4.5 g/L of glucose, which was 51% of the 8.9 g/L glucose supplied in the start medium.

The harvested cells were heat inactivated at a temperature of 95° C. for 3 min as before, yielding 584 g wet weight of biomass corresponding to 114 g dry weight. Lipid was extracted from freeze-dried samples and analysed as before. The total lipid content of the 16 h freeze-dried cells was 3.4%. The TLC analysis showed that more polar lipid was present than in experiment B012. The ARA level in the polar lipid and TAG fractions were 10.2% and 13.3%, respectively. The data for the fatty acid compositions are provided in Table 9.

It was concluded that experiment B013 had provided a useful biomass content and a reasonable level of ARA incorporation into polar lipid, even though further optimisation of both parameters was desired. The cell biomass produced in B013 and lipids extracted from these cells were used in Maillard reactions simulating food preparations as described in Examples 8 to 10 below.

TABLE 7
Comparison of Y. lipolytica W29 cultures
under different fermentation conditions

| Experiment | B005 | B009 | B012 | B013 |
|---|---|---|---|---|
| Base medium (g/L) | YPD | YPD | YPD | YPD |
| Lipases | 0 | 3 lipases | 0 | 0 |
| TritonX-100 (mL/ L) | 2 | 2 | 2 | 2 |
| Ara oil (mL/L) | 4 | 4 | 4 | 4 |
| Ara-FFA (g/25 L) | 12.5 | 10 | 10 | 10 |
| OD600 of inoculum | 4.2 | 13.3 | 9.2 | 9.2 |
| Starting OD600 | 0.07 | 0.1 | 0.08 | 0.08 |
| Harvest time (h) | 15.5 | 16 | 16 | 16 |
| Start pH | 7.01 | 6.7 | 7.08 | 7.08 |
| Harvest pH | 5.8 | 3.5 | 5.11 | 5.11 |
| Backpressure (psi) | 11 | 15 | 10 | 10 |
| Stirrer (%) | 5 | 30 | 30 | 30 |
| Air Flow | 10 | 20 | 15 | 15 |
| Dissolved Oxygen | 7.92 | 6.29 | 19.13 | 19.13 |
| Biomass (g dry weight) | 75 | 260 | 220 | 220 |
| ARA (%) in polar lipid | 16 | 2.5 | 4.1 | 10.2 |

TABLE 8
Fatty acid composition of polar lipids and TAG in Y. lipolytica
after culturing with ARA, for experiments B005, B009, B012 and B013.

| | B005 | | B012 | | B013 | |
|---|---|---|---|---|---|---|
| | Polar | | Polar | | Polar | |
| | lipid | TAG | lipid | TAG | lipid | TAG |
| C14:0 | 0.2 | 0.0 | 0.0 | 0.0 | 0.2 | 0.0 |
| C15:0 | 0.0 | 0.0 | 0.2 | 0.2 | 0.8 | 0.0 |
| C16:0 | 12.7 | 14.1 | 10.1 | 7.5 | 12.0 | 8.0 |
| C16:1Δ9 | 7.4 | 0.0 | 7.7 | 9.3 | 9.3 | 5.6 |
| C18:0 | 0.5 | 11.3 | 1.6 | 0.3 | 0.8 | 5.6 |
| C18:1Δ9 | 32.7 | 35.7 | 50.6 | 48.5 | 29.2 | 35.7 |
| C18:1Δ11 | 0.4 | 0.0 | 0.6 | 0.5 | 0.5 | 0.9 |
| C18:2 (LA) | 25.0 | 16.7 | 20.8 | 24.3 | 33.5 | 17.1 |
| C18:3ω3 (ALA) | 0.2 | 0.0 | 0.0 | 0.0 | 0.0 | 0.0 |
| C18:3ω6 (GLA) | 1.6 | 0.0 | 0.4 | 0.5 | 0.4 | 0.0 |
| C19:0 | 0.0 | 0.0 | 0.4 | 0.9 | 0.8 | 0.0 |
| C20:0 | 0.0 | 0.0 | 0.3 | 0.4 | 0.4 | 0.0 |
| C20:1Δ11 | 0.3 | 0.0 | 0.0 | 0.0 | 0.0 | 0.0 |
| C20:1Δ5 | 0.4 | 0.0 | 0.0 | 0.0 | 0.0 | 0.0 |
| C20:2ω6 (EDA) | 0.2 | 0.0 | 0.0 | 0.0 | 0.0 | 0.0 |
| C20:3ω6 (DGLA) | 1.9 | 0.0 | 0.6 | 0.5 | 0.7 | 0.6 |
| C20:4ω6 (ARA) | 16.4 | 22.1 | 4.1 | 4.0 | 10.2 | 13.3 |
| C24:0 | 0.2 | 0.0 | 1.0 | 2.8 | 1.2 | 12.9 |
| % of DCW | 2.0 | 0.1 | | | | |

### Example 5. Maillard Reaction and Volatiles Tests Using Polar Lipids Having ω6 FA

As described in Example 4, polar lipids including PL with one or more of the ω6 fatty acids GLA, DGLA or ARA were produced in yeast cells, extracted and purified. In an initial experiment to see if a Maillard reaction could be induced with these lipid extracts and what properties the resultant products would have, polar lipid preparations including GLA or ARA were mixed with cysteine and ribose in glass vials and heated in an oven at 140° C. for 1 h. This Example describes these experiments and the results.

### Experiment 1. Maillard Reactions

Polar lipid samples were prepared by extraction from yeast cells supplemented with GLA or ARA and fractionation as described in Example 4. Samples of 8.0 mg of polar lipid from the ARA-fed cells, 7.6 mg from the GLA-fed cells, 9.0 mg from the control cells and 16.0 mg of polar lipid extracted from pork meat, each dissolved in chloroform, were transferred to 20 ml glass vials. The solvent was evaporated under nitrogen flow at room temperature. 2 ml of 0.1 M potassium phosphate buffer, pH 7.2, containing 4.5 mg/ml ribose (Catalog No. R9629, Sigma-Aldrich) and 5.0 mg/ml cysteine (Catalog No. 30089, Sigma-Aldrich) was added to each vial, and the vials tightly closed with metal lids having PTTF liners. A control vial had the buffer but no polar lipid. The vials were subjected to ultrasonication in a water bath at 40° C. for 1 h and then heated in an oven at 140° C. for 1 h by placing the vials on the bottom metal surface of the oven. After the heating, the mixtures all appeared orange-brown in colour, suggesting that a chemical reaction had occurred. Serendipitously, the vial containing the polar lipid from the ARA-fed cells leaked and a distinct roast meat-like aroma was noticed that spread inside and even outside the laboratory. The other vials were then cooled, opened and smelled. The heated mixtures having the ARA-fed and pork polar lipids gave off pleasant, meat-like aromas, while the mixture including the GLA-fed polar lipid had a mild garlic-like aroma. In contrast, the mixture having the polar lipid from Y. lipolytica that had not been fed the amino acids (control) and the control mixture lacking lipid emitted a sulphurous aroma. The inventors concluded that the polar lipid containing ARA provided a more meat-like aroma than the polar lipid containing GLA, even though the GLA was present at a 3-fold greater amount in the polar lipid than the ARA. The inventors also concluded that the presence of ARA in the polar lipid provided the meat-like aroma, which did not occur with the corresponding polar lipid lacking the ARA.

These observations prompted the inventors to carry out further tests with extracted lipids containing ω6 fatty acids to determine their capacity to provide meat-like flavour and aroma compounds and to measure the volatiles by GC-MS, as follows. Experiments were also carried out with whole cells having the ω6 fatty acids in their polar lipids, rather than with extracted lipids from the cells, as follows.

### Experiment 2

Encouraged by the results of the first experiment, a second experiment was performed, including a sensory evaluation by a panel of volunteers to detect aromas. Polar lipid was extracted from Y. lipolytica strain W29 cells as before. The fatty acid composition of the polar lipid was determined by GC-FID of FAME, showing the presence of 16.3% ARA (Table 9). Samples of 15 mg of polar lipid were treated in the same manner as in Experiment 1. Additional, control mixtures having buffer with ribose but without the cysteine were prepared to test the effect of omitting the sulphur-containing amino acid. Other mixtures were prepared including either soy lecithin (The Ingredients Centre, VIC, Australia) or an ARA-containing oil (Jinan Boss Chemical Industry Co, China) containing 50% ARA as a percentage of the total fatty acid content (Example 4). As before, 2.0 ml of 0.1 M potassium phosphate buffer, pH 7.2, containing 4.5 mg/ml ribose and in some cases 5.0 mg/ml cysteine was added to each 10 mL SPME vial, and the vials tightly closed with PTFE-lined screw top caps. The vials were then subjected to ultrasonication for 1 h in a water bath at 40° C. and heated at 140° C. for 1 h, as before. After the heat treatment, the mixtures having ribose without cysteine had a dark brown, coffee-like colour, whereas those having both ribose and cysteine were lighter brown in colour.

After the vials were cooled to room temperature, sensory analysis was carried out by nine volunteers, consisting of 5 males and 4 females aged 30 to 65 years, of different backgrounds. The sample identities were not revealed until after the completion of the sensory evaluation. Each vial was gently shaken and the lid was opened to sniff the aroma. The vials containing the lipid and ribose without cysteine were presented first, followed by the vials containing the lipid, ribose and cysteine, in the order vials 1, 4, 6, 2, 5, 7 and 3. The volunteers' reactions were recorded (Table 10). It was clear that although there was some diversity in the responses, vial 3 was consistently referred to as providing a pleasant, meaty or roast beef aroma.

The samples were then analysed by HS-SPME-GCMS for volatiles as described in Example 1. The GC-MS analyses revealed volatile compounds which were present in the mixtures containing the ARA-fed polar lipid but absent from the mixtures containing the polar lipid from the non-fed cells. These compounds were: 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; and 2,3,4,5-Tetramethylcyclopent-2-en-1-ol. It was concluded that these compounds were associated with the roasted meat-like aroma for the mixture in vial 3.

In a repeat of the experiment, the concentrations of ribose and cysteine were halved, attempting to reduce sulphurous aromas. Similar results were obtained as before, with some reduction in the sulphurous component of the aromas. The responses from 6 other volunteers confirmed that the polar lipid from the ARA-fed Y. lipolytica provided roasted beef-like aromas, different to the aromas from the soy lecithin and ARA oil mixtures. Notably, one of the volunteers had a pet dog which showed great interest in the aroma.

TABLE 9
Fatty acid composition of polar lipids extracted from Y. lipolytica cultures fed with GLA, ARA or no added fatty
acid (control). The polar lipid fraction of Experiment 8 also contained 0.3% C20:1Δ11, 0.4% C20:1Δ5 and 0.2% C20:2ω6.

| | | | | | | | | | |
|---|---|---|---|---|---|---|---|---|---|
| Sample | C14:0 | C15:0 | C16:0 | C16:1Δ7 | C16:1Δ9 | C17:1 | C18:0 | C18:1 | C18:1Δ11 |

Experiment 1

| | | | | | | | | | |
|---|---|---|---|---|---|---|---|---|---|
| Control | 0.2 | 0.3 | 9.4 | 4.0 | 10.4 | 1.8 | 0.8 | 49.9 | 0.3 |
| GLA-fed polar lipid | 0.1 | 2.0 | 17.9 | 0.1 | 2.0 | 0.6 | 2.9 | 8.5 | 0.1 |
| ARA-fed polar lipid | 0.3 | 1.0 | 14.1 | 0.5 | 9.5 | 1.6 | 1.1 | 42.7 | 0.5 |
| Pork polar lipid | 0.2 | 6.6 | 19.0 | 0.6 | 0.9 | 0.6 | 14.0 | 16.7 | 4.4 |
| Pork TAG | 1.9 | 0.0 | 28.0 | 0.0 | 2.7 | 0.3 | 14.9 | 41.2 | 4.8 |

Experiments 2-4

| | | | | | | | | | |
|---|---|---|---|---|---|---|---|---|---|
| ARA-fed polar lipid (1 L) | 0.2 | 0.8 | 15.6 | 0.8 | 8.6 | 0.2 | 1.4 | 46.9 | 0.4 |
| ARA-fed polar lipid (3 L) | 0.0 | 0.0 | 15.5 | 0.9 | 8.4 | 0.6 | 1.7 | 44.8 | 0.4 |
| Soy lecithin (unpurified) | 0.1 | 0.0 | 20.5 | 0.0 | 0.1 | 0.0 | 4.3 | 8.4 | 1.4 |
| Soy lecithin (TLC pure) | 0.1 | 0.0 | 20.9 | 0.0 | 0.1 | 0.0 | 4.0 | 9.0 | 1.4 |

Experiment 6

| | | | | | | | | | |
|---|---|---|---|---|---|---|---|---|---|
| ARA-fed polar lipid | 0.2 | 0.2 | 14.7 | 2.9 | 0.9 | 1.0 | 0.7 | 28.0 | 0.7 |
| ARA-fed free fatty acid | 0.1 | 0.1 | 6.8 | 0.4 | 0.2 | 0.1 | 53.1 | 4.8 | 0.2 |

Experiment 7

| | | | | | | | | | |
|---|---|---|---|---|---|---|---|---|---|
| GLA-fed polar lipid (3 L) | 0.0 | 0.0 | 16.2 | 0.4 | 7.1 | 0.0 | 2.4 | 19.5 | 0.2 |

Experiment 8

| | | | | | | | | |
|---|---|---|---|---|---|---|---|---|
| ARA-fed whole cells | 0.2 | 0.0 | 12.7 | 7.4 | 0.0 | 0.5 | 32.7 | 0.4 |

| | C18:2 | C18:3ω6 | C18:3ω3 | | C20:3ω6 | C20:4ω6 | | |
|---|---|---|---|---|---|---|---|---|
| Sample | (LA) | (GLA) | (ALA) | C20:0 | (DGLA) | (ARA) | C22:0 | C24:0 |
| Experiment 1 | | | | | | | | |
| Control | 22.8 | 0.0 | 0.0 | 0.0 | 0.0 | 0.0 | 0.0 | 0.1 |
| GLA-fed polar lipid | 3.2 | 61.3 | 0.0 | 0.1 | 0.6 | 0.3 | 0.0 | 0.3 |
| ARA-fed polar lipid | 9.8 | 0.0 | 0.0 | 0.0 | 0.6 | 18.2 | 0.0 | 0.2 |
| Pork polar lipid | 25.2 | 0.3 | 0.5 | 0.2 | 0.8 | 6.6 | 0.3 | 0.2 |
| Pork TAG | 5.1 | 0.1 | 0.3 | 0.2 | 0.0 | 0.1 | 0.0 | 0.0 |
| Experiments 2-4 | | | | | | | | |
| ARA-fed polar lipid (1 L) | 9.1 | 1.4 | 0.0 | 0.0 | 0.3 | 14.1 | 0.0 | 0.2 |
| ARA-fed polar lipid (3 L) | 9.6 | 1.2 | 0.0 | 0.0 | 0.3 | 16.3 | 0.0 | 0.2 |
| Soy lecithin (unpurified) | 57.5 | 0.0 | 6.8 | 0.1 | 0.0 | 0.0 | 0.4 | 0.3 |
| Soy lecithin (TLC pure) | 56.5 | 0.0 | 6.9 | 0.2 | 0.0 | 0.0 | 0.6 | 0.4 |
| Experiment 6 | | | | | | | | |
| ARA-fed polar lipid | 44.6 | 2.1 | 0.0 | 0.0 | 0.8 | 3.2 | 0.1 | 0.1 |
| ARA-fed free fatty acid | 4.6 | 0.5 | 0.0 | 0.0 | 1.3 | 7.3 | 12.6 | 8.1 |
| Experiment 7 | | | | | | | | |
| GLA-fed polar lipid (3 L) | 1.6 | 51.8 | 0.0 | 0.1 | 0.5 | 0.0 | 0.0 | 0.2 |
| Experiment 8 | | | | | | | | |
| ARA-fed whole cells | 25.0 | 1.4 | 0.2 | 0.0 | 1.9 | 16.4 | 0.0 | 0.2 |

TABLE 10
Aromas of mixtures of polar lipid, ribose and cysteine after heat
treatment, as detected by a sensory panel of 9 volunteers.

| Vial | | Ribose | Cysteine | |
|---|---|---|---|---|
| No. | Lipid source | (mg/ml) | (mg/ml) | Description of aromas |
| 1 | Control (non-fed) polar lipid | 4.5 | 0 | caramel-like, baked, biscuity |
| 2 | Control (non-fed) polar lipid | 4.5 | 5.0 | smoky, garlic, burnt |
| 3 | ARA-fed polar lipid | 4.5 | 5.0 | Roasted beef, meaty, pleasant |
| 4 | Soy lecithin | 4.5 | 0 | baked, fishy |
| 5 | Soy lecithin | 4.5 | 5.0 | smoky, garlic, burnt popcorn |
| 6 | ARA oil (TAG) | 4.5 | 0 | something raw, raw fish |
| 7 | ARA oil (TAG) | 4.5 | 5.0 | lighter aroma, old beef roast |

### Experiment 3

In another experiment, 15 mg samples of the extracted polar lipid preparations, the soy lecithin or the ARA oil were separately mixed with 2 ml of the potassium phosphate buffer containing 2.25 mg/ml ribose and 2.5 mg/ml cysteine, pH 7.2, in 12 ml glass tubes rather than the SPME vials. The fatty acid compositions for the Y. lipolytica-derived preparations and the soy lecithin (unpurified and TLC-purified) are provided in Table 10. The lipids tested were:
1. Polar lipid from Y. lipolytica grown in the presence of ARA (Yl ARA)
2. Polar lipid from Y. lipolytica grown in the absence of ARA (Yl) (Control)
3. Soy lecithin (The Ingredients Centre)
4. ARA oil (Jinan Boss Chemical Industry Co, China)
5. No lipid (control)

In an initial attempt, the mixtures were sonicated in the 12 ml Pyrex glass tubes with plastic caps lined with PTFE seals and then heated for 1 h at 140° C. The tubes were placed in a rack in the oven rather than in contact with a metal surface of the oven. This time, the mixtures were not brownish in colour, instead appeared rather turbid but colourless. GC-MS analysis showed only low levels of volatiles, indicating that the Maillard reactions had not gone to completion. The inventors thought that insufficient heating or the smaller surface area of the mixtures in the tubes may have contributed to the reduced reaction. The remaining mixtures were therefore transferred to SPME vials and heated again at 140° C. for 2 h by placing the vials on an aluminium foil inside the oven. This time, the colour of the mixtures changed to pale brown as in previous experiments. The vials were cooled down and stored at −20° C. For GC-MS analysis of volatile compounds, 0.5 ml of each sample was transferred into new SPME vials for injection in the split 1:20 mode and other 0.2 ml transferred into new vials for injection by splitless mode.

### Volatile Compounds Released by the Treatment

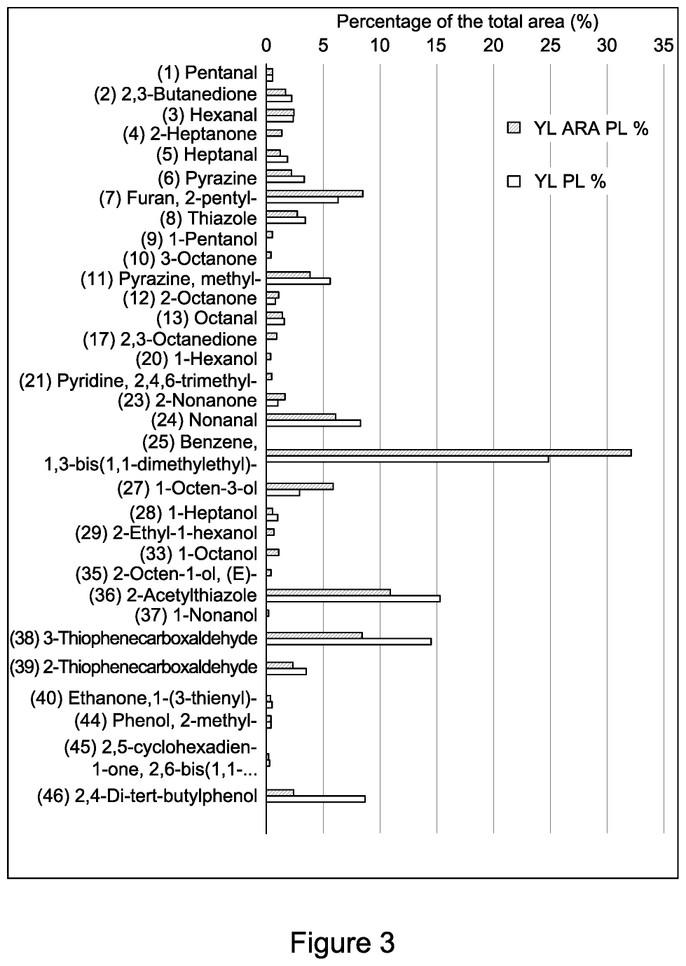

The profile of volatile compounds released by heating the extracted lipids with the mixture of ribose and cysteine was evaluated by headspace solid-phase microextraction coupled with gas chromatography-mass spectrometry (HS-SPME-GCMS) as described in Example 1. The majority of volatiles are generated by a combination of lipid oxidation and other degradation processes, as well as Strecker reaction and Maillard reaction products, including production of aldehydes, alcohols, ketones, pyrazines and furans. The GC-MS data are shown in Table 12 and FIG. 3 which presents the levels of each of the identified compounds as the area percentage (%) of total identified compounds for reaction mixtures containing the ARA-polar lipid (YL ARA) or non-fed polar lipid (YL).

The sample containing polar lipid from the Y. lipolytica cells fed with ARA, heated in the presence of cysteine and ribose under conditions to produce the Maillard reaction, produced specific volatile compounds including 2-heptanone, 3-octanone, 2,3-octanedione, 1-pentanol, 1-hexanol, 2-ethyl-1-hexanol, 1-octanol, trans-2-octen-1-ol and 1-nonanol. These compounds were not detected in the Maillard products from the polar lipid extracts from the control Y. lipolytica cells grown in the absence of ARA (YL). Of these, 3-octanone and 1-nonanol were detected only in the reaction having the YL ARA polar lipid i.e. not in any of the other vials. Other compounds, namely 2-heptanone, 2,3-octanedione, 1-hexanol and 1-octanol were detected only in the reactions having YL ARA and the soy lecithin. The ω6 fatty acid in the reactions with polar lipid containing ARA clearly created a chemical difference which was associated with the sensory difference observed by the volunteers, with an increased amount of lipid oxidation products and reduced amounts of heterocyclic compounds, such as pyrazines. The presence of certain ketone and alcohol compounds registered here were also observed in volatile profiles for meat flavours as a result of lipid oxidation. The ketone 2-heptanone present in the samples with YL ARA and soy lecithin was thought to be due to lipid oxidation and related to ethereal, butter or spicy flavours. The volatile compound 1,3-bis(1,1-dimethylethyl)-benzene was the main compound produced (FIG. 3) and was significantly increased in amount in the reaction mixture made with the ARA-polar lipid relative to the control polar lipid from Y. lipolytica cells. That compound has a characteristic beef-like aroma.

Results from the experiment indicated that the compound acetylthiazole, common to all samples tested and shown in Table 11, has a sulphurous and roast meat aroma resulted from the reaction with cysteine and ribose. The aldehydes hexanal and nonanal, which were produced from all mixtures except the ‘no lipid’ control sample, were produced from the lipid oxidation. Hexanal is associated with oxidation of ω6 fatty acids such as LA and ARA. Nonanal contributes to tallow and fruity flavour and it is one of the key volatiles in cooked beef together with octanal. Octanal was produced from the samples containing YL ARA, YL and soy lecithin, i.e. all three polar lipid samples, but not produced from the ARA-Oil and no lipid samples. The unsaturated alcohol 1-octen-3-ol, also produced in all the oil-containing samples tested (YL ARA, YL, soy lecithin and ARA Oil) may contribute to an herbaceous aroma resulted from thermal decomposition of methyl linoleate hydroperoxide. The compound 2-pentylfuran, present in all but the no lipid mixture, was derived from LA. Furan-containing compounds were also possibly produced from the thermal degradation of sugars.

TABLE 11
Detection and identification by HS-SPME-GC-MS of volatile compounds produced in Maillard reactions
with Y. lipolytica polar lipids after feeding the cells with ARA (YL ARA) or not fed ARA as a control
(YL), indicating the detected presence (Y) or absence (N) of the compounds in each mixture.

| | | | | | YL | | Soy | ARA | No |
|---|---|---|---|---|---|---|---|---|---|
| No. | Compound | Reported LRI | Observed LRI | ID | ARA | YL | lecithin | oil | lipid |
| 1 | Pentanal | 990 | 990 | MS, RI, S | Y | Y | Y | N | N |
| 2 | 2,3-Butanedione | 986 | 991-992 | MS, RI | Y | Y | Y | Y | Y |
| 3 | Hexanal | 1078 | 1075-1076 | MS, RI, S | Y | Y | Y | Y | N |
| 4 | 2-Heptanone | 1185 | 1181-1182 | MS, RI | Y | N | Y | N | N |
| 5 | Heptanal | 1188 | 1183 | MS, RI, S | Y | Y | N | N | N |
| 6 | Pyrazine | 1209 | 1214-1220 | MS, RI | Y | Y | Y | N | Y |
| 7 | Furan, 2-pentyl- | 1236 | 1229-1232 | MS, RI, S | Y | Y | Y | Y | N |
| 8 | Thiazole | 1210-1270 | 1251-1257 | MS, RI | Y | Y | Y | Y | Y |
| 9 | 1-Pentanol | 1254 | 1256 | MS, RI, S | Y | N | N | Y | N |
| 10 | 3-Octanone | 1251 | 1256 | MS, RI | Y | N | N | N | N |
| 11 | Pyrazine, methyl- | 1238-1309 | 1266-1276 | MS, RI | Y | Y | Y | Y | Y |
| 12 | 2-Octanone | 1280 | 1280-1287 | MS, RI | Y | Y | Y | Y | N |
| 13 | Octanal | 1294 | 1290-1291 | MS, RI, S | Y | Y | Y | N | N |
| 14 | 2-Propanone, 1-hydroxy- | 1266-1340 | 1311-1322 | MS, RI | N | N | N | Y | Y |
| 15 | 2-Heptenal, (Z)- | 1320 | 1326 | MS, RI | N | N | Y | N | N |
| 16 | Pyrazine, 2,6-dimethyl- | 1325 | 1328-1331 | MS, RI | N | N | Y | N | Y |
| 17 | 2,3-Octanedione | 1320-1376 | 1330-1331 | MS, RI | Y | N | Y | N | N |
| 18 | Pyrazine, ethyl- | 1292-1359 | 1334-1337 | MS, RI | N | N | Y | Y | Y |
| 19 | Pyrazine, 2,3-dimethyl- | 1345 | 1346 | MS, RI | N | N | Y | N | N |
| 20 | 1-Hexanol | 1354 | 1357 | MS, RI | Y | N | Y | N | N |
| 21 | Pyridine, 2,4,6-trimethyl- | 1369 | 1367 | MS, RI, S | Y | N | N | N | N |
| 22 | Pyrazine, 2-ethyl-6-methyl- | 1353-1420 | 1384 | MS, RI | N | N | Y | N | N |
| 23 | 2-Nonanone | 1394 | 1391 | MS, RI | Y | Y | Y | N | N |
| 24 | Nonanal | 1399 | 1396 | MS, RI, S | Y | Y | Y | Y | N |
| 25 | Benzene, 1,3-bis(1,1-dimethylethyl)- | 1420-1454 | 1429-1431 | MS, RI | Y | Y | N | N | N |
| 26 | 2-Octenal, (E)- | 1400-1441 | 1427-1432 | MS, RI | N | N | Y | Y | N |
| 27 | 1-Octen-3-ol | 1456 | 1455-1457 | MS, RI, S | Y | Y | Y | Y | N |
| 28 | 1-Heptanol | 1460 | 1460 | MS, RI | Y | Y | N | N | N |
| 29 | 2-Ethyl-1-hexanol | 1492 | 1495 | MS, RI | Y | N | N | N | Y |
| 30 | 2-Decanone | 1484 | 1499 | MS, RI | N | N | Y | N | N |
| 31 | Decanal | 1502 | 1503 | MS, RI | N | N | Y | N | N |
| 32 | trans-3-Nonen-2-one | 1495-1547 | 1516 | MS, RI | N | N | Y | N | N |
| 33 | 1-Octanol | 1561 | 1562 | MS, RI | Y | N | Y | N | N |
| 34 | Pyridine, 2-pentyl- | 1527-1592 | 1573 | MS, RI | N | N | Y | N | N |
| 35 | 2-Octen-1-ol, (E)- | 1610-1645 | 1615-1620 | MS, RI | Y | N | Y | Y | N |
| 36 | 2-Acetylthiazole | 1661 | 1650-1651 | MS, RI | Y | Y | Y | Y | Y |
| 37 | 1-Nonanol | 1668 | 1665 | MS, RI | Y | N | N | N | N |
| 38 | 3-Thiophenecarboxaldehyde | 1653-1693 | 1683-1685 | MS, RI | Y | Y | Y | Y | Y |
| 39 | 2-Thiophenecarboxaldehyde | 1655-1734 | 1698-1700 | MS, RI | Y | Y | Y | Y | Y |
| 40 | Ethanone, 1-(3-thienyl)- | 1725-1785 | 1779-1780 | MS, RI | Y | Y | N | N | N |
| 41 | Ethanone, 1-(2-thienyl)- | 1735-1785 | 1771-1773 | MS, RI | N | N | Y | Y | Y |
| 42 | 2,4-Decadienal, (E,E)- | 1790-1821 | 1811 | MS, RI | N | N | N | Y | N |
| 43 | 3-Methyl-2-thiophenecarboxaldehyde | 1761-1815 | 1815 | MS, RI | N | N | N | N | Y |
| 44 | Phenol, 2-methyl- | 1992-2025 | 2012 | MS, RI | Y | Y | N | N | N |
| 45 | 2,5-cyclohexadien-1-one, 2,6-bis(1,1- | 2094-2117 | 2107 | MS, RI | Y | Y | N | N | N |
| | dimethylethyl)-4-hydroxy-4-methyl- | | | | | | | | |
| 46 | 2,4-Di-tert-butylphenol | 2280-2327 | 2321-2322 | MS, RI | Y | Y | Y | Y | Y |

LRI: linear retention index for polar columns obtained from literature and standards (S); observed LRI: linear retention index calculated from tested samples; ID: methods of identification; MS: mass spectrum; RI: retention index; S: standard mass spectrum.

### Experiment 4. Optimisation of the Amount of Lipid in the Reactions

An experiment was carried out varying the amount of lipid used in the Maillard reactions, to test whether smaller amounts of polar lipid could be heat treated and the reaction products still be detected by GC-MS. The purpose of the experiment was to define optimal amounts which produced a chromatogram that detected most of the compounds at the same time and that produced maximum overall intensity. Samples containing 0.5, 2.5, 5.0 or 7.5 mg of 18:0/18:1-phosphatidylcholine (Catalog No. 850467C, Avanti Polar Lipids) in chloroform were transferred to 20 ml SPME vials. Aliquots of 2.5 or 7.5 mg of soy lecithin powder, or 2.5 or 7.5 mg polar lipid extracted from the soy lecithin powder by TLC, were also transferred to SPME vials. The fatty acid composition of the unpurified and TLC-purified soy lecithin is provided in Table 10; the purification had little effect on the fatty acid composition. After evaporation of the chloroform under a flow of nitrogen, 1 ml of 0.2 M potassium phosphate buffer, pH 7.2, containing 2.25 mg/ml ribose and 2.5 mg/ml cysteine were added to the vials and the lids were tightly closed. The vials were subjected to ultrasonication for 1 h at 40° C. in a water bath to emulsify the mixtures and then incubated at 140° C. for 1 h by placing the vials on aluminium foil inside an oven. After the vials were cooled, the volatile compounds in the headspace of each vial were analysed by solid-phase microextraction coupled with gas chromatography-mass spectrometry (HS-SPME-GCMS) as before. The reaction mixtures including 0.5 mg lipid showed peaks for the volatile compounds but at lower intensities than desired with some compounds being undetected. Intermediate lipid amounts (2.5 and 5.0 mg) showed an increased response for most of the compounds, while the maximum amount tested (7.5 mg) of polar lipid showed an overall reduction in intensities perhaps due to overloading. Therefore, the mixtures having 2.5 mg polar lipid in 1 ml reaction volume showed optimal performance without suffering from either disadvantage. That amount of polar lipid was considered optimum for future experiments. Mixtures having 5.0 mg polar lipid in 1 ml reaction volume were also considered suitable for the analyses.

A comparison of reaction products in the mixtures having soy lecithin, either purified through TLC or not purified, revealed the presence of several hydrocarbon compounds in the reaction mixture having the purified soy lecithin that were absent from the corresponding reaction mixture made with the non-purified soy lecithin, therefore considered to be artefacts of the preparation method. These hydrocarbons in the GC-MS chromatogram included both short and long-chain alkanes. The inventors concluded that other polar lipid preparations that had been purified by TLC might also have yielded these hydrocarbon compounds, and therefore these compounds were excluded from the quantitation of the GC-MS traces for the Y. lipolytica polar lipids as resulting from the sample preparation method. The hydrocarbons not considered in the analysis for this reason were: Hexane, 2,4-dimethyl-; Dodecane, 4,6-dimethyl-; Hexadecane; Heptadecane; Undecane, 3,8-dimethyl-; Triacontane; Hentriacontane; Tetradecane, 5-methyl-; Decane, 3,3,6-trimethyl-; and Hexadecane, 2,6,10,14-tetramethyl-.

### Experiment 5. Maillard Reactions for ARA-PC and 18:1—PC.

Another experiment was carried out to compare the reaction products from Maillard reactions for mixtures including pure ARA-phosphatidylcholine (PC) or 18:0/18:1—PC as a comparison, to identify volatile compounds arising specifically from the ARA-PC. Samples containing 2.5 or 5.0 mg of 18:0/18:1-PC (Catalog No. 850467C, Avanti Polar Lipids) or ARA-PC (Avanti Polar Lipids) were treated in 1 ml volumes as for the previous experiment. HS-SPME-GCMS analysis of the volatiles produced after the heating step showed the presence of numerous compounds which were either increased or decreased in the mixtures having ARA-PC relative to the mixtures having 18:0/18:1-PC or were present in one mixture and absent or not detected in the other mixture.

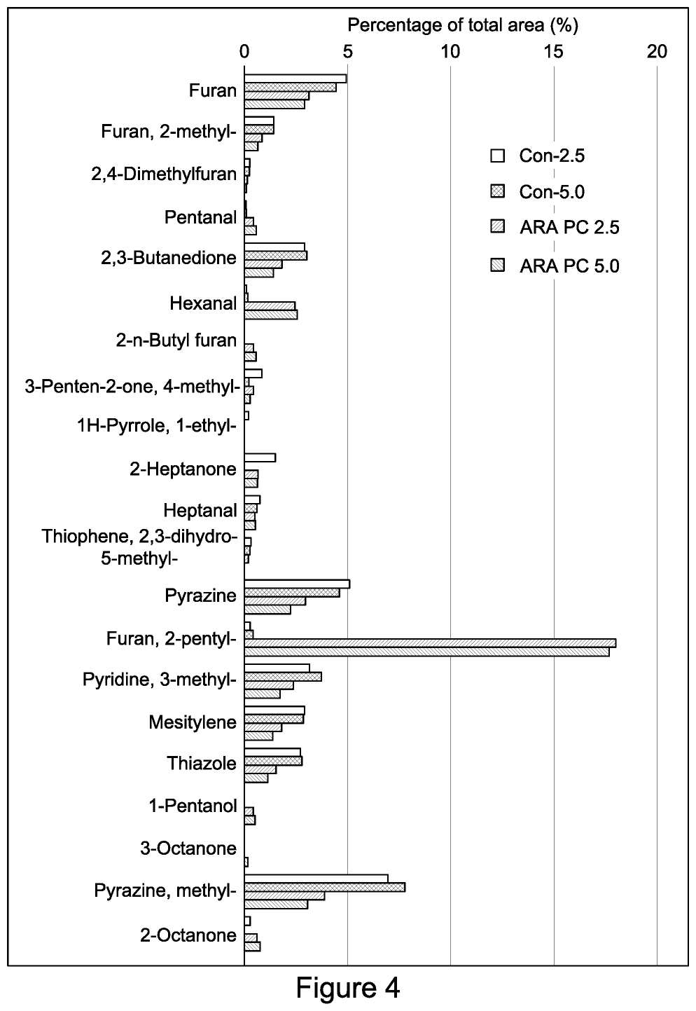

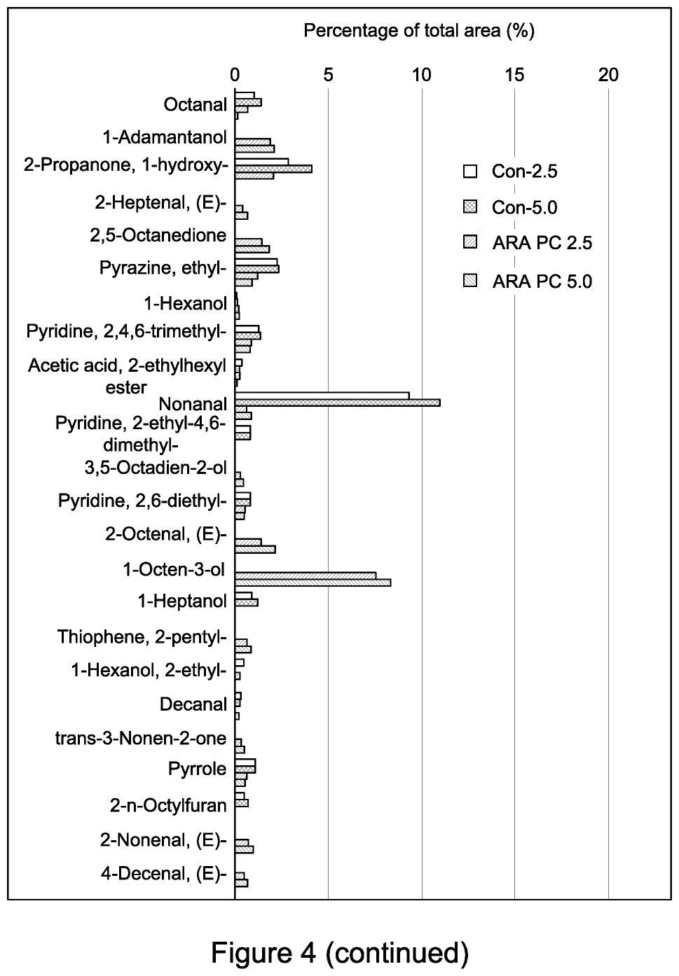

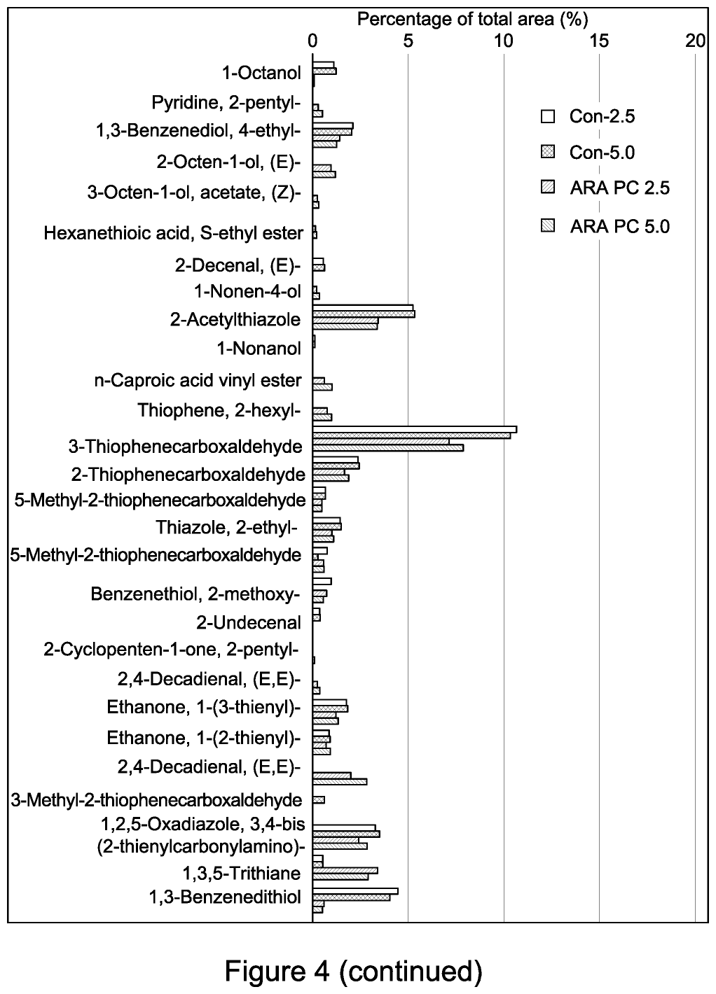

The results are presented in FIG. 4. The results of this experiment demonstrated that alcohols, aldehydes, furans and thiophenes were important volatile compounds found in the reaction mixtures having the ARA-PC lipid. The mixtures derived from ARA-PC showed compounds matching those observed in the earlier experiment, including 1-pentanal, 3-octanone, 2-octen-1-ol, 1-nonanol and 1-octanol. The presence of other compounds was also observed, namely: adamantanol-like compound, hexanal, 2-pentyl furan, 1-octen-3-ol, 2-pentyl thiophene, 1,3,5-thitriane. The compound 2-pentyl thiophene has a characteristic aroma which has been described as chicken, roasted hazelnut or meaty. In contrast, the compound 2-pentyl furan has an aroma described as a fruity, earthy or having a vegetable aroma.

### Experiment 6.

Larger scale cultures of Y. lipoytica strain W29 were grown in the presence of ARA and harvested as described in Example 4 and polar lipid isolated using hexane/ethanol extraction from wet cell pellets. The yield of extracted lipid from the ethanol phase was 6.4 g, of which 1.974 g (30.7%) was lipid. Of that lipid, 95% was polar lipid and 5% was free fatty acid (FFA); the extracted lipid did not appear to have any TAG. The level of ARA in the total fatty acid content of the polar lipid fraction was only 3.2% (Table 9), so lower than optimal. The inventors nevertheless tested this polar lipid in Maillard reactions, with the conditions as in Experiment 5 except using 15, 30 or 60 mg polar lipid per reaction in 2 ml volumes to increase the amount of ARA-polar lipid. The control polar lipid extract had been prepared from Y. lipolytica cells which had not been fed ω6 fatty acids in the medium. Control reactions were also set up having aliquots of the polar lipid extracts but lacking the ribose and cysteine.

The aromas from the reactions were smelled by three volunteers. The mixtures having the ARA-PL provided mild aromas that were described as “pork like, pork crackling, meaty, fatty” or “broiled chicken, milder aroma” or “like broiled fish” whereas the control mixtures having the polar lipid from Y. lipolytica not fed the ARA was described as being sulphurous or “burnt” in their aroma. The mixtures lacking ribose and cysteine were described as “burnt vegetable”.

The inventors concluded that the polar lipid extract having the lower ARA level at 3.2% could provide meat-like aromas but that levels of 10% ARA or greater in the total fatty acid content of the polar lipid were better at providing stronger aromas.

### Experiment 7. Production of Aromas Using Whole Cells Containing ω6-Polar Lipids

As described in Example 4, Y. lipolytica strain W29 cells producing polar lipids including PL were grown in 25 L cultures, either in the presence of ARA (Yl-ARA) in the growth medium or in the absence of ARA (Yl). The fatty acid composition of the polar lipid in the Y. lipolytica cells is shown in Table 10 for Experiments 2 and 3. Notably, ARA was present at 16.4% of the total fatty acid content of the polar lipid, with GLA at 1.4% and DGLA at 1.9%. The harvested cells were then freeze dried and the dried material milled to a powder. The cells were not heat treated or otherwise treated to kill or inactivate the cells. The inventors wished to test the dried yeast cells for the capacity to provide aroma compounds after the cells were heated in the presence of a sugar, for example D-xylose, and an amino acid, for example L-cysteine. A series of reactions were prepared to test the effect of different amounts of the sugar, the amino acid and varying amounts of freeze-dried cells (Table 13). Briefly, L-cysteine powder (Catalog No. 30089, Sigma-Aldrich), D-xylose powder (Catalog No. X1500, Sigma-Aldrich), sodium citrate dihydrate (Catalog No. W302600, Sigma-Aldrich), and wheat flour were weighed into 10 ml GC headspace analysis vials (Catalog No. 23084, Restek, USA) at the indicated amounts before the addition of freeze-dried yeast cells from cultures with or without added ARA. Water (2 ml or 3 ml) was added to each vial and the lids tightly closed before mixing by brief vortexing. The pH of the mixture for vial number 1 was 6.0, based on the buffering by the sodium citrate. The vials were then incubated in an oven pre-heated to 120° C. for either 60 or 45 min before being cooled on ice. The vials were warmed to room temperature before they were opened, and the contents smelled. The aroma for each vial was recorded (Table 12). It was noticed that the cap to vial 13 had been loosened, so that reaction was repeated as vial 19. The loosened cap on vial 13 was presumed to have allowed escape of some of the volatile compounds during heating. Duplicate samples for vials 18-20 were prepared without the water, kept at ambient temperature for 5 or 7 days before the addition of water and then heated to 120° C. for 60 min. These vials provided the same aroma results as vials 18-20 that had been prepared and heated immediately, then frozen for the week, showing that the mixtures can be stored stably for at least one week at room temperature.

Several observations were noteworthy. Reaction vials 2-4 compared to 5-7 were designed to test the effect of whole yeast cells containing ARA in their lipid, compared to yeast cells that did not contain ARA in their lipid. The difference was clearly noticeable with the production of roast meat aroma from the cells having ARA, compared to vials 2-4 where the aroma was not discernible. When the amount of cysteine was lowered to 0.05 g per vial (e.g. vial 17), it was also difficult to detect the desired roast meat aroma. In contrast, when cysteine was at the highest level (e.g. vial 20), the roast meat aroma was more discernible but overpowered or masked to some extent by a sulphurous aroma. A similar effect was noted with the amount of xylose i.e. a lower xylose concentration resulted in a less noticeable undesirable aroma even in the presence of relatively high cysteine concentration (e.g. vial 13), so this was associated with the cysteine concentration. It was concluded that the levels of the amino acid and sugar could be balanced empirically to provide the optimal aroma, i.e. to achieve adequate production of aroma volatiles from the ω6-PUFA without them being masked by stronger smelling undesirable compounds.

The heating time was also a factor to consider. Vials 14-16 and 17-19 were designed to compare this variable with 45 or 60 min heating. The shorter heating time resulted in a noticeably lighter coloured mixture while the longer cooking time produced considerably browner colour. This darkening effect also appeared to be correlated with cysteine levels with more cysteine generally resulting in a darker reaction as long as adequate sugar was present.

In similar fashion the concentration of whole cells was important for desirable aroma generation as demonstrated by vials 8-10. The lower amount of whole cells used in vial 8 resulted in the production of a faint meaty aroma while increasing the amount (vials 9 and 10) yielded a more readily discernible roast meat aroma. It was therefore important to use adequate amounts of whole cells to provide enough ω6-PUFA incorporated into polar lipids for desirable aromas. Again, this feature can be determined empirically.

This experiment also tested whether the yeast cells in the presence of a more complex, food-like material would change the aroma profile. Most of the tested reactions had simple chemical mixtures but vials 8-10 also contained added whole wheat flour to mimic the effect of the presence of plant proteins, carbohydrates, nucleic acids and other components. The aroma from these vials was noticeably different to corresponding vials without the added flour. The aroma of unpleasant sulphur compounds was moderated while the roast meat aroma was still present, providing a more pleasant aroma. The inventors concluded that the use of whole cells producing ω6 fatty acids in the PL when the cells were incorporated in a food containing plant protein was likely to provide the desirable aroma.

A primary conclusion from this experiment was that the addition of ω6 PUFA-containing phospholipids in whole yeast cells worked as well in producing meat-like aroma as the addition of extracted lipid containing the phospholipids with ω6 PUFA. That is, this experiment indicated that it was not necessary to extract ω6-containing phospholipids from the producing cells in order for them to be effective in Maillard or Amadori reactions to produce desirable aroma volatiles.

TABLE 12
Aroma of Maillard reaction products from mixtures comprising Y. lipolytica W29 cells
cultured either in the presence of ARA (Yl-ARA) or in the absence of ARA (Yl). The reactions
contained L-cysteine, D-xylose at the indicated amounts. Vials 1-24 and 28-33 were incubated
at 120° C. for 60 min, whereas vials 25-27 were incubated at 120° C. for 45 min.

| | | | | Na | Wheat | Yeast | |
|---|---|---|---|---|---|---|---|
| Vial | Water | Cysteine | Xylose | citrate | flour | cells | |
| No. | (ml) | (g) | (g) | (g) | (g) | (g) | Odour |
| 1 | 2 | 0.5 | 0.2 | 0.3 | 0 | none | strong sulphur, unpleasant |
| 2 | 2 | 0.5 | 0.2 | 0.3 | 0 | 0.10 Yl | garlic, sulphur |
| 3 | 2 | 0.5 | 0.2 | 0.3 | 0 | 0.25 Yl | garlic, sulphur |
| 4 | 2 | 0.5 | 0.2 | 0.3 | 0 | 0.50 Yl | garlic, sulphur |
| 5 | 2 | 0.5 | 0.2 | 0.3 | 0 | 0.10 Yl-ARA | garlic, mild meat aroma |
| 6 | 2 | 0.5 | 0.2 | 0.3 | 0 | 0.25 Yl-ARA | garlic, beefy aroma |
| 7 | 2 | 0.5 | 0.2 | 0.3 | 0 | 0.50 Yl-ARA | garlic, beefy aroma, smoky |
| 8 | 2 | 0.5 | 0.2 | 0.3 | 0.25 | 0.10 Yl-ARA | garlic, mild meat aroma |
| 9 | 2 | 0.5 | 0.2 | 0.3 | 0.25 | 0.25 Yl-ARA | lighter aroma, old beef roast |
| 10 | 2 | 0.5 | 0.2 | 0.3 | 0.25 | 0.50 Yl-ARA | lighter aroma, old beef roast |
| 11 | 2 | 0.05 | 0.13 | 0.1 | 0 | 0.25 Yl-ARA | low sulphur, low garlic, beefy aroma |
| 12 | 3 | 0.1 | 0.05 | 0.1 | 0 | 0.25 Yl-ARA | low sulphur, low garlic, beefy aroma, pleasant |
| 13 | 3 | 0.2 | 0.05 | 0.1 | 0 | 0.25 Yl-ARA | garlic, mild meat aroma |
| 14 | 3 | 0.05 | 0.05 | 0.1 | 0 | 0.25 Yl-ARA | very faint meaty aroma/colour, |
| 15 | 3 | 0.1 | 0.05 | 0.1 | 0 | 0.25 Yl-ARA | lighter aroma/colour, |
| 16 | 3 | 0.15 | 0.05 | 0.1 | 0 | 0.25 Yl-ARA | lighter aroma/colour, |
| 17 | 3 | 0.05 | 0.05 | 0.1 | 0 | 0.25 Yl-ARA | very faint meaty aroma/colour, |
| 18 | 3 | 0.1 | 0.05 | 0.1 | 0 | 0.25 Yl-ARA | low sulphur, low garlic, beefy aroma, pleasant |
| 19 | 3 | 0.15 | 0.05 | 0.1 | 0 | 0.25 Yl-ARA | sulphur, garlic, strong meaty aroma |
| 20 | 3 | 0.3 | 0.18 | 0.18 | 0 | 0.25 Yl-ARA | sulphur, garlic, strong meaty aroma |

### Example 6. Isolation of Mortierella and Mucor Strains from Soil Samples

Mortierella alpina is a filamentous and saprophytic fungus of the family Zygomycete which is commonly found to inhabit soils from temperate grasslands. Some strains of this species are used commercially to produce oils containing polyunsaturated fatty acids (PUFA), specifically the ω6 fatty acids arachidonic acid (C20:4; ARA), linoleic acid (C18:2; LA) and γ-linolenic acid (C18:3; GLA). Another fungal species, Mucor hiemalis is a zygosporic fungus of the Order Mucorales that is ubiquitous in nature and can be found, for example, in unspoiled foods. It has also been used industrially as a biotransforming agent of pharmacological and chemical compounds, as well as being a potential source of ω6 fatty acids. The present inventors therefore sought to isolate strains of Mortierella alpina, Mucor hiemalis and related species from soil samples obtained from some temperate regions of Australia.

The Biomes of Australian Soil Environments (BASE) project database is a database that contains integrated information about microbial diversity and function for microbial isolates from more than 1,400 soil samples taken from 902 locations across Australia. It includes associated metadata for all of the soil samples across extensive environmental gradients, including information from phylogenetic marker sequencing of bacterial 16S rRNA, archaeal 16S rRNA and eukaryotic 18S rRNA genes to characterise the diversity of microorganisms in community assemblages. Fungal diversity was informed by the 18S rRNA gene amplicon sequences. However, because fungi are an important group of organisms of soils, and because the internal transcribed spacer (ITS) region is more informative than 18S rRNA for many fungal groups, ITS sequences were also included by sequencing fungal-specific ITS amplicons to characterise fungal community assemblages. These amplicons cover the diverse range of microorganisms resident in soils.

The BASE database was therefore interrogated to identify soil samples from the BASE archive that might contain fungal species in the Mortierella or Mucor genera. The interrogation used a M. alpina strain ATCC 32222 internal transcribed spacer 1 (ITS; SEQ ID NO: 1) as a query. More than 12 soil samples were identified as candidates containing these strains from these genera. One such soil sample, designated 102.100.100/14183, was identified and retrieved from the archive for isolation of fungal strains. In addition, two other soil samples, designated Namadgi sample I and Namadgi sample II, were collected from an open grassland field from the temperate Namadgi region of the Australian Capital Territory, Australia. About 5-10 mg of fine soil from each sample was suspended in 3 ml of PBS and vortexed for 2 min. For each soil sample, 100 μl of soil suspension was spread on each of 10 plates of malt extract agar (MEA), containing 20 g/l malt extract and 20 g/l agar, and incubated at 4° C. in the dark (Botha et al. 1998). The plates were observed periodically for growth of fungal colonies. After 8-12 days, mycelia from the edge of distinct colonies were transferred through agar slices to fresh MEA plates and incubated at 4° C. until colonies were 1 to 4 cm in diameter. To further purify the colonies, mycelia from the edge of each colony were transferred through agar slices to fresh MEA plates and incubated at ambient temperatures for 4 days. Colonies that appeared pure through visual inspection were inoculated into 5 ml of malt extract broth and grown at ambient temperature in a static culture for 5 days. A total of 67 fungal strains were thereby isolated from the three soil samples.

Genomic DNA was isolated from each hyphal biomass using the YeaStar Genomic DNA kit (Zymo research, Catalog No. D2002). An internal transcribed spacer (ITS) was amplified through PCR as described by Ho and Chen, (2008) using oligonucleotide primers xMaF1 GGAAGTAAAAGTCGTAACAAGG (SEQ ID NO: 2) and xMaF2 TCCCCGCTTATTGATATGC (SEQ ID NO: 3). The nucleotide sequence of the ITS from the amplicons from each isolate were determined by Sanger sequencing. The obtained sequences were compared to sequences within the NCBI repository using BLAST. The closest hits, with at least 95% nucleotide sequence identity for each isolate and often at 98% or 99% identity, were used to identify the species for each fungal isolate. Classification of species such as Mortierella using ITS homology is standard in the art.

At least four different fungal species were identified based on the ITS homology, which correlated with the four distinctly different morphological features observed when the fungal colonies were grown on the MEA plates. Interestingly, three of the species were isolated mostly from one of the three soil samples but not the others: Mucor hiemalis was found predominantly in Namadji I soil, Mortierella alpina in soil from sample 102.100.100/14183 and isolates of presumed Mortierella sp. in the Namadji II soil. A single colony of Mortierella elongata was isolated from each of the Namadji I and II soil samples. The ITS sequences from the presumed Mortierella sp. isolates identified from the Namadji II soil sample were not found in the NCBI database at a 95% identity level as a minimum. Nevertheless, based on lower homology hits of the ITS sequences, these isolates were considered to most likely be of Mortierella sp. or a species closely related to the Mortierella genus. The nucleotide sequences for the ITS regions for 43 fungal isolates and the deduced species names are listed in Table 14. Selected isolates were designated as strains yNI0121 to yNI0131 and yNI0133 to yNI0135 (Table 13).

The ITS regions amplified with primers xMaF1 and xMaF2 produced amplicons having a length of between 639 and 647 basepairs for the Mucor hiemalis strains, between 668 and 672 basepairs for the Mortierella alpina strains, between 628 and 652 basepairs for the Mortierella sp. isolates, and between 640 and 659 for the two Mortierella elongata strains. The length of this ITS amplicon was therefore useful in helping to distinguish between the four species.

TABLE 13
Species identities of isolated soil fungi.

| | ITS sequence | ITS highest homology | Designated |
|---|---|---|---|
| Identifier | (SEQ ID NO) | to | strain ID |
| 14183 isolate 1 | 4 | Mucor hiemalis | — |
| 14183 isolate 2 | 5 | M. alpina | yNI0133 |
| 14183 isolate 3 | 6 | M. alpina | yNI0134 |
| 14183 isolate 4 | 7 | M. alpina | yNI0135 |
| 14183 isolate 21 | 8 | M. alpina | — |
| 14183 isolate 22 | 9 | M. alpina | — |
| 14183 isolate 23 | 10 | M. alpina | — |
| 14183 isolate 24 | 11 | Possibly Trichoderma | — |
| | | asperellum | |
| 14183 isolate 25 | 12 | M. alpina | — |
| Namadji I isolate 1 | 13 | Mucor hiemalis | yNI0121 |
| Namadji I isolate 3 | 14 | Mucor hiemalis | yNI0122 |
| Namadji I isolate 4 | 15 | Mucor hiemalis | yNI0124 |
| Namadji I isolate 5 | 16 | Mucor hiemalis | yNI0123 |
| Namadji I isolate 6 | 17 | Mucor hiemalis | — |
| Namadji I isolate 8 | 18 | Mucor hiemalis | — |
| Namadji I isolate 9 | 19 | Mucor hiemalis | — |
| Namadji I isolate 10 | 20 | Mucor hiemalis | — |
| Namadji I isolate 11 | 21 | Mortierella elongata | yNI0125 |
| Namadji I isolate 12 | 22 | Mucor hiemalis | — |
| Namadji I isolate 14 | 23 | Mucor hiemalis | — |
| Namadji I isolate 15 | 24 | Mucor hiemalis | — |
| Namadji I isolate 21 | 25 | Mucor hiemalis | — |
| Namadji II isolate 1 | 26 | Mortierella sp. | yNI0126 |
| Namadji II isolate 2 | 27 | Mortierella sp. | yNI0127 |
| Namadji II isolate 3 | 28 | Mortierella sp. | yNI0128 |
| Namadji II isolate 4 | 29 | Mortierella sp. | yNI0129 |
| Namadji II isolate 5 | 30 | Mortierella sp. | yNI0130 |
| Namadji II isolate 6 | 31 | Mortierella sp. | — |
| Namadji II isolate 7 | 32 | Mortierella sp. | — |
| Namadji II isolate 8 | 33 | Mortierella sp. | — |
| Namadji II isolate 9 | 34 | Mortierella elongata | yNI0131 |
| Namadji II isolate 10 | 35 | Mortierella sp. | — |
| Namadji II isolate 11 | 36 | Mortierella sp. | — |
| Namadji II isolate 12 | 37 | Mortierella sp. | — |
| Namadji II isolate 13 | 38 | Mortierella sp. | — |
| Namadji II isolate 14 | 39 | Mortierella sp. | — |
| Namadji II isolate 15 | 40 | Mortierella sp. | — |
| Namadji II isolate 16 | 41 | Mortierella sp. | — |
| Namadji II isolate 17 | 42 | Mortierella sp. | — |
| Namadji II isolate 18 | 43 | Mortierella sp. | — |
| Namadji II isolate 19 | 44 | Mortierella sp. | — |
| Namadji II isolate 20 | 45 | Mortierella sp. | — |
| Namadji II isolate 21 | 46 | Mortierella sp. | — |

### Fatty Acid Composition and Oil Content of Fungal Isolates

For analysis of lipid in these fungal isolates, agar slices from the edges of colonies were placed on fresh SD agar plates and allowed to grow for 4-6 days at ambient temperature, until the colonies exceeded 3 cm in diameter. SD medium was used in this experiment as it does not have yeast extract which may have some lipid that might contaminate the fungal biomass. Hyphal biomass was harvested from the plates and suspended in sterile water for pelleting. After washing the hyphal biomass with ethanol, lipid was extracted using a chloroform/methanol solvent (Bligh and Dyer, 1959) and fractionated on TLC plates to obtain TAG and polar lipid fractions. The fatty acid composition of the TAG and polar lipid fractions were determined by GC analysis of FAME as described in Example 1.

The data for strains yNI0121 to yNI0131 and yNI0133 to yNI0135 are presented in Table 14. The fatty acid compositions showed distinct differences between the four species, but with some similarities as well. All four species produced polyunsaturated ω6 fatty acids having 18 or 20 carbons and 3 or 4 desaturations in the acyl chains, namely GLA alone of the ω6 fatty acids in the case of Mucor hiemalis, or all three of GLA, DGLA and ARA for all of the Mortierella isolates. Nearly all of the isolates produced at least 20% such PUFA (sum of GLA, DGLA and ARA) in the polar lipid fraction, up to about 38%, as a percentage by weight of the total fatty acid content in those fractions. The Mucor hiemalis strains yNI0121 to yNI0124 all produced GLA at about 10% in the total fatty acid content of the TAG and between 26% and 30% GLA in the polar lipids. It was concluded that these Mucor strains preferentially accumulated the ω6 PUFA in their polar lipids. These strains did not produce ARA or DGLA levels at detectable levels, or only at trace amounts in the polar lipid fractions, indicating that they did not have the ability to elongate GLA to DGLA i.e. they lacked a fatty acid A6 elongase. This is consistent with published reports for Mucor strains (Certik et al., 1993). LA (C18:2ω6) was the most abundant fatty acid in the TAG fraction of the Mucor strains, but not in any of the three Mortierella species. These strains produced 12-18% TAG and a relatively high amount of polar lipid, up to about 7% by dry weight, under the growth conditions on SD agar plates used to culture the strains for this analysis. Considering that the extraction and recovery of lipid fractions from the process including TLC fractionation would have been less than 100%, the total lipid content of these Mucor hiemalis strains was greater than 20% and therefore these strains are oleaginous.

In contrast to the Mucor hiemalis strains, the Mortierella alpina strains yNI0133 to yNI0135 produced abundant ARA as well as GLA and DGLA. The ARA level in both the TAG and polar lipids was about 30% by weight of the total fatty acid content in those fractions. These M. alpina strains therefore did not exhibit any preference for accumulating the ω6 PUFA in polar lipid relative to TAG. The GLA and DGLA levels were about 2% and about 6%, respectively, in TAG, and about 4-7% and about 2-4%, respectively, in the polar lipid. Compared to the ARA levels, this indicated that the M. alpina strains have efficient Δ6 elongase and Δ5 desaturase enzymes. Genes encoding such enzymes have been isolated from other strains of M. alpina (Huang et al., 1999; Knutzon et al., 1998). The Mortierella alpina strains also produced about 4-5% of C24:0 in the TAG fractions

Again in contrast to Mucor, the presumed Mortierella sp. strains yNI0126 to yNI0130 produced ARA and DGLA in addition to GLA and accumulated these ω6 PUFA in both TAG and polar lipids. However, in contrast to the M. alpina strains, the Mortierella sp. strains accumulated 2- to 4-fold more ARA in their polar lipid than in their TAG. It was concluded that these Mortierella sp. strains, like the Mucor strains, preferentially accumulated their ω6 PUFA in the polar lipid relative to the TAG. The two Mortierella elongata strains yNI0125 and yNI0131 were similar in many features to the Mortierella sp. strains, including that they produced ARA and DGLA in addition to GLA and accumulated these ω6 PUFA in both TAG and polar lipids. They also showing a preference for accumulated more ARA in their polar lipid than in their TAG. The Mortierella elongata strains could be distinguished from the Mortierella sp. in the levels of some of the other fatty acids, or the ratios between pairs of related fatty acids, i.e. reflecting the conversion rate of one fatty acid to another e.g. GLA to DGLA. Nevertheless, further phylogenetic analyses need to be done to establish the relationship of the Mortierella sp. strains to the Mortierella elongata strains.

All of the strains tested had considerable amounts of monounsaturated and saturated fatty acids in both the TAG and polar lipid fractions. Oleic acid was the most abundant fatty acid in both the TAG and polar lipid fractions of the Mucor hiemalis, Mortierella sp. and Mortierella elongata strains, but not in the Mortierella alpina strains where ARA was the most abundant fatty acid. Palmitic acid was the most abundant SFA in both the TAG and polar lipid fractions in all of the strains examined. With one or two exceptions, the amount of stearic acid was relative low at about 3-10% in TAG and about 2-6% in polar lipid. The other SFA present in all strains were myristic acid (C14:0), pentadecanoic acid (C15:0), arachidic acid (C20:0), behenic acid (C22:0) and lignoceric acid (C24:0). The monounsaturated fatty acids C16:1Δ7, C17:1, C18:1Δ11 (vaccenic acid) and C22:1 were present at low but detectable levels in all of the strains.

The inventors next cultured selected strains yNI0121 (Mucor hiemalis), yNI0125 (Mortierella elongata), yNI0127 (Mortierella sp.) and yNI0132 (Mortierella alpina) in order generate larger quantities of fungal biomass to evaluate mycelium disruption methods and to produce sufficient amounts of extracted lipid for food incorporation experimentation. yNI0132 had also been isolated from the 102.100.100/14183 soil sample, identified as M. alpina on the basis of ITS homology (where the yNI0132 ITS is set forth in SEQ ID NO:47) and exhibited similar fatty acid profile in the polar lipid and TAG fractions as yNI0133, yNI0134 and yNI0135. Fungal biomass from yNI0121, yNI0125, yNI0127 and yNI0132 was also tested in order to determine whether whole cell biomass, either in a wet form or dried as a powder, could be used in Maillard-type reactions to produce meat-like aromas from these fungi containing PL having ω6 fatty acids. This would also allow a comparison of strains that had about equal levels of ω6 fatty acids in the TAG and polar lipid fractions with those that had more ω6 fatty acids in their polar lipids relative to the TAG.

To prepare seed cultures for the larger cultures, the fungal strains yNI0121, yNI0125, yNI0127 and yNI0132 were freshly propagated by agar slice growth, taking 0.5×0.5 cm agar pieces with fungal mycelium from the edge of colonies and placed them in the centre of a fresh MEA plate. The plates were kept at ambient temperature for 3 to 5 days until the new colonies were at least 3 cm in diameter. For each strain, intermediate cultures were then prepared by inoculating six 0.5×0.5 cm agar pieces containing mycelium into 10 ml malt extract medium and incubating these with shaking for 3 days at 26° C. and then kept stationary for 2 days. The complete cultures were then used to inoculate 50 ml of malt extract medium in 250 ml baffled flasks and incubated with shaking at 26° C. for 3 days. These cultures were then used to inoculate 600 ml of medium containing (per litre) 60 g glucose, 10 g yeast extract, 5 g malt extract, 4 g KH₂PO₄, 3 g (NH₄)₂HPO₄ and 0.6 g MgSO₄ with the pH adjusted to 6.0 with 2 M NaOH. These larger cultures were incubated with shaking at 26° C., the cultures sampled after 2 days and the biomass harvested by centrifugation after 3 days, freeze dried and then frozen.

TABLE 14
Fatty acid composition of TAG and polar lipids from Mortierella and Mucor isolates from soil samples

| Strain ID | Species | C14:0 | C15:0 | C16:0 | C16:1Δ9 | C16:1Δ7 | C17:1? | C18:0 | C18:1Δ9 | C18:1Δ11 |
|---|---|---|---|---|---|---|---|---|---|---|
| | | TAG fatty acid composition | | | | | | | | |
| yNI0121 | Mucor hiemalis | 1.4 | 0.1 | 17.4 | 0.5 | 0.2 | 0 | 10.8 | 37.9 | 0.1 |
| yNI0122 | Mucor hiemalis | 2.3 | 0.2 | 22.9 | 1.3 | 0.1 | 0 | 8.0 | 39.2 | 0.3 |
| yNI0123 | Mucor hiemalis | 2.6 | 0.2 | 24.8 | 0.8 | 0.1 | 0 | 5.4 | 37.0 | 0.1 |
| yNI0124 | Mucor hiemalis | 2.3 | 0.2 | 23.9 | 0.9 | 0.2 | 0 | 4.9 | 36.7 | 0.2 |
| yNI0125 | Mortierella | 3.0 | 0.2 | 27.6 | 0.6 | 0.1 | 0.1 | 9.4 | 38.9 | 0.5 |
| | elongata | | | | | | | | | |
| yNI0133 | Mortierella | 1.5 | 0.3 | 19.1 | 0.4 | 0.1 | 0.1 | 5.1 | 18.8 | 0.8 |
| | alpina | | | | | | | | | |
| yNI0134 | Mortierella | 2.3 | 0.3 | 19.1 | 0.4 | 0.1 | 0.1 | 6.7 | 20.9 | 0.5 |
| | alpina | | | | | | | | | |
| yNI0135 | Mortierella | 1.6 | 0.3 | 20.4 | 0.4 | 0.1 | 0.1 | 5.1 | 21.0 | 0.9 |
| | alpina | | | | | | | | | |
| yNI0126 | Mortierella sp | 2.9 | 0.2 | 31.1 | 0.6 | 0.1 | 0.1 | 6.3 | 44.3 | 0.6 |
| yNI0127 | Mortierella sp | 3.5 | 0.2 | 28.3 | 1.1 | 0.2 | 0.1 | 3.5 | 42.7 | 0.9 |
| yNI0128 | Mortierella sp | 3.2 | 0.2 | 29.9 | 1 | 0.2 | 0.1 | 4.2 | 43.3 | 0.9 |
| yNI0129 | Mortierella sp | 3.4 | 0.2 | 28.4 | 1.1 | 0.2 | 0.1 | 3.1 | 42.0 | 1.0 |
| yNI0130 | Mortierella sp | 3.5 | 0.2 | 25.7 | 1.0 | 0.2 | 0.1 | 3.6 | 46.6 | 0.7 |
| yNI0131 | Mortierella | 2.1 | 0.3 | 19.1 | 0.3 | 0.2 | 0.1 | 10.6 | 38.9 | 0.2 |
| | elongata | | | | | | | | | |
| | | Polar lipid fatty acid composition | | | | | | | | |
| yNI0121 | Mucor hiemalis | 0.7 | 0.2 | 13.8 | 0.4 | 0.2 | 0.1 | 2.5 | 20.4 | 0.1 |
| yNI0122 | Mucor hiemalis | 1.1 | 0.3 | 13.6 | 1.2 | 0.1 | 0 | 1.9 | 14.9 | 0.2 |
| yNI0123 | Mucor hiemalis | 1.1 | 0.3 | 15.3 | 0.6 | 0.2 | 0 | 1.8 | 15.6 | 0.1 |
| yNI0124 | Mucor hiemalis | 1.1 | 0.4 | 16.9 | 0.8 | 0.3 | 0.1 | 1.9 | 18.5 | 0.1 |
| yNI0125 | Mortierella | 1.1 | 0.2 | 15.3 | 0.5 | 0.1 | 0.1 | 4.7 | 30.3 | 0.9 |
| | elongata | | | | | | | | | |
| yNI0133 | Mortierella | 0.5 | 0.5 | 13.8 | 0.3 | 0.1 | 0.1 | 3.7 | 18.4 | 2.7 |
| | alpina | | | | | | | | | |
| yNI0134 | Mortierella | 0.8 | 0.4 | 11.8 | 0.3 | 0 | 0 | 3.2 | 26.3 | 1.5 |
| | alpina | | | | | | | | | |
| yNI0135 | Mortierella | 0.5 | 0.5 | 13.8 | 0.4 | 0.1 | 0.1 | 3.1 | 19.5 | 3.0 |
| | alpina | | | | | | | | | |
| yNI0126 | Mortierella sp | 0.9 | 0.1 | 13.6 | 0.3 | 0.1 | 0.1 | 19.0 | 28.8 | 1.8 |
| yNI0127 | Mortierella sp | 1.0 | 0.2 | 14.1 | 0.5 | 0.1 | 0.1 | 3.6 | 28.5 | 3.0 |
| yNI0128 | Mortierella sp | 1.0 | 0.2 | 14.5 | 0.5 | 0.1 | 0.1 | 3.7 | 31.9 | 2.3 |
| yNI0129 | Mortierella sp | 1.0 | 0.2 | 14.5 | 0.5 | 0.1 | 0.1 | 3.8 | 29.7 | 2.8 |
| yNI0130 | Mortierella sp | 0.9 | 0.2 | 13.4 | 0.4 | 0.1 | 0.1 | 3.1 | 29.2 | 2.4 |
| yNI0131 | Mortierella | 1.0 | 0.4 | 14.5 | 0.2 | 0.2 | 0.1 | 6.0 | 29.7 | 0.9 |
| | elongata | | | | | | | | | |

| | | | | | | | | | | TAG |
|---|---|---|---|---|---|---|---|---|---|---|
| | | | | | | | | | | or |
| | C18:2Δ9, | | | | C20:4ω6 | | | | | PL |
| Strain ID | 12 | C18:3ω6 | C20:0 | C20:3ω6 | (ARA) | C22:0 | C22:1 | C24:0 | Rest | (%) |
| | TAG fatty acid composition | | | | | | | | | |
| yNI0121 | 18.3 | 10.3 | 0.6 | 0 | 0 | 0.5 | 0.1 | 0.6 | 1.4 | 17.9 |
| yNI0122 | 13.2 | 8.9 | 0.3 | 0 | 0 | 0.5 | 0.1 | 0.9 | 1.6 | 14.2 |
| yNI0123 | 15.5 | 10.1 | 0.2 | 0 | 0 | 0.4 | 0.1 | 0.9 | 1.7 | 14.6 |
| yNI0124 | 16.3 | 11.1 | 0.2 | 0 | 0 | 0.4 | 0.1 | 0.9 | 1.7 | 12.6 |
| yNI0125 | 4.1 | 1.9 | 0.4 | 1.8 | 6.1 | 0.5 | 0.7 | 1.5 | 2.6 | 19.9 |
| yNI0133 | 5.8 | 1.9 | 0.5 | 5.9 | 30.7 | 1.6 | 0.5 | 4.5 | 2.5 | 14.4 |
| yNI0134 | 5.6 | 2.3 | 0.7 | 5.7 | 25.4 | 2.1 | 0.5 | 5.0 | 2.5 | 8.9 |
| yNI0135 | 6.2 | 1.8 | 0.5 | 5.8 | 26.5 | 1.5 | 0.5 | 4.3 | 2.7 | 17.9 |
| yNI0126 | 3.2 | 1.0 | 0.4 | 0.8 | 4.0 | 0.6 | 0.7 | 0.4 | 2.8 | 26.2 |
| yNI0127 | 4.0 | 1.8 | 0.2 | 1.0 | 7.3 | 0.4 | 1.1 | 0.4 | 3.2 | 17.5 |
| yNI0128 | 4.0 | 1.3 | 0.2 | 0.9 | 5.7 | 0.4 | 1.0 | 0.4 | 3.0 | 17.9 |
| yNI0129 | 4.1 | 1.9 | 0.2 | 1.0 | 8.2 | 0.4 | 1.1 | 0.3 | 3.3 | 17.6 |
| yNI0130 | 4.5 | 1.7 | 0.2 | 0.9 | 5.2 | 0.3 | 1.7 | 0.4 | 3.6 | 20.5 |
| yNI0131 | 4.2 | 2.9 | 0.4 | 2.2 | 11.2 | 0.7 | 2.0 | 1.3 | 3.4 | 16.9 |
| | Polar lipid fatty acid composition | | | | | | | | | |
| yNI0121 | 32.4 | 26.0 | 0.1 | 0.1 | 0 | 0.5 | 0.1 | 1.2 | 1.3 | 5.5 |
| yNI0122 | 33.1 | 29.4 | 0.1 | 0.1 | 0 | 0.5 | 0.1 | 1.8 | 1.5 | 6.4 |
| yNI0123 | 32.3 | 28.9 | 0.1 | 0.1 | 0 | 0.4 | 0.1 | 1.6 | 1.5 | 7.1 |
| yNI0124 | 29.2 | 27.0 | 0.1 | 0.1 | 0 | 0.4 | 0.2 | 1.6 | 1.5 | 2.0 |
| yNI0125 | 11.5 | 6.5 | 0.1 | 1.8 | 21.4 | 0.2 | 2.0 | 1.4 | 2.0 | 2.6 |
| yNI0133 | 16.7 | 4.0 | 0.5 | 2.8 | 30.1 | 0.3 | 0.9 | 1.5 | 3.0 | 1.7 |
| yNI0134 | 12.0 | 7.3 | 0 | 3.5 | 26.7 | 0.5 | 1.0 | 1.9 | 3.0 | 1.0 |
| yNI0135 | 18.1 | 4.1 | 0.4 | 2.7 | 28.9 | 0.3 | 0.5 | 1.3 | 2.6 | 2.2 |
| yNI0126 | 9.7 | 3.9 | 0.1 | 1.2 | 15.4 | 0.2 | 1.7 | 0.4 | 2.9 | 3.0 |
| yNI0127 | 13.5 | 6.3 | 0 | 1.2 | 21.6 | 0.1 | 2.5 | 0.4 | 3.2 | 2.3 |
| yNI0128 | 13.8 | 4.8 | 0.1 | 1.3 | 19.5 | 0.1 | 2.7 | 0.5 | 3.1 | 2.3 |
| yNI0129 | 12.8 | 5.6 | 0 | 1.1 | 21.8 | 0.1 | 2.3 | 0.4 | 3.2 | 2.3 |
| yNI0130 | 16.7 | 7.4 | 0.9 | 1.1 | 16.2 | 0.1 | 3.6 | 0.5 | 3.7 | 2.2 |
| yNI0131 | 10.1 | 6.7 | 0.1 | 2.1 | 19.1 | 0.3 | 4.4 | 1.3 | 2.9 | 2.4 |

The wet weights and corresponding dry weights for the three Mortierella species in a first culture, and for the three Mortierella species and the Mucor hiemalis strain are shown in Table 15.

TABLE 15
Biomass weights obtained from larger scale
culture of Mortierella strains

| | | | | |
|---|---|---|---|---|
| | 48 h wet | 72 h wet | 48 h dry | 72 h dry |
| Strain | weight (g) | weight (g) | weight (g) | weight (g) |

Experiment 1

| | | | | |
|---|---|---|---|---|
| yNI0125 | nd | 10.3 | 2.81 | 4.91 |
| yNI0127 | 5.27 | 13.7 | 2.96 | 4.64 |
| yNI0132 | 6.08 | 16.7 | 2.64 | 4.3 |

Experiment 2

| | |
|---|---|
| yNI0121 | 38.5 |
| yNI0125 | 13.25 |
| yNI0127 | 10.84 |
| yNI0132 | 15.85 |

### Improved Biomass Production for Fungal Strains

In an attempt to improve biomass weights achieved in the cultures, two culture media were compared. To test a first medium, the three Mortierella isolates yNI0125, yNI0127 and yNI0132 and Mucor hiemalis strain yNI00121 were grown in a seed culture containing (per litre) 20 g glucose, 6 g yeast extract, 5 g malt extract, 3 g KH₂PO₄, 3 g (NH₄)₂HPO₄ and 3 g MgSO₄·7H₂O. The seed cultures were used to inoculate 600 ml cultures in Medium 1 (per litre): 20 g glucose, 5 g yeast extract, 10 g peptone, incubated at 26° C. with shaking at 200 rpm for aeration. Parallel cultures of 800 ml were also grown at the same time in a second medium, Medium 2, containing 30 g glycerol, 0.85 g yeast extract, 8.7 g KH₂PO₄, 1.9 g (NH₄)₂HPO₄, pH 6.2, cultured at 26° C. with shaking at 200 rpm for aeration. Growth was significantly faster in Medium 1, reaching about 14 g/l dry weight at 70 h.

Dried whole cell biomass from these strains were used in Maillard reactions (Example 7).

Extraction of Total Lipid from Fungal Biomass

Total lipid was extracted from harvested wet fungal biomass (Table 15, Experiment 2) using hexane as solvent, as follows. Most of the water was removed by washing the cell biomass with ethanol, using 2 ml of ethanol per gram of cell biomass (wet weight) followed by centrifugation each time to recover the cell biomass. The pelleted cells were resuspended in hexane, using 5 ml hexane per gram of cell biomass. The suspensions were homogenised and the cells disrupted with the UltraTurrax (IKA, Malaysia) for 3 min followed by sonication for 5 min, which pair of treatments was repeated twice for a total of three times. The mixtures were shaken for 3 h at room temperature, although later experiments showed that shaking the mixtures overnight extracted more lipid. Observation by microscopy of samples from the mixtures showed that many but not all of the cells had been disrupted by the treatments. The mixtures were centrifuged and the hexane phase collected. The hexane was evaporated from each extraction using a flow of nitrogen, and the dried lipid extracts weighed. This resulted in 0.99 g from yNI0121 (Mucor hiemalis), 1.33 g from yNI0125 (Mortierella elongata), 0.69 g from yNI0127 (Mortierella sp.) and 0.78 g from yNI0132 (Mortierella alpina). Samples of these lipids were chromatographed on TLC plates and the polar lipids extracted from the silica for analysis of the fatty acid composition by GC of FAME as described in Example 1.

Extraction of Partially Purified Polar Lipid from Fungal Biomass

An alternative extraction method was tested as a means of preferentially extracting polar lipids by extraction into ethanol, based on the greater solubility of polar lipid in ethanol relative to neutral lipids. Harvested dry biomass following fermentation of Mortierella alpina (45.63 g) and 60 mL of ethanol were blended and at least partially disrupted using an UltraTurrax homogeniser. The sample was then mixed with stirring for 30 min and centrifuged. The ethanol supernatant was removed. This extraction of the M. alpina biomass with ethanol was repeated twice and the supernatants combined. The precipitate can be retained for extraction of neutral lipids if desired. The ethanol was evaporated from the combined supernatants in a rotary evaporator, programmed as follows: vacuum pump at 15 mbar, chiller at −16° C., water bath at 37° C. and 400 rpm. From an initial input of 45.63 g of M. alpina dry biomass, 5.7 g of phospholipid enriched precipitate was recovered. The precipitate at this stage also contained some TAG. The phospholipid enriched precipitate was dissolved in 30 ml hexane and cooled in an ice bath at 0° C. Next, 120 ml of cold acetone (−20° C.) was added into the stirred mixture to precipitate phospholipids. The precipitate was washed 5 times with 30 ml portions of cold acetone (−20° C.). The residual solvent in the extracted and purified phospholipid preparation was removed in a rotary evaporator at room temperature for 10 h. The polar lipid yield was measured gravimetrically and a small aliquot used for FAME analysis. Another aliquot was chromatographed on TLC to check for purity. From an initial input of 45.63 g of M. alpina dry biomass, 1.1 g of relatively pure phospholipid was recovered.

### Example 7. Production of Fungal Biomass at Larger Scale

The inventors next produced whole cell biomass and lipid extracts from the biomass including PL containing ω6 fatty acids such as ARA from the fungal isolates described in Example 6. The fungal isolates were cultured at 35 L scale, the fungal mass harvested from the cultures and lipids extracted. In some experiments, the lipids were fractionated to isolate the polar lipids, including the PL, and both whole cells and extracted lipids used in Maillard reactions and food preparations.

### Larger Scale Production of Fungal Biomass and Extraction of Lipids Having ω6 Fatty Acid (B017)

In a larger scale experiment producing 35 L of culture, Mortierella alpina strain yNI0132 was grown in a Braun fermenter in a rich medium containing glucose as the main carbon source, seeking to produce more cell biomass and a suitable polar lipid:TAG ratio having ω6 fatty acid incorporated into polar lipids. The growth medium was based on a rich yeast extract-malt extract medium which favoured biomass production rather than TAG production, even though M. alpina is an oleaginous species that naturally is capable of producing abundant TAG. The medium used for the seed culture for inoculation and for the first phase of culture contained (per litre) 60 g glucose, 10 g yeast extract, 5 g malt extract, 3 g (NH₄)₂SO₄, 1 g KH₂PO₄, 0.6 g MgSO₄·7H₂O, 0.06 g CaCl₂ and 0.001 g of ZnSO₄, pH 6.2. The second stage of culturing used a feed solution of 5 L containing (per litre) 5 g malt extract, 7.5 g (NH₄)₂SO₄, 1 g KH₂PO₄, 6.0 g MgSO₄·7H₂O, 0.3 g CaCl₂ and 0.005 g of ZnSO₄ but no yeast extract. These media used ammonium sulphate as the nitrogen source rather than urea. The first phase culture medium was prepared and sterilised in the fermenter by autoclaving in situ at 121° C. for 15 min, then cooled by direct cooling to the fermenter jacket. The glucose stock solution (438 g glucose monohydrate plus 563 ml water) was autoclaved separately as a 40% solution and, while still warm at 45° C., was added to the fermenter.

An inoculum culture was prepared in 4×200 ml YM broth in 500 ml flasks using starter cultures from agar plates. The inoculum culture was incubated for 71.5 h at 30° C. with shaking at 180 rpm, at which time the inoculum cultures showed luxuriant growth. The inoculum culture was introduced into the fermenter without homogenisation of the culture. In the first phase of culturing with the aim of maximising biomass production, a high aeration rate was maintained at about 0.6 to 1.0 vvm (18-30 l/min) and mixing was low at 50-150 rpm to maintain dissolved oxygen at greater than 1 ppm without excessive shear forces being applied to the culture. After 76 h cultivation, the nutrient feed solution was added to the fermenter. The pH was controlled at 6.0 throughout by addition of NaOH and the temperature was maintained at 30° C. The culture was sampled (50 ml) every 24 h post inoculation. The parameters that were measured daily were cell density (dry cell weight), glucose level by HPLC, total nitrogen level by the Kjeldahl method, phosphate and sulphate levels by colorimetric strips, and the appearance of the fungus by light microscopy. Dry weight (dry cell weight) was measured by weighing the material collected on a glass microfibre obtained by filtering 20 ml of culture using a Buchner funnel and a vacuum pump before being dried in an oven and then weighed. The culture was harvested at 94 h when the cell density had reached 19.5 g/l (wet weight/w). The biomass was harvested by filtration through a nylon gauze (200 micron). The biomass was resuspended and washed twice, each time with two volumes of cold water relative to the volume of biomass. The mycelial biomass was grey-white in colour. Excess water was removed by squeezing the wet mycelial cake through the filter cloth by hand. This yielded 2.27 kg of washed biomass having a dry weight of approximately 590 g. The biomass cake was spread to a 1-2 cm layer in ziploc bags and frozen.

TABLE 16
Raw data from fermentation experiment B017 for M. alpina strain yNI0132

| Time | Glucose | Sulphate | Phosphate | DW | | Glucose | Nitrogen |
|---|---|---|---|---|---|---|---|
| (h) | (g/l) | (mg/l) | (mg/l) | g/l | Nitrogen | (% start) | (% start) |
| 0 | 59.6 | 400 | 250 | 0.47 | 0.5 | 100 | 100 |
| 24.2 | 56.7 | 400 | 250 | 2.0 | 0.49 | 95 | 98 |
| 46.0 | 40.2 | 400 | 250 | 15.8 | 0.07 | 67 | 14 |
| 70.3 | 38.4 | 400 | 250 | 17.0 | 0 | 64 | 0 |
| 94.0 | 23.4 | 400 | 250 | 19.4 | 0.03 | 39 | 6 |

As indicated by the DW and pH, most of the fungal growth occurred between 20 and 50 h. The culture reached a stationary phase at about 50 h, presumably due to depletion of nitrogen, with no further pH adjustment occurring after that time point. Nitrogen by the Kjeldahl method was depleted at 70.3 h. Further addition of nitrogen at 76 h by the feed solution provided a further increase in glucose consumption and a further increase in DW of the biomass. The final glucose concentration was 23.37 g/1, so only 60% of the initial amount was consumed. Further adjustment of the nitrogen to carbon ratio was therefore considered to optimise biomass production. Table 17 shows the fatty acid profile of the TFA, TAG and PL fractions of the biomass.

TABLE 17

| Sample | C14:0 | C15:0 | C16:0 | C16:1 | C18:0 | C18:1 | C18:1iso | C18:2 | C18:3n6 | C20:0 | C20:3 | ARA |
|---|---|---|---|---|---|---|---|---|---|---|---|---|
| PL | 0.5 | 0 | 24.04 | 0 | 15.09 | 10.1 | 0.74 | 11.07 | 5.51 | 0.74 | 4.55 | 25.26 |
| TAG | 0.7 | 0.18 | 21.86 | 0.27 | 7.81 | 10.31 | 0.55 | 8.97 | 3.31 | 0.68 | 4.82 | 31.35 |
| TFA | 0.69 | 0.15 | 22.54 | 0.28 | 9.83 | 10.43 | 0.6 | 8.92 | 3.45 | 0.64 | 5 | 29.36 |

Larger Scale Production of Fungal Biomass with Inactivation

Mortierella alpina strain yNI0132 was grown in a Braun fermenter in a rich medium containing glucose as the main carbon source, and harvested at 65 hrs. The mediumused for the seed culture for inoculation and for the first phase of culture contained (per litre) 65.6 g glucose, 10 g yeast extract, 5 g malt extract, 3 g (NH₄)₂SO₄, 1 g KH₂PO₄, 0.6 g MgSO₄·7H₂O, 0.06 g CaCl₂ and 0.001 g of ZnSO₄, pH 6.2, as well as Polyglycol P2000 at 1.0% as an antifoam. The second stage of culturing used a feed solution of (per litre) 0.833 g malt extract, 1.25 g (NH₄)₂SO₄, 0.167 g KH₂PO₄, 1 g MgSO₄·7H₂O, 0.05 g CaCl₂ and 0.0008 g of ZnSO₄ but no yeast extract. The first phase culture medium was prepared and sterilised in the fermenter by autoclaving in situ at 122° C. for 30 min, then cooled by direct cooling to the fermenter jacket. The glucose stock solution was sterilised separately at 121° C. for 15 min and transferred to the fermenter after the broth and glucose had cooled to 45° C.

A 1 L inoculum culture was prepared in YM broth in 5 L ml flasks using colonies from agar plates. The inoculum culture was incubated for 24 h at 28° C. with shaking at 150 rpm. The fermenter was sterilised with batch medium (no glucose) and antifoam (at 1.0% in the fermenter). Glucose was separately sterilised and added to the sterile batch medium by peristaltic pump, and the pH was adjusted to pH 6.0 and temperature to 30° C. The fermenter was inoculated with 1000 mL starter culture by peristaltic pump when the bottle showed luxurious growth of pumpable colonies (16-24 hours). The fermenter was sampled at TO (after adding inoculum) and everyday (50 mL) and measured for cell density (oven dry weight (DW)), glucose by HPLC and phosphate and sulphate (strips). DW was measured by weighing the dry pellet on glass microfibre obtained by filtering approximately 15 g of sample using a Buchner funnel and a vacuum pump before being dried in an oven and then weighed. Nutrient feed without glucose was transferred as bolus at 43.1 hours post inoculation. The fermenter was harvested at 65.2 hours post inoculation, and the harvested culture was processed using a wine press (100 kPA, 10 minutes). The biomass was resuspended in sterile water and reprocessed using the wine press at 100 kPA for 10 minutes. The biomass cake was wrapped in aluminium foil in thin layers, and the paste wet weight in each wrap was recorded and the biomass yield per litre of culture calculated. The wrapped biomass samples were placed in ziplock bags and frozen. The frozen biomass was rehydrated in sterile water (1:4) and suspended using the Silversson high shear mixer. The paste was then homogenised using APV homogeniser at 10 000 psi, 10 minutes until a free-flowing liquid was produced. The homogenised sample was pasteurised at >76° C., with a pump rate of 20-30 rpm. A portion of the pasteurised sample was packed in sterile containers and frozen, another of the sample was freeze dried. The biomass pastes (pre-homogenised, homogenised and pasteurised) were sub sampled for microtests, with 100 μL of each sample/treatment plated on YM plates and incubated at 25° C. for 96 hours.

The fermentation process yielded 2.2. kg wet biomass (435 gm dry biomass). Homogenisation (optionally with pasteurisation) completely sterilised the biomass with a 0 CFU count observed upon plating. Table 18 shows the fatty acid analysis of the TFA, TAG and PL fractions of the freeze-dried biomass (before homogenization).

TABLE 18
Fatty acid composition (% mol)

| Sample | C14:0 | C15:0 | C16:0 | C16:1 | C18:0 | C18:1 | C18:1iso | C18:2 | C18:3 | C20:0 | ARA |
|---|---|---|---|---|---|---|---|---|---|---|---|
| TFA | 2.36 | 1.26 | 23.26 | 0.57 | 5.25 | 15.83 | 0.94 | 14.91 | 0.67 | 1.01 | 33.66 |
| TAG | 2.87 | 1.20 | 24.88 | 0.60 | 6.99 | 17.03 | 0.92 | 10.08 | 0.58 | 2.87 | 31.68 |
| PL | 1.20 | 1.09 | 18.49 | 0.00 | 2.09 | 13.59 | 1.02 | 20.30 | 0.72 | 4.06 | 37.45 |

### Example 8. Maillard Reactions Using Fungal Biomass and Extracted Lipid

The inventors tested the M. alpina cells and the extracted lipid obtained from the cells, enriched for polar lipid and containing the ω6 fatty acids ARA, DGLA and GLA, in Maillard reactions. The experiment also tested a combination of cells and the extracted lipid, all produced as described in Example 7. These reactions had L-cysteine, D-ribose, thiamine hydrochloride, iron fumarate and glutamic acid present in a phosphate buffer at pH 6.0, and either had added yeast extract or lacked the yeast extract. These reactions were intended to approximate the use of flavouring mixes having multiple components which are often added to food preparations for flavouring and other sensory attributes. The presence or absence of yeast extract was intended to test whether it would either mask, or enhance, the aroma produced by the M. alpina cells or extracted lipid having PL, or have little effect.

The base medium used for the Maillard reactions, designated “Matrix A” lacking yeast extract and “Matrix B” including yeast extract, had the following composition in aqueous buffer at final concentrations: 10 mM L-cysteine, 10 mM D-(−)-ribose, 2 mM thiamine hydrochloride, 35 μg/ml of iron fumarate (Apohealth, NSW, Australia) and 2 mM L-glutamic acid monosodium salt hydrate. These components were dissolved in 32.6 mM potassium phosphate buffer pH 6.0 for Matrix A or 12.6 mM phosphate buffer, pH 6.0 for Matrix B, prepared from potassium dihydrogen phosphate and dipotassium hydrogen phosphate. Yeast extract was added to Matrix B at a final concentration of 30 mg/ml. Reactions were carried out in 2 ml volumes in 20 ml glass vials with tightly sealing screw top lids. The reaction mixtures were made up with M. alpina dry biomass (150 mg) or extracted polar lipid (20 mg, 50 mg or 70 mg), or a combination of cells and lipid as indicated in Table 19. As controls for the presence of M. alpina biomass or polar lipid, other vials were made up with 150 mg of S. cerevisiae cells or 70 mg extracted lipid from the S. cerevisiae cells, both of which did not contain ω6 fatty acids (Reactions #8 to #12). Additional reaction mixtures (Reactions #5, #9) were prepared and vortexed, but then frozen overnight before being thawed and heat treated with the other reaction mixtures.

The mixtures were vortexed vigorously for 2 min and then heated for 75 min in an oven set at 140° C. The vials were tightly sealed during the heat treatment. It was estimated that the samples took about 15 min to warm to the oven temperature, so the heat treatment included about 60 min at 140° C. The vials were cooled until warm to the touch about 15 min later, and then opened briefly for sniffing by a panel of 5 volunteers (P1 to P5). These included males and females and ranged from 24-65 years in age. The volunteers did not know the composition of any of the vials prior to sniffing the contents and the vials were sniffed in a random order as selected by the volunteers. Their descriptions of the aromas were recorded without any comments being shared; the data are shown in Table 20. The descriptions of the aromas for reactions #4 to #7 were combined in Table 20 while still indicating any preference within reactions #4 to #7. The responses to reactions #8 to #11 were similarly combined for volunteers P3 and P4.

Reactions #4 to #7 containing M. alpina biomass and/or extracted lipid were described by all five volunteers as having a meaty aroma, but with different aroma notes recorded by the volunteers, whilst the descriptions of the aromas from reactions having the S. cerevisiae biomass were more variable between the volunteers. The control reaction mixtures lacking the lipid extract, and the mixtures having the lipid extract without any cell biomass, were generally perceived to have a lower intensity of aromas compared to the corresponding samples that contained biomass or a combination of biomass and extracted lipid from M. alpina. Reaction mixtures containing biomass spiked with the extracted lipid from M. alpina were described as having similar or enhanced aromas compared to reactions containing only M. alpina biomass. Mixtures that had been frozen and thawed and then heated resulted in similar aroma responses to freshly prepared mixtures heated in the same manner. The inventors concluded that the M. alpina biomass, containing polar lipids incorporating ω6 fatty acids, provided meaty aromas, particularly for beef aromas such as roast beef. Extracted lipid enriched for PL containing ω6 fatty acids also provided meaty aromas, enhancing the aromas when applied with the biomass. When used without the cell biomass, the responses for the extracted lipids were weaker but this could be countered by applying larger amounts of the lipid.

TABLE 19
Composition of Maillard reactions using
fungal biomass or extracted lipid

| Reaction | | | Extracted | Freeze- |
|---|---|---|---|---|
| ID | Matrix | Cell biomass | lipid | thaw? |
| 1 | A | none | none | No |
| 2 | B | none | none | No |
| 3 | B | none | 70 mg from | No |
| | | | M. alpina | |
| 4 | B | 150 mg M. alpina | none | No |
| 5 | B | 150 mg M. alpina | none | Yes |
| 6 | B | 150 mg M. alpina | 20 mg from | No |
| | | | M. alpina | |
| 7 | B | 100 mg M. alpina | 50 mg from | No |
| | | | M. alpina | |
| 8 | B | 150 mg S. cerevisiae | none | No |
| 9 | B | 150 mg S. cerevisiae | none | Yes |
| 10 | B | 120 mg S. cerevisiae | 30 mg from | No |
| | | | S. cerevisiae | |
| 11 | B | 100 mg S. cerevisiae | 50 mg from | No |
| | | | S. cerevisiae | |
| 12 | B | none | 70 mg from | No |
| | | | S. cerevisiae | |

TABLE 20
Reactions of volunteers to sniffing the products of Maillard reactions

| Reaction | | | | | |
|---|---|---|---|---|---|
| ID | P1 | P2 | P3 | P4 | P5 |
| 1 | Some light | Pork belly | Similar | Meaty smell, | Blank, pleasant |
| | meaty aroma | | between #1- | a bit stronger | |
| | | | 2, cannot | than #2 | |
| 2 | Some light | Pork belly | relate to a | Meaty smell | Blank, pleasant |
| | meaty aroma | | specific | | |
| | (lighter | | aroma | | |
| | than #8) | | | | |
| 3 | Pleasant | Light roast | Meaty aroma, | Light beef | Vegetable and |
| | aroma, but | meat | but do not | aroma | chicken |
| | not a | | like much | | |
| | distinguished | | | | |
| | aroma note | | | | |
| 4 | Roast meat, | Roast meat, | Meat aroma, | Beefy aroma, | Meaty aroma, very |
| 5 | similar | similar for | very pleasant, | similar | nice and pleasant. #5 |
| 6 | between | #4 to 7 | quite similar, | between #4 | is better than #4, #6 is |
| 7 | reactions | | but like #4 | to 7 | the best, #7 was |
| | #4 to 7 | | the most | | pleasant, but not as |
| | | | | | good as #6, somewhat |
| | | | | | like charred meat |
| 8 | Light meat | Meaty, | Meaty smell, | Beef cooked | Stew meat |
| | aroma | chicken, | like #10 the | with | |
| | | similar to #9 | best | mushroom | |
| 9 | Light meaty | Chicken, meaty, | | however, | Light meat, chicken |
| | aroma | pleasant | | there are | like flavour |
| 10 | Meaty and | Medium beefy | | some slight | Good roast beef/pork |
| | buttery | | | differences: | |
| 11 | Meaty and | Medium beefy, | | #11 smoky, | Similar aroma note |
| | buttery | similar to #10 | | #12 strongest | with #10, but slightly |
| | | | | | lighter |
| 12 | Light meaty | Beefy, pork | | | Vegetables |
| | aroma | lard smell | | | |

Several experiments were carried out to test variations of the Maillard reactions in terms of the composition of the base medium. In one experiment, four different base media were prepared either with or without the L-glutamic acid at 5 mM, or with or without an added Fenugreek (Trigonella foenum-graecum) leaf powder at 10 mg per 2 ml reaction. Fenugreek leaf powder was tested as this herb has long been used in food cooking to enhance the flavour of dishes such as in curries or in combination with other herbs or spices such as cumin and coriander. All of the base media included a yeast extract at 30 mg/ml. The reactions were set up including Y. lipolytica cells incorporating ARA in its polar lipid (cells from experiments B012 or B013) or M. alpina cells. The cells were applied as wet cells at 200 mg per 2 ml reaction in 20 ml glass vials, tightly sealed. Control reactions had the same base media compositions but lacked the Y. lipolytica or M. alpina cells. The reaction mixes were sonicated as a batch by placing all vials in a floating foam and placed in a sonicator (Soniclean, Thermoline) set up at a medium power for 30 min and then heat treated in an oven at 140° C. for 60 min. The vials containing the reaction mixtures were cooled slowly over about 15 min until warm to the touch. The contents were sniffed in random order by nine volunteers who did not know the composition of each mixture. The reactions had been coded with random 3-digit numbers to avoid bias, and the volunteers sniffed coffee beans between samples to reset the olefactory senses.

Although the descriptions of the aromas were variable between the nine volunteers, the aromas from mixtures having glutamic acid were generally described as more associated with meaty aromas compared to the reactions lacking glutamic acid. For example, a reaction mixture having glutamic acid was described as providing meaty aroma by 5 of the 9 participants whereas the corresponding sample lacking glutamic acid was described as having a meaty aroma by only 2 participants. Addition of fenugreek leaf powder in the reactions was generally described as generating a pleasant, sweet herb or vegetable aroma, but addition of the herb powder also moderated the meaty aroma in the presence of the Y. lipolytica or M. alpina cells. Some of the participants described the meaty aromas as “roast chicken”, “chicken broth” or “crispy chicken”, so identifying the aromas as like chicken in some form.

In another experiment, samples were prepared in either Matrix A or Matrix B as the base medium and containing either 100 mg wet M. alpina cells or 15 mg extracted lipid enriched for polar lipid. These reaction mixtures were prepared in 1 ml volumes in 20 ml glass vials and were each vortexed vigorously for 2 min. The mixtures were heat treated as before. Parallel mixtures were heated at a lower temperature, namely 115° C. for 25 min. The responses from three volunteers were consistent with the other experiments, in that the aromas generated by the whole cell biomass generated stronger meaty aromas than the extracted lipid on its own. The samples treated at 115° C. for the shorter time were evaluated as providing a weaker or lighter aroma, indicating that the treatment at 140° C. was more efficient at generating the meaty aroma than treatment at 115° C.

In another experiment, the M. alpina biomass as a dried powder was compared to several commercial plant-based and meat flavouring products on the market in Australia, including Deliciou plant-based beef, Deliciou plant-based chicken, Deliciou plant-based pork, Massel plant-based stock cube—beef, Massel plant-based stock cube—chicken, Oxo stock cube-beef, Oxo stock cube-chicken and Bonox beef stock. Reaction mixtures were prepared in 2 ml volumes using 150 mg of dry product or 200 mg of product as a wet paste and heated at 140° C. for about 60 min. When sniffed by four volunteers, the samples containing the M. alpina cell biomass were described as comparable or superior in their meaty aroma to the commercially-available flavouring products.

In another experiment, reaction mixes were prepared and then dried down by placing the vials in an oven at 115° C. for 2 h followed by 82° C. for a further 2 h. In a parallel experiment, corresponding samples were dried overnight at 70° C. After the heating, all of the samples were reconstituted in 2 ml of water, mixing them well to dissolve the dried powder, and subjected to sniffing by volunteers. The samples treated at the higher temperature generally provided a burnt smell, whereas the samples subjected to the lower temperature drying still provided some meaty aromas. This indicated that lower temperature drying was better than the higher temperature for retaining the meaty aroma. Further investigation is carried out to optimise the drying conditions.

The inventors concluded that a variety of compositions can be used with the yeast or fungal biomass containing ω6 fatty acids to enhance meaty aromas when heated, including in the presence of other flavouring components as commonly used in food preparations.

### Example 9. Further Maillard Reactions Using Fungal Biomass and Extracted Lipid

The inventors further tested the M. alpina cells and the extracted lipid obtained from the cells in further Maillard reactions under modified conditions. From the previous experiments, the samples containing M. alpina biomass were considered to have the strongest meat-like aroma, often described as having a roast meat/BBQ meat aroma. Several volunteers in the aroma tests, however, described that to them the aroma was like an overcooked or even burnt meat with a charred note. A “fatty aroma” was also noted by some. In another experiment, when the mixtures were tasted after heating, some volunteers described a sourness or bitterness in the samples including the matrix bases A and B, in particular bitterness for samples containing M. alpina. By tasting the individual solutions and ingredients to make up the matrix bases A and B, it was concluded that the thiamine hydrochloride contributed to the bitterness and to a lesser extent the yeast extract solution. The iron and cysteine solutions, both dissolved in 1 N HCl, contributed to the sourness. Several new experiments using different approaches reducing or eliminating these two components were therefore performed to improve both aroma and taste, aiming to reduce the burnt smell as well as the sourness and bitterness but retaining or even enhancing the meat-like aroma.

### Experiment 1

In this experiment, the M. alpina biomass was partially substituted with S. cerevisiae cells that did not contain ω6 fatty acids to see whether the burnt smell of the mixture was reduced after heat treatment compared to M. alpina alone. To do this, samples were prepared containing 150 mg of either dry M. alpina cells or S. cerevisiae cells in 2 ml of matrix B, or 75 mg of each of the fungal biomasses. The samples were heated in an oven set at 140° C. for 75 min. As before, the M. alpina sample generated a roast meat aroma, while the S. cerevisiae sample generated more of a gravy or chicken broth aroma. The mixed sample produced an intermediate aroma of roast and gravy meat. The volunteers described that the burnt smell was lessened for the mixture, but a decreased roast meat aroma was also noted. The sourness and bitterness were still found in all samples. The results suggested that some of the M. alpina biomass could be replaced with other cells to generate an intermediate meaty aroma more like a roast beef or gravy.

### Experiment 2

In this experiment, an alternative base medium was used to compare it to the Matrix B base. This alternative medium contained a mixture of amino acids, including cystine (33%), glutamine, alanine, leucine, glutamic acid, lysine, valine, proline and methionine as well as 2.7% dextrose by weight. This mixture was added at 7.5% (w/v) to the aqueous medium, as was an additional 0.5% (w/v) cystine and 0.5% (w/v) dextrose. The samples for the Maillard reactions used either 150 mg of dry M. alpina biomass or 300 mg of wet slurry of S. cerevisiae cells. Control samples had only the amino acids and sugars and no cells added. These mixtures were heated in an oven at 140° C. for 75 min. The control sample having Matrix B was described as having a light meaty aroma and some umami after taste, but was also perceived as having sourness and bitterness. In contrast, the samples containing M. alpina generated a meaty aroma. The control sample having the alternative base medium without fungal biomass had a pleasant aroma which was not related to a specific type of meat. When the M. alpina biomass was added, it generated different meaty notes and an umami/sweet taste perceived as an after taste. A slight sourness and bitterness was still perceived in these samples. It was considered that the slight sourness and bitterness could be masked by increasing the amount of dextrose.

### Experiment 3

In another experiment, the alternate base medium was used at two concentrations: 7.5% (w/v) or 0.75% (w/v). Another sample had an additional 100 mg dextrose added per 2 ml mixture. Some samples contained 200 mg of extracted polar lipid, mostly PL, from M. alpina. A shortened heat treatment of 45 min at 140° C. was applied for samples containing M. alpina while the standard heat treatment of 75 min at 140° C. was used for other samples. The volunteers described that the mixtures having the higher concentration of base medium had a more distinguished meat-like and pleasant aroma compared to the samples prepared at the low concentration. Further, the higher concentration samples had a browny/golden brown colour after the heat treatment, whereas the lower concentration samples did not have that colour. Based on these results, the future experiments used the higher concentration of base medium. The samples with the PL isolated from M. alpina generated a weaker, but “purer” meaty aroma compared to the use of M. alpina biomass at 150 mg/2 ml. Significantly, heating the samples containing M. alpina for 45 min rather than 75 min reduced the burnt smell and substantially reduced the bitterness. Increasing the amount of dextrose also decreased the perception of bitterness, although some was still noted.

### Experiment 4

This experiment compared the use of a wet form of the fungal biomass compared to the dry form. In this experiment, the sample containing M. alpina dry biomass based on the findings of the previous experiments had a roast aroma with no burnt smell and a light bitterness after the heat treatment. The sample with wet biomass generated a pleasant roast meat aroma with no hint of a burnt smell and a very subtle bitterness similar to the control samples lacking the biomass. This subtle bitterness was similar to the taste of the control mixture having only the base medium, most likely due to its amino acid constituents or the thiamine hydrochloride. It was considered that the increased moisture in the biomass had slowed down the burning process when the biomass was exposed to the high temperature treatment, but still provided sufficient conditions for a Maillard reaction to occur.

### Example 10. Food Products Using Fungal Biomass and Extracted Lipid

The inventors next tested the yeast and M. alpina cells and the extracted lipid obtained from the cells in exemplary food products to test their aroma and taste. The chemicals and ingredients used for the taste mixtures included L-cysteine hydrochloride monohydrate (Fermopure, Wacker, Germany), D-ribose (Epin Biotech Co, China), thiamine hydrochloride (Chem Supply, SA, Australia), monosodium glutamate (Ajinomoto), Yeast extract (Sigma) and an amino acid/sugars blend (provided by V2Foods). The oils and plant-based fats used were canola oil, “Heart Smart” safflower cooking oil and copha vegetable shortening from a supermarket and a plant-based ghee (Emkai Lite Interesterified vegetable fat, Sai food products, Gujarat, India). The food items tested by applying the taste mixtures were a macro firm tofu obtained from a local supermarket, dried bean curd (tofu skin, Shenzhen Ming Lee Food Manufacturing Co. Ltd., Guandong Province, China), a plant-based mince (V2 Foods, Australia) and textured vegetable protein high fibre slices (TVP, Lamyong, NSW, Australia). The fungal biomasses used were a wet slurry of S. cerevisiae having about 10% ARA (B013, see Example 4), or M. alpina biomass in either a wet or dry form (Example 7).

### Experiment 1

This experiment used the B013 yeast biomass, containing ARA in both the polar lipid and TAG (Example 4). A mixture (mixture A) was prepared containing 2 ml of a Matrix B2 base medium. Matrix B2 contained one tenth the concentration of thiamine hydrochloride compared to Matrix B but otherwise had an identical composition. Mixtures were prepared having 0.5 ml of B013 cell slurry and 0.5 ml of a chicken flavoured yeast extract (2.5 g/3 ml water, Flavex). Control mixtures lacked either the B013 cell slurry or the Matrix B2 base medium. Tofu pieces were marinated in the mixtures for 45 min and cooked on a baking tray in an oven set at 180° C. for about 6 min. When smelt and tasted, all of the tofu pieces had a salty/sweet/umami taste but only the test pieces treated with mixture A exhibited a light roast chicken aroma and taste. It was considered that the umami taste was most likely brought by the flavoured yeast extract whereas the B013 yeast biomass contributed to the chicken aroma.

### Experiment 2

It was considered that cooking the tofu pieces for only 6 min was not long enough to induce a complete Maillard reaction with the mixtures used, so, in a following experiment, the basting mixtures were heated at 140° C. for 75 min prior to application to the tofu pieces. A taste mixture was prepared containing the B013 yeast biomass having ARA in its lipid, for which 2 ml Matrix B2 was mixed with 300 mg of wet yeast biomass. This taste mixture was then heated in an oven set at 140° C. for 75 min. Tofu pieces and tofu skin pieces were marinated in 1 ml of the taste mixture for 1 h and then oven baked at 180° C. for 6 min. A meaty aroma was perceived during the marination step and before putting the sample into the oven. However, after heating in the oven, the meaty aroma was no longer perceived. It was concluded that the volatile compounds that imparted the meaty aroma had evaporated during the heating in the oven.

### Experiment 3

In this experiment, the yeast biomass was substituted with 200 mg of M. alpina wet biomass, having about 30% ARA in its lipid. The composition of the mixtures and baking conditions were otherwise the same as in Experiment 2 except that the mixtures were heated for 45 min rather than 75 min prior to application to the tofu pieces. After heating them in the oven, the tofu pieces were sniffed and tasted. The volunteers described that the control tofu marinated in Matrix B2 without the M. alpina biomass had a pleasant, light meaty aroma, whereas the tofu treated with the mixture having the M. alpina biomass had a strong meaty aroma and taste.

### Example 11. Effect of Biomass Concentration on Meaty Aroma and Taste

### Experiment 1

The effect of M. alpina biomass on aroma and taste in a Maillard reaction composition was investigated. 8 samples were prepared containing wet M. alpina biomass (approx. 75% moisture) and matrix base C. The samples included M. alpina biomass in weight percentages of from 0.1% to 15% (0.025% to 3.75% dry weight equivalent), as well as one control (no biomass, only matrix. Sample compositions are shown in Table 21 below, and the composition ofmatrix C is shown in Table 22 below.

TABLE 21
Samples comprising wet biomass and matrix C

| | | Biomass % | Equivalent wet | |
|---|---|---|---|---|
| | Matrix | (wet form) | biomass (g) | |
| Control | 2 mL matrix C | 0% biomass | 0 | mg |
| Sample 1 | 2 mL matrix C | 0.1% biomass | 2 | mg |
| Sample 2 | 2 mL matrix C | 0.5% biomass | 10 | mg |
| Sample 3 | 2 mL matrix C | 1% biomass | 20 | mg |
| Sample 4 | 2 mL matrix C | 2.5% biomass | 50 | mg |
| Sample 5 | 2 mL matrix C | 5% biomass | 100 | mg |
| Sample 6 | 2 mL matrix C | 7.5% biomass | 150 | mg |
| Sample 7 | 2 mL matrix C | 10% biomass | 200 | mg |
| Sample 8 | 2 mL matrix C | 15% biomass | 300 | mg |

TABLE 22
Composition of matrix C.

| | Volume | Final | |
|---|---|---|---|
| Stock solutions | (μL) | concentration | |
| Cysteine•HCL (400 mM) | 250 | 50 | mM |
| Ribose (400 mM) | 250 | 50 | mM |
| Thiamine•HCL (44 mM) | 90.9 | 2 | mM |
| Yeast extract (general) (30 g/100 mL) | 1000 | 0.15 | g/mL |
| Monosodium glutamate (400 mM) | 125 | 25 | mM |

| | |
|---|---|
| Water | 284.1 |
| Total | 2000 |

Samples were vigorously mixed for 2 minutes at room temperature and subjected to heating at 140° C. for 45 minutes. After the heat treatment, samples were cooled and tempered at 45° C. throughout subsequent sensory evaluation.

For sensory evaluation, a total of six participants (both male and female, aging from 25-65) were asked to sniff and taste the samples in order as indicated in Table 20. Between samples, the participants were asked to sniff coffee and drink water to neutralize/clear the nose and tongue. The participants were asked to evaluate both aroma and taste for meatiness and pleasantness based on a five-point hedonic scale, with the higher score indicating increased meatiness and pleasantness.

Addition of biomass was found to significantly increase the meatiness perceived by the participants, and this was observed at 0.1% addition of wet biomass (equivalent to 0.025% dry biomass). Within the concentration range of 0.1 to 15% wet biomass (equivalent to 0.025% to 3.75% dry biomass), the 5% wet biomass was ranked as the most meaty sample, followed by the 15% and 10% samples. By further statistical analysis, a strong positive correlation (r=0.70) was found between biomass and the meatiness of the samples. No clear trend in correlation between biomass and pleasantness was found. When considering the total score of pleasantness and meatiness, the sample containing 5% wet biomass had the highest score.

### Experiment 2

The effect of M. alpina biomass concentration on aroma and taste in food was investigated. 8 samples were prepared containing wet M. alpina biomass at varying concentrations (approx. 75% moisture), matrix base C and textured vegetable protein (‘TVP’, V2Foods, Australia), as well as a control comprising no biomass.

To prepare the samples, vials were prepared comprising M. alpina wet biomass (in non-control samples, concentrations varying from 0.10%-10% biomass/TVP w/w %), matrix C (as described above in Table 20) and, in one sample, water instead of matrix C. The vials were vortexed at 20000 rpm for 2 minutes before being subjected to a heat treatment at 140° C. for 45 minutes. The composition of the vials is shown below in Table 23.

TABLE 23
Composition of samples

| Vial | 1 | 2 | 3 | 4 | 5 | 6 | 7 | 8 | 9 |
|---|---|---|---|---|---|---|---|---|---|
| Biomass/TVP | 0% | 0.10% | 0.50% | 1% | 2.50% | 5% | 7.50% | 10% | 1%* |
| (w/w %) | | | | | | | | | |
| M. Alpina_wet (mg) | 0 | 50 | 250 | 500 | 1250 | 2500 | 3750 | 5000 | 500 |
| Matrix C (ml) | 5 | 5 | 5 | 5 | 5 | 5 | 5 | 5 | 0 |
| Water | 0 | 0 | 0 | 0 | 0 | 0 | 0 | 0 | 5 |

450 g of rehydrated TVP was prepared by adding 320 g of water to 130 g of TVP and leaving to rehydrate for 30 minutes. The rehydrated TVP was then divided into 9 portions, and each portion added to one of the vials of Table 21 and mixed and marinated thoroughly for approximately 5 minutes. Each portion of the marinated TVP was then cooked on a frying pan at a medium heat setting (1000 W) with 3 mL of canola oil for 2 minutes.

For sensory evaluation, a total of six participants (both male and female, aging from 25-65) were asked to sniff and taste the TVP food samples in order as indicated in Table 23. Between samples, the participants were asked to sniff coffee and drink water to neutralize/clear the nose and tongue. The participants were asked to evaluate both aroma and taste for the meatiness and pleasantness based on a five-point hedonic scale, with the higher score indicating increased meatiness and pleasantness.

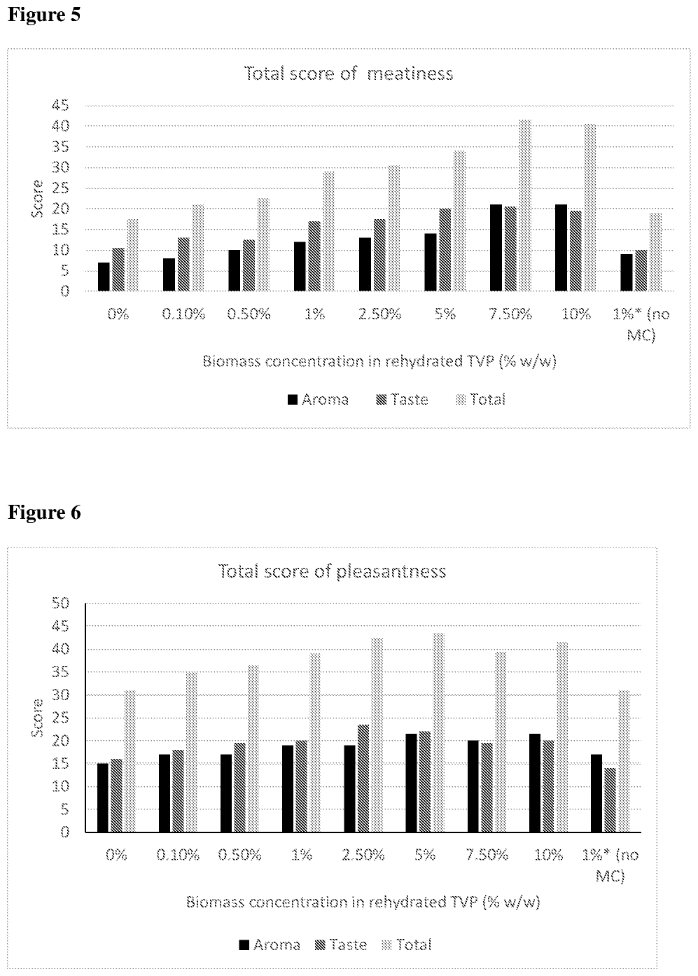

Results for meatiness, pleasantness, and combined meatiness and pleasantness are shown in FIGS. 5, 6 and 7 respectively.

Overall, an increase in biomass resulted in an increase in meatiness perceived by the 6 participants (FIG. 5). The increase in meatiness was observed when 0.1% wet biomass (equivalent to 0.025% dry biomass) was added. The sample was perceived as having the most meatiness when wet biomass was added at the rate of 7.5% (equivalent to 1.88% dry biomass), and the meatiness intensity perceived slightly decreased at 10% wet biomass inclusion.

Similarly, the addition of biomass was also found to increase the pleasantness of the samples (FIG. 6). Sample 6 (5% wet biomass, equivalent to 1.25% dry biomass) achieved the highest pleasantness score, following by Sample 5 (2.5% wet biomass). Overall, the pleasantness for both aroma and taste increased when the concentration of biomass increased (0% to 5%), and decreased above 5%.

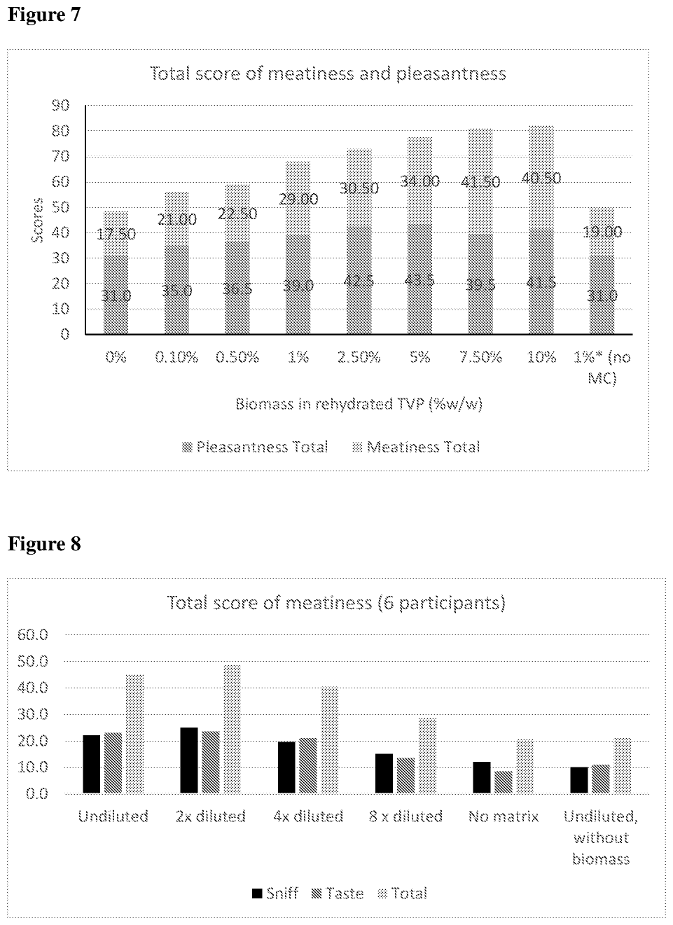

When considering both meatiness and pleasantness, samples containing 7.5% wet biomass showed the highest total score (FIG. 7).

The above results demonstrate an effect of biomass concentration on the meatiness and pleasantness of the samples. An increase in wet biomass concentration from 0.1% to 5% (equivalent to 0.025% to 1.25% dry biomass) increased both the pleasantness and meatiness of the samples. The meatiness reached a maximum level at 7.5% wet biomass inclusion, where the pleasantness started to decrease.

### Example 12. Effect of Matrix Component Concentration on Meaty Aroma and Taste

Matrix C as defined in Example 11 was mixed at different levels of dilution with the same concentration of wet M. alpina biomass (10% w/v). The six samples prepared are provided in Table 24 below. The associated compositions of matrix C in each of the samples is provided in Table 25 below.

TABLE 24
Composition of samples

| | Matrix | Wet biomass (g) |
|---|---|---|
| Sample 1 | 2 mL matrix C- undiluted | 200 mg |
| Sample 2 | 2 mL matrix C- 2x diluted | 200 mg |
| Sample 3 | 2 mL matrix C -4x diluted | 200 mg |
| Sample 4 | 2 mL matrix C - 8x diluted | 200 mg |
| Sample 5 | No matrix | 200 mg |
| Sample 6 | 2 mL matrix C- undiluted | No biomass |

TABLE 25
Composition of matrix C at different dilution levels for samples 1-6 (amounts shown in μL)

| | Sample 1 | Sample 2 | Sample 3 | Sample 4 | Sample 5 | |
|---|---|---|---|---|---|---|
| Stock | Undiluted + 1% | 2x diluted + 1% | 4x diluted + 1% | 8x diluted + 1% | No matrix + 1% | Sample 6 |
| solutions (μL) | biomass | biomass | biomass | biomass | biomass | Undiluted |
| Cysteine•HCL | 250 | 125 | 62.5 | 31.25 | 0 | 250 |
| (400 mM) | | | | | | |
| Ribose | 250 | 125 | 62.5 | 31.25 | 0 | 250 |
| (400 mM) | | | | | | |
| Thiamine•HCL | 90.9 | 45.45 | 22.72 | 11.36 | 0 | 90.9 |
| (44 mM) | | | | | | |
| Yeast extract | 1000 | 500 | 250 | 125 | 0 | 1000 |
| (general) | | | | | | |
| (30 g/100 mL) | | | | | | |
| Monosodium | 125 | 62.5 | 31.25 | 15.62 | 0 | 125 |
| glutamate | | | | | | |
| (400 mM) | | | | | | |
| Water | 284.1 | 1142.05 | 1571.03 | 1785.5 | 2000 | 284.1 |
| Total | 2000 μL | 2000 μL | 2000 μL | 2000 μL | 2000 μL | 2000 μL |

The samples were vigorously mixed for 2 minutes at room temperature and subjected to heating at 140° C. for 45 minutes. After the heat treatment completed, the samples were cooled down and tempered at 45° C. throughout sensory evaluation.

For sensory evaluation of the samples, a total of six participants (both male and female, aging from 25-65) were asked to sniff and taste the samples in order of 5 to 1 and then 6. Between samples, the participants were asked to sniff coffee and drink water to neutralize/clear the nose and tongue. The participants were asked to evaluate both aroma and taste for the meatiness and pleasantness based on a five-point hedonic scale, with the higher score indicating increased meatiness and pleasantness.

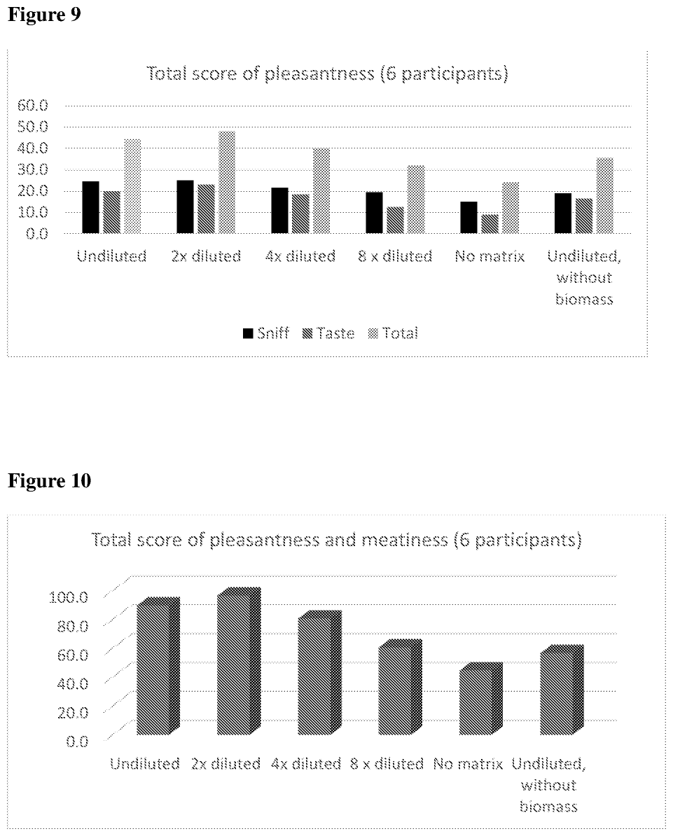

The total score of 6 participants for meatiness, pleasantness and combined pleasantness and meatiness are presented in FIGS. 8, 9 and 10 respectively. It was found that a slight increase in meatiness and pleasantness of the sample was observed when the matrix was diluted 2×, but further dilutions of matrix more than 2× decreased both meatiness and pleasantness of the samples.

As expected, the undiluted matrix sample with biomass had a higher score for meatiness and pleasantness compared to the undiluted matrix sample without the biomass, confirming that the addition of biomass into the matrix enhances the meaty aroma as observed in previous experiments.

### Example 13. Assessment of Additional Mortierella Spp in Maillard Reaction

Additional Mortierella spp. isolates were identified from various soil samples and assessed in Maillard reactions.

### Experiment 1

One isolate (labelled Myu1) was identified as M. elongata based on ITS homology (wherein the ITS of Myu1 is set forth in SEQ ID NO:48) and another isolate (labelled S′2-1) was identified as M. exigua based on ITS homology (wherein the ITS of S′2-1 is set forth in SEQ ID NO:49). The isolates were cultured and the resulting biomass analysed for fatty acid content in the lipid fraction. As shown in Table 26, ARA was present in an amount of about 33% of the total fatty acid content of the lipid of the Myu1 lipid, and in an amount of about 24% of the total fatty acid content of the S′2-1 lipid. The percentage oil by weight of the Myu1 isolate was 10.06%, while the percentage oil by weight of the S′2-1 isolate was 4.08%.

TABLE 26
Fatty acid composition of lipids from Mortierella isolates
Fatty acid composition (% mol)

| Sample | C14:0 | C16:0 | C16:1 | C18:0 | C18:1 | C18:1iso | C18:2 | C18:3 | C20:0 | ARA |
|---|---|---|---|---|---|---|---|---|---|---|
| S′2 -1 | 1.40 | 16.78 | 0.63 | 5.43 | 21.46 | 1.99 | 13.51 | 5.56 | 0.39 | 24.21 |
| Myu1 | 0.35 | 6.263 | 0 | 3.94 | 20.50 | 0.82 | 12.55 | 7.82 | 0.68 | 33.55 |

To assess the ability of each isolate to impart meaty aromas and flavours, a biomass equivalent to 50 mg dry matter of each isolate, as well as the M. alpina isolate, was weighed and transferred into a 20 mL glass vial. Matrix C (2 mL; defined in Table 53 above) was then added into each vial. A Matrix C only (i.e. no biomass) negative control was also included. The samples were then vigorously mixed for 2 min at room temp and subjected to heating at 140° C. for 45 min. After the heat treatment was completed, the samples were cooled down and tempered at 45° C.

A total of six participants (both male and female, aging from 25-65) were asked to sniff the M. alpina sample first and use it as the reference for testing other blinded samples (at any order preferred by the participants). Between samples, the participants were requested to sniff the coffee to neutralize/clear the nose. The participants were requested to evaluate the aroma for the meatiness and pleasantness based on a five-point hedonic scale, with the higher score indicating the increased meatiness and pleasantness.

As shown in Table 27 below, each of the Mortierella isolates imparted a meaty aroma when heated in the presence of the Matrix components, above and beyond what was detected with the Matrix C only. While the M. alpina isolate imparted higher levels of meatiness, participants considered the M. elongata isolate to have the most pleasant aroma.

TABLE 27
Sensory assessment of Maillard reactions

| | | M. alpina + | Myu1 + | S′2-1 + |
|---|---|---|---|---|
| | Matrix C only | Matrix C | Matrix C | Matrix C |
| Pleasantness | 14.0 | 16.0 | 22.0 | 18.5 |
| Meatiness | 12.0 | 22.0 | 18.5 | 20.0 |
| Overall | 26.0 | 38.0 | 40.5 | 38.5 |

### Experiment 2

Additional Mortierella spp. isolates were isolated and tested in Maillard reactions essentially as described above, and compared to NI0132 (M. alpina) and matrix only control. These isolates included S1-3, identified as M. elongata based on ITS homology; S2-2, identified as M. minutissima based on ITS homology; S2-3, identified as M. minutissima or M. zonata based on ITS homology; SS3, identified as M. M. minutissima based on ITS homology; Myu3, identified as M. elongata based on ITS homology; Burnsen1, identified as M. elongata based on ITS homology; and Burnsen2, also identified as M. elongata based on ITS homology. Analysis of the TFA in the lipids from the isolates is shown in Table 28.

TABLE 28
Fatty acid composition of lipids from Mortierella isolates

| Fatty acid composition (% mol) | | | | | | | | | | | |
|---|---|---|---|---|---|---|---|---|---|---|---|
| Sample | C14:0 | C16:0 | C16:1 | C18:0 | C18:1 | C18:1iso | C18:2 | C18:3 | C20:0 | ARA | % oil |
| S1-3 | 1.9 | 11.7 | 0.2 | 3.3 | 23.5 | 1.0 | 12.5 | 7.5 | 0.2 | 23.7 | 5.88 |
| S2-2 | 0.4 | 8.1 | 0.1 | 10.2 | 12.9 | 0.6 | 14.7 | 6.5 | 1.0 | 34.1 | 2.79 |
| S2-3 | 0.9 | 11.0 | 2.3 | 10.4 | 12.8 | 4.5 | 13.6 | 6.7 | 1.1 | 23.2 | 2.21 |
| S′2-3 | 0.9 | 13.0 | 0.6 | 2.7 | 15.5 | 2.1 | 13.7 | 9.7 | 0.4 | 27.1 | 19.14 |
| Myu3 | 0.6 | 7.8 | 0.1 | 3.4 | 18.4 | 0.8 | 15.2 | 11.3 | 0.6 | 27.4 | 7.96 |
| Burnsen1 | 1.1 | 13.6 | 0.4 | 9.1 | 28.9 | 0.9 | 10.1 | 7.1 | 0.5 | 22.7 | 7.63 |
| Burnsen2 | 0.9 | 14.2 | 0.5 | 5.3 | 26.6 | 0.9 | 12.7 | 7.6 | 0.3 | 25.5 | 6.61 |
| NI0132 | 0.6 | 25.2 | 0.2 | 12.6 | 13.4 | 0.3 | 11.2 | 4.1 | 0.0 | 32.0 | 14.42 |
| (B17) | | | | | | | | | | | |

Table 29 shows the results of the blinded sensory assessment of the Maillard reactions, where the total score from six participants is shown. All of the Mortierella strains imparted increased meatiness aroma to the reactions, compared to the matrix only control.

TABLE 29
Sensory assessment of Maillard reaction

| | S1-3 | S2-2 | S2-3 | S′2-1 | S′2-3 | Myu1 | Myu3 | Bn1 | Bn2 | NI-0132 | Matrix |
|---|---|---|---|---|---|---|---|---|---|---|---|
| Pleasant | 22.0 | 19.0 | 18.0 | 20.0 | 22.0 | 23.0 | 22.5 | 21.0 | 22.0 | 16.0 | 19.0 |
| Meaty | 19.0 | 16.0 | 16.0 | 17.5 | 17.5 | 21.0 | 21.5 | 19.0 | 20.0 | 21.0 | 13.0 |

### Example 14. Comparison of M. alpina Biomass and ARA Oil in Maillard Reaction

Previous studies indicated that ARA oil (i.e. TAG comprising about 40% ARA) produced a less meaty and less pleasant aroma when heated with a sugar and an amino acid than polar lipid containing ARA (see Example 4). To assess this further, the inventors compared the aromas generated by M. alpina biomass and ARA oil in Maillard reactions.

A set of 4 samples were prepared according to Table 30, using ARA oil from NuCheck Inc. (Cat #NC0632549). Sample C contained equivalent ARA to that in the biomass, while sample D contained 10% ARA oil. The samples were then vigorously mixed for 2 min at room temp and subjected to heating at 140° C. for 45 min. After the heat treatment was completed, the samples were cooled down and tempered at 45° C. throughout the sensory evaluation.

TABLE 30
Sample preparation

| Sample | Matrix C | M. alpina biomass | ARA oil |
|---|---|---|---|
| A | 2 mL matrix C | NO biomass | No ARA oil |
| B | 2 mL matrix C | 200 g wet biomass | NO ARA oil |
| C | 2 mL matrix C | NO biomass | 13.1 mg ARA oil |
| D | 2 mL matrix C | NO biomass | 204.4 mg ARA oil |

A total of five participants (both male and female, aging from 25-65) were asked to sniff the samples in order of A to D. Between samples, the participants were requested to sniff the coffee to neutralize/clear the nose. The participants were requested to evaluate the aroma for the meatiness and pleasantness based on a five-point hedonic scale, with the higher score indicating the increased meatiness and pleasantness.

Samples having ARA oil added into the Maillard base were perceived as less pleasant and meaty (and more fatty/oily) compared to the samples having biomass, resulting in lower scores (Table 29). Thus, Mortierella biomass with polar lipid (and neutral lipid) containing ARA produced more meaty and pleasant aromas than purified neutral lipid containing ARA.

TABLE 31
Sensory assessment

| | | Sample B | Sample C | Sample D |
|---|---|---|---|---|
| | Sample A | Matrix + | Matrix + | Matrix + ARA |
| | Matrix only | biomass | ARA oil | oil (10%) |
| Pleasantness | 14.0 | 17.0 | 13.0 | 12.0 |
| Meatiness | 13.0 | 19.0 | 11.5 | 13.0 |
| Total | 27.0 | 36.0 | 24.5 | 25.0 |

### Example 15. Comparison of M. alpina and M. isabellina in Maillard Reaction

To assess the importance of arachidonic acid in the biomass, Maillard reactions using M. isabellina, which has no arachidonic acid, were performed. Briefly, M. isabellina was cultured and an amount of approximately 200 mg of M. isabellina wet biomass was transferred into a 15 mL Falcon tube and then dried in the oven set at 80° C. The samples were dried until the weight remained unchanged or up to 42 hours. Moisture content was then determined and fatty acid content in the total lipid fraction assessed. M. alpina biomass prepared as previously described was also utilised in the study.

Analysis of the fatty acid content in the total lipid fraction confirmed that ARA was absent from the lipid of M. isabellina (see Table 32). The biomass had a total oil content of 3.97% by weight.

TABLE 32
Fatty acid content of lipid

| C14:0 | C14:1 | C15:0 | C15:1 | C16:0 | C16:1 | C18:0 | C18:1 | C18:1iso | C18:2 | C18:3 | C20:0 | C22:0 | ARA |
|---|---|---|---|---|---|---|---|---|---|---|---|---|---|
| 0.76 | 0.00 | 0.33 | 0.00 | 16.15 | 2.13 | 1.86 | 36.20 | 0.49 | 31.27 | 10.62 | 0.17 | 0.00 | 0.00 |

A set of 8 vial samples were prepared according to Table 33. In each 20 mL glass vial, a biomass equivalent to 50 mg (or less, as detailed in the table) dry matter based on an estimated moisture content was weighed and transferred into the vial. An amount of 2 mL matrix C was then added into each vial. A negative control with matrix C only and no biomass was also included. The samples were then vortex at 2000 rpm to mix for 2 min at room temperature and subjected to heating at 140° C. for 45 min. After the heat treatment was completed, the samples were cooled down and tempered at 45° C. throughout the sensory evaluation.

TABLE 33
Sample preparation

| | | Wet | Dry biomass | |
|---|---|---|---|---|
| Sample | | Biomass | equivalent | Matrix |
| Code | Variation | (mg) | (mg) | C |
| 1 | Matrix C only | 0 | | 2 ml |
| 2 | M. alpina + Matrix C | 200 | 50 | 2 ml |
| 3 | M. isabellina + Matrix C | 166.7 | 13.0 | 2 ml |
| 4 | M. isabellina + Matrix C | 200.0 | 15.6 | 2 ml |
| 5 | M. isabellina + Matrix C | 250.0 | 19.5 | 2 ml |
| 6 | M. isabellina + Matrix C | 333.3 | 26.0 | 2 ml |
| 7 | M. isabellina + Matrix C | 500.0 | 39.0 | 2 ml |
| 8 | M. isabellina only | 200.0 | 15.6 | 2 ml of |
| | | | | water |

A preference testing method using 5-point hedonic scales and a scaling test with labelled magnitude scales were used in this evaluation. A total of six participants (both male and female, aging from 25-65) were asked to sniff the samples in order of #8, then #1 to #7 and use #1 as the reference. Between samples, the participants were requested to sniff the coffee to neutralize/clear the smelling sense and avoid the carry-over effect from the previous sample. The participants were requested to evaluate the aroma for the meatiness and pleasantness based on a five-point hedonic scale, with the higher score indicating the increased meatiness and pleasantness.

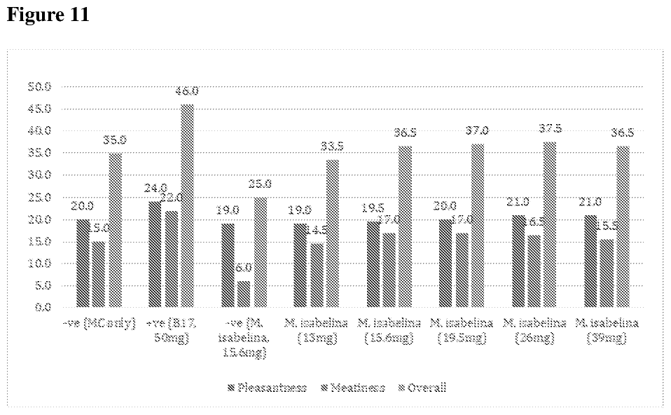

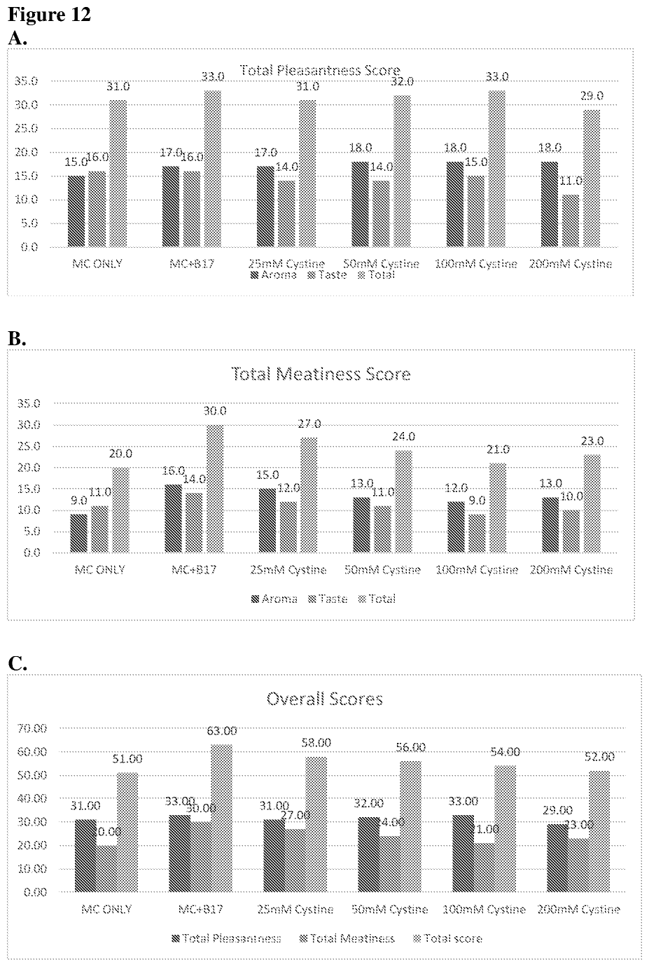

As shown in FIG. 11, M. isabellina was associated with lower meatiness and pleasantness scores compared to M. alpina, suggesting that arachidonic acid was important for generating meaty aromas. When comparing the positive control (M. alpina “B17” biomass) and the 39 mg M. isabellina sample, the latter was scored lower in pleasantness and the meatiness level lower by an average of 1.1 on a scale from 1 to 5. While that the dry mass equivalent weight for this sample was lower than the 50 mg used for M. alpina, notably as the concentration of M. isabellina increased, an associated slight decrease in the meatiness aromas was observed. It could therefore be assumed that a 50 mg sample of M. isabellina would have an even lower meatiness aroma.

### Example 16. Further Assessment of Maillard Reaction Components

A series of studies were performed to assess the various Maillard reaction components.

Experiment 1. Replacement of Cysteine with Cystine

In this experiment, the effect of replacing cysteine with cystine was assessed by aroma and taste tests.

### Sample Preparation—Concentrate

A set of 6 samples, containing a positive control/reference sample (Matrix C (MC) and 10% wet M. alpina biomass (M. alpina strain yNI0132, B017 culture as described above [“B17 wet” ]), a negative control (Matrix C only) and 4 samples containing 10% biomass and Matrix C but with cystine of differing concentration ranging from 0 to 200 m were prepared according to Table 34.

TABLE 34
Concentrate Preparation

| | Sample Number | | | | | |
|---|---|---|---|---|---|---|
| | 1 | 2 | 3 | 4 | 5 | 6 |
| | −ve | +ve | 25 mM | 50 mM | 100 mM | 250 mM |
| Cysteine•HCl | 250 | 250 | 0 | 0 | 0 | 0 |
| (400 mM) (μL) | | | | | | |
| Cystine (powder) (mg) | 0 | 0 | 12.015 | 24.03 | 48.06 | 96.12 |
| Ribose (400 mM) (μL) | 250 | 250 | 250 | 250 | 250 | 250 |
| Thiamine•HCl | 90.9 | 90.9 | 90.9 | 90.9 | 90.9 | 90.9 |
| (44 mM) (μL) | | | | | | |
| Yeast extract | 1000 | 1000 | 1000 | 1000 | 1000 | 1000 |
| (30 g/100 mL) (μL) | | | | | | |
| Monosodium glutamate | 125 | 125 | 125 | 125 | 125 | 125 |
| (400 mM) (μL) | | | | | | |
| Water (μL) | 284.1 | 284.1 | 534.1 | 534.1 | 534.1 | 534.1 |
| Total (μL) | 2000 | 2000 | 2000 | 2000 | 2000 | 2000 |
| B17_wet (mg) | 0 | 200 | 200 | 200 | 200 | 200 |

After adding all ingredients to the 20 ml GC headspace vials, samples were vigorously mixed (2000 rpm) for 2 min at room temperature (22-24° C.) and subjected to heating at 140° C. for 45 min. After the heat treatment was completed, the samples were cooled down and tempered at 45° C. throughout the sensory evaluation.

### Sample Preparation—Food

A set of 5 samples were prepared as detailed in Table 35 below. Because of the low solubility of cystine in water and the undesirable taste of undissolved cystine, 30 mg of cystine was added in the matrix in the initial oven heating stage then additional cystine powder was added directly into the TVP during the marination step to make up the set concentration.

TABLE 35
Recipe of food preparation

| | Sample Number | | | | |
|---|---|---|---|---|---|
| | 1 | | | | |
| | −ve (TVP | 2 | 3 | 4 | 5 |
| | plain) | 25 mM | 50 mM | 200 mM | MC only |
| Cysteine•HCl (400 mM) (μL) | 0 | 0 | 0 | 0 | 625 |
| Cystine powder to add in vial (mg) | 0 | 30.0 | 30.0 | 30.0 | 0 |
| Cystine powder to add during marination | 0 | 0 | 30.0 | 210.3 | 0 |
| (mg) | | | | | |
| Ribose (400 mM) (μL) | 0 | 625 | 625 | 625 | 625 |
| Thiamine•HCl (44 mM) (μL) | 0 | 227.25 | 227.25 | 227.25 | 227.25 |
| Yeast extract (30 g/100 mL) (μL) | 0 | 2500 | 2500 | 2500 | 2500 |
| Monosodium glutamate (400 mM) (μL) | 0 | 312.5 | 312.5 | 312.5 | 312.5 |
| Water (μL) | 5000 | 1335.25 | 1335.25 | 1335.25 | 710.25 |
| Total (μL) | 5000 | 5000 | 5000 | 5000 | 5000 |
| B17_wet (mg) | 0 | 500 | 500 | 500 | 0 |

After adding all ingredients to the 20 ml GC headspace vials, samples were vigorously mixed (2000 rpm) for 2 minutes at room temp (22-24° C.) and subjected to heating at 140° C. for 45 min. After the heat treatment completed, the samples were left on the bench to cool down completely. After cooling down, each taste mixture was added to 50 grams of rehydrated TVP flakes, mixed thoroughly, and let sit to marinate for 15 minutes.

After marinating, each marinated TVP sample was cooked on an oiled frying pan (3 ml of safflower oil) at 1000 W for 2 minutes, stirring occasionally. Between each sample, the frying pan was cleaned and dried. All cooked samples were stored in individual closed containers and kept at 60° C. for no longer than 1 hour until tasting.

### Sensory Evaluation Methods

To assess the aroma and taste of the concentrate, five participants (both male and female, aging from 25-65) were asked to sniff and taste the samples in the order from sample 1 to sample 6. Between samples, the participants were requested to sniff the coffee and drink water to neutralize/clear the nose and tongue. The participants were requested to evaluate both aroma and taste for the meatiness and pleasantness based on a five-point hedonic scale, with the higher score indicating the increased meatiness and pleasantness.

To assess the food sample, five participants (both male and female, aging from 25-65) were asked to sniff and taste the samples in the order from sample 7 to sample 1. Between samples, the participants were requested to sniff the coffee and drink water to neutralize/clear the nose and tongue. The participants were requested to evaluate both aroma and taste for the meatiness and pleasantness based on a five-point hedonic scale, with the higher score indicating the increased meatiness and pleasantness.

### Results

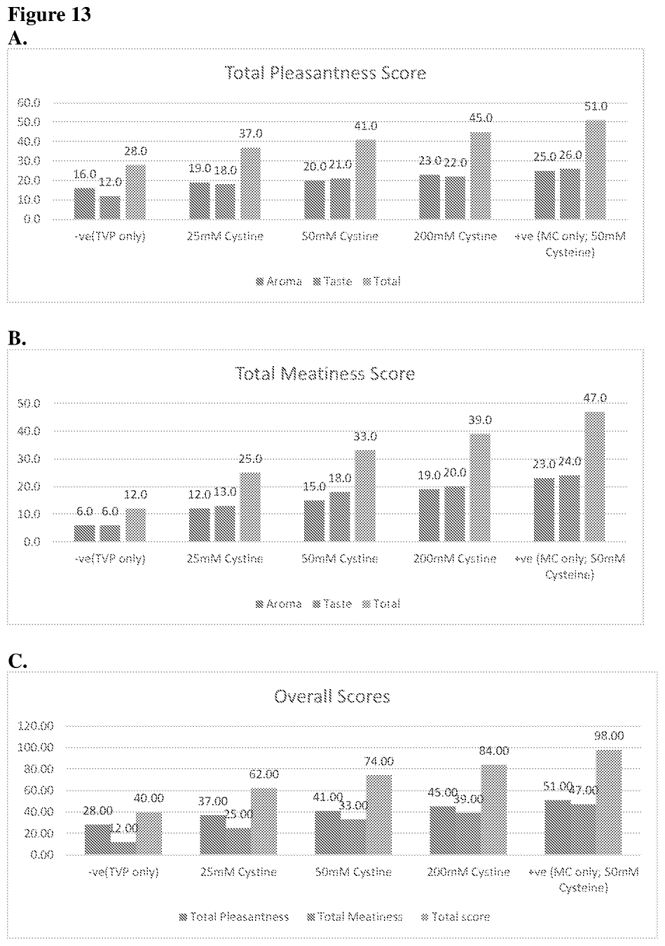

The results are shown in FIGS. 12 and 13. It was observed that the cysteine in Matrix C could be replaced with cystine while still faciliating the production of meaty tastes and aromas when combined with the M. alpina biomass. However, there did appear to be some more desirable sensory attributes associates with the use of cysteine.

Experiment 2. Replacement of Ribose with Dextrose

In this experiment, the effect of replacing ribose with dextrose was assessed by aroma and taste tests.

### Sample Preparation—Concentrate

A set of 8 samples, containing a negative control (MC only), two positive controls (MC+10% wet M. alpina biomass (M. alpina strain yNI0132, B017 culture as described above [“B17 wet” ]); Mix A having 12 mg cystine+10% B17_wet) and a negative control (Matrix C only) and 5 samples containing 10% biomass and Matrix C but with dextrose of differing concentration ranging from 12.5 to 200 mM were prepared according to Table 36.

TABLE 36
Concentrate Preparation

| | Sample Number | | | | | | | |
|---|---|---|---|---|---|---|---|---|
| | 1 | 2 | 3 | | | | | |
| | −ve | +ve | +ve | | | | | |
| | (MC | (MC + | (Mix | 4 | 5 | 6 | 7 | 8 |
| | only) | B17) | A + B17) | 0.25x | 0.5x | 1x | 2x | 4x |
| Cysteine•HCl | 250 | 250 | 12.015 mg | 250 | 250 | 250 | 250 | 250 |
| (400 mM)/μL | | | Cystine | | | | | |
| Ribose (400 mM) (μL) | 250 | 250 | 250 | 0 | 0 | 0 | 0 | 0 |
| Dextrose (800 mM) (μL) | 0 | 0 | 0 | 31.25 | 62.5 | 125 | 250 | 500 |
| Final Dextrose | 0 | 0 | 0 | 12.5 | 25 | 50 | 100 | 200 |
| conc. (mM) | | | | | | | | |
| Thiamine•HCl | 90.9 | 90.9 | 90.9 | 90.9 | 90.9 | 90.9 | 90.9 | 90.9 |
| (44 mM) (μL | | | | | | | | |
| Yeast Extract | 1000 | 1000 | 1000 | 1000 | 1000 | 1000 | 1000 | 1000 |
| (30 g/100 ml) (μL | | | | | | | | |
| Monosodium Glutamate | 125 | 125 | 125 | 125 | 125 | 125 | 125 | 125 |
| (400 mM) (μL) | | | | | | | | |
| Water (μL) | 284.1 | 284.1 | 534.1 | 502.85 | 471.6 | 409.1 | 284.1 | 34.1 |
| Total (μL) | 2000 | 2000 | 2000 | 2000 | 2000 | 2000 | 2000 | 2000 |
| B17_wet (mg) | 0 | 200 | 200 | 200 | 200 | 200 | 200 | 200 |

After adding all ingredients to the 20 ml GC headspace vials, samples were vigorously mixed (2000 rpm) for 2 min at room temp (22-24c) and subjected to heating at 140° C. for 45 min. After the heat treatment completed, the samples were cooled down and tempered at 45° C. throughout the sensory evaluation.

### Sample Preparation—Food

A set of total 6 samples, with one negative control (TVP) and one positive control (MC+B17), and four selected samples with the concentration of dextrose from 25 to 200 mMv (as specified in Table 37 below) were prepared.

TABLE 37
Recipe of food preparation

| | Sample Number | | | | | |
|---|---|---|---|---|---|---|
| | 1 | | | | | 6 |
| | Plain | 2 | 3 | 4 | 5 | +ve |
| | TVP | 0.25x | 0.5x | 2x | 4x | (MC + B17) |
| Cysteine•HCl | 0 | 625 | 625 | 625 | 625 | 625 |
| (400 mM) (μL) | | | | | | |
| Ribose (400 mM) (μL) | 0 | 0 | 0 | 0 | 0 | 625 |
| Dextrose (800 mM) (μL) | 0 | 78.125 | 156.25 | 625 | 1250 | 0 |
| Final Dextrose conc. (mM) | 0 | 25 | 50 | 100 | 200 | 0 |
| Thiamine•HCl | 0 | 227.25 | 227.25 | 227.25 | 227.25 | 227.25 |
| (44 mM) (μL) | | | | | | |
| Yeast Extract | 0 | 2500 | 2500 | 2500 | 2500 | 2500 |
| (30 g/100 ml) (μL) | | | | | | |
| Monosodium Glutamate | 0 | 312.5 | 312.5 | 312.5 | 312.5 | 312.5 |
| (400 mM) (μL) | | | | | | |
| Water (μL) | 5000 | 1257 | 1179 | 710.25 | 85.25 | 710.25 |
| Total (μL) | 5000 | 5000 | 5000 | 5000 | 5000 | 5000 |
| B17_wet (mg) | 0 | 500 | 500 | 500 | 500 | 500 |

After adding all ingredients to the 20 ml GC headspace vials, samples were vigorously mixed (2000 rpm) for 2 minutes at room temp (22-24° C.) and subjected to heating at 140° C. for 45 min. After the heat treatment completed, the samples were left on the bench to cool down completely. After cooling, each taste mixture was added to 50 grams of rehydrated TVP flakes, mixed thoroughly, and let sit to marinate for 15 minutes.

After marinating, each marinated TVP sample was cooked on an oiled frying pan (3 ml of safflower oil) at 1000 W for 2 minutes, stirring occasionally. Between each sample, the frying pan was cleaned and dried. All cooked samples were stored in individual closed containers and kept at 60° C. for no longer than 1 hour until tasting.

### Sensory Evaluation Method

For assessment of the concentrate, six participants (both male and female, aging from 25-65) were asked to sniff and taste the samples in the following order Sample 1, Sample 4 to 8, Sample 2, and, lastly, Sample 3. Between samples, the participants were requested to sniff the coffee and drink water to neutralize/clear the nose and tongue. The participants were requested to evaluate both aroma and taste for the meatiness and pleasantness based on a five-point hedonic scale, with the higher score indicating the increased meatiness and pleasantness.

For assessment of the food samples, five participants (both male and female, aging from 25-65) were asked to sniff and taste the samples in the order from sample 7 to sample 1. Between samples, the participants were requested to sniff the coffee and drink water to neutralize/clear the nose and tongue. The participants were requested to evaluate both aroma and taste for the meatiness and pleasantness based on a five-point hedonic scale, with the higher score indicating the increased meatiness and pleasantness.

### Results

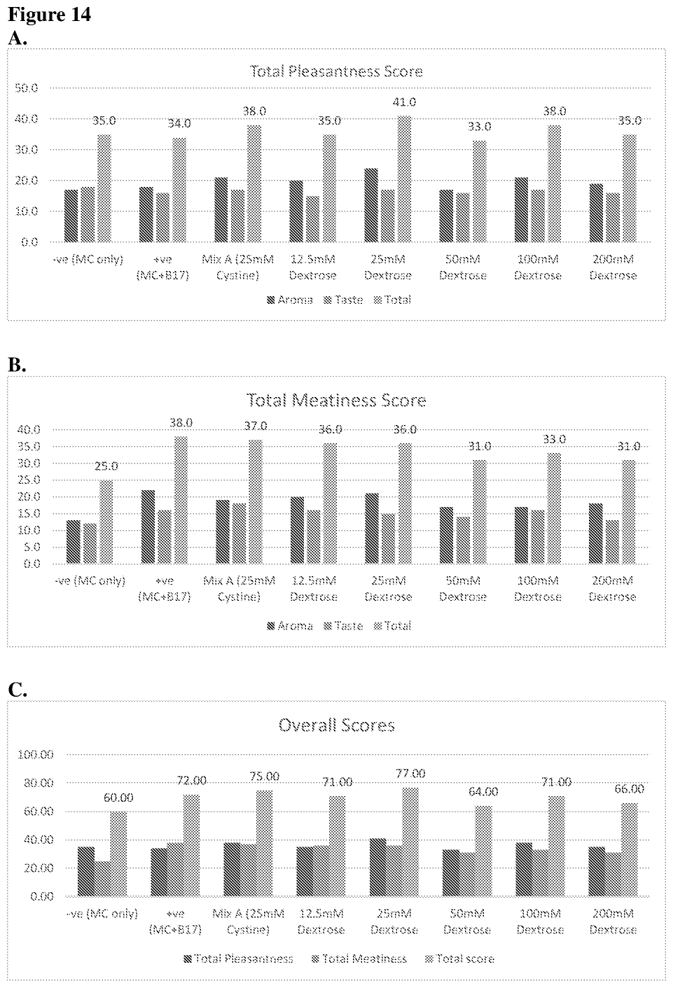

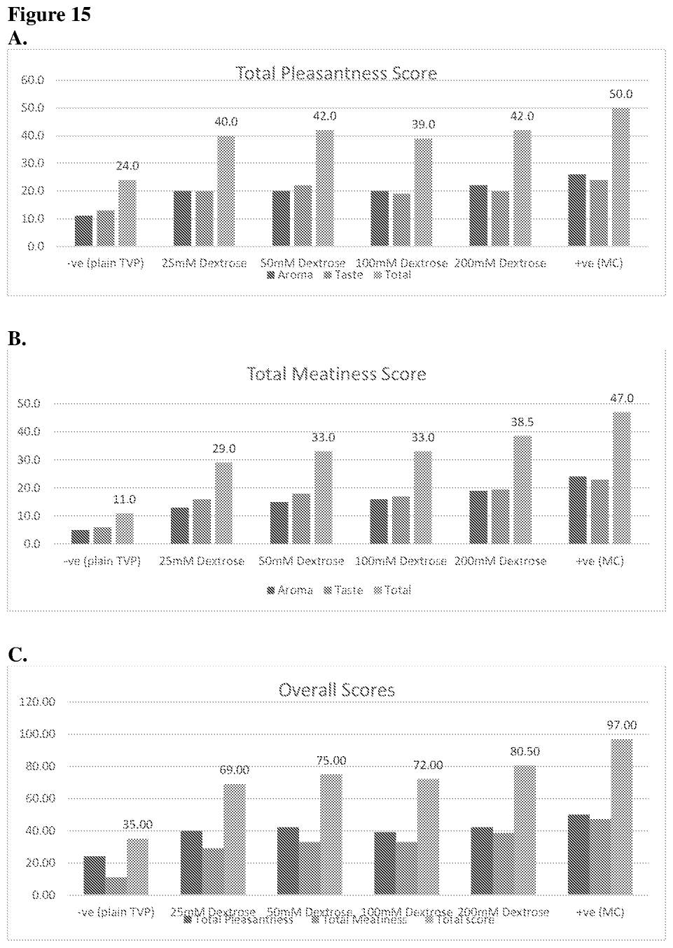

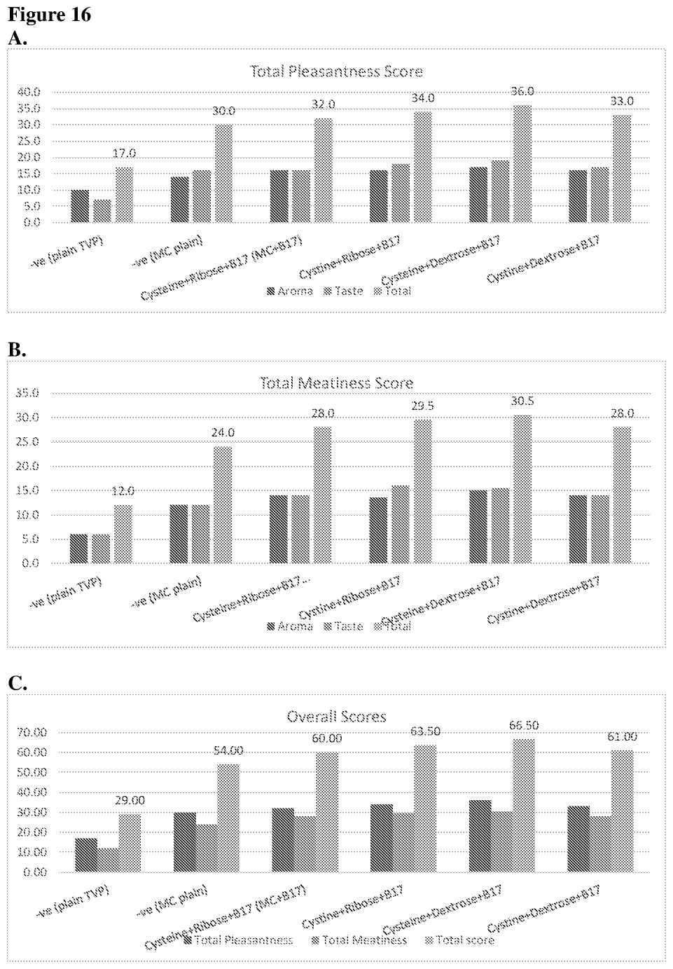

The results are shown in FIGS. 14 and 15. It was observed that the ribose in Matrix C could be replaced with dextrose while still faciliating the production of meaty tastes and aromas when combined with the M. alpina biomass. The use of higher concentrations of dextraose (e.g. 200 mM) appeared more effective in producing strong meaty aromas and tastes.

### Experiment 3—Amino Acid and Sugar Pairings

Four different combinations of amino acid and sugar were assessed: Cysteine, Ribose; Cysteine, Dextrose; Cystine, Ribose; and Cystine, Dextrose.

### Sample Preparation

Food samples were prepared as shown in Table 38. Cystine in this experiment was added in two portions: the first portion was added into the 5 ml taste mixture and undergone a heat treatment process and the second portion was added during the marination stage.

TABLE 38
Recipe of food preparation

| | Sample | | | | | |
|---|---|---|---|---|---|---|
| | | | 3 | 4 | 5 | 6 |
| | | 2 | Cysteine + | Cystine + | Cysteine + | Cystine + |
| | 1 | −ve | Ribose + | Ribose + | Dextrose + | Dextrose + |
| | Plain TVP | (MC only) | B17) | B17) | B17) | B17 |
| Cysteine•HCl | 0 | 625 | 625 | 0 | 625 | 0 |
| (400 mM) (μL) | | | | | | |
| Cystine (mg) | 0 | 0 | 0 | 240.3 | 0 | 240.3 |
| Cystine powder in | 0 | 0 | 0 | 30.0375 | 0 | 30.0375 |
| vial (mg) | | | | | | |
| Cystine powder to add | 0 | 0 | 0 | 210.2625 | 0 | 210.2625 |
| before cooking (mg) | | | | | | |
| Ribose (400 mM) (μL) | 0 | 625 | 625 | 625 | 0 | 0 |
| Dextrose (800 mM) (μL) | 0 | 0 | 0 | 0 | 1250 | 1250 |
| Thiamine•HCl | 0 | 227.25 | 227.25 | 227.25 | 227.25 | 227.25 |
| (44 mM) (μL) | | | | | | |
| Yeast Extract (DSM) | 0 | 2500 | 2500 | 2500 | 2500 | 2500 |
| (30 g/100 ml) (μL) | | | | | | |
| Monosodium Glutamate | 0 | 312.5 | 312.5 | 312.5 | 312.5 | 312.5 |
| (400 mM) (μL) | | | | | | |
| Water (μL) | 5000 | 710.25 | 710.25 | 1335.25 | 85.25 | 710.25 |
| Total (μL) | 5000 | 5000 | 5000 | 5000 | 5000 | 5000 |
| B17_wet (mg) | 0 | 0 | 500 | 500 | 500 | 500 |

After adding all ingredients to the 20 ml GC headspace vials, samples were vigorously mixed (2000 rpm) for 2 minutes at room temp (22-24° C.) and subjected to heating at 140° C. for 45 min. After the heat treatment completed, the samples were left on the bench to cool down completely. After cooling, each taste mixture was added to 50 grams of rehydrated TVP flakes, mixed thoroughly, and let sit to marinate for 15 minutes.

After marinating, each marinated TVP sample was cooked on an oiled frying pan (3 ml of safflower oil) at 1000 W for 2 minutes, stirring occasionally. Between each sample, the frying pan was cleaned and dried. All cooked samples were stored in individual closed containers and kept at 60° C. for no longer than 1 hour until tasting.

### Sensory Evaluation Method

Five participants (both male and female, aging from 25-65) were asked to sniff and taste the samples in the order from Sample 1 to Sample 6. Between samples, the participants were requested to sniff the coffee and drink water to neutralize/clear the nose and tongue. The participants were requested to evaluate both aroma and taste for the meatiness and pleasantness based on a five-point hedonic scale, with the higher score indicating the increased meatiness and pleasantness.

### Results

Results are shown in FIG. 16. While all combination of amino acid and sugar produced meaty aromas and flavours when combined with the M. alpina biomass, the combination of cysteine and dextrose was the most preferred combination and had the highest meatiness score. This sample was noted with dark chicken meat such as chicken thigh, strong umami notes and slight aftertaste without any strong bitter tastes.

Experiment 4. Replacement of MSG with Glutamic Acid

In this experiment, the effect of replacing MSG with glutamic acid was assessed by aroma and taste tests.

### Sample Preparation—Concentrate

Two samples, one with 50 mM Monosodium Glutamate and one with 10 mM Glutamic Acid were prepared in triplicate (10 mL mixtures) according to the recipe shown in Table 39.

TABLE 39
Concentrate Preparation

| | Sample A | Sample B |
|---|---|---|
| | (MSG 50 | (Glutamic |
| | mM) | Acid 10 mM) |
| Cysteine•HCl (400 mM) (μL) | 1250 | 0 |
| Cysteine•HCl (400 mM) + Glutamic | 0 | 1250 |
| Acid (80 mM) (μL) | | |
| Dextrose (800 mM) (μL) | 2500 | 2500 |
| Thiamine•HCl (44 mM) (μL) | 454.5 | 454.5 |
| Yeast Extract (DSM) (30 g/100 ml) | 5000 | 5000 |
| (μL) | | |
| Monosodium Glutamate (400 mM) (μL) | 625 | 0 |
| Water (μL) | 170.5 | 795.5 |
| Total (μL) | 10000 | 10000 |
| B17_wet (mg) | 1000 | 1000 |

After adding all ingredients to the 20 ml GC headspace vials, samples were vigorously mixed (2000 rpm) for 2 minutes at room temp (22-24° C.) and subjected to heating at 140° C. for 45 min. After the heat treatment completed, the samples were left on the bench to cool down completely. After cooling, each 10 ml of taste mixture was added to 100 grams of rehydrated TVP flakes, mixed thoroughly, and let sit to marinate for 15 minutes.

After marinating, each marinated TVP sample was cooked on an oiled frying pan (3 ml of safflower oil) at 1000 W for 2 minutes, stirring occasionally. Between each sample, the frying pan was cleaned and dried. All cooked samples were stored in individual closed containers and kept at 60° C. for no longer than 1 hour until tasting.

### Sensory Evaluation

A triangle test was utilised in this sensory evaluation. There are six permutations/variations in a triangle test: AAB, ABA, BAA, BBA, BAB, ABB. In this experiment, Sample A contained 25 mM of Monosodium Glutamate and Sample B contained 10 mM of Glutamic Acid. Each participant was provided with two sets of samples and each set contained three samples. Among the three samples, two of them were the same and the third one was different.

Nine participants were involved in tasting and each was provided with two sets of three samples. They were instructed to assess the samples from the first set from left to right and identify the odd sample through differences in taste and aroma and repeat with the next set. Between samples, the participants were requested to sniff the coffee and drink water to neutralize/clear the nose and tongue.

### Results

While some participants could detect a difference in taste and aroma due to the substitution of MSG with glutamic acid, it did not appear to eb a significant difference, indicating that glutamic acid could be used instead of MSG.

### Experiment 4. Omission of Various Components

In this study, various components of the Maillard reaction mix were omitted and sensory evaluations performed to assess the effect. A set of 8 samples was prepared in the form of 5 ml taste mixtures according to the recipe shown in Table 40. A positive control (OM, containing 50 mM cysteine, 10 mM glutamic acid, 200 mM glucose, 2 mM thiamine and 15 mg/100 mL (0.15 mg/mL) yeast extract), and negative controls (biomass only and plain TVP) were included. Each 5 ml was then added to 50 grams of rehydrated TVP (i.e. a 1/10 dilution).

TABLE 40
Concentrate preparation for addition to food.

| | | | No | | | No | | |
|---|---|---|---|---|---|---|---|---|
| | | No | Glutamic | No | No | Yeast | BM | |
| | OM | Cysteine | Acid | Glucose | Thiamine | Extract | only | TVP |
| Sample # | 1 | 2 | 3 | 4 | 5 | 6 | 7 | 8 |
| Cysteine•HCl | 625 | 0 | 0 | 625 | 625 | 625 | 0 | 0 |
| (400 mM) + Glutamic | | | | | | | | |
| Acid (80 mM) (μL) | | | | | | | | |
| Cysteine•HCl | 0 | 0 | 625 | 0 | 0 | 0 | 0 | 0 |
| (400 mM) (μL) | | | | | | | | |
| Glutamic Acid | 0 | 7.35 | 0 | 0 | 0 | 0 | 0 | 0 |
| (mg) (μL) | | | | | | | | |
| Glucose (800 mM) | 1250 | 1250 | 1250 | 0 | 1250 | 1250 | 0 | 0 |
| (μL) | | | | | | | | |
| Thiamine•HCl | 227.25 | 227.25 | 227.25 | 227.25 | 0 | 227.25 | 0 | 0 |
| (44 mM) (μL) | | | | | | | | |
| Yeast Extract | 2500 | 2500 | 2500 | 2500 | 2500 | 0 | 0 | 0 |
| (DSM) | | | | | | | | |
| (30 g/100 ml) (μL) | | | | | | | | |
| Water (μL) | 397.75 | 1022.75 | 397.75 | 1647.75 | 625 | 2897.75 | 5000 | 5000 |
| Total (μL) | 5000 | 5000 | 5000 | 5000 | 5000 | 5000 | 5000 | 5000 |
| B17_wet (mg) | 500 | 500 | 500 | 500 | 500 | 500 | 500 | 0 |

### Sensory Evaluation Method

The sensory evaluation of this experiment was done in the form of group discussion. A total of 6 participants were included in the discussion and each member would comment on the aroma and the taste of each sample starting from the TVP only sample (#8), the biomass only (BM) sample (#7), then in the order from #1 to #6, comparing each sample to #1.

### Results

Yeast Extract, when absent: The sample contained a light chicken aroma with more intense vegetable aroma. It was also noted to have similar aroma notes to the positive control (OM) but without the depth/intensity.

Cysteine, when absent: When compared to the positive control (OM), this sample had a sweet aroma but lack of meaty notes. The dextrose in the sample would likely be the main contributor to the sweet aroma and the caramelisation during heating. The sample also had a bitter aftertaste with key taste notes such as mushroom and metallic.

Glutamic Acid, when absent: The meaty aroma was present but at a much lower intensity when compared to the positive control (OM), with traceable sulphur notes. The sample had a savoury taste note with a bitter aftertaste, with taste profile reminiscent of chicken.

Dextrose, when absent: Bland in aroma without the umami and meaty note, and a stronger salty aroma note. Compared to the OM sample, this sample had a weaker umami taste but still had a sweet aftertaste. The sweet aftertaste was different from the thiamine-removed sample which contained 200 mM of dextrose.

Thiamine, when absent: The sample was noted to have different aroma note compared to the sample without glutamic acid. This sample was commented to have a more pleasant taste than the yeast extract removed sample. It was noted with a roast meat taste and a savoury note.

In summary, the absence of cysteine and dextrose appeared most notable in their affects on meaty aromas. Yeast extract and glutamic acid also appeared to be important for a full meaty flavour. Thiamine appeared less important to the generation of a meaty flavour.

### Example 17. Assessment of Volatiles Generated from M. alpina Fractions

Studies were performed to analyse the volatile aroma compounds from M. alpina biomass and fractions of interest, to further understand the contribution of each fraction to the meaty and off-note aromas within the overall aroma profile. Biomass from M. isabellina, which lacks arachidonic acid (ARA)-containing lipids, was also assessed.

### Fractionation

All extraction solvents were food grade and were de-gassed prior to use by sonication for at least 20 min. An ethanol lipid extract was first prepared by defrosting 250 g wet biomass in a sealed plastic bag. To this, 1000 mL ethanol was added (4:1 ratio solvent:biomass, mL: g) and the mixture homogenized for 15 min at speed 5 using a Ultra-Turex T10 handheld homogeniser. The mixture was then stirred for 15 min at 1500 RPM on an IKA RW 20 digital overhead stirrer. The biomass was filtered out using a Buchner funnel and watmans no.6 filter paper, and the filtrate retained. The extraction was repeated a further 3 times (total 4 washes) by adding a further 500 mL ethanol to the filtered biomass and repeating the steps. The washed and filtered biomass was transferred to a wash glass and the ethanol dried off. The resulting biomass was the ‘defatted biomass’ fraction.

The ethanol washes were pooled and 200 mL the solvent evaporated under vacuum on a Buchi Rotor Evaporator. Sufficient water was then added such that the water part of the aqueous phase was ˜40% of the phase. This was then washed with hexane by adding 0.2 v/v of hexane to the hydroalcoholic phase, before being shaken on a platform vortex for 10 min and centrifuged (2000 g, 10 min) to separate the phases. The lower (aqueous) phase and upper (hexane, lipid containing) phase were collected. The aqueous phase was washed a further 3 times with hexane, with the hexane phases pooled. The pooled hexane phases were dried on a Buchi rotor evaporator to produce the ‘total lipid’ fraction.

To produce the neutral lipid fraction (“TAG”), total lipid fraction was weighed out and resuspended in 30 volumes acetone, (w/v) before being vortexed for 30 sec) and sonicated for 30 secs repeatedly until visibly resuspended. The sample was then precipitated at −20° C. overnight before being centrifuged at 3900 g for 10 min at 4° C. The supernatant was collected and the sample returned to −20° C. for at least 72 hours, with intermittent repeat of the aqueous phase wash. The supernatant was then collected and the solvent evaporated on a rotor evaporator, thus producing the TAG fraction.

To produce the polar lipid (PL) fraction, total lipid fraction was weighed and resuspended in 30 volumes acetone (w/v) before being vortexed for 30 sec) and sonicated for 30 secs repeatedly until visibly resuspended. The sample was then precipitated at 4° C. for 30 min and then centrifuged at 3900 g for 10 min at 4° C. The supernatant was removed and the precipitate washed twice more with another 30×volumes ice cold acetone. The precipitate was dried under nitrogen gas until it reached a constant weight, thereby producing the ‘PL’ fraction.

All fraction samples were analysed via TFA GC-FID analysis, while all lipid fractions were run on both solvent system TLCs. Table 41 shows the fatty acid profiles of the lipid fractions as analysed via TFA GC-FID.

TABLE 41

| Fraction | Rep | C14:0 | C14:1 | C15:0 | C16:0 | C16:1 | C18:0 | C18:1 | C18:1iso | C18:2 | C18:3n6 |
|---|---|---|---|---|---|---|---|---|---|---|---|
| Ethanol | 1 | 1.68 | 0.00 | 0.60 | 23.56 | 0.42 | 6.21 | 15.77 | 0.77 | 10.14 | 6.00 |
| extract | 2 | 1.61 | 0.00 | 0.58 | 22.73 | 0.37 | 5.97 | 15.26 | 0.56 | 9.81 | 5.75 |
| Total | 1 | 1.76 | 0.00 | 0.62 | 23.76 | 0.45 | 6.01 | 16.95 | 0.00 | 9.68 | 5.57 |
| lipid | 2 | 1.72 | 0.00 | 0.61 | 23.43 | 0.42 | 6.53 | 15.79 | 0.74 | 9.44 | 5.32 |
| TAG | 1 | 1.93 | 0.00 | 0.62 | 20.68 | 0.46 | 3.47 | 18.17 | 0.00 | 10.29 | 6.61 |
| | 2 | 1.88 | 0.00 | 0.61 | 20.35 | 0.43 | 4.91 | 17.06 | 0.00 | 10.06 | 6.28 |
| PL | 1 | 0.68 | 0.00 | 0.48 | 18.85 | 0.25 | 2.53 | 15.59 | 1.00 | 18.63 | 10.57 |
| | 2 | 0.66 | 0.00 | 0.46 | 18.27 | 0.00 | 2.32 | 15.23 | 0.84 | 18.05 | 10.31 |
| Defatted | 1 | 1.07 | 0.00 | 0.55 | 21.71 | 0.31 | 4.67 | 13.74 | 0.72 | 15.08 | 7.72 |
| biomass | | | | | | | | | | | |

| Fraction | Rep | C18:3n3 | C20:0 | C20:1 | C20:3 | C20:4Ara | C20:5 | C22:0 | C22:6 |
|---|---|---|---|---|---|---|---|---|---|
| Ethanol | 1 | 0.35 | 0.51 | 0.95 | 5.06 | 22.89 | 1.69 | 0.14 | 3.26 |
| extract | 2 | 0.36 | 0.51 | 5.38 | 4.77 | 21.62 | 1.59 | 0.00 | 3.11 |
| Total | 1 | 0.36 | 0.20 | 0.47 | 7.57 | 21.26 | 1.71 | 0.14 | 3.46 |
| lipid | 2 | 0.36 | 0.53 | 1.75 | 5.02 | 22.95 | 1.75 | 0.20 | 3.44 |
| TAG | 1 | 0.43 | 0.44 | 0.65 | 6.47 | 27.37 | 0.97 | 0.17 | 1.23 |
| | 2 | 0.44 | 0.33 | 1.90 | 5.53 | 27.72 | 0.96 | 0.22 | 1.30 |
| PL | 1 | 10.44 | 0.09 | 0.70 | 4.56 | 24.56 | 0.27 | 0.04 | 0.77 |
| | 2 | 10.49 | 0.00 | 2.18 | 4.33 | 23.04 | 0.46 | 2.64 | 0.72 |
| Defatted | 1 | 0.35 | 0.37 | 0.81 | 5.45 | 21.66 | 1.36 | 0.22 | 4.22 |
| biomass | | | | | | | | | |

### Volatile Analysis

### Sample Preparation

5 mL samples were prepared according to Table 42 in 20 ml GC headspace vials. The components were combined by vigorously mixing (2000 rpm) for 2 minutes at room temp (22-24 (C) before being subjected to heating at 140° C. for 45 mm. After the heat treatment, the samples were left on the bench to cool down completely and then purged with Nitrogen gas to prevent oxidation.

TABLE 42
The components of the fractionation samples, adding each fraction proportionally
to its content in 50 mg DCW of the M. alpina biomass.

| | | | Weight | Component | Weight |
|---|---|---|---|---|---|
| | Sample | Biomass Component | (mg) | Lipid | (mg) |
| S1 | Matrix OM | — | — | — | — |
| S2 | Matrix OM + BMI (M. | M. alpina wet whole | 171.5 | — | — |
| | alpina NI044) | biomass | | | |
| S3 | Matrix OM + M. isabellina | M. isabellina wet whole | 353.1 | — | — |
| | | biomass | | | |
| S4 | Matrix OM + biomass | M. alpina de-lipidated | 29.5 | — | — |
| | minus fat | biomass (dry) | | | |
| S5 | Matrix OM + biomass | M. alpina de-lipidated | 29.5 | PL | 7.7 |
| | minus TAG | biomass (dry) | | | |
| S6 | Matrix OM + biomass | M. alpina de-lipidated | 29.5 | TAG | 12.8 |
| | minus PL | biomass (dry) | | | |
| S7 | Matrix OM + PL | — | — | PL | 7.7 |
| S8 | Matrix OM + TAG | — | — | TAG | 12.8 |

### GC Analysis

The samples were analysed at the CASS Food Research Centre, Deakin University, using headspace solid phase microextraction (HS-SPME) method combined with GC-MS (Gas Chromatography Mass Spectrometry).

Gas Chromatographic analysis of samples was performed using an Agilent 7890B GC system fitted with a BP-5MS capillary column (30 m length×0.250 mm i.d×0.25 mm film thickness) (Trajan Scientific and Medical, Melbourne, Australia) and coupled with Mass Selective detector (Agilent Technologies, USA). GC inlet temperature was set at 250° C. The carrier gas was helium gas (99.999% pure) at constant flow rate of 1.4 mL/min. The initial column temperature was set at 40° C. with 1 min hold time, and the temperature was increased to 240° C. at a rate of 3° C./min. Total analysis time for each sample was 67.7 min. The MS conditions were: scan range of 40-450 m.u., solvent cut time 0 min, electron impact mode at 70 eV, MS source 230° C. and MS quad 150° C.

Each chromatogram was processed with Total Ion Chromatogram mode for peak identification and retention indices. The tentative identification of separated compounds was performed using NIST (National Institute of Standards and Technology) databases (minimum similarity match of 750). After identification of each peak, the results were exported as csv files into Microsoft Excel version 1708 (Microsoft Corporation) for data cleaning and further analysis.

### Results

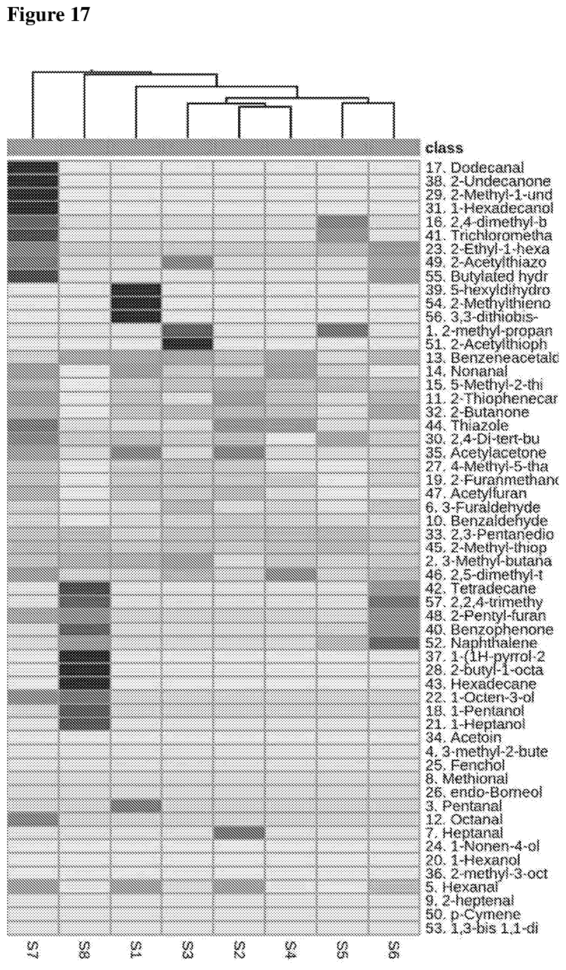

A total fifty-seven (57) volatile compounds were identified by GC-MS (Table 43). The S1 (‘Matrix OM’) and S4 (‘Matrix OM+biomass minus fat’) samples clustered together in a PCA plot (not shown), suggesting that fats may be an important and differentiating fraction in the development of particular aromas. Indeed, as shown in FIG. 17, there were distinct differences in the volatiles generated by each sample, although also some similarities. Of particular note, it was apparent that the volatiles generated by the purified neutral lipid (TAG) fraction were quite distinct from the volatiles generated by the samples that contained phospholipids (e.g. whole biomass or PL fraction), which is consistent with the very different aroma of the neutral lipid fraction as compared to the polar lipid fraction or whole cell biomass, as detected in the sensory evaluations (see e.g. ARA oil vs biomass in Example 14).

It was observed that Benzeneacetaldehyde and Nonanol were not present in S1, S4 or S8 (‘Matrix OM+TAG’) but were present in all other samples. Benzeneacetaldehyde has been identified in beef (Specht et al. J Ag Food Chem 1994; 42(10):2246-53) and as a desirable aroma for meatiness (Zhang et al. Food Sci Tech. 2017; 82:184-91). Nonanol is one of the major volatiles in lamb (Luo et al. Food Sci Nutrition. 2019 (7):2796-805) and it has been suggested that is produced via lipid (oleic acid) oxidation. The lack of these volatiles in fractions not containing lipid suggests that the lipid fraction (and particularly the polar lipid fraction given the lack of these volatiles in S8) may be important for meaty aroma. The presence of volatiles such as 3-Methyl-butanal, which has been identified as key as an important aroma in fatty boiled beef (Grosch. Chem Senses. 2001; 26(5):533-45), in all samples except S1 and S8 further reinforces the importance of the polar lipid fraction.

Additionally, in S1 the presence of volatiles such as 5-hexyldihydro-2(3H)-Furanone; 2-Methylthieno [2,3-b]thiophene; and 3,3-dithiobis-2-methyl-Furan were found that were not detected in any other sample. The latter two in particular have been identified as contributing meaty aroma (Raza et al. Food Funct. 2020; 11(10):8583-601; Zhao et al. Food Chem. 2019; 270:436-44) in Maillard systems, but are not generally identified in genuine meat samples.

While the S2 (‘Matrix OM+M. alpina’) and S3 (‘Matrix OM+M. isabelina’) samples were clustered closely on the PCA plot, there are distinct differences in both the Sensory and Volatile analysis, suggesting that ARA is playing an important role in “meatiness”. There is a large increase in the volatile Heptanal in the S2 compared to the S3 sample. Heptanal has been identified in chicken fat (Zhao et al. Food Chem. 2019; 270:436-44), beef fat (Song et al. Meat Sci. 2014; 96(3):1191; Um et al. J Ag Food Chemistry. 1992; 40(9):1641-6.) and shallow fried beef (Specht et al. J Ag Food Chem 1994; 42(10):2246-53) and is considered one of the main volatile compounds in lamb (Luo et al. Food Sci Nutrition. 2019 (7):2796-805). Moreover, other volatiles such as Thiazole, which is meaty (Raza et al. Food Funct. 2020; 11(10):8583-601; Zhao et al. Food Chem. 2019; 270:436-44), 2,4-Di-tert-butylphenol and Acetylacetone are found in S2 and other PL containing samples such as S5 (Matrix OM+biomass minus TAG’) and S7 (‘Matrix OM+PL’), but are not present in S3. The opposite is true of Hexanal and 2,5-dimethyl-thiophene, which are detected at decreased levels in the S3, S4 and S8 samples when compared to the S2 and PL-containing samples. This suggest that the ARA containing PLs may be playing a key role in the aroma profile of M. alpina. There are also volatiles that are detected in S3 but not in S2 that may be contributing to off or non-meat aromas, examples being 2-methyl-propanal (wet cereal), 2-Acetylthiazole (popcorn) and 2-Acetylthiophene (sulfur, nutty).

In support of the sensory results that TAG fractions are contributing to off-notes there are volatiles that are detected in greater abundance in S6 (‘Matrix OM+biomass minus PL’) and S8 when compared to the whole biomass or sample not containing TAG, such as Tetradecane (waxy), Naphthalene (Mothball) and 2,2,4-trimethyl-1,3-pentanediol diisobutyrate (plasticiser).

The non-lipid components of the biomass also appear to play an importan trole in the aroma profile, likely interacting with the lipids to modulate their effect on the aroma. This is seen in the PCA plot where S7 and S8 are positioned away from the biomass containing samples S2-6 which are clustered closer together. Additionally, there are volatiles that are detected in S8 or S7 in much higher abundance than in other samples. Examples of these for S7 are Dodecanal; 2-Undecanone; 2-Methyl-1-undecanol; 1-Hexadecanol 2,4-dimethyl-benzaldehyde; and 2-Ethyl-1-hexanol. Examples of these for S are 1-(1H-pyrrol-2-yl)-ethanone; 2-butyl-1-octanol; Hexadecane; 1-Octen-3-ol; 1-Pentanol and 1-Heptanol, many of which have been identified as mushroom and/or off-notes (2, 6, 7, 9) which aligns with the sensory data.

TABLE 43
List of aroma volatile compounds identified
and their aroma description.

| Number | Compound | Aroma description |
|---|---|---|
| 1 | 2-methyl-propanal | wet cereal |
| 2 | 3-Methyl-butanal | cocoa |
| 3 | Pentanal | fermented, bready |
| 4 | 3-methyl-2-butenal | sweet, almond |
| 5 | Hexanal | green, cut grass |
| 6 | 3-Furaldehyde | no aroma identified |
| 7 | Heptanal | fruity fat |
| 8 | Methional | cooked potato |
| 9 | 2-heptenal | green |
| 10 | Benzaldehyde | almond, cherry |
| 11 | 2-Thiophenecarboxaldehyde | no aroma identified |
| 12 | Octanal | fat, soap, lemon, green |
| 13 | Benzeneacetaldehyde | honey-like, sweet, rose |
| 14 | Nonanal | fat, citrus, green |
| 15 | 5-Methyl-2- | almond, cherry |
| | thiophenecarboxaldehyde | |
| 16 | 2,4-dimethyl-benzaldehyde | almond, cherry |
| 17 | Dodecanal | soapy, waxy |
| 18 | 1-Pentanol | fruit |
| 19 | 2-Furanmethanol | burnt |
| 20 | 1-Hexanol | green |
| 21 | 1-Heptanol | mushroom |
| 22 | 1-Octen-3-ol | mushroom |
| 23 | 2-Ethyl-1-hexanol | rose, green |
| 24 | 1-Nonen-4-ol | fat, citrus, green |
| 25 | Fenchol | camphor |
| 26 | endo-Borneol | camphor |
| 27 | 4-Methyl-5-thaizoleethanol | meaty |
| 28 | 2-butyl-1-octanol | no aroma identified |
| 29 | 2-Methyl-1-undecanol | Balsam |
| 30 | 2,4-Di-tert-butylphenol | no aroma identified |
| 31 | 1-Hexadecanol | fatty |
| 32 | 2-Butanone | solvent, sweet |
| 33 | 2,3-Pentanedione | cream butter |
| 34 | Acetoin | butter, cream |
| 35 | Acetylacetone | solvent like |
| 36 | 2-methyl-3-octanone | nut? |
| 37 | 1-(1H-pyrrol-2-yl)-ethanone | musty |
| 38 | 2-Undecanone | orange, fresh |
| 39 | 5-hexyldihydro-2(3H)-Furanone | caramel, fruity |
| 40 | Benzophenone | no aroma identified |
| 41 | Trichloromethane | solvent |
| 42 | Tetradecane | waxy |
| 43 | Hexadecane | waxy |
| 44 | Thiazole | mold, rubber |
| 45 | 2-Methyl-thiophene | onion, roasted |
| 46 | 2,5-dimethyl-thiophene | nutty |
| 47 | Acetylfuran | sweet, balsamic |
| 48 | 2-Pentyl-furan | earthy, green, |
| | | vegetable |
| 49 | 2-Acetylthiazole | popcorn |
| 50 | p-Cymene | terpene like |
| 51 | 2-Acetylthiophene | sulfur, nutty |
| 52 | Naphthalene | mothball |
| 53 | 1,3-bis(1,1-dimethylethyl)-Benzene | no aroma identified |
| 54 | 2-Methylthieno [2,3-b]thiophene | no aroma identified |
| 55 | Butylated hydroxytoluene | antioxidant |
| 56 | 3,3-dithiobis-2-methyl-Furan | meaty, chicken, savory |
| 57 | 2,2,4-trimethyl-1,3-pentanediol | plasticiser |
| | diisobutyrate | |

### Sensory Analysis of Fractions

Matrix OM (5 mL) was mixed with either the Mortierella biomass (BM) or fraction thereof according to Table 44 in 20 ml GC headspace vials. The samples were vigorously mixed (2000 rpm) for 2 minutes at room temp (22-24° C.) and subjected to heating at 140° C. for 45 min.

TABLE 44

| | | | Weight | Lipid | Weight |
|---|---|---|---|---|---|
| | Sample | Biomass Component | (mg) | Component | (mg) |
| S1 | Matrix OM | — | — | — | — |
| S2 | Matrix OM + M. | M. alpina wet whole | 171.5 | — | — |
| | alpina biomass | biomass | | | |
| S3 | Matrix OM + M. | M. isabellina wet whole | 353.1 | — | — |
| | isabellina | biomass | | | |
| S4 | Matrix OM + M. | M. alpina de-lipidated | 29.5 | — | — |
| | alpina biomass minus fat | biomass (dry) | | | |
| S5 | Matrix OM + M. | M. alpina de-lipidated | 29.5 | PL | 7.7 |
| | alpina biomass minus TAG | biomass (dry) | | | |
| S6 | Matrix OM + M. | M. alpina de-lipidated | 29.5 | TAG | 12.8 |
| | alpina biomass minus PL | biomass (dry) | | | |
| S7 | Matrix OM + M. | — | — | PL | 7.7 |
| | alpina PL | | | | |
| S8 | Matrix OM + TAG M. | — | — | TAG | 12.8 |
| | alpina | | | | |

Four experienced participants (all female, aging from 20-45) were asked to sniff the blinded samples in the order of Sample 1 to Sample 8. Between samples, the participants were requested to sniff the coffee to neutralize/clear the nose. The participants were requested to evaluate aroma for the meatiness and pleasantness and discuss, and to compare samples to the wet biomass and matrix only controls.

The sample with the most pleasant and meaty aroma was the wet M. alpina biomass, with the PL containing samples also demonstrating some meatiness: the biomass minus TAG′ sample showed a meaty note that was not as complex and had less intensity of the wet biomass; and the ‘PL’ sample was also identified as having some meatiness, but at approximately with even less intensity than the wet biomass. In contrast, the TAG-containing fraction sample were not identified as meaty: the ‘biomass minus PL’ was described as floral and pleasant, while the ‘TAG’ alone sample was unpleasant with descriptors of “rancid” and “chemical”. This suggested that the TAG fraction was contributing off-notes that could be mitigated by other fractions in the biomass (as in the biomass or biomass minus PL samples). The ‘defatted biomass’ sample was pleasant but lacked the meaty notes of the wet biomass.

These results indicate that the PL fraction contributes to meaty notes in the aroma of M. alpina, but that the PL requires other components in the biomass to achieve the full potential of this meatiness. It appears likely that the TAG fraction contributes to off aromas.

### Example 18. Assessment of M. alpina Biomass in Other Plant-Based Foods

To assess the ability of the M. alpina biomass to impart a meat-like aroma, flavour and mouthfeel (e.g. lingering fattiness and flavour) to different commercially available meat substitutes was tested. Commercially available, plant-based “meatball” (Company A meatballs and Company B meatballs), and one commercially-available plant-based burger patty (Company A patties) were combined with a pre-heated (140° C., 45 mins) concentrated Maillard solution comprising M. alpina biomass and OM matrix essentially as described in previous Examples. In one set of sample, the concentrated Maillard mix also included an additional herb mixture. The wet biomass was present in the food samples at concentrations of 0.8% (w/w) or 0.6% (w/w).

All samples were cooked on an oiled frying pan (3 ml of safflower oil) at 1000 W for 3 minutes, stirring occasionally. Between samples, the pan was cleaned and dried. All cooked samples were stored in individual closed containers and kept at 60° C. for no longer than 1 hour until tasting. A panel of men and women tasted each sample (blinded) and were asked to vote for the most preferred, second most preferred and third most preferred samples, and include any tasting notes. Each vote was counted, with most preferred being assigned a value of 3, second most preferred being assigned a value of 2, and third most preferred being assigned a value of 1.

As shown in Table 45, panellists were more likely to prefer the meatballs or burgers that contained the biomass, with comments of roasted chicken or pork flavours, with fatty and juicy mouthfeel. In contrast, the meatballs and burgers without the biomass were often noted to have a distinct nutty, beany or artificial flavour. Interestingly, many panellists preferred the samples with the lower inclusion rate of biomass.

TABLE 45

| | | | | | | |
|---|---|---|---|---|---|---|
| Sample | 1^{st} | 2^{nd} | 3^{rd} | Total | Average | Notes |

Company A meatballs

| | | | | | | |
|---|---|---|---|---|---|---|
| Meatballs | 0 | 0 | 1 | 1 | 0.10 | *Nutty and beany (TVP) |
| Meatballs + | 3 | 5 | 0 | 19 | 1.90 | *Roasted chicken/thigh/skin |
| BM (0.8%) + | | | | | | *some said artificial and had a strong |
| OM | | | | | | aftertaste |
| Meatballs + | 5 | 1 | 2 | 19 | 1.90 | *Roasted chicken/porky |
| BM (0.6%) + | | | | | | *less artificial, more natural |
| OM | | | | | | *pleasant |
| Meatballs + | 1 | 4 | 3 | 14 | 1.40 | *Meaty and herby |
| BM (0.8%) + | | | | | | *less artificial |
| OM + herbs | | | | | | |
| Meatballs + | 1 | 1 | 3 | 8 | 0.8 | *similar to above |
| BM (0.6%) + | | | | | | *very herby |
| OM + herbs | | | | | | |

Company A burger patties

| | | | | | | |
|---|---|---|---|---|---|---|
| Patty | 1 | 0 | 8 | 11 | 1.22 | *Artificial |
| | | | | | | *Beany, bland |
| Patty + BM | 0 | 8 | 1 | 17 | 1.89 | *Smoky, chicken light |
| (0.8%) + | | | | | | *Strong aftertaste (bitter) |
| OM | | | | | | *Some mushroom |
| | | | | | | *artificial |
| Patty + BM | 8 | 1 | 0 | 26 | 2.89 | *Roasted fatty meaty |
| (0.6%) + | | | | | | *no beany/green taste |
| OM | | | | | | *juicy |

Company B meatballs

| | | | | | | |
|---|---|---|---|---|---|---|
| Meatballs | 0 | 0 | 0 | 0 | 0.00 | *gamey, artificial, strange |
| | | | | | | *lamb like, |
| Meatballs + | 1 | 3 | 2 | 11 | 1.22 | *meaty, chicken like, |
| BM (0.8%) + | | | | | | *rich |
| OM | | | | | | |
| Meatballs + | 3 | 2 | 2 | 15 | 1.67 | *Dark chicken, meaty, |
| BM (0.6%) + | | | | | | *Fatty and juicy |
| OM | | | | | | |
| Meatballs + | 2 | 1 | 1 | 9 | 1.00 | *Herby (too much) |
| BM (0.8%) + | | | | | | *Nice |
| OM + | | | | | | *Meaty, lamb-like |
| herbs | | | | | | |
| Meatballs + | 0 | 0 | 3 | 3 | 0.33 | *Herby |
| BM (0.6%) + | | | | | | *chicken-like |
| OM + | | | | | | *Pleasant |
| herbs | | | | | | |

### Example 19. Assessment of M. alpina Biomass in Plant-Based Foods without Pre-Heating

In the previous studies, the M. alpina biomass and Maillard matrix were pre-heated (e.g. at 140° C. for 45 mins), before being incorporated into food. A study was performed to assess the effect when the Maillard composition was not heated before the application to food.

Maillard composition concentrates were prepared with 5 mL matrix OM and 500 mg wet M. alpina biomass. This concentrate was then added to 50 g rehydrated TVP, which was allowed to marinate before being pan fried with 3 mL safflower oil for 2 min (heat setting at 1000 w). A blinded sensory assessment of the samples was then performed, with the participants evaluating aroma, taste and mouthfeel.

It was observed that the in the absence of the pre-heating step, TVP samples with the biomass and matrix OM had a fatty mouthfeel and strong animalic note, which although was unpleasant to some participants imparted an authentic “meat” experience. The meatiness and pleasantness were reduced compared to samples in which biomass and matrix OM had been pre-heated. This indicated while pre-heating the biomass and matrix results in a strong and pleasant meaty taste and aroma, with fatty mouthfeel, the pre-heating step is not necessary to provide a more authentic “animal” or “meat” experience for these tasting plant-based proteins.

A follow-up study was performed in which the TVP samples were marinated with a composition comprising matrix OM or matrix C with wet biomass (without a pre-heating step) at amounts of 0.25%, 0.5% and 1% in the final food composition. It was observed that reducing the amount of biomass resulted in a less stringent “animalic” note, while retaining meaty flavour and fatty mouthfeel, suggesting that altering the inclusion rate can result in a balance that is desirable for a particular application or market.

### Example 20. Amino Acid Profiles of Samples Before and After Maillard Heat Treatment

Amino acids and sugars are key components in a Maillard reaction. In this study, the changes in amino acid profiles of the samples containing yNI0132 biomass and matrix, from before to after the Maillard reaction, was investigated.

Samples comprising 10 mL Matrix C, with or without 1 g wet biomass were analysed for their amino acid content (i.e. the amount of each of the 20 amino acids) before and after being heated for 140° C. for 45 mins. A significant reduction in the amount of cysteine (approximately 25%) was observed in both the matrix C control and the matrix C+biomass composition following heating, suggesting that this amino acid in particular was being utilised a Maillard reaction. The levels of arginine and histidine were also reduced in the heating process, although to lesser degrees (11-16%, and 9-12%, respectively).

### Example 21. Assessment of Mortierella Biomasses Using Additional Attributes

A sensory analysis of various Mortierella spp. strains was performed using a different array of attributes. Rather than a two-pronged approach of assessing “pleasantness” and “meatiness”, a five-pronged approach of assessing “meatiness”, “roastiness”, “animalic”, “rancid” and “sulphury” was used. The intensity for each attribute was recorded with a value, with a higher score indicating increased intensity.

Biomass of M. zonata SS3 (identified as M. minutissima by ITS homology, ITS sequence set forth in SEQ ID NO:50, and M. alpina ATCC32223, M. alpina S11-2 (identified as M. alpina by ITS homology, ITS sequence set forth in SEQ ID NO:51) was mixed with Matrix OM (equivalent to 50 mg dry biomass in 2 mL Matrix OM) in a 20 ml GC headspace vial as shown. The samples were then vigorously mixed (2000 rpm) for 2 minutes at room temp (22-24° C.) and subjected to heating at 140° C. for 45 minutes.

After heating, all samples were allowed to cool and to be tempered in an oven at 45° C. throughout the sensory evaluation. A total of six participants (both male and female, aging from 25-65) were asked to sniff the samples, with the reference sample (OM laone, without biomass) assessed first. Participants were instructed to evaluate the sample acceptance first, then proceed to evaluating the descriptive attributes while comparing to the reference on a 9-point scale. Between samples, the participants were requested to sniff the back of their hands to neutralize/clear the nose.

The aroma attributes evaluated in this experiment was overall meatiness, roastiness, animalic, rancid and sulphury. The intensity for each attribute for the reference was given “5”, with a higher score indicating increased intensity compared to the reference and a lower score indicating decreased intensity compared to the reference.

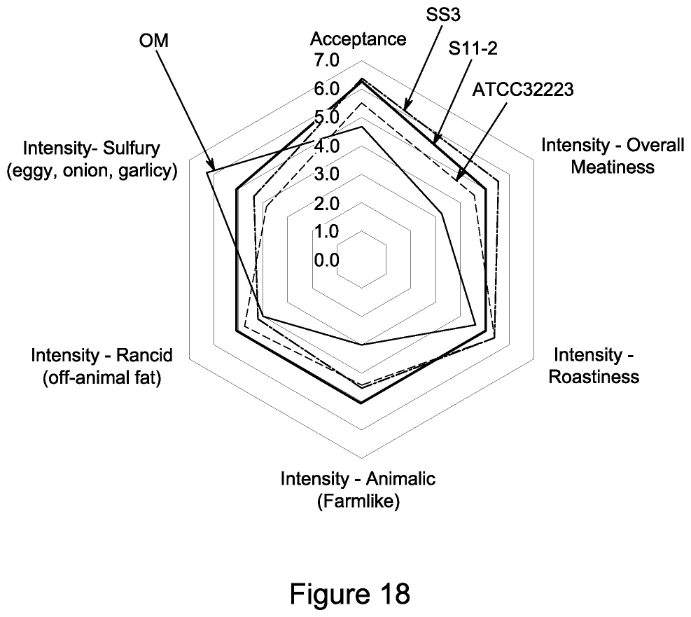

As shown in FIG. 18, the control Maillard matrix (OM) had the lower acceptance score, lower overall meatiness, roastiness and animalic (farmlike) note but a higher sulfury notes compared to other samples containing the biomass, re-confirming the importance of biomass in the meaty aroma formation.

In general, the differences in aroma characteristics of tested biomasses were minimal. Sample ATCC32223 had the lowest acceptance score, with the lowest scores in meatiness, sulfury and animalic notes when compared to other biomasses. Biomass from SS3 had the highest overall meatiness and roastiness scores, the lowest rancid (off-animal fat) notes compared to ATCC32223 and S11-2. S11-2 had the lowest roastiness notes and the highest animalic, rancid and sulfury notes.

### Example 22. Phylogenetic Analysis of M. alpina Isolates

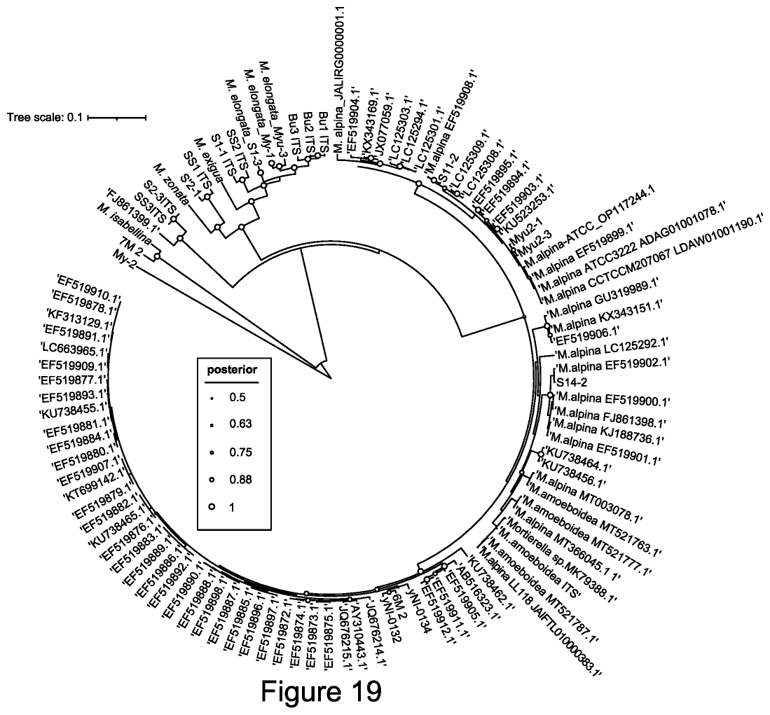

The new Mortierella spp. isolates described above, as well as additional isolates 514-4, Myu2-1 and Myu2-3 identified from various soil samples, had been morphologically identified as falling within the Mortierellaceae family and subsequently classified as various Mortierella species based on ITS homology with known Mortierella species. Phylogenetic analysis was performed by comparing the ITS sequences of some of these isolates with 81 M. alpina ITS sequences retrieved from GenBank, as well as other Mortierella species such as M. isabellina, M. zonata and M. exigua. Sequencing alignment was performed using the software Geneious Prime v.2022.2.2. (https://www.geneious.com). The phylogenetic analysis shown in FIG. 19 was performed using the BEAST phylogenetics software v. 2022 (Bouckaert et al., 2019), and the visualization of the resulting tree was carried out using the Interactive Tree of Life (iTOL) software v6.6 (Letunic and Bork, 2019). Circles indicate the bootstrap supporting as Bayesian posterior probability.

Isolates yNI0132, yNI0134, S11-2, Myu2-1, Myu2-3 and S14-4 were grouped into the large clade formed by 75 M. alpina from the database (this clade also included a subclade of four M. amoeboidea), evidencing their classification as M. alpina. In contrast, other isolates such as S′2-1, S2-3, SS3, S1-3, Myu1 and Myu3 etc. clusterered with other Mortierella species, as expected.

### REFERENCES

Belitz et al., (2009). Food chemistry (4th revised edition). Springer-Verlag, Berlin, Germany.
Bligh and Dyer (1959) Can. J. Biochem. Physiol. 37:911-918.
Botha et al., (1998) Antonie van Leeuwenhoek 75: 253-256.
Bouckaert et al., (2019) PLOS Computational Biology, 15(4), e1006650
Casaregola et al., (2000). FEBS Lett. 487:95-100.
Christie, W. W. (1996). Lipid analysis. Trends in Food Science & Technology, 11(7), 145.
Certik et al., (1993). Acta Biotechnol 13:193-196.
Dashdorj et al., (2015). Eur Food Res technol 241:157-171.
Gerhard Feiner (2006) Meat Products Handbook
Gladkowski et al (2011). Food chemistry 126:1013-1018.
Grosch. Chem Senses. 2001; 26(5):533-45
Huang et al., (1999). Lipids 34:649-659.
Knutzon et al., (1998). J. Biol. Chem. 273:29360-29366.
Luo et al. Food Sci Nutrition. 2019 (7):2796-805
Letunic and Bork (2019) Nucleic Acids Research, 47:W256-W259
Mottram (1998). Food Chemistry 62:415-424.
Palacios and Wang (2005). J Am Oil Chem Soc 82:571-578.
Patel et al. (2018) Molecules 23:1562.
Raza et al. Food Funct. 2020; 11(10):8583-601
Song et al. Meat Sci. 2014; 96(3):1191
Specht et al. J Ag Food Chem 1994; 42(10):2246-53
Um et al. J Ag Food Chemistry. 1992; 40(9):1641-6
Zhang et al., (2014). Cell Mol Life Sci 71:3767-3778.
Zhang et al. Food Sci Tech. 2017; 82:184-91
Zhao et al. Food Chem. 2019; 270:436-44
Zhou et al. (2007). Phytochemistry 68:785-796.
Zhou et al. (2008). Insect Mol. Biol. 17:667-676

1. A composition capable of producing a food-like aroma and/or flavour when heated, the composition comprising:
a) Mortierella spp. biomass comprising phospholipids;
b) one or more sugars, sugar alcohols, sugar acids, or sugar derivatives; and
c) one or more amino acids or derivatives or salts thereof, or thiamine
wherein the composition is in the form of a food product, beverage product or feedstuff, and wherein the composition comprises less than 2.5% dry Mortierella spp. biomass by weight, or an equivalent amount of wet biomass.

2. The composition according to claim 1, comprising at least about 0.005% dry Mortierella spp. biomass, based on the volume or weight of the composition excluding the Mortierella spp. biomass.

3. The composition according to claim 1 or 2, wherein the food-like aroma and/or flavour is a meaty aroma and/or flavour.

4. The composition according to any one of claims 1 to 3, wherein the phospholipids comprise one or more esterified ω6 fatty acids.

5. The composition according to claim 4, wherein the one or more esterified ω6 fatty acids comprise arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA).

6. The composition according to claim 5, wherein the one or more esterified ω6 fatty acids comprise arachidonic acid (ARA), optionally wherein the ARA is present as at least about 10%, at least about 15%, or at least about 20%, of the total fatty acid content of the polar lipid of the biomass.

7. The composition according to any one of claims 1 to 6, wherein the one or more sugars, sugar alcohols, sugar acids, or sugar derivatives and the one or more amino acids or derivatives or salts thereof are present in the composition in amounts sufficient to produce a food-like aroma and/or flavour when the composition is heated.

8. The composition according to any one of claims 1 to 7, wherein the one or more sugars, sugar alcohols, sugar acids, or sugar derivatives and the one or more amino acids or derivatives or salts thereof are present in the composition in amounts sufficient to produce one or more volatile compounds selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane when the composition is heated.

9. The composition according to claim 8, wherein the one or more sugars, sugar alcohols, sugar acids, or sugar derivatives and the one or more amino acids or derivatives or salts thereof are present in the composition in amounts sufficient to produce one or more volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol when the composition is heated.

10. The composition according to any one of claims 1 to 9, wherein the one or more sugars, sugar alcohols, sugar acids, or sugar derivatives are present in the composition in an amount of from about 0.1 mmol to about 100 mmol per kg or per L of composition, based on the volume or weight of the composition excluding the Mortierella spp. biomass.

11. The composition according to any one of claims 1 to 10, wherein the one or more amino acids or derivatives or salts thereof are present in the composition in an amount of from about 0.1 mmol to about 50 mmol, based on the volume or weight of the composition excluding the Mortierella spp. biomass.

12. The composition according to any one of claims 1 to 11, wherein the one or more sugars, sugar alcohols, sugar acids or sugar derivatives comprise glucose and/or ribose.

13. The composition according to claim 12, wherein the one or more sugars, sugar alcohols, sugar acids or sugar derivatives comprise ribose.

14. The composition according to claim 13, wherein the one or more sugars, sugar alcohols, sugar acids or sugar derivatives comprise ribose and glucose.

15. The composition according to any one of claims 1 to 14, wherein the one or more amino acids or derivatives or salts thereof comprise cysteine and/or cystine.

16. The composition according to claim 15, wherein the one or more amino acids or derivatives or salts thereof comprise cysteine.

17. The composition according to any one of claims 1 to 16, wherein the one or more amino acids comprise glutamic acid or a salt thereof.

18. The composition according to any one of claims 1 to 17, wherein composition comprises glutamic acid or a salt thereof and a further amino acid, derivative or salt thereof.

19. The composition according to any one of claims 1 to 18, further comprising a source of iron.

20. The composition according to any one of claims 1 to 19, further comprising a yeast extract.

21. The composition according to any one of claims 1 to 20, wherein the composition does not further comprise a yeast extract.

22. The composition according to any one of claims 1 to 21, further comprising thiamine.

23. The composition according to any one of claims 1 to 22, further comprising one or more herbs and/or spices.

24. The composition according to any one of claims 1 to 23, wherein the composition comprises an aqueous component.

25. The composition according to any one of claims 1 to 24, comprising:
a) Mortierella spp. biomass comprising phospholipids;
b) glucose and/or ribose;
b) cysteine and/or cystine;
d) yeast extract;
e) glutamic acid or a salt thereof;
f) thiamine; and
g) an aqueous component.

26. The composition according to any one of claims 1 to 25, wherein the composition further comprises an extracted lipid from Mortierella spp. comprising phospholipids.

27. The composition according to any one of claims 1 to 26, wherein the composition produces a food-like aroma and/or flavour when heated, optionally wherein the food-like aroma and/or flavour is a meaty aroma and/or flavour.

28. The composition according to any one of claims 1 to 27, wherein the food product, beverage product or feedstuff is a meat or meat-like product.

29. The composition according to any one of claims 1 to 28, wherein the food product, beverage product or feedstuff is a burger, sausage, hot dog, mince or ground meat, steak, streak, strip, fillet, roast, breast, thigh, wing, meatloaf, finger, nugget, cutlet, cube, bacon, soup, gravy, sliced meat, meatballs, fish, fried fish or seafood or imitation thereof.

30. The composition according to any one of claims 1 to 29, wherein the food product, beverage product or feedstuff is free from any animal or animal-derived ingredients.

31. The composition according to any one of claims 1 to 29, wherein the food product, beverage product or feedstuff comprises an animal or animal-derived ingredient, optionally wherein the animal or animal-derived ingredient is meat, optionally wherein the animal or animal-derived ingredient is cultivated meat.

32. A method for producing a food product, beverage product or feedstuff comprising combining Mortierella spp. biomass comprising phospholipids; one or more sugars, sugar alcohols, sugar acids, or sugar derivatives; and one or more amino acids or derivatives or salts thereof, or thiamine; with one or more additional consumable ingredients, wherein the food product, beverage product or feedstuff comprises about 2.5% or less dry Mortierella spp. biomass by weight, or an equivalent amount of wet biomass.

33. A method for producing food-like aromas and/or flavours, comprising heating a composition according to any one of claims 1 to 32.

34. A method of imparting a food-like aroma and/or flavour to a food product, beverage product or feedstuff or increasing food-like aromas and/or flavours associated with a food product, beverage product or feedstuff, comprising contacting the food product, beverage product or feedstuff with a) Mortierella spp. biomass comprising phospholipids, b) one or more sugars, sugar alcohols, sugar acids, or sugar derivatives; and c) one or more amino acids or derivatives or salts thereof, or thiamine, and heating the food product, beverage product or feedstuff and Mortierella spp. biomass or composition, wherein the heated food product, beverage product or feedstuff comprises about 2.5% or less dry Mortierella spp. biomass by weight, or an equivalent amount of wet biomass.

35. A method of imparting a food-like aroma and/or flavour to a food product, beverage product or feedstuff or increasing food-like aromas and/or flavours associated with a food product, beverage product or feedstuff, comprising:
a) heating a composition comprising i) Mortierella spp. biomass comprising phospholipids, ii) one or more sugars, sugar alcohols, sugar acids, or sugar derivatives; and iii) one or more amino acids or derivatives or salts thereof, or thiamine; and then
b) contacting a food product, beverage product or feedstuff with the composition obtained in step a).

36. The method according to any one of claims 33 to 35, wherein the food-like aroma and/or flavour is a meaty aroma and/or flavour.

37. The method of any one of claims 33 to 36, wherein the composition, food product, beverage product or feedstuff is heated to at least about 130° C.

38. The method of claim 35, wherein the composition is heated for at least about 1 hour.

39. Use of Mortierella spp. biomass comprising phospholipids in a food product, beverage product or feedstuff, wherein the composition, food product, beverage product or feedstuff comprises less than 2.5% dry Mortierella spp. biomass by weight, or an equivalent amount of wet biomass.

40. Use according to claim 39 for imparting a food-like aroma and/or flavour to said food product, beverage product or feedstuff upon heating.

41. Use according to claim 39 or 40, wherein the food-like aroma and/or flavour is a meaty aroma and/or flavour.

42. Use according to any one of claims 39 to 41, wherein the phospholipids comprise one or more esterified ω6 fatty acids.

43. Use according to claim 42, wherein the one or more esterified ω6 fatty acids comprise arachidonic acid (ARA), dihomo-gammalinolenic acid (DGLA), eicosadienoic acid (EDA), docosatetraenoic acid (DTA), docosapentaenoic acid-ω6 (DPA-ω6) or γ-linolenic acid (GLA).

44. Use according to claim 43, wherein the one or more esterified ω6 fatty acids comprise arachidonic acid (ARA).

45. Use according to any one of claims 39 to 44, wherein the food product, beverage product or feedstuff is a meat or meat-like product.

46. Use according to any one of claims 39 to 45, wherein the food product, beverage product or feedstuff is a burger, sausage, hot dog, mince or ground meat, steak, streak, strip, fillet, roast, breast, thigh, wing, meatloaf, finger, nugget, cutlet, cube, bacon, soup, gravy, sliced meat, meatballs, fish, fried fish or seafood or imitation thereof.

47. Use according to any one of claims 39 to 46, wherein the food product, beverage product or feedstuff is free from any animal or animal-derived ingredients.

48. Use according to any one of claims 39 to 46, wherein the food product, beverage product or feedstuff comprises an animal or animal-derived ingredients, optionally wherein the an animal or animal-derived ingredient is meat.

49. Use according to any one of claims 39 to 48, wherein the food product, beverage product or feedstuff comprises: one or more sugars, sugar alcohols, sugar acids, or sugar derivatives; and one or more amino acids or derivatives or salts thereof.

50. Use according to claim 49, wherein the one or more sugars, sugar alcohols, sugar acids, or sugar derivatives and the one or more amino acids or derivatives or salts thereof are present in the composition, food product, beverage product or feedstuff in amounts sufficient to produce a food-like aroma and/or flavour when the composition, food product, beverage product or feedstuff is heated.

51. Use according to claim 49 or 50, wherein the one or more sugars, sugar alcohols, sugar acids, or sugar derivatives and the one or more amino acids or derivatives or salts thereof are present in the composition, food product, beverage product or feedstuff in amounts sufficient to produce one or more volatile compounds selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, heptanal, benzeneacetaldehyde, thiazole, 2,4-Di-tert-butylphenol, acetylacetone and 1,3,5-thitriane when the composition, food product, beverage product or feedstuff is heated.

52. Use according to any one of claims 49 to 51, wherein the one or more sugars, sugar alcohols, sugar acids, or sugar derivatives and the one or more amino acids or derivatives or salts thereof are present in the composition, food product, beverage product or feedstuff in amounts sufficient to produce one or more volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol when the composition, food product, beverage product or feedstuff is heated.

53. Use according to any one of claims 39 to 52, wherein the food product, beverage product or feedstuff further comprises an extracted lipid from Mortierella spp. comprising phospholipids.


SEQUENCE LISTING (51 sequences)

SEQ ID NO: 1 - DNA, 178 bp, Mortierella alpina
  source 1..178: mol_type = genomic DNA
cataatcaaagtgtttttatggcacttttaaaaaaatccatatccaccttgtgtgcaatgtcatctcactggaggccggcggctgtaaaaagcccgtttggtcacctttgggatttatactactcagaactttagtgattttgtctgaaaaatattatgaataacttaattcaaaata

SEQ ID NO: 2 - DNA, 22 bp, synthetic construct
  misc_feature 1..22: note = Synthetic sequence
  source 1..22: mol_type = other DNA
ggaagtaaaagtcgtaacaagg

SEQ ID NO: 3 - DNA, 19 bp, synthetic construct
  misc_feature 1..19: note = Synthetic sequence
  source 1..19: mol_type = other DNA
tccccgcttattgatatgc

SEQ ID NO: 4 - DNA, 640 bp, Mucor hiemalis
  source 1..640: mol_type = genomic DNA
tctggtagtgactgcggagacattatatgatttatatggtctccgtagaacttgttctaattaggttcattcttttttacagtgaactgtttcaattttcgatgtttggggactgtcttttaggaataaggatagactattaaaatgttaaccgaactgaagtcaggtttagacctggtatcctattaattatttaccaaatgaatttcctattattattgaaagataagcgtgaaaaccttgggatccaacttttaacaacggatctcttgtttctcgcatcgattaataacgtatttaagcgccataactagtgtgaattgcatattcattgaatcatctagtctttgaaaacaacttgcgctcaatggtattccattgagcacgcctgtttcagtatcaaaaacaccccacattcataattttgttgtgaatggaactgaaaatttccacttgtttgaattctttaaacttattacgcctgaactattgttctttctgcctgaacatttttttaatataaaggaatgctctagtattaaaactatctctggggcctcccaaataaaactttcttaaatttgatctgaaatcaggcgggattacccgctgaacttaagcatatcataaaaaggggaaa

SEQ ID NO: 5 - DNA, 669 bp, Mortierella alpina
  source 1..669: mol_type = genomic DNA
gcgtaaggttactgcggatgacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatatctaaaagggggggaaa

SEQ ID NO: 6 - DNA, 671 bp, Mortierella alpina
  source 1..671: mol_type = genomic DNA
ttcgtagtgactgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatatcaaaaacggggggagaaaa

SEQ ID NO: 7 - DNA, 672 bp, Mortierella alpina
  source 1..672: mol_type = genomic DNA
gtcgtcagtgactgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatactaaaaggggggggaaatag

SEQ ID NO: 8 - DNA, 668 bp, Mortierella alpina
  source 1..668: mol_type = genomic DNA
gtcgtagttactgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatataaaaatgggggggaaa

SEQ ID NO: 9 - DNA, 671 bp, Mortierella alpina
  source 1..671: mol_type = genomic DNA
ttcgtcagttacctgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatatctaaagcgggggggaaa

SEQ ID NO: 10 - DNA, 670 bp, Mortierella alpina
  source 1..670: mol_type = genomic DNA
ttcgatagtgacctgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatacaaaagggggggggaaa

SEQ ID NO: 11 - DNA, 824 bp, Trichoderma asperellum
  source 1..824: mol_type = genomic DNA
ttcggtaggtgactgcggagacattaagaatttaataattttgaagatggatttttttttttttggtttgggcaaaagcaggaaaccttttaggggggaaaaaaacaaaaaagggaaagcccccccgggccgggccccaaggggggggcctggccgggctggtgagtttcttcctgcctttcccaaagggggaaaaattcctgggttttggttaaggaacaataaaaaccgttcacccccaccccccggggggttttcttaaccttggcactttttccttggggcttccaacaaacggggccccaaaggtcaccaaaccccaccatttttttctttcctttaatttttggcaaaaaacaaaatttttccaaccgggaatttttaaaaattaaaaaattttcaccaccggtcctttgggtccccccaccaagaaaaaacccaccaaaaggcaaaccgaaggggaaatggcaaaatcccgggaaccaccaaacctttaaccccccttggcccccctgggtttcccggggggcagcccggttaaacccctttttcctccccaaactttcggttgggaaggggggaaccctcttgggagtaaactggaaatggcgggcttttccattggagttttttttttcccaaaaagggtttcccctgggggttggaggaaaaggccagacggccttttagttttcccccgggggtaatctttttatccggggggatggaacgtatcaaaaaaaaaagccccaggcaaaaatggtcttaaatttgccctcaatcgggaggaacccccctgacttagcaatcaaaaagggggaaaa

SEQ ID NO: 12 - DNA, 668 bp, Mortierella alpina
  source 1..668: mol_type = genomic DNA
tcgatagtgactgcggagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatatctaaaagggggggaaaa

SEQ ID NO: 13 - DNA, 644 bp, Mucor hiemalis
  source 1..644: mol_type = genomic DNA
tctggtagtgactgcggagacattaataatttagatggcctttgctagttttctagcgaatggttcattcttttttactgtgaactgttttaatttttcagcgtctgaggaatgtcttttagccatagggataggctactagaatgttaaccgagctgaaagtcaggcttaggcctggtatcctattaattatttaccaaaagaattcagtattataattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagagtttcggctttattgctgaattctttaaaattattaggcctgaactattgttctttctgcctgaacatttttttaatataaaggaatgctctagtaaaaagactatctctggggcctcccaaataaatcattcttaaatttgatctgaaatcaggcgggattacccgctgaacttaagcattcaaaaaggggggggaaa

SEQ ID NO: 14 - DNA, 639 bp, Mucor hiemalis
  source 1..639: mol_type = genomic DNA
cttaagtgactgcggaagacatttaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatactaaaatggggggaaaa

SEQ ID NO: 15 - DNA, 647 bp, Mucor hiemalis
  source 1..647: mol_type = genomic DNA
ttcggaaagtgaactgcggaagacattaaataatttagatggcctccctagaacttgttctagttaggttcatttcttttttactgtgaactgttttaatttttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatactaaaagggggggggaaa

SEQ ID NO: 16 - DNA, 639 bp, Mucor hiemalis
  source 1..639: mol_type = genomic DNA
tcctgtaagtgactgcggagacattaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaagccgggggaa

SEQ ID NO: 17 - DNA, 640 bp, Mucor hiemalis
  source 1..640: mol_type = genomic DNA
tcgttagtgactgcggacgacattaataatttagatgacctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatctaaaaggggggggaaa

SEQ ID NO: 18 - DNA, 639 bp, Mucor hiemalis
  source 1..639: mol_type = genomic DNA
tccgtagtgactgcggagacattaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatctaaaagggggggggaa

SEQ ID NO: 19 - DNA, 646 bp, Mucor hiemalis
  source 1..646: mol_type = genomic DNA
cttaagtgactgcggaagacattaataatttagatggcctttgctagttttctagcgaatggttcattcttttttactgtgaactgttttaatttttcagcgtctgaggaatgtcttttagccatagggataggctactagaatgttaaccgagctgaaagtcaggcttaggcctggtatcctattaattatttaccaaaagaattcagtattataattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaaatgagagtttcggctttattgctgaattctttaaaattattaggcctgaactattgttctttctgcctgaacatttttttaatataaaggaatgctctagtaaaaagactatctctggggcctcccaaataaatcattcttaaatttgatctgaaatcaggcgggattacccgctgaacttaagcatatctaaaaaggcgggggggaa

SEQ ID NO: 20 - DNA, 645 bp, Mucor hiemalis
  source 1..645: mol_type = genomic DNA
tctggtaagtgactgcggaagacattaataatttagatgacctccctagaacttgttctagttaggttcattcttttttactgtgaactcgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatctaaagtggggggggaaa

SEQ ID NO: 21 - DNA, 659 bp, Mortierella elongata
  source 1..659: mol_type = genomic DNA
ttcgactagtgaacttgccggaaggatcattcataataagtgtttttattggcactttttttttaaattccatattccacctttgtgtgcaatgtcagtcgatcttcttcatggagatcgaccaaacatcaacctttattttttaactctttgtctgaaaaatattatgaataaacaattcaaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcttttggattttttaatcgaaaagctttggacttgagcaatcccaacaccaatctttttgagatcggtggcgggttgcttgaaatgcaggtgcagctggacattctcctgagctaaaagcatattcatttagtcccgtcaaacggattattacttttgctgcagctaacataaagggagtttgaccgtattggctgactgatgcaggatttcacaagggtcggcaacgattcttgttaaactcgatctcaaatcaagtaagactacccgctgaacttaagcattaaaaagggggggggagaa

SEQ ID NO: 22 - DNA, 639 bp, Mucor hiemalis
  source 1..639: mol_type = genomic DNA
cctgtagtgactgcggagacattaataatttagatgacctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaaaccggggggaaa

SEQ ID NO: 23 - DNA, 640 bp, Mucor hiemalis
  source 1..640: mol_type = genomic DNA
cactgtaagtgactgcggagacattaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaaaaccgggggaa

SEQ ID NO: 24 - DNA, 639 bp, Mucor hiemalis
  source 1..639: mol_type = genomic DNA
tctgtagtgactgcggagacattaataatttagatggcctcctagaacttgttctagttaggttcattcttttttactgtgaactgttttaattttcagtgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttattgtgaatggaattgagaatttcgacttgtttgaattctttaaacctattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaaaaccgggggaaa

SEQ ID NO: 25 - DNA, 639 bp, Mucor hiemalis
  source 1..639: mol_type = genomic DNA
cctgaaggtgactgcggagacattaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaaaaccgggggaa

SEQ ID NO: 26 - DNA, 637 bp, Mortierella sp
  source 1..637: mol_type = genomic DNA
ttggacagtgaactgcggaagatcattcataataagtgtttatggcacttccttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcataataaaaacgggggggagaaa

SEQ ID NO: 27 - DNA, 640 bp, Mortierella sp
  source 1..640: mol_type = genomic DNA
tcgaatagtgaacttgccggaaggatcattcataataagtgtttattggcactttcttaaattccatattccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatacaaaacggggggaaaac

SEQ ID NO: 28 - DNA, 629 bp, Mortierella sp
  source 1..629: mol_type = genomic DNA
cctgtagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatcaaaaaggggggggaa

SEQ ID NO: 29 - DNA, 640 bp, Mortierella sp
  source 1..640: mol_type = genomic DNA
tcgaatagtgaacttgcggaagatcattcataataagtgttttatggcacttttccttaaatcccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatacaaaaaaggggggggaaa

SEQ ID NO: 30 - DNA, 640 bp, Mortierella sp
  source 1..640: mol_type = genomic DNA
ttcgaatagtgaacttgcggaaggacattcataataagtgttttatggcactttcttaaatcccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaagggggggagaaaa

SEQ ID NO: 31 - DNA, 630 bp, Mortierella sp
  source 1..630: mol_type = genomic DNA
ctgtcagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatctaaaaggggggggaaa

SEQ ID NO: 32 - DNA, 646 bp, Mortierella sp
  source 1..646: mol_type = genomic DNA
ttcgaataggtgaacttgccggaaggatcattcataataagtgttttattggcactttccttaaattccatattccacctttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaaggggggggaaaa

SEQ ID NO: 33 - DNA, 630 bp, Mortierella sp
  source 1..630: mol_type = genomic DNA
tcctgtagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatcaaaaaacggggggaa

SEQ ID NO: 34 - DNA, 643 bp, Mortierella elongata
  source 1..643: mol_type = genomic DNA
tcgttaggtgactgcggagacattcataataagtgttttatggcactttttaaatccatatccaccttgtgtgcaatgtcagtcgatcttcttcatggagatcgaccaaacatcaacctttatcttttaactctttgtctgaaaaatattatgaataaacaattcaaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcttttggattttttaatcgaaaagctttggacttgagcaatcccaacaccaatctttttgagatcggtggcgggttgcttgaaatgcaggtgcagctggacattctcctgagctaaaagcatattcatttagtcccgtcaaacggattattacttttgctgcagctaacataaagggagtttgaccgtattggctgactgatgcaggatttcacaagggtcggcaacgattcttgttaaactcgatctcaaatcaagtaagactacccgctgaacttaagcatatctaaaagcggggggaaa

SEQ ID NO: 35 - DNA, 652 bp, Mortierella sp
  source 1..652: mol_type = genomic DNA
gcggtagtgactgcggagacattcataataagtgttttatggcacttttaaaaatccatatccaccttgtgtgaaatgtctgtcgcttttctcttttttccttggaaaaaaacgaagcggctcaacatcaactcattttaattactttgtctgaaaactattatgaataaactaattcaaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaacactctcgatttgtttcgaatgagtattggatttgagcaaacccaacgcctttctgctagccagacttgcggcgggttgcttgaaatgcaggtgcagctgagttttcttctgagctaatagcatacttaattagtcccgtaaaaaggattattaatctgctgaagcttacaccaaaggaagagttctctggctgactgatgcagaattacgcatagccggtaaaacggctttgtaaaactcgatctcaaatcaagtaagactacccgctgaacttaagcatatcaaaaaaccgggggaaa

SEQ ID NO: 36 - DNA, 633 bp, Mortierella sp
  source 1..633: mol_type = genomic DNA
ttgttaggttactgcggaagacattcataataagtgtttatggcactttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatctaaaaggggggggaaa

SEQ ID NO: 37 - DNA, 639 bp, Mortierella sp
  source 1..639: mol_type = genomic DNA
tcgactgtgaactgccggaagatcttcataataagtgtttatggcactttcttaaatcctatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagctttcttcgtaaaactcgatctcaaatcaagtaagactacccgctgaactttaagcatttaaaaatgggggaaaaaatgc

SEQ ID NO: 38 - DNA, 638 bp, Mortierella sp
  source 1..638: mol_type = genomic DNA
tcgaacagtgaactgccggaaggatcattcataataagtgtttatggcactttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaagggggggagaaaa

SEQ ID NO: 39 - DNA, 644 bp, Mortierella sp
  source 1..644: mol_type = genomic DNA
ttcgaatagtgaacttgcggaaggatcattcataataagtgttttattggcactttccttaaattccatattccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaacggggggagaaaa

SEQ ID NO: 40 - DNA, 644 bp, Mortierella sp
  source 1..644: mol_type = genomic DNA
ttcgaatagtgaacttgccggaaggacattcataataagtgttttattggcactttccttaaattccatattccacctttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaaggggggggaaaa

SEQ ID NO: 41 - DNA, 641 bp, Mortierella sp
  source 1..641: mol_type = genomic DNA
tcgaatagtgaacttgccggaaggatcattcataataagtgtttatggcactttcttaaattccatattccacctttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcattaaaaaagggggggaaaaa

SEQ ID NO: 42 - DNA, 640 bp, Mortierella sp
  source 1..640: mol_type = genomic DNA
ttcgacagtgaacttgccggaaggatcattcataataagtgttttatggcactttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttccttgtaaaactcgatctcaaatcaagtaagactacccgctgaacttaagcatttaaaacggggggaggaaa

SEQ ID NO: 43 - DNA, 645 bp, Mortierella sp
  source 1..645: mol_type = genomic DNA
tcgaatagtgaacttgccggaaggatcattcataataagtgttttattggcacttttccttaaattccatattccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatacaaaaaggggggggggaaa

SEQ ID NO: 44 - DNA, 643 bp, Mortierella sp
  source 1..643: mol_type = genomic DNA
ttcggaatagtgaactgcggaagatcattcataataagtgttttatggcacttttccttaaattccatattccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaagggggggggaaaa

SEQ ID NO: 45 - DNA, 629 bp, Mortierella sp
  source 1..629: mol_type = genomic DNA
tctgtagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatcaaaaagcggggggaa

SEQ ID NO: 46 - DNA, 628 bp, Mortierella sp
  source 1..628: mol_type = genomic DNA
gcgtagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatcaaaaagcggggggaa

SEQ ID NO: 47 - DNA, 671 bp, Mortierella alpina
  source 1..671: mol_type = genomic DNA
tcggtaggtgactgcggagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatatcaaaaaaccgggggaaata

SEQ ID NO: 48 - DNA, 641 bp, Mortierella elongata
  source 1..641: mol_type = genomic DNA
tctgtagggactgcggagacattcataataagtgttttatggcactttttaaatccatatccaccttgtgtgcaatgtcagtcgatcttcttcatggagatcgaccaaacatcaacctttattttttaactctttgtctgaaaaatattatgaataaacaattcaaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcttttggattttttaatcgaaaagctttggacttgagcaatcccaacaccaatctttttgagatcggtggcgggttgcttgaaatgcaggtgcagctggacattctcctgagctaaaagcatattcatttagtcccgtcaaacggattattacttttgctgcagctaacataaagggagtttgaccgtattggctgactgatgcaggatttcacaagggtcggcaacgattcttgttaaactcgatctcaaatcaagtaagactacccgctgaacttaagcatatcaaaaatgggggggaa

SEQ ID NO: 49 - DNA, 633 bp, Mortierella exigua
  source 1..633: mol_type = genomic DNA
gtgttttatggcactttttaaatccatatccaccttgtgtgcaatgtcagtcggttttctttttcgtcaagaaaaaaaggatcgaccaaacatcaacttattctaattgaacttttgtctgaaactattatgaataaactaattcaaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaaactcttgatctttttcaagcgttttggacttgagcaaacccaacgccttttcttttaagatctgttgcggcgggttgcttgaaatgcaggtgcagctggacaatctcctgagctaaaagcatatttatttagtcccgtcaaacggattattactttttgctgcagctaacataaagggagttttgaccattatggctgactgatgcaggatttcacagagaaaaggccggcaacggttttgtctctgtcaaactcgatctcaaatcaagtaagactacccgctgaacttaagcatatcaaaaagcgggggaaa

SEQ ID NO: 50 - DNA, 642 bp, Mortierella minutissima
  source 1..642: mol_type = genomic DNA
gcggttagtgactgcggagacattcacaaacgagtgttttatggcactctttaaaaatccatatccaccttgtgtgcaatgtttgttgggaaagtcttttctttcccttcataaatatcaacctatatctttaacaacattcgtctgataacatattatgaatatacttaattcaaaatataactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaacctctcttttttgataaaaaaaagggagggtggacttgagctatcccaacgaccttcaccggtaggcgggcggcttgaaatgcaggtgcagctggacttttatctgagctaaaagcatatctatttagtcctcgtcaaacaggattattacttttgctgcagctaacataaaggataattgtcctctttgctgactgatgcaggattttacgacactttatgtgttgttcaactcgatctcaaatcaagtaagactacccgctgaacttaagcatatcaaaaaagcgggggaaa

SEQ ID NO: 51 - DNA, 676 bp, Mortierella alpina
  source 1..676: mol_type = genomic DNA
tcgggagtgactgcggagacattcataatcaaagtgtttttatggcacttttaaaaaaatccatatccaccttgtgtgcaatgtcatctcactggaggccagcggctgtaaaaagcccgtttggtcgcctttgggatttatatctactcagaactttagtgattttgtctgaaaaatattatgaataacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaactccctttttcttttttgaaatgagggagctggacttgagtgatcccaacacttttctcactgaaaagtggcgggtcacttgaaatgcaggtgcagctggacttttctctgagctataagcatatctatttagtctgcctaaaaaacagattattacctttgctgcagctaacataaaggagatgagttcttgtgctgactgatgcaggattcacagagacagctttacggctgactttgtaaactcgatctcaaatcaagtaagactacccgctgaacttaagcatatcataaagccgggggaaa